(19) United States
(12) Patent Application Publication
(10) Pub. No.: US 2023/0270881 A1
(43) Pub. Date: Aug. 31, 2023

(54) ONCOGENIC TRIM37 IS A TARGETABLE EPIGENETIC DRIVER OF METASTASIS AND LINKS CHEMORESISTANCE AND METASTATIC FATE IN TRIPLE-NEGATIVE BREAST CANCER

(71) Applicant: University of Virginia Patent Foundation, Charlottesville, VA (US)

(72) Inventors: Sanchita Bhatnagar, Davis, CA (US); Jogender Tushir-Singh, Davis, CA (US)

(21) Appl. No.: 17/794,432

(22) PCT Filed: Jan. 21, 2021
(86) PCT No.: PCT/US2021/014438

§ 371 Date: Jul. 21, 2022

Related U.S. Application Data
(60) Provisional application No. 63/067,712, filed on Aug. 19, 2020.
(60) Provisional application No. 62/963,883, filed on Jan. 21, 2020.

(51) Int. Cl. A61K 47/69; A61K 47/68; C12N 15/113; A61P 35/00

(52) U.S. Cl. CPC A61K 47/6913; A61K 47/6849; A61K 47/6915; A61P 35/00; C12N 15/1138; C12N 2310/11; C12N 2310/531

(57) ABSTRACT

Methods for sensitizing tumors and/or cancers in subjects to therapeutic agents are provided. In some embodiments, the methods include administering to the subject one or more compositions that include an effective amount of an inhibitor of TRIM37 activity. Also provided are methods for sensitizing tumors and/or cancers in subjects to therapeutic agents by administering to the subjects one or more compositions that include an effective amount of an inhibitor of TRIM37 activity and purified and isolated antibodies and fragments thereof that have at least one paratope and further have a linker sequence through which the antibody can be conjugated to a carrier in which the linker sequence includes the amino acid sequence ((X)₃Cys(X)₃, wherein each X is independently any amino acid.

### CROSS-REFERENCE TO RELATED APPLICATIONS

This application claims the benefit of U.S. Provisional Pat. Application Serial No. 62/963,883, filed Jan. 21, 2020, and of U.S. Provisional Pat. Application Serial No. 63/067,712, filed Aug. 19, 2020, the disclosure of each of which is incorporated herein by reference in its entirety.

### GOVERNMENT INTEREST

This invention was made with government support under Grant Nos. W81XWH-18-1-0049 and W81XWH-20-1-0021 awarded by the Department of Defense. The government has certain rights in the invention.

### REFERENCE TO SEQUENCE LISTING SUBMITTED ELECTRONICALLY

The content of the electronically submitted sequence listing in ASCII text file (Name: 3062_97_PCT_ST25.txt; Size: 424 kilobytes; and Date of Creation: Jan. 21, 2021) filed with the application is incorporated herein by reference in its entirety.

### BACKGROUND

Triple negative breast cancer (TNBC) is an aggressive breast cancer subtype that accounts for ~20% of all breast cancer cases with the annual incidence rate in the United States estimated to be 40,000 (Silver et al., 2010; Pal et al., 2011). TNBC patients are disproportionately associated with the highest frequency of chemoresistance, relapse, and metastasis to visceral organs. Consequentially, the 5-year survival rate of TNBC is 77% relative to 93% for other breast cancer subtypes (Pal et al., 2011). Despite the high mortality rate in women worldwide, chemotherapy remains the standard of care for TNBC patients. Although chemotherapy is effective initially in TNBC patients, it is often accompanied by resistance, relapse, and severe side effects (Liedtke et al., 2008; Cortazar et al., 2014). Therefore, new and effective targeted therapies to prevent and ultimately cure TNBC are a clinical priority. While lifestyle, epidemiologic, and cultural factors shape TNBC clinical outcome, the disease etiology is also dependent on biogeographical ancestry (Bauer et al., 2007). Thus, lack of targeted therapies for TNBC is fraught with multiple challenges attributed to limited understanding of genetic complexities, metastatic biology, and drivers of metastatic traits.

An unresolved question in cancer biology is what drives a primary tumor to become metastatic? This is a clinically relevant question because metastatic, not primary tumors, are fatal. In general, numerous oncogenes and tumor suppressors are genetically or epigenetically altered in cancer and accumulate during tumorigenesis. But whether drivers of tumorigenesis are also the causal factor of the metastatic transition remains to be addressed. To this end, extensive transcriptomic and genetic scans of evolving carcinomas revealed mutations that were represented in premalignant biopsies but not in tumor biopsies, suggesting divergence of genetic alterations during the transition from primary to regional metastases (Bailey et al., 2018; Bertucci et al., 2019; De Mattos-Arruda et al., 2019; Gibson et al., 2016; Yates et al., 2017). Additionally, dynamic epigenetic mechanisms that cause varying chromatin states and transcriptional heterogeneity are also intimately linked to metastatic transitions (Ryan & Bernstein, 2012; Shen & Laird, 2013). For example, TNBC tumors harbor a high frequency of hypermethylated promoters in commonly targetable drivers, such as TP53, BRAF, KRAS, and EGFR (Nik-Zainal et al., 2016; Shah et al., 2012; Stephens et al., 2012). Alterations in epigenetic factors causing neomorphic mutations (e.g., EZH2, DNMT3A) or translocations (e.g., NSD2, MMSET) are also frequent in cancer patients (Roy et al., 2014; Shen & Laird, 2013). As such, several small molecules targeting epigenetic regulators have entered clinical trials, for example, Estinosat (Batlevi et al., 2016), Belinostat (Kirschbaum et al., 2014), and Panobinostat (Yee and Raje, 2018). Understandably, these drug treatments are not mutation-specific and therefore, pose a significant toxicity risk to untransformed cells, underscoring the critical need for targeted therapies.

We have previously described tripartite motif-containing protein 37 (TRIM37) as a breast cancer oncoprotein that can epigenetically silence tumor suppressors (Bhatnagar et al., 2014; Bhatnagar & Green, 2015). Clinically, TRIM37 is highly expressed in breast cancer tissue and is associated with poor overall survival (Bhatnagar et al., 2014). Mechanistically, TRIM37 monoubiquitinates histone H2A at Lys119 (H2Aub) to down-regulate target genes (Bhatnagar et al., 2014). Functionally, TRIM37 over-expression renders non-transformed breast cells tumorigenic, and inhibition of TRIM37 function reduces tumor growth in xenograft mouse models (Bhatnagar et al., 2014). While TRIM37 promotes tumorigenesis, its function in breast cancer progression and metastasis as well as the therapeutic implications of TRIM37 targeting remain to be demonstrated.

Metastasis is a multistep process that includes signaling cues from the tumor microenvironment, as well as pathways regulating the epithelial-mesenchymal transition (EMT), survival in circulation, infiltration of distant sites, and metastatic growth (Lambert et al., 2017). Given the majority of TNBC patients receive chemotherapy, the ability to resist therapy-induced cell death is perhaps the first step towards a metastatic phenotype. Indeed, recent evidence revealed synchronized expression of genes involved in surviving the stress of chemotherapy as well as overcoming the natural barriers of invasion, infiltration, and metastatic growth. For example, a CXCL½ paracrine pathway alters the tumor microenvironment to accentuate chemoresistance and a metastatic cascade (Acharyya et al., 2012). Likewise, amplification of the 8q22 region harboring MTDH was identified with dual functionality in metastasis and chemoresistance (Hu et al., 2009). Here, we uncover that TRIM3 7 alters DNA damage response to prevent therapy-induced cell death, and enforces a transcriptional program favoring metastasis. In particular, we report that selective TRIM37 inhibition in TNBC tumors suppress lung metastases in vivo. Together, these data reveal that TRIM37 is a new epigenetic driver of aggressive TNBC biology, which can be targeted to simultaneously increase chemotherapy efficacy and reduce metastasis risk in TNBC patients.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for sensitizing tumors and/or cancers in subjects to therapeutic agents. In some embodiments, the methods comprise administering to a subject in need thereof one or more compositions comprising an effective amount of an inhibitor of TRIM37 activity. In some embodiments, the tumor and/or cancer is triple negative breast cancer (TNBC) and/or a metastatic lesion derived therefrom. In some embodiments, the therapeutic agent is a chemotherapeutic agent. In some embodiments, the inhibitor of TRIM37 activity is selected from the group consisting of an anti-sense oligonucleotide, a small molecule inhibitor, and a combination thereof.

In some embodiments, the composition comprises a nanoparticle, a targeting moiety, or a combination thereof. In some embodiments, the nanoparticle is liposome-based. In some embodiments, the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide(polyethyleneglycol-2000)] (DSPE-PEG2000-Maleimide), or any combination thereof.

In some embodiments, the targeting moiety comprises an anti-FOLR1 antibody. In some embodiments, the antibody comprises a linker sequence through which the antibody is conjugated to the nanoparticle. In some embodiments, the linker sequence comprises an Fc-linkered sequence containing a cysteine. In some embodiments, the Fc-linkered sequence is present at the C-terminus of a heavy chain of the antibody, optionally conjugated to an amino acid present at the C-terminus of a heavy chain of the antibody.

In some embodiments, the presently disclosed methods further comprise administering the therapeutic agent to which the subject has been sensitized to the subject.

The presently disclosed subject matter also relates in some embodiments to methods for treating tumors and/or cancers in subjects in need thereof. In some embodiments, the methods comprise administering to the subject (a) one or more compositions comprising an effective amount of an inhibitor of TRIM37 activity; and (b) one or more anti-cancer therapeutic agents. In some embodiments, the tumor and/or the cancer is triple negative breast cancer (TNBC) and/or a metastatic lesion derived therefrom. In some embodiments, the therapeutic agent is a chemotherapeutic agent. In some embodiments, the inhibitor of TRIM37 activity is selected from the group consisting of an anti-sense oligonucleotide, a small molecule inhibitor, and a combination thereof. In some embodiments, the composition comprises a nanoparticle, a targeting moiety, or a combination thereof. In some embodiments, the nanoparticle is liposome-based. In some embodiments, the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide (polyethyleneglycol-2000)] (DSPE-PEG2000-Maleimide), or any combination thereof.

In some embodiments, the targeting moiety comprises an antibody that targets the composition or a component thereof to the tumor and/or the cancer. In some embodiments, the antibody comprises an anti-FOLR1 antibody. In some embodiments, the antibody comprises a linker sequence through which the antibody is conjugated to the nanoparticle. In some embodiments, the linker sequence comprises an Fc-linkered sequence containing a cysteine. In some embodiments, the Fc-linkered sequence is present at the C-terminus of a heavy chain of the antibody.

The presently disclosed subject matter also relates in some embodiments to purified and isolated antibodies, or fragments thereof comprising at least one paratope, comprising a linker sequence through which the antibody can be conjugated to a carrier, wherein the linker sequence comprises the amino acid sequence ((X)₃Cys(X)₃, wherein each X is independently any amino acid. In some embodiments, the linker sequence comprises an Fc-linkered sequence containing a cysteine. In some embodiments, the Fc-linkered sequence is present at the C-terminus of a heavy chain of the antibody. In some embodiments, the purified and isolated antibody or the fragment thereof specifically binds to a folate receptor.

In some embodiments of the presently disclosed purified and isolated antibodies and fragments thereof, the carrier is a nanoparticle. In some embodiments, the carrier comprises an inhibitor of TRIM37 activity. In some embodiments, the inhibitor of TRIM37 activity is selected from the group consisting of an anti-sense oligonucleotide, a small molecule inhibitor, and a combination thereof. In some embodiments, the nanoparticle is liposome-based. In some embodiments, the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide(polyethyleneglycol-2000)] (DSPE-PEG2000-Maleimide), or any combination thereof.

Accordingly, it is an object of the presently disclosed subject matter to provide compositions and methods for sensitizing tumors and/or cancers to therapeutic agents.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A-1J: TRIM37-directed mono-ubiquitination of H2A decreases chemotherapy-induced DNA damage and associates with DSB repair proteins in TNBC. (FIG. 1A) Gene ranking of DNA repair genes according to cooccurrence with high-TRIM37 in breast cancer patients. The inset shows genes that have the highest correlation with TRIM37. A complete list of DNA repair genes that correlate with TRIM37 is presented in Table 4. (FIGS. 1B-1 to 1B-3) Heat map for the expression of TRIM37 and 28 DSB repair genes in breast cancer patients stratified by TNBC (n=299; FIG. 1B-1), non-TNBC (n=1605; FIG. 1B-2), and normal (n=100; FIG. 1B-3). n, number of samples. (FIG. 1C), non-TNBC (n=1605), and NAT (n=100). n, number of samples. (FIG. 1C-FIG. 1D) Forest plot of HR in TNBC (FIG. 1C) and non-TNBC (FIG. 1D) patients stratified for high- and low-TRIM37 expression using METABRIC cohorts. (FIG. 1E.) Immunoblot monitoring XRCC5, XRCC6, NBS1, RAD51C, and TRIM37 in protein complexes pulled down by either anti-TRIM37 (Left), the indicated DSB proteins (Right), or an IgG control. Input, ~1-5% of whole cell lysates. (FIG. 1F) ChIP monitoring TRIM37 and H2Aub binding at Gapdh in MDA MB 468 cells expressing either Cas9 alone or with Gapdh site-specific sgRNA. (FIG. 1G) ChIP monitoring TRIM37 and BMI1 binding at FRA3B, HOXA3, and Actin in MDA MB 468 cells treated with doxorubicin (Dox). (FIG. 1H) ChIP monitoring H2Aub, XRCC5, XRCC6, NBS1, and RAD51c binding at FRA3B and Actin in MDA MB 468 cells expressing a non-silencer (NS) or TRIM37 shRNA. (FIG. 1I) HR (Left) and NHEJ (Right)-mediated DSB-repair activity in MDA MB 468 cells expressing control or TRIM37 shRNA (#1, #2; SEQ ID NOs: 77 and 78, respectively). Error bars indicate standard deviation and range of at least three biological replicates. * p<0.05; ** p<0.01; *** p<0.001. (FIG. 1J) Immunoblot monitoring XRCC5, KU70, NBS1, RAD51C, and TRIM37 in protein complexes pulled down by either anti-TRIM37 (Left), indicated DSB proteins (Right), or an IgG control. Input, ~1-5% of whole cell lysates.

FIGS. 2A-2I. TRIM37-catalyzed H2Aub is required for chemoresistance in TNBC. (FIG. 2A) Top, Tail moment in DMSO or Dox-treated MDA MB 468 cells expressing non-silencer (NS) or TRIM37 shRNA (#1, #2; SEQ ID NOs: 77 and 78, respectively). Bottom, Representative images of the tails for each group are shown. Scale bars, 100 µm. (FIG. 2B) Top, Quantification of γ-H2AX foci in MDA MB 468 cells expressing NS or TRIM37 shRNA (#1, #2; SEQ ID NOs: 77 and 78, respectively) following treatment with Dox. Bottom, Representative immunofluorescence images of γ-H2AX foci (Green or lighter gray) in Dox-treated MDA MB 468 cells expressing NS or TRIM37 shRNA (#1, #2; SEQ ID NOs: 77 and 78, respectively). DAPI (Blue or darker gray) stains the nucleus. Scale bars, 50 µm. (FIG. 2C) Caspase 3 activity assay (Top) and immunoblot for PARP and cleaved PARP (c-PARP) (Middle) in DMSO or Dox-treated MDA MB 468, HCC1806, MDA MB 231, and MCF7 cells expressing NS or TRIM37 shRNA (#1, #2; SEQ ID NOs: 77 and 78, respectively). Quantification of PARP cleavage relative to total PARP is shown (Bottom). (FIG. 2D) Quantification of the fold change in chemotherapeutic drug-resistant colonies obtained for MDA MB 468 cells expressing either NS or TRIM37 shRNA (#1, #2; SEQ ID NOs: 77 and 78, respectively) by a clonogenic assay. Cells were treated with DMSO, Dox, temozolomide (TMZ), etoposide (EPO), daunorubicin (Daun), and cisplatin (CPN). Results were normalized to the colony forming unit (cfu) for DMSO. (FIG. 2E) ChIP monitoring TRIM37 and H2Aub binding at FAR3B and Actin in MCF10AT cells expressing vector control (VC), TRIM37, or mutant TRIM37 (TRIM37(C18R)). (FIG. 2F) Top, Tail moment in DMSO or Dox-treated VC-, TRIM37-, or TRIM37(C18R)-expressing MCF10AT cells. Bottom, Representative images of the tail moment for each group are shown. Scale bars, 100 µm. (FIGS. 2G-H) Caspase 3 activity assay (FIG. 2G) and immunoblot for PARP and c-PARP (FIG. 2H, Left) in DMSO or Dox-treated VC, TRIM37 or TRIM37(C18R) expressing MCF10AT cells. Quantification of PARP cleavage relative to total PARP is shown (Right). (FIG. 2I) Quantification of the fold change in chemotherapeutic drug resistant colonies obtained for VC, TRIM37, or TRIM37(C18R) by clonogenic assay. Cells were treated with DMSO, Dox, temozolomide (TMZ), etoposide (EPO), daunorubicin (Daun), and cisplatin (CPN). Results were normalized to the cfu for DMSO. Error bars indicate standard deviation and range of at least three biological replicates. * p<0.05; ** p<0.01; *** p<0.001.

FIGS. 3A-3I. Oncogenic TRIM37 reduces cytotoxicity of chemotherapy in the absence of functional p53. (FIG. 3A) Heat map for expression of TRIM37 and DSB genes in TNBC patients stratified by TP53 status (wild type (n=238) and mutant (n=61)). n, number of patients. A complete list of genes correlating with TRIM37 is presented in Table 4. (FIG. 3B-FIG. 3C) Forest plot of hazard ratio (HR) for TNBC patients with mutant TP53 (FIG. 3B), or wild type TP53 (FIG. 3C) stratified for high- and low-TRIM37 expression. (FIG. 3D) Immunoblots in Dox-treated MDA MB 468 and HCC1806 cells expressing NS, TRIM37 shRNA, or TP53. Tubulin is the loading control. Bottom, Quantification of c-PARP relative to total PARP. (FIG. 3E) Caspase 3 activity assay in Dox-treated MDA MB 468 and HCC1806 expressing NS, TRIM37 shRNA, or TP53. (FIG. 3F) Quantification of the fold change in cfu for Dox-treated MDA MB 468 and HCC1806 cells expressing NS, TRIM37 shRNA, or TP53 by clonogenic assay. (FIG. 3G) Left, Immunoblots in Dox-treated p53-/- MCF10A cells expressing TRIM37. Tubulin is the loading control. Right, Quantification of c-PARP relative to total PARP. (FIG. 3H) Caspase 3 activity assay in Dox-treated p53-/- MCF10A cells expressing either vector control or TRIM37. (FIG. 3I) Quantification of the fold change in cfu for p53-/- MCF10A cells expressing TRIM37 plated after Dox treatment by clonogenic assay. Error bars indicate standard deviation and range of at least three biological replicates. * p<0.05; ** p<0.01; *** p<0.001.

FIGS. 4A-4K. Chemotherapy amplifies oncogenic TRIM37 network in TNBC. (FIG. 4A) Top, Immunoblots in MDA MB 468, HCC1806, MDA MB 231, MCF7, and MCF10A cells treated with Dox for 0, 24, 48 and 72 hrs. Tubulin is the loading control. Bottom, Quantification of TRIM37 relative to Tubulin. (FIG. 4B-1) Left, immunoblot monitoring TRIM37 in HCC1806 (Top) or MDA MB 231 (Bottom) cells treated with daunorubicin (Daun), cisplatin (CPN), etoposide (EPO) or temozolomide (TMZ). Tubulin is the loading control. Right, Quantification of TRIM37 relative to Tubulin. (FIG. 4B-2) Top, Immunoblots in MDA MB 468, HCC1806, MDA MB 231, MCF7, and MCF10A cells treated with 0.2, 0.4 and 0.6 µM of Dox at 24 hrs. Tubulin is the loading control. Bottom, Quantification of TRIM37 relative to tubulin. (FIG. 4C) Schematic showing that NSG mice were treated with intraperitoneal injection of 2 mg/kg Dox once tumor reached the size of ~200 mm³. Tumors were harvested postmortem. (FIG. 4D-1) qRT-PCR monitoring TRIM37 expression in HCC1806 subcutaneous tumors-derived from NSG mice following treatment with Dox. n=6 animals per group. (FIG. 4D-2) Left, Immunoblots for TRIM37, PARP and c-PARP in HCC1806 subcutaneous tumors-derived from NSG mice following treatment with Dox. Tubulin is the loading control. Right, Quantification of TRIM37 relative to tubulin. n=4 animals per group. (FIG. 4E) Schematic of ATM signaling with the downstream effectors of ATM, E2F1, and STAT. Specific small molecule inhibitors of the ATM signaling are also indicated (Red or Grey). (FIG. 4F) qRT-PCR monitoring TRIM37 expression in MDA MB 468 cells following treatment with KU555933, HLM006474 or AG490 in combination with Dox. (FIG. 4G - FIG. 4I) ChIP analysis monitoring the binding of E2F1 (FIG. 4G), STAT1 (FIG. 4H), and STAT3 (FIG. 4I) to TRIM37 and Actin in MDA MB 468 cells treated with HLM006474 or AG490 in combination with Dox. (FIG. 4J) Top, Immunoblots in MDA MB 468 cells treated with Dox and the indicated inhibitors of ATM signaling. Tubulin is the loading control. Bottom, Quantification of TRIM37 relative to Tubulin. (FIG. 4K) Heat map for TRIM37 expression in mutant and wild type TP53 TNBC tumor tissue samples pre- and post-chemotherapy. The type of TP53 mutation is indicated on the right. NA, not available. (n = 17). Error bars indicate standard deviation and range of at least three biological replicates. * p<0.05; ** p<0.01; ***p<0.001.

FIGS. 5A-5L. TRIM37 promotes anti-anoikis signaling in TNBC. (FIG. 5A - FIG. 5B) Representative images of control and TRIM37-knockdown (#1, #2; SEQ ID NOs: 77 and 78, respectively) MDA MB 231 (FIG. 5A, Left) and HCC1806 (FIG. 5B, Left) cells replated after growth in suspension for 5 days and stained with crystal violet. Quantification of the fraction of surviving cells obtained for control and TRIM37-knockdown (#1, #2; SEQ ID NOs: 77 and 78, respectively) MDA MB 231 (FIG. 5A, Right) and HCC11806 (FIG. 5B, Right) by a colony formation assay. (FIG. 5C - FIG. 5D) Caspase 3 activity assay in MDA MB 231 (FIG. 5C) and HCC1806 (FIG. 5D) cells expressing NS or TRIM37 shRNA (#1, #2; SEQ ID NOs: 77 and 78, respectively) cultured in suspension for 5 days. (E-F) Immunoblots in MDA MB 231 (FIG. 5E) and HCC1806 (#1, #2) (FIG. 5F) cells expressing an NS or TRIM37 shRNA (#1, #2; SEQ ID NOs: 77 and 78, respectively) for BIM post-detachment. Tubulin is the loading control. Bottom, Quantification of BIM relative to tubulin. (FIG. 5G - FIG. 5H) Cellular viability of MDA MB 231 (FIG. 5G) and HCC1806 (FIG. 5H) expressing NS or TRIM37 shRNA (#1, #2; SEQ ID NOs: 77 and 78, respectively) in non-adherent culture conditions. (FIG. 5I) Representative images of the vector control (VC) or wild type TRIM37 (TRIM37) or TRIM37 mutant (TRIM37(C18R)) expressing MCF10AT cells replated after growth in suspension for 5 days and stained with crystal violet. Quantification of the fraction of surviving cells obtained for VC, TRIM37 and TRIM37(C18R) by a colony formation assay (Bottom). (FIG. 5J) Caspase3 activity assay in VC, TRIM37, and TRIM37(C18R)-expressing MCF10AT cells cultured in suspension for 5 days. (FIG. 5K) Immunoblots for BIM in MCF10AT cells expressing VC or TRIM37 or TRIM37(C18R) post-detachment. Tubulin is the loading control. Bottom, Quantification of BIM relative to tubulin. (FIG. 5L) Cellular viability of MCF10AT cells expressing either VC, or TRIM37, or TRIM37(C18R) in non-adherent culture conditions.

FIGS. 6A-6O. TRIM37 alters the transcription program to favor metastatic growth of TNBC tumors. (FIG. 6A) MA plot illustrates differential gene expression in TRIM37-ASO-treated compared to control 231-2b cells. Medium grey or red are significantly upregulated genes (n=1126), Dark grey or blue are significantly downregulated genes (n=1502) and grey are genes not significantly changed (n=12,440). FDR < 0.05. (FIG. 6B) Hierarchical clustering of median-centered gene expression in control or TRIM37-ASO treated 231-2b cells. Each colored line in the dendrogram identifies a different gene. n=3. (FIG. 6C) Pathways significantly downregulated (Darker grey or Blue; bars that proceed to the left) or upregulated (Medium Grey or Red; bars that proceed to the right) in TRIM37-ASO treated cells relative to control 231-2b cells identified by GSEA. (FIG. 6D - FIG. 6E) qRT-PCR monitoring TRIM37-regulated metastasis suppressor genes in TRIM37 overexpressing p53-/- MCF10A cells relative to vector control (FIG. 6D) and TRIM37-ASO treated 231-2b tumors relative to control tumors (FIG. 6E). (FIG. 6F) ChIP monitoring BMI1, EZH2, TRIM37, and H2Aub binding at KISS1, BRMS1, and Actin in control and TRIM37-ASO treated 231-2b cells. (FIG. 6G - FIG. 6H) qRT-PCR monitoring TRIM37 target genes in 231-2b cells expressing KISS1 shRNA (FIG. 6G) and BRMS1 shRNA (FIG. 6H) relative to NS shRNA. (FIG. 6I - FIG. 6J) qRT-PCR monitoring TRIM37 target genes in TRIM37 overexpressing p53-/-MCF10A cells relative to vector control (FIG. 6I) and TRIM37-ASO treated 231-2b tumors relative to control tumors (FIG. 6J). (FIG. 6K) Schematic showing that mice were injected with control or TRIM37-ASO treated 231-2b cells intracardially and monitored for metastatic tumor burden. n=8 animals per group. (FIG. 6L) Representative ventral BLI of 231-2b expressing control or TRIM37-knockdown (TRIM37-ASO) at day 21. (FIGS. 6M-6N) Analysis of metastatic tumor growth in mice tissues measured by H&E staining post-mortem in indicated tissues, tumor marked by arrow (FIG. 6M) and relative luciferase signal for lungs and number of metastatic lesions in bones, brain, and liver (FIG. 6N). (FIG. 6O) Kaplan-Meier survival curve for mice injected with control or TRIM37-ASO-treated 231-2b cells. Error bars indicate standard deviation and range of at least three biological replicates. * p <0.05; ** p<0.01; ***p<0.001.

FIGS. 7A-7P. Design, structural and functional characterization of smart nanoparticles in TNBC cellular and xenograft mouse models. (FIG. 7A) A scheme of site-specific covalent conjugation of Farletuzumab to DSPE-PEG2000-Maleimide. An Fc-linkered sequence harboring a Cys at the extended C-terminal hole chain in Farletuzumab and conjugated to DSPE-PEG2000-Maleimide. (FIG. 7B) Structural model of smart nanoparticles with CaP core encapsulating TRIM37-ASO. Each nanoparticle has a lipid bilayer comprising of DOPA, DOTAP, and DSPE-PEG2000-Maleimide conjugated to Farletuzumab encapsulated in the core. (FIG. 7C) Density of Farletuzumab on smart nanoparticles (Smart NP). (FIG. 7D) Binding assay for relative avidity index of Farletuzumab or nanoparticles. (FIG. 7E - FIG. 7F) Dynamic light scattering (FIG. 7E) and zeta potential (FIG. 7F) for empty, control, and smart nanoparticles measured by zetasizer. (FIG. 7G) Schematic of co-culturing experiments described in (FIG. 7H - FIG. 7I). HCC1806RR (Red) and MCF7 or HCC1806RR (Red) and MCF10A cells were co-cultured in the ratio of 1:1 and treated with the IR800-labeled smart nanoparticles. Nanoparticle uptake was analyzed at 1- and 24-hours using fluorescence microscope. (FIGS. 7H - 7I) Representative images for the uptake of smart nanoparticles by HCC1806RR and MCF7 (FIG. 7H) and HCC1806RR and MCF10A (FIG. 7I) as described in (FIG. 7G). Right, Results are quantified. Scale bars represent 50 µm. (FIG. 7J) Schematic showing that NSG mice were treated with intratumor injections of smart or control nanoparticles in combination with either vehicle or Dox. At day 21, tumors were harvested post-mortem. (FIG. 7K) Tumor growth curve of the subcutaneous tumors derived from 231-2b in the response to treatments described in (FIG. 7J). n=9 animals per group. (FIG. 7L) qRT-PCR monitoring expression of TRIM37 in 231-2b tumors treated with either control or smart nanoparticles. Error bars indicate standard deviation and range of at least three biological replicates. * p <0.05; ** p<0.01; ***p<0.001. (FIG. 7M) Representative fluorescent images of tumor bearing mice after treatment with IR800-labeled smart nanoparticles at indicated times. The color scale depicts the fluorescence counts emitted from the tumor cells. (FIG. 7N) Necropsies from animals in (FIG. 7M) were analyzed by fluorescent imaging for detailed organ-specific distribution of IR800-labeled smart nanoparticles. (FIG. 7O) qRT-PCR monitoring expression of TRIM37 in 231-2b cells treated with either TRIM37-ASO or control-ASO or nanoparticles for indicated times. (FIG. 7P) qRT-PCR monitoring expression of TRIM37 in 231-2b tumors treated with control or smart nanoparticles. Error bars indicate standard deviation and range of at least three biological replicates. n=6 animals per group. * p<0.05; ** p<0.01; ***p<0.001.

FIGS. 8A-8M. Targeting of TRIM37 suppresses metastatic lung tumors in vivo. (FIG. 8A) Schematic showing that female Balb/c mice bearing mammary fat pad 4T1 tumors were treated with intranasal and intratumor injections of smart or control nanoparticles. n=8 animals per group. (FIG. 8B) Representative dorsal and ventral BLI images of tumor bearing mice at day 30 after treatment with either control or smart nanoparticles. The color scale depicts the luminescence counts emitted from the metastasis cells. n=4 animals per group. (FIG. 8C) Representative 10X H&E staining images of the lung metastases for control and smart nanoparticle treated animals. Scale bar, 0.5 mm. Arrows indicate lung metastatic nodules. (FIG. 8D) Lung necropsies from animals in (FIG. 8B) were analyzed by fluorescent imaging for tumor burden. (FIG. 8E) Quantification of metastasis incidence in the lung tissue after control or smart nanoparticles treatment. n=8 animals per group. (FIG. 8F) Ki67 staining of lung tumors derived from mice treated with control or smart nanoparticles. Scale bar, 0.5 mm. Arrows indicate highly proliferative lung metastatic nodules. (FIG. 8G) Model depicting TRIM37 function in multiple steps of TNBC metastasis. (FIG. 8H) Schematic showing that NSG mice bearing subcutaneous 231-2b tumors were treated with intranasal injections of smart or control nanoparticles in combination with Dox post-tumor resection. n=8 animals per group. (FIG. 8I) Representative BLI images for tumor-bearing mice at day 1 and day 30 post-primary tumor resection. The color scale depicts the luminescence counts emitted from the metastasis cells. n=4 animals per group. (FIG. 8J) Representative 10X H&E staining images of the lung sections of control and smart nanoparticle treated animals. Scale bar, 0.5 mm. Arrows indicate lung metastatic nodules. (FIG. 8K) Lung necropsies from animals in (FIG. 8I) were analyzed for tumor burden. (FIG. 8L) Quantification of accumulated luciferase signal from the lung tissue after control and smart nanoparticles treatment. n=8 animals per group. (FIG. 8M) Caspase 3 staining of lung tumors derived from mice treated with control or smart nanoparticles in combination with Dox. Scale bar, 0.5 mm. Error bars indicate standard deviation and range of at least three biological replicates. * p<0.05; ** p<0.01; *** p<0.001.

FIGS. 9A-9M. TRIM37 interacts with DSB proteins. (FIG. 9A-1) Table summarizing ranking of DNA repair Gene Ontology (GO) classes according to the correlation with TRIM37 expression in TNBC and non-TNBC patients. (FIG. 9A-2) Proportion of TRIM37-associated genes (Pearson’s coefficient > 0.2; Table 4) involved in cell proliferation (GO:0008283) and DSB repair (GO:0006302). Horizontal line indicates expectation by chance. (FIG. 9B) Box-plot showing TRIM37 (Left) and 28 DSB genes (Right) expression profile in TNBC patients, non-TNBC patients and normal samples using TCGA datasets. The box area spans between 1st and 3rd quartile, and whiskers represent the minimum and maximum values that do not exceed 1.5x interquartile range. (FIG. 9C - FIG. 9D) Forest plot of hazard ratios for risk of TNBC (FIG. 9C) and non-TNBC (FIG. 9D) patients stratified for high and low TRIM37 expression using METABRIC patient cohorts. (FIG. 9E) Violin plot of differential HR for 28 DSB genes in TNBC patients. (FIG. 9F) Immunoblots on sucrose gradient fractions. (FIG. 9G) Immunoblot monitoring TRIM37 expression in MCF10A, HCC1806, MDA MB 231, MDA MB 468 and murine 4T1 cells. Tubulin is the loading control. (FIG. 9H) Top, Immunoblot in MDA MB 468, MDA MB 231 and HCC1806 expressing non-silencer (NS) or TRIM37 shRNA (#1, #2; SEQ ID NOs: 77 and 78, respectively). Tubulin is the loading control. Bottom, Quantification of TRIM37 relative to Tubulin. (FIG. 9I) Top, Quantification of γ-H2AX foci in MDA MB 468 cells expressing NS or TRIM37 shRNA (#1, #2; SEQ ID NOs: 77 and 78, respectively) following treatment with Dox. Bottom, Representative immunofluorescence images of γ-H2AX foci (Green or light grey) in Dox treated MDA MB 468 cells expressing NS or TRIM37 shRNA (#1, #2; SEQ ID NOs: 77 and 78, respectively). DAPI (Blue or dark grey) stains the nucleus. Scale bars, 50 µm. (FIG. 9J) Immunoblot monitoring TRIM37, XRCC5, XRCC6, NBS1, and RAD51C pull-down efficiencies for the indicated proteins, or an IgG control. Input, ~1-5% of whole cell lysates. (FIG. 9K) Representative images for the nuclear localization of XRCC5, XRCC6, NBS1, RAD51c and TRIM37 in DMSO- (Left) or Doxorubicin- (Dox, Middle) treated cells. γ-H2AX foci indicate DNA damage. Scale bars represent 50 µm. Results are quantified (Right) (FIG. 9L) Top, Immunoblot in MDA MB 468 expressing NS or TRIM37 shRNA (#1, #2; SEQ ID NOs: 77 and 78, respectively). Tubulin is the loading control. Bottom, Quantification of TRIM37 relative to Tubulin. (FIG. 9M) qRT-PCR monitoring expression of XRCC5, XRCC6, NBS1 and RAD51c in TRIM37-knockdown HCC1806 cells. Error bars indicate standard deviation and range of at least three biological replicates. * p<0.05; ** p<0.01; ***p<0.001.

FIGS. 10A-10D. TRIM37-mediated H2A mono-ubiquitination promotes TNBC cell survival following chemotherapy. (FIG. 10A) Top, Immunoblot in MDA MB 231 and HCC1806 expressing NS or TRIM37 shRNA (#1, #2; SEQ ID NOs: 77 and 78, respectively). Tubulin is the loading control. Bottom, Quantification of TRIM37 relative to Tubulin. (FIG. 10B) Representative images of clonogenic assay for MDA MB 468 cells expressing either NS or TRIM37 shRNA (#1, #2; SEQ ID NOs: 77 and 78, respectively). Cells were treated with DMSO, Dox, temozolomide (TMZ), etoposide (EPO), daunorubicin (Daun) or cisplatin (CPN). (FIG. 10C) Top, immunoblot monitoring TRIM37 expression in MCF10AT cells expressing either vector control (VC), TRIM37 (TRIM37) or mutant TRIM37 (TRIM37(C18R)). Tubulin is the loading control. Bottom, Quantification of TRIM37 and H2Aub relative to Tubulin. (FIG. 10D) Representative images of clonogenic assay for MCF10AT cells expressing either VC, TRIM37 or TRIM37(C18R) following treatment with Dox, TMZ, EPO, Daun or CPN. Error bars indicate standard deviation and range of at least three biological replicates. * p<0.05; ** p<0.01; *** p<0.001.

FIGS. 11A-11F. TRIM37 promotes TNBC cell survival following chemotherapy in the absence of functional p53. (FIG. 11A) Histogram showing the percent of patients with TP53 mutant (p53 mut) and wild type (p53 wild type) in TNBC and non- TNBC tissue samples. (FIG. 11B) Heat map for expression of TRIM37 and 28 DSB repair genes in breast cancer tissue patients stratified by p53 status in non-TNBC (n=1605), and normal (n=100). n, number of samples. (FIG. 11C - FIG. 11D) Box-plot showing expression of DSB genes that positively correlated in TNBC, non-TNBC and normal tissue samples stratified based on mutant (FIG. 11C) and wild type (FIG. 11D) TP53. The box area spans between 1st and 3rd quartile, and whiskers represent the minimum and maximum values that do not exceed 1.5x interquartile range. (FIG. 11E) Representative images of MDA MB 468 and HCC1806 cells expressing NS, TRIM37 shRNA (shRNA), or TP53 plated after Dox treatment and stained with crystal violet. (FIG. 11F) Representative images of p53-/-MCF10A expressing TRIM37 plated after Dox treatment and stained with crystal violet.

FIGS. 12A-12O. Chemotherapy activates ATM/E2F1/STAT axis to upregulate TRIM37 in TNBC. (FIG. 12A-1) Left, Immunoblot monitoring TRIM37 expression in MDA MB 468 cells treated with Dox, daunorubicin (Daun), cisplatin (CPN), etoposide (EPO) or temozolomide (TMZ). Tubulin is the loading control. Right, Quantification of TRIM37 relative to tubulin. (FIG. 12A-2) Immunoblot in MCF7 (Left) and MCF10a (Right) cells treated with Dox for 0, 2, 4 and 8 hrs. Gapdh is the loading control. (FIG. 12B) Immunoblot in MDA MB 468 cells expressing either vector control (VC), or wild type TP53 (TP53) following treatment with Dox. Tubulin is the loading control. (FIG. 12C) qRT-PCR monitoring TRIM37 expression in HCC1806 subcutaneous tumors-derived from NSG mice following treatment with Dox. (FIGS. 12D - 12E) List of putative E2F- (FIG. 12D) and STAT- (FIG. 12E) binding sites in TRIM37 promoter region (-1500 to +1500). (FIG. 12E) ChIPSeq tracks of E2F1, STAT1 and STAT3 on the TRIM37 promoter identified by ENCODE project viewed in UCSC genome Browser. (FIGS. 12G - 12K) Immunoblots in MDA MB 468 cells treated with KU555933 (FIG. 12G), HLM006474 (FIG. 12H), or AG490 (FIG. 12I - FIG. 12K). Tubulin is the loading control. (FIG. 12L) Immunoblot in MDA MB 468 cells expressing NS or E2F1 shRNA (#1, #2; SEQ ID NOs: 83 and 84, respectively) following treatment with Dox. Gapdh is the loading control. (FIG. 12M) Immunoblot in MDA MB 468 cells expressing NS or STAT1 (#1, #2; SEQ ID NOs: 85 and 86, respectively) and STAT3 shRNA (#1, #2; SEQ ID NOs: 87 and 88, respectively) following treatment with Dox. Gapdh is the loading control. (FIG. 12N - FIG. 12O) Immunoblot in MCF10AT cells over expressing E2F1 (FIG. 12N) or STAT⅓ (FIG. 12O). Gapdh is the loading control. Error bars indicate standard deviation and range of at least three biological replicates. * p<0.05; ** p<0.01: *** p<0.001.

FIGS. 13A-13O. Antisense oligonucleotide-mediated knockdown of TRIM37. (FIG. 13A) Quantitation of the fraction of surviving MCF10A cells after staining colonies with crystal violet.-(FIG. 13B) Cellular viability (%) of HCC1806 (Left) and MDA MB 468 (Right) cells expressing NS or TRIM37 shRNA (#1, #2; SEQ ID NOs: 77 and 78, respectively) in adherent culture conditions at 48 hrs. (FIG. 13C) qRT-PCR analysis monitoring knockdown efficiencies of TRIM37 antisense oligonucleotides (TRIM37-ASO, #1 and #2; SEQ ID NOs: 77 and 78, respectively) in HCC1806, MDA MB 468 and MDA MB 231 cells. (FIG. 13D) Immunoblots monitoring TRIM37 expression HCC1806 treated with TRIM37-ASO (#1, #2; SEQ ID NOs: 77 and 78, respectively). Tubulin is the loading control. (FIG. 13E) Immunoblots monitoring TRIM37 expression in MDA MB 231 treated with TRIM37-ASO at indicated time. Tubulin is the loading control. (FIG. 13F) Percentage of intra-cardially injected mice with tumors at the indicated sites. (FIG. 13G) Necropsies from animals were analyzed by fluorescent imaging for tumor burden. (FIG. 13H) Analysis of available datasets from Echeverria et. al., (Echeverria et al., 2018) showing TRIM37 expression in primary mammary fat pad (Blue or darker grey) and lung metastatic tumors (Red or lighter grey) derived from the PDX model PIM001-P. n=7. (FIG. 13I) Enrichment plots for gene signatures identified through GSEA analysis of RNA-seq data. (FIG. 13J) Top 10 KEGG pathways upregulated (Top) and downregulated (Bottom) in 231-2b cells. Gene list was based on a consensus between three biological replicates. (FIG. 13K) qRT-PCR monitoring BMI1 and EZH2 in 231-2b expressing NS or BMI1 shRNA (#1, #2; SEQ ID NOs: 91 and 92, respectively; Left) or EZH2 shRNA (#1, #2; SEQ ID NOs: 89 and 90, respectively; Right). (FIG. 13L) qRT-PCR monitoring KISS1 (Left) and BRMS1 (Right) in 231-2b cells expressing BMI1 shRNA (#1, #2; SEQ ID NOs: 91 and 92, respectively) and EZH2 shRNA (#1, #2; SEQ ID NOs: 89 and 90) relative to NS shRNA. (FIG. 13M) qRT-PCR monitoring KISS1 and BRMS1 in 231-2b expressing NS or KISS1 shRNA (#1, #2; SEQ ID NOs: 79 and 80, respectively; Left) or BRMS1 shRNA (#1, #2; SEQ ID NOs: 81 and 82, respectively; Right). (FIG. 13N) qRT-PCR monitoring TRIM37 target genes in MCF7 cells expressing TRIM37 shRNA (SEQ ID NOs: 77 and 78) relative to NS shRNA. (FIG. 13O) Analysis of metastatic tumor growth in mice tissues measured by H&E staining post-mortem in lungs and liver sections. Error bars indicate standard deviation and range of at least three biological replicates. * p<0.05; ** p<0.01; ***p<0.001.

FIGS. 14A-14N. Smart nanoparticle mediated inhibition of TRIM37. (FIG. 14A) Box-plot showing expression of FOLR1 in healthy, non-TNBC and TNBC tissue samples. The box area spans between 1^{st} and 3^{rd} quartile, and whiskers represent the minimum and maximum values that do not exceed 1.5x interquartile range. (FIG. 14B) SDS-PAGE analysis of Farletuzumb and Fc-linkered Farletuzumb in reducing conditions. (FIG. 14C) Size exclusion chromatography of Fc-linkered Farletuzumb on a Superdex column. The molecular mass standard are as follows: 670, 158, 44, 17 and 1.3 kDa. (FIG. 14D) SDS-PAGE analysis of smart nanoparticles, control nanoparticles and Fc-linkered Farletuzumb in reducing conditions. (FIG. 14E) Transmission electron micrograph of Farletuzumab-conjugated nanoparticles with colloidal gold marking the Farletuzumb on the surface of nanoparticles (shown by arrowheads). (FIG. 14F) Size distribution of empty, control and smart nanoparticles. (FIG. 14G) Release rate of TRIM37-ASO from smart nanoparticles over a period of 48 hours with a biphasic release pattern. A curve fit line is represented by dashed line. (FIG. 14H) Immunoblot monitoring FOLR1 expression in MCF7, MCF10A and HCC1806RR cells. Tubulin is the loading control. (FIG. 14I) qRT-PCR monitoring expression of TRIM37 in 231-2b cells treated with either control or smart nanoparticles for indicated times. (FIG. 14J) qRT-PCR monitoring expression of TRIM37 in 231-2b cells treated with either TRIM37-ASO or smart nanoparticles. (FIG. 14K) Immunoblot monitoring TRIM37 in 231-2b cells treated with either control or smart nanoparticles. Tubulin is the loading control. (FIG. 14L) A bliss synergy score>0 for the synergistic effect of smart nanoparticles and Dox in vivo. (FIG. 14M) Immunoblot monitoring TRIM37 in 231-2b subcutaneous tumors treated with either control or smart nanoparticles. n=3 animals per group. Tubulin is the loading control. Quantification of TRIM37 relative to tubulin (Right). (FIG. 14N) Fold change in the body weight of NSG mice in different groups. Error bars indicate standard deviation and range of at least three biological replicates. * p <0.05; ** p<0.01: *** p<0.001.

FIGS. 15A-15O. Targeting of TRIM37 suppresses TNBC metastasis. (FIG. 15A) A scheme of site-specific and covalent conjugation of murine cross-reactive anti-FOLR1 to DSPE-PEG2000-Maleimide. An Fc-linkered sequence harboring a Cys at the extended C-terminal hole chain was engineered in murine cross-reactive anti-FOLR1 and conjugated to DSPE-PEG2000-Maleimide by utilizing maleimide chemistry. (FIG. 15B) SDS-PAGE analyses of murine cross-reactive anti-FOLR1 and Fc-linkered anti-FOLR1 in reducing conditions. (FIG. 15C) qRT-PCR monitoring expression of TRIM37 in 4T1 cells treated with either control or smart nanoparticles. (FIG. 15D) Frequency of primary and lung metastases for 231-2b cells engrafted in NSG mice and treated with either control or smart nanoparticles. (FIG. 15E - FIG. 15F) Representative 10X H&E (FIG. 15E) and TRIM37 (FIG. 15F) staining images of the lung sections from control and smart nanoparticles treated Balb/c mice. Scale bar, 0.5 mm. Arrow indicate lung metastatic nodules. (FIG. 15G) Representative 10X H&E staining images of liver sections from control and smart nanoparticles treated NSG mice. Scale bar, 0.5 mm. (FIG. 15H) ALT and AST assays monitoring hepatotoxicity in control and smart nanoparticles treated animals. n=8 animals per group. (FIG. 15I) Frequency of primary and lung metastases for 231-2b cells engrafted in NSGmice and treated with either control or smart nanoparticles. n=8 animals per group. (FIG. 15J) Ki67 staining of lung tumors derived from NSG mice treated with control or smart nanoparticles. Scale bar, 0.5 mm. Arrow indicate highly proliferative lung metastatic nodules. (FIG. 15K) Representative 10X H&E staining images of the lung sections from control and smart nanoparticles treated NSG mice. Scale bar, 0.5 mm. Arrow indicates lung metastatic nodules. (FIG. 15L) Gel filtration analysis of Fc-linkered anti-FOLR1 on a Superdex column. The molecular mass standard are as follows: 670, 158, 44, 17 and 1.3 kDa. (FIG. 15M) SDS-PAGE analysis of control and smart nanoparticles along with murine cross-reactive Fc-linkered anti-FOLR1 in reducing conditions. (FIG. 15N) qRT-PCR monitoring expression of TRIM37 in 4T1 cells treated with either control or smart nanoparticles. (FIG. 15O) Immunoblots monitoring TRIM37 expression in 4T1 cells treated with control or smart nanoparticles. Tubulin is the loading control. Error bars indicate standard deviation and range of at least three biological replicates. * p<0.05; ** p<0.01; *** p<0.001.

### DETAILED DESCRIPTION

The majority of clinical deaths in TNBC patients are due to aggressive metastases, but women of African ethnicities experience worst clinical outcomes and highest mortality. While tumorigenic drivers are numerous and varied, the drivers of metastatic traits remain largely unknown. Here, we uncovered a molecular dependence of TNBC tumors on oncogenic TRIM37, an epigenetic transcriptional repressor that renders them highly metastatic. Functional characterization of TRIM37 revealed that its overexpression promotes chemoresistance, anti-anoikis signaling, and metastatic transcriptional program. We show that chemotherapeutic drugs trigger an ATM/E2F1/STAT loop, which amplifies the TRIM37 network, thereby reducing therapy-induced cancer cell death. Transcriptome profiling of TNBC cells identified TRIM37 as a leading gene driving the expression of metastatic effectors. Selective delivery of TRIM37-specific antisense oligonucleotides using anti-folate receptor 1-conjugated nanoparticles inhibit TRIM37 and attenuates lung metastasis in spontaneous metastatic murine models. These findings establish TRIM37 as a clinically relevant target with opportunities for therapeutic interventions.

### BRIEF DESCRIPTION OF THE SEQUENCE LISTING

SEQ ID NO: 1 is the nucleotide sequence of Accession No. NM_015294.6 of the GENBANK® biosequence database, and corresponds to the human tripartite motif containing 37 (TRIM37), transcript variant 1. SEQ ID NO: 2 is the amino acid sequence of Accession No. NP_056109.1 of the GENBANKⓇ biosequence database, and is encoded by SEQ ID NO: 1.

SEQ ID NO: 3 is the nucleotide sequence of Accession No. NM_001005207.5 of the GENBANK® biosequence database, and corresponds to the human tripartite motif containing 37 (TRIM37), transcript variant 2. SEQ ID NO: 4 is the amino acid sequence of Accession No. NP_001005207.1 of the GENBANKⓇ biosequence database, and is encoded by SEQ ID NO: 2.

SEQ ID NO: 5 is the nucleotide sequence of Accession No. NM_001320987.3 of the GENBANK® biosequence database, and corresponds to the human tripartite motif containing 37 (TRIM37), transcript variant 3. SEQ ID NO: 6 is the amino acid sequence of Accession No. NP_001307916.1 of the GENBANKⓇ biosequence database, and is encoded by SEQ ID NO: 5.

SEQ ID NO: 7 is the nucleotide sequence of Accession No. NM_001320988.3 of the GENBANK® biosequence database, and corresponds to the human tripartite motif containing 37 (TRIM37), transcript variant 4. SEQ ID NO: 8 is the amino acid sequence of Accession No. NP_001307917.1 of the GENBANKⓇ biosequence database, and is encoded by SEQ ID NO: 7.

SEQ ID NO: 9 is the nucleotide sequence of Accession No. NM_001320989.3 of the GENBANK® biosequence database, and corresponds to the human tripartite motif containing 37 (TRIM37), transcript variant 5. SEQ ID NO: 10 is the amino acid sequence of Accession No. NP_01307918.1 of the GENBANKⓇ biosequence database, and is encoded by SEQ ID NO: 9.

SEQ ID NO: 11 corresponds to nucleotides 58,998,202 to 59,106,880 of the human chromosome 17 sequence as set forth in Accession No. NC_000017.11 of the GENBANKⓇ biosequence database. The reverse complement of SEQ ID NO: 11 corresponds to the human TRIM37 genomic locus, and is set forth in SEQ ID NO: 12.

SEQ ID NOs: 13-92 are the nucleotide sequences of various oligonucleotides that were employed in the compositions and methods of the presently disclosed subject matter as described in Table 5.

SEQ ID NOs: 93 and 94 are the amino acid sequences of the heavy chain and the light chain, respectively, of antibody LK26.

SEQ ID NOs: 95 and 96 are the amino acid sequences of the heavy chain and the light chain, respectively, of antibody Farletuzumab.

SEQ ID NOs: 97 and 98 are the nucleotide and amino acid sequences, respectively, of an exemplary non-cysteine linker of the presently disclosed subject matter.

SEQ ID NOs: 99 and 100 are the nucleotide and amino acid sequences, respectively, of an exemplary cysteine linker of the presently disclosed subject matter.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1 % from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 1:

TABLE 1

Amino Acids and Codes Therefor

| | | | | | |
|---|---|---|---|---|---|
| Full Name | 3-Letter Code | 1-Letter Code | Full Name | 3-Letter Code | 1-Letter Code |
| Aspartic Acid | Asp | D | Threonine | Thr | T |
| Glutamic Acid | Glu | E | Glycine | Gly | G |
| Lysine | Lys | K | Alanine | Ala | A |
| Arginine | Arg | R | Valine | Val | V |
| Histidine | His | H | Leucine | Leu | L |
| Tyrosine | Tyr | Y | Isoleucine | Ile | I |
| Cysteine | Cys | C | Methionine | Met | M |
| Asparagine | Asn | N | Proline | Pro | P |
| Glutamine | Gln | Q | Phenylalanine | Phe | F |
| Serine | Ser | S | Tryptophan | Trp | W |

The expression “amino acid” as used herein is me\ant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide’s circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

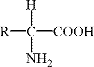

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H} -C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Patent Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in Table 2:

TABLE 2

Conservative Amino Acid Substitutions

| | | |
|---|---|---|
| Group | Characteristics | Amino Acids |
| A. | Small aliphatic, nonpolar or slightly polar residues | Ala, Ser, Thr, Pro, Gly |
| B. | Polar, negatively charged residues and their amides | Asp, Asn, Glu, Gln |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is a tumor or a cancer, which in some embodiments is Triple Negative Breast Cancer (TNBC).

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal’s health continues to deteriorate.

In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence such as SEQ ID NO: 1, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990, and can be accessed, for example at the National Center for Biotechnology Information (NCBI) world wide web site. BLAST nucleotide searches can be performed with the NBLAST program (designated “blastn” at the NCBI web site), using the following parameters: gap penalty = 5; gap extension penalty = 2; mismatch penalty = 3; match reward = 1; expectation value 10.0; and word size = 11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethyl ester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxy succinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2X standard saline citrate (SSC), 0.1% SDS at 50° C.; in some embodiments in 7% (SDS), 0.5 M NaPO4, 1 mM EDTA at 50° C. with washing in 1X SSC, 0.1% SDS at 50° C.; in some embodiments 7% SDS, 0.5 M NaPO4, 1 mM EDTA at 50° C. with washing in 0.5X SSC, 0.1% SDS at 50° C.; and more in some embodiments in 7% SDS, 0.5 M NaPO4, 1 mM EDTA at 50° C. with washing in 0.1X SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

One of ordinary skill in the art will appreciate that based on the sequences of the components of the antibodies disclosed herein they can be modified independently of one another with conservative amino acid changes, including, insertions, deletions, and substitutions, and that the valency could be altered as well. Amino acid changes (fragments and homologs) can be made independently in an antibody as well when they are being used in a therapy.

### II. Compositions

### II.A. Generally

The presently disclosed subject matter provides in some embodiments compositions, optionally pharmaceutical compositions, that comprise, consist essentially of, or consist of an effective amount of an inhibitor of TRIM37 activity and in some embodiments further comprise one or more theapeutic agents, including but not limited to chemotherapeutically active agents.

As used herein, the phrase “inhibitor of TRIM37 activity” refers to a compound, composition, and/or molecule that, when it contacts a TRIM37 gene product (e.g., a TRIM37 polypeptide or a nucleic acid encoding the same) results in an observable reduction in at least one biological activity of the TRIM37 gene product. TRIM37 gene products are known to be involved in mono-ubiquitination of histone H2A at lysine 119, which itself appears to be associated with transcriptional repression. As such, an inhibitor of TRIM37 activity is any compound, composition, and/or molecule that, when it contacts a TRIM37 gene product results in reduced TRIM37 biological activity. The biological activity of the TRIM37 gene product can be assayed directly as set forth herein or, as a proxy, can be assayed by observing reduced chemoresistance of a tumor and/or cancer cell in the presence of the inhibitor of TRIM37 activity than in its absence under otherwise identical conditions. By way of example and not limitation, the cancer can in some embodiments be triple negative breast cancer (TNBC).

Any inhibitor of TRIM37 activity can be employed in the compositions and methods of the presently disclosed subject matter. Exemplary inhibitors of TRIM37 activity include, but are not limited to anti-sense oligonucleotides that target TRIM37 gene products, anti-TRIM37 antibodies and fragments thereof, small molecule inhibitors, and a combination thereof. As used herein, the phrase “TRIM37 gene product” refers to any transcription product of a TRIM37 genetic locus or any post-transcriptionally modified derivative thereof, whether that transcription product or post-transcriptionally modified derivative thereof is produced in vivo, ex vivo, or in vitro. Additionally, the transcription product or post-transcriptionally modified derivative thereof can be a naturally occurring gene product from a normal cell or a tumor and/or cancer cell can be a modified version thereof. Furthermore, the phrase “TRIM37 gene product” also refers to a translation product that is encoded by any transcription product of a TRIM37 genetic locus or any post-transcriptionally modified derivative thereof. Therefore, in some embodiments the phrase “TRIM37 gene product” refers to nucleic acids an amino acid sequences encoded thereby.

It will be understood by those skilled in the art that the targeting sequence may make up the entirety of an antisense oligomer of this disclosure, or it may make up just a portion of an antisense oligomer of this disclosure. For example, in an oligomer consisting of 30 nucleotides, all 30 nucleotides can be complementary to a 30 contiguous nucleotide target sequence. Alternatively, for example, only 20 contiguous nucleotides in the oligomer may be complementary to a 20-contiguous nucleotide target sequence, with the remaining 10 nucleotides in the oligomer being mismatched to nucleotides outside of the target sequence. In some embodiments, oligomers of this disclosure have a targeting sequence of at least 10 nucleobases, at least 11 nucleobases, at least 12 nucleobases, at least 13 nucleobases, at least 14 nucleobases, at least 15 nucleobases, at least 16 nucleobases, at least 17 nucleobases, at least 18 nucleobases, at least 19 nucleobases, at least 20 nucleobases, at least 21 nucleobases, at least 22 nucleobases, at least 23 nucleobases, at least 24 nucleobases, at least 25 nucleobases, at least 26 nucleobases, at least 27 nucleobases, at least 28 nucleobases, at least 29 nucleobases, or at least 30 nucleobases in length.

It will be understood by those skilled in the art that the inclusion of mismatches between a targeting sequence and a target sequence is possible without eliminating the activity of the oligomer (e.g., modulation of splicing). Moreover, such mismatches can occur anywhere within the antisense interaction between the targeting sequence and the target sequence, so long as the antisense oligomer is capable of specifically hybridizing to the targeted nucleic acid molecule. Thus, antisense oligomers of this disclosure may comprise up to about 20% nucleotides that are mismatched, thereby disrupting base pairing of the antisense oligomer to a target sequence, as long as the antisense oligomer specifically hybridizes to the target sequence. In some embodiments, antisense oligomers comprise no more than 20%, no more than about 15%, no more than about 10%, no more than about 5% or not more than about 3% of mismatches, or less. In some embodiments, there are no mismatches between nucleotides in the antisense oligomer involved in pairing and a complementary target sequence. In some embodiments, mismatches do not occur at contiguous positions. For example, in an antisense oligomer containing 3 mismatch positions, in some embodiments the mismatched positions are separated by runs (e.g., 3, 4, 5, etc.) of contiguous nucleotides that are complementary with nucleotides in the target sequence

The use of percent identity is a common way of defining the number of mismatches between two nucleic acid sequences. For example, two sequences having the same nucleobase pairing capacity would be considered 100% identical. Moreover, it should be understood that both uracil and thymidine will bind with adenine. Consequently, two molecules that are otherwise identical in sequence would be considered identical, even if one had uracil at position x and the other had a thymidine at corresponding position x. Percent identity may be calculated over the entire length of the oligomeric compound, or over just a portion of an oligomer. For example, the percent identity of a targeting sequence to a target sequence can be calculated to determine the capacity of an oligomer comprising the targeting sequence to bind to a nucleic acid molecule comprising the target sequence. In some embodiments, the targeting sequence is at least 80% identical, at least 85% identical, at least 90% identical, at least 95% identical, at least 97% identical, at least 98% identical or at least 99% identical over its entire length to a target sequence in a target nucleic acid molecule. In some embodiments, the targeting sequence is identical over its entire length to a target sequence in a target nucleic acid molecule. In some embodiments, a target nucleic acid molecule comprises, consists essentially of, or consists of a nucleic acid sequence as set forth in any of SEQ ID NOs: 1, 3, 5, 7, 9, 11, and 12. In some embodiments, a target nucleic acid molecule comprises, consists essentially of, or consists of a nucleic acid sequence that is an ortholog, hom,olog, or paralog of any of SEQ ID NOs: 1, 3, 5, 7, 9, 11, and 12

It is understood by those skilled in the art that an antisense oligomer need not be identical to the oligomer sequences disclosed herein to function similarly to the antisense oligomers described herein. Shortened versions of antisense oligomers taught herein, or non-identical versions of the antisense oligomers taught herein, fall within the scope of this disclosure. Non-identical versions are those wherein each base does not have 100% identity with the antisense oligomers disclosed herein. Alternatively, a non-identical version can include at least one base replaced with a different base with different pairing activity (e.g., G can be replaced by C, A, or T). Percent identity is calculated according to the number of bases that have identical base pairing corresponding to the oligomer to which it is being compared. The non-identical bases may be adjacent to each other, dispersed throughout the oligomer, or both. For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art. Thus, antisense oligomers of this disclosure comprise oligonucleotide sequences at least 80% identical, at least 85% identical, at least 90% identical, at least 92% identical, at least 94% identical at least 96% identical or at least 98% identical to sequences disclosed herein, as long as the antisense oligomers are able to modulate splicing of a desired mRNA molecule.

Accordingly, in some embodiments an inhibitor of TRIM37 activity comprises an antisense oligonucleotide that targets a TRIM37 nucleic acid gene product such as but not limited to a TRIM37 transcription product. By way of example and not limitation, the human TRIM 37 genetic locus (SEQ ID NO: 12) is present on chromosome 17 and corresponds to the reverse-complement of nucleotides 58,998,202 to 59,106,880 of Accession No. NC_000017.11 of the GENBANK® biosequence database (presented as SEQ ID NO: 11). Multiple transcription products are derived from this locus, and include but are not limited to those set forth in Accession Nos. NM_015294.6 (transcript variant 1 (SEQ ID NO: 1); encoding GENBANK® Accession No. NP_056109.1 (SEQ ID NO: 2)), NM_001005207.5 (transcript variant 2 (SEQ ID NO: 3); encoding GENBANK® Accession No. NP_001005207.1 (SEQ ID NO: 4)), NM_001320987.3 (transcript variant 3 (SEQ ID NO: 5); encoding GENBANK® Accession No. NP_001307916.1 (SEQ ID NO: 6)), NM_001320988.3 (transcript variant 4 (SEQ ID NO: 7); encoding GENBANK® Accession No. NP_001307917.1 (SEQ ID NO: 8)), and NM_001320989.3 (transcript variant 5 (SEQ ID NO: 9); encoding GENBANK® Accession No. NP_01307918.1 (SEQ ID NO: 10)) of the GENBANK® biosequence database. Orthologs of the human TRIM37 gene product include, but are not limited to Accession No. NM_001108288.1 encoding Accession No. NP_001101758.1 (Rattus norvegicus), Accession No. NM_001363025.1 encoding Accession No. NP_001349954.1 (Mus musculus), Accession No. XM_038677132.1 encoding Accession No. XP_038533060.1 (Canis lupus familiaris), Accession No. XM_011289104.3 encoding Accession No. XP_01128746.1 (Felis catus), and Accession No. NM_{_}001110182.1 encoding Accession No. NP_001103652.1 (Bos taurus) of the GENBANK® biosequence database.

By way of example and not limitation, in some embodiments the human TRIM37 gene product comprises a nucleotide sequence as set forth in Accession No. NM_015294.6 of the GENBANK® biosequence database or a precursor thereof. Inhibitors of TRIM37 activity can thus include an anti-sense oligonucleotide that targets a human TRIM37 gene product comprising a nucleotide sequence as set forth in Accession No. NM_015294.6 of the GENBANKⓇ biosequence database. Such anti-sense oligonucleotides can include sequences that target exon sequences, intron sequences, and/or intron/exon boundary sequences.

By way of example and not limitation and in order to exemplify approaches to target exon sequences, intron sequences, and/or intron/exon boundary sequence of a TRIM37 gene product, the exons of Accession No. NM_015294.6 of the GENBANK® biosequence database (i.e., SEQ ID NO: 1) and the corresponding exonic sequences of SEQ ID NO: 12 are presented in Table 3.

TABLE 3

Exon Locations of an Exemplary TRIM37 Gene Product: GENBANKⓇ Accession No. NM 015294.6

| Exon Number | Nucleotides of NC_000017.11* | Nucleotides of SEQ ID NO: 1 | Nucleotides of SEQ ID NO: 12 |
|---|---|---|---|
| 1 | 59106441-59106880 | 1-440 | 1-440 |
| 2 | 59104293-59104394 | 441-542 | 2487-2588 |
| 3 | 59091300-59091340 | 543-583 | 15541-15581 |
| 4 | 59088291-29088407 | 584-700 | 18474-18590 |
| 5 | 59084002-59084089 | 701-788 | 22792-22879 |
| 6 | 59081097-59081219 | 789-911 | 25662-25784 |
| 7 | 59079754-59078977 | 912-1035 | 27004-27127 |
| 8 | 59075647-59075714 | 1036-1103 | 31167-31234 |
| 9 | 59070823-59070947 | 1104-1228 | 35934-36058 |
| 10 | 59064355-59064405 | 1229-1279 | 42476-42526 |
| 11 | 59062567-59062648 | 1280-1361 | 44233-44314 |
| 12 | 59061032-59061108 | 1362-1438 | 45773-45849 |
| 13 | 59056875-59057054 | 1439-1618 | 49827-50006 |
| 14 | 59051214-59051328 | 1619-1733 | 545553-55667 |
| 15 | 59049178-59049393 | 1734-1949 | 57488-57703 |
| 16 | 59047683-59047819 | 1950-2086 | 59062-59198 |
| 17 | 59041810-59041898 | 2087-2175 | 64983-65071 |
| 18 | 59031896-59032087 | 2176-2367 | 74794-74985 |
| 19 | 59028415-59028723 | 2368-2676 | 78158-78466 |
| 20 | 59017296-59017424 | 2677-2805 | 89457-89585 |
| 21 | 59015610-59015799 | 2806-2995 | 91082-91271 |
| 22 | 59012328-59012446 | 2996-3114 | 94435-94553 |
| 23 | 59001598-59001714 | 3115-3231 | 15167-105283 |
| 24 | 58998202-58999459 | 3232-4489 | 107422-108679 |

*reverse complement of the recited nucleotides

Targeting intron/exon boundary sequences of a TRIM37 gene product can include use of an antisense oligonucleotide that spans an intron/exon boundary sequence such as, but not limited to an antisense oligonucleotide that includes at least 5, 10, 15, or more nucleotides of an exon and at least 5, 10, 15, or more contiguous nucleotides in the adjacent intron in the genome and/or at least 5, 10, 15, or more nucleotides of an intron and at least 5, 10, 15, or more contiguous nucleotides in the adjacent exon. Such an approach can result in “exon skipping”, which is discussed, for example, in U.S. Pat. Nos. 8,361,979; 9,777,727; 9,972,015; and 10,781,451, each of which is incorporated herein by reference in its entirety.

Thus, in some embodiments the inhibitor of TRIM37 activity is an inhibitory nucleic acid that targets a TRIM37 gene product. As used herein, the phrase “inhibitory nucleic acid” refers to any nucleic acid molecule capable of mediating RNA interference (RNAi) or gene silencing. As used herein, inhibitory nucleic acid molecules need not be limited to those molecules containing only RNA, but further encompass chemically modified nucleotides and non-nucleotides. See e.g., Bass, 2001; Elbashir et al., 2001; and PCT International Publication Nos. WO 99/07409; WO 99/32619; WO 00/01846; WO 00/44895; WO 00/44914; WO 01/36646; and WO 01/29058. Exemplary inhibitory nucleic acids include small interfering RNAs, short interfering RNAs, siRNAs, and miRNAs. In some embodiments, the inhibitory nucleic acid comprises a double stranded polynucleotide molecule comprising complementary sense and antisense regions, wherein the antisense region comprises a sequence complementary to a region of a target nucleic acid molecule. For example, in some embodiments the inhibitory nucleic acid comprises, consists essentially of, or consists of an antisense region complementary to a region of a transcription product of a TRIM37 gene, optionally wherein the transcription product comprises, consists essentially of, or consists of a nucleotide sequence as set forth in any of SEQ ID NOs: 1, 3, 5, 7, 9, 11, and 12. In some embodiments, the inhibitory nucleic acid comprises a single stranded polynucleotide having self-complementary sense and antisense regions, wherein the antisense region comprises a sequence complementary to a region of a target nucleic acid molecule. In some embodiments, the inhibitory nucleic acid comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active inhibitory nucleic acid capable of mediating RNAi. In some embodiments, the inhibitory nucleic acid is an siRNA or an shRNA, which in some embodiments comprises, consists essentially of, or consists of a nucleotide sequence as set forth in any of SEQ ID NO: 77 (TRIM37 shRNA #1) and 78 (TRIM37 shRNA #2).

In some embodiments, the inhibitor of TRIM37 activity is a small interfering RNA (siRNA) or short hairpin RNA (shRNA) that targets a transcription product of a TRIM37 gene, optionally wherein the transcription product comprises, consists essentially of, or consists of a nucleotide sequence amino acids set forth in any of SEQ ID NOs: 1, 3, 5, 7, 9, 11, and 12.

Exemplary shRNAs that target TRIM37 gene products include the oligonucleotides set forth as SEQ ID NOs: 77 and 78, although it would be understood that any shRNA that targets a TRIM37 gene product could be employed in the compositions and methods of the presently disclosed subject matter.

Antisense technology has been demonstrated to be an effective method for modifying the expression levels of gene products (see for example, U.S. Pat. No. 8,765,703, U.S. Pat. No. 8,946,183, and U.S. Pat. Publication No. 2015/0376615, which are incorporated herein by reference in their entirety). In some embodiments, antisense technology works by interfering with the translation of mRNAs, and in some embodiments by interfering with the known steps in the normal processing of mRNA. Briefly, RNA molecules are transcribed from genomic DNA in the nucleus of the cell. These newly synthesized mRNA molecules, called primary mRNA or pre-mRNA, must be processed prior to transport to the cytoplasm for translation into protein at the ribosome. Such processing includes the addition of a 5′ methylated cap and the addition of a poly(A) tail to the 3′ end of the mRNA.

Maturation of 90-95% of mammalian mRNAs then occurs with splicing of the mRNA. Introns (or intervening sequences) are regions of a primary transcript (or the DNA encoding it) that are not included in the coding sequence of the mature mRNA. Exons (expressed sequences) are regions of a primary transcript (or the DNA encoding it) that remain in the mature mRNA when it reaches the cytoplasm. During the splicing process, exons in the pre-mRNA molecule are spliced together to form the mature mRNA sequence. Splice junctions, also referred to as splice sites, are utilized by cellular apparatus to determine which sequences are removed and where the ends to be joined start and stop. Sequences on the 5′ side of the junction are called the 5′ splice site, or splice donor site, whereas sequences on the 3′ side the junction are referred to as the 3′ splice site, or the splice acceptor site. In splicing, the 3′ end of an upstream exon is joined to the 5′ end of the downstream exon. Thus, the un-spliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Cryptic splice sites are those which are less often used but may be used when the usual splice site is blocked or unavailable. The use of different combinations of exons by the cell can result in multiple mRNA transcripts from a single gene.

In one application of antisense technology, an antisense oligonucleotide (AON) binds to a mRNA molecule transcribed from a gene of interest and inactivates (“turns off”) the mRNA by increasing its degradation or by preventing translation or translocation of the mRNA by steric hindrance. The end result is that expression of the corresponding gene (i.e., final production of the protein encoded by the corresponding gene) is prevented.

Alternatively, antisense technology can be used to affect splicing of a gene transcript. In this application, the antisense oligonucleotide binds to a pre-spliced RNA molecule (pre-messenger RNA or pre-mRNA) and re-directs the cellular splicing apparatus, thereby resulting in modification of the exon content of the spliced mRNA molecule. Thus, the overall sequence of a protein encoded by the modified mRNA differs from a protein translated from mRNA, the splicing of which was not altered (i.e., the full length, wild-type protein). The protein that is translated from the altered mRNA may be truncated and/or it may be missing critical sequences required for proper function. Typically, the compounds used to affect splicing are, or contain, oligonucleotides having a base sequence complementary to the mRNA being targeted. Such oligonucleotides are referred to herein as “antisense oligonucleotides” (AONs).

This disclosure provides antisense technology to modulate splicing of mRNA encoding a TRIM37 protein, thereby causing a decrease in the amount or “level” of TRIM37 protein expressed by a cell. Accordingly, a method as set forth herein can generally be accomplished by contacting a cell expressing a TRIM37 transcript, with an antisense oligomer targeted to a region of the TRIM37 pre-mRNA. Such contact results in uptake of the antisense oligomer by the cell, hybridization of the oligomer to the TRIM37 mRNA, and subsequent modulation of splicing of the TRIM37 pre-mRNA. In some embodiments, such modulation of splicing of the TRIM37 mRNA decreases expression of TRIM37, optionally expression of TRIM37 in a tumor and/or a cancer cell.

Methods and compositions for designing and administering antisense oligomers to cells and to subjects are described in, for example, U.S. Pat. Nos. 7,973,015; 8,236,557; 8,268,962; 8,304,398; 8,361,979; 8,802,645; 9,080,170; 9,238,042; 9,598,703; 9,738,891; 9,862,945; 10,030,894; 10,188,633; 10,590,420; and 10,781,541, the entire disclosure of each of which is incorporated by reference in its entirety.

In some embodiments, the composition is part of a nanoscale or microscale delivery vehicle, wherein the delivery vehicle is optionally selected from the group consisting of a liposome, a lipo/polymer, a microparticle, and a nanoparticle, or any combination thereof. In some embodiments, the delivery vehicle comprises a nanoparticle, wherein the nanoparticle encompasses the inhibitor of TRIM37 activity and, in some embodiments, one or more chemotherapeutically active agents.

In some embodiments, the delivery vehicle is designed to degrade in the subject in order to release the inhibitor of TRIM37 activity and/or one or more chemotherapeutically active agents to the subject over a period of time. In some embodiments, the delivery vehicle releases the one or more inhibitors of TRIM37 activity and one or more chemotherapeutically active agents to the subject’s circulation and/or a cell, tissue, and/or organ of subject over the period of time. In some embodiments, the delivery vehicle is designed to degrade subsequent to contact with the subject’s digestive system or circulatory system. In some embodiments, the delivery vehicle is designed to degrade in the subject to release at least about 50% of the one or more inhibitors of TRIM37 activity and one or more chemotherapeutically active agents over a period of time of at least 30 minutes, at least 1 hour, at least 6 hours, at least 12 hours, at least 24 hours, or longer than 24 hours.

In some embodiments, the presently disclosed subject matter provides for the use of compositions comprising liposomes. Liposomes can be prepared by any of a variety of techniques that are known in the art. See e.g., Betageri et al., 1993; Gregoriadis, 1993; Janoff, 1999; Lasic & Martin, 1995; and U.S. Pat. Nos. 4,235,871; 4,551,482; 6,197,333; and 6,132,766, each of which is incorporated herein by reference in its entirety. Temperature-sensitive liposomes can also be used, for example THERMOSOMES™ as disclosed in U.S. Pat. No. 6,200,598, which is incorporated herein by reference in its entirety. Entrapment of an active agent within liposomes of the presently disclosed subject matter can also be carried out using any conventional method in the art. In preparing liposome compositions, stabilizers such as antioxidants and other additives can be used.

In some embodiments, the liposome and/or the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide(polyethyleneglycol-2000)] (DSPE-PEG2000-Maleimide), or any combination thereof.

In some embodiments, the composition comprises a targeting moeity that targets the composition or a component thereof to a desired location (referred to as a “target” or a “targeted site”) such as, but not limited to a cell (e.g., a tumor cell and/or cancer cell), tissue, or organ. Exemplary targeting moieties include antibodies and antigen-binding fragments thereof. In some embodiments, a targeting moiety comprises an antibody or antigen-binding fragment thereof that binds to a FOLR1 gene product. As used herein, the term “FOLR1 gene product” refers to a peptide, polypeptide, or protein that is a product of the folate receptor-α (FOLR1) gene including but not limited to a human FOLR1 polypeptide. The human FOLR1 genetic locus is present on chromosome 11 and is associated with several transcription products including, but not limited to Accession Nos. NM_016724.3, NM_016725.3, NM_000802.3, and NM_0167829.3 of the GENBANKⓇ biosequence database, which encode Accession NOs. NP_057936.1, NP_057937.1, NP_000793.1, and NP_057941.1 present therein, respectively. By way of example and not limitation, an anti-FOLR1 antibody or a fragment or derivative thereof can be Farletuzumab, which is a humanized anti-folate receptor-α antibody described, for example, in PCT International Patent Application Publication Nos. WO 2012/119077and WO 2019/099374, in U.S. Pat. No. 9,599,621, in U.S. Pat. Application Publication No. 2020/0283537, and in Konner et al., 2010, each of which is incorporated by reference in its entirety.

In some embodiments, the TRIM37 inhibitor is encompassed by a targeting moieity (e.g., an antibody) is conjugated to and/or otherwise associated with a liposome, a microparticle, or a nanoparticle. In some embodiments, the targeting moiety (e.g., the antibody) is conjugated to the liposome, microparticle, or nanoparticle via a linker, which in some embodiments can comprise a peptide sequence through which the targeting moiety (e.g., the antibody) is conjugated to the liposome, microparticle, or nanoparticle. In some embodiments, the linker sequence comprises an Fc-linkered peptide sequence comprising a cysteine. In some embodiments, the Fc-linkered peptide sequence is present at the C-terminus of a heavy chain of the targeting antibody and/or the fragment or derivative thereof.

The presently disclosed subject matter also relates in some embodiments to purified and isolated antibodies, or fragments thereof comprising at least one paratope, comprising a linker sequence through which the antibody can be conjugated to a carrier, wherein the linker sequence comprises the amino acid sequence ((X)₃Cys(X)₃, wherein each X is independently any amino acid. In some embodiments, the linker sequence comprises an Fc-linkered sequence containing a cysteine. In some embodiments, the Fc-linkered sequence is present at the C-terminus of a heavy chain of the antibody. In some embodiments, the purified and isolated antibody or the fragment thereof specifically binds to a folate receptor.

In some embodiments, to generate a unique and effective antibody conjugate platform for siRNA or nanoparticles linkage, we have engineered a novel linker sequence ((X)₃Cys(X)₃ amino acid sequence), which is in continuation of the carboxy terminal of heavy chain Fc (called Fc-linkered). In some examples, we have engineered a unique cysteine (Cys) residue in the Fc-linkered sequence (examples presented below) of the hole chain of both Farletuzumab and LK26 antibodies. The knob chain of both Farletuzumab and LK26 antibodies is exactly similar to hole chain except for the presence of this unique cysteine residue. With the newly engineered cysteine residue in Fc-linkered hole chain, unlike described THIOMAB-siRNA conjugate platform (Cuellar et al., 2015), any chemical-linker can be easily covalently linked (site specific, plug-play manner) without the reduction of antibody using maleimide chemistry (additional data disclosed herein). Importantly, we have further characterized that addition of Fc-linkered sequence with unique cysteine does not affect binding affinity, activity and Fc effector functions of these antibodies (data disclosed herein).

In some embodiments of the presently disclosed purified and isolated antibodies and fragments thereof, the carrier is a nanoparticle. In some embodiments, the carrier comprises an inhibitor of TRIM37 activity. In some embodiments, the inhibitor of TRIM37 activity is selected from the group consisting of an anti-sense oligonucleotide, a small molecule inhibitor, and a combination thereof. In some embodiments, the nanoparticle is liposome-based. In some embodiments, the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide(polyethyleneglycol-2000)] (DSPE-PEG2000-Maleimide), or any combination thereof.

Other lipid carriers can also be used in accordance with the presently disclosed subject matter, such as lipid microparticles, micelles, lipid suspensions, and lipid emulsions. See e.g., Labat-Moleur et al., 1996; U.S. Pat. Nos. 5,011,634; 6,056,938; 6,217,886; 5,948,767; and 6,210,707, each of which is incorporated herein by reference in its entirety.

Delivery time frames can be provided according to a desired treatment approach. By way of example and not limitation, the first delivery vehicle can deliver substantially all of the provided active agent within 24 hours after administration wherein the second delivery vehicle can deliver a certain much smaller amount within the first 24 hours, first 3 days, first week, and substantially all within the first 2, 3, 4, 5, 6, or 7 weeks, as desired. Thus, the duration of the delivery can be altered with the chemistry of the delivery vehicle.

The delivery vehicles can comprise nano-, submicron-, and/or micron-sized particles. In some embodiments, the delivery vehicles are about 50 nm to about 1 µm in their largest dimensions. Thus, in some embodiments the delivery vehicle can comprise a nanoparticle, a microparticle, or any combination thereof. As used herein, the terms “nano”, “nanoscopic”, “nanometer-sized”, “nanostructured”, “nanoscale”, and grammatical derivatives thereof are used synonymously and interchangeably and mean nanoparticles and nanoparticle composites less than or equal to about 1,000 nanometers (nm) in diameter. Similarly, the terms “micro”, “microscopic”, “micrometer-sized”, “microstructured”, “microscale”, and grammatical derivatives thereof are used synonymously and interchangeably and mean microparticles and microparticle composites that are larger than 1,000 nanometers (nm) but less than about 5, 10, 25, 50, 100, 250, 500, or 1000 micrometers in diameter.

The term “delivery vehicle” as used herein thus denotes a carrier structure which is biocompatible with and sufficiently resistant to chemical and/or physical destruction by the environment of use such that a sufficient amount of the delivery vehicles remain substantially intact after deployment at a site of interest. If the active agent is to enter a cell, tissue, or organ in a form whereby it is adsorbed to the delivery vehicle, the delivery vehicle must also remain sufficiently intact to enter the cell, tissue, or organ. Biodegradation of the delivery vehicle is permissible upon deployment at a site of interest.

As used herein, the term “biodegradable” means any structure, including but not limited to a nanoparticle, which decomposes or otherwise disintegrates after prolonged exposure to physiological conditions. To be biodegradable, the structure should be substantially disintegrated within a few weeks after introduction into the body.

Biodegradable biocompatible polymers can be used in drug delivery systems (Soppimath et al., 2001; Song et al., 1997; U.S. Pat. Application Publication Nos. 2011/0104069, 2013/0330279, 2018/0078657, 2019/0091280, and 2020/0038452, and U.S. Pat. Nos. 7,332,586; 7,901,711; 8,137,697; 8,449,915; and 8,663,599, each of which is incorporated herein by reference in its entirety). The biodegradability and biocompatibility of poly(lactic acid) (PLA), poly(lactide-co-glycolide) (PLGA), and polyanhydrides (PAH) have been demonstrated. Some of the advantages of these materials include administration in high concentrations of the drug locally with low systemic levels, which reduces systemic complications and allergic reactions (Calhoun et al., 1997). Additionally, no follow-up surgical removal is required once the drug supply is depleted (Mandal et al., 2002). Biodegradation occurs by simple hydrolysis of the ester backbone in aqueous environments such as body fluids. The degradation products are then metabolized to carbon dioxide and water (de Faria et al., 2005). Several techniques have been developed to prepare nanoparticles loaded with a broad variety of drugs using PLGA and to some extent with PAH (Lamprecht et al., 1999; Astete et al., 2006; Hans et al., 2002; Kumar et al., 2004; Laurencin et al., 2001; Gonsalves et al., 1998; Kwon et al., 2001).

In some embodiments, the composition can comprise a pharmaceutically acceptable carrier, diluent, or excipient. As used herein, the term “pharmaceutically acceptable” and grammatical variations thereof, as it refers to compositions, carriers, diluents and reagents, means that the materials are capable of administration to or upon a vertebrate subject without the production of undesirable physiological effects such as nausea, dizziness, gastric upset, fever and the like. In some embodiments, the “pharmaceutically acceptable” refers to pharmaceutically acceptable for use in human beings.

Compositions in accordance with the presently disclosed subject matter generally comprise an amount of the desired delivery vehicle (which can be determined on a case-by-case basis), admixed with an acceptable pharmaceutical diluent or excipient, such as a sterile aqueous solution, to give an appropriate final desired concentration in accordance with the dosage information set forth herein, and/or as would be apparent to one of ordinary skill in the art upon a review of the instant disclosure, with respect to the antibiotic. Such formulations will typically include buffers such as phosphate buffered saline (PBS), or additional additives such as pharmaceutical excipients, stabilizing agents such as BSA or HSA, or salts such as sodium chloride. Such components can be chosen with the preparation of composition for local, and particularly topical, administration in mind.

In some embodiments, an antibody or a fragment or derivative thereof is conjugated to a delivery vehicle (e.g., a liposome, a microparticle, and/or a nanoparticle). Methods of conjugating polypeptides such as antibodies and antibody fragments and derivatives to delivery vehicles are known and include, for example, U.S. Pat. Nos. 8,364,234; 9,603,798; 9,624,303; and 10,031,134, each of which is incorporated herein by reference in its entirety.

However, it is noted that generally employed conjugation methodologies can result in negative impacts to the biological activities of the antibodies and antibody fragments and derivatives, such as but not limited to the binding affinities, biological activities, and/or Fc effector functions of the antibodies and antibody fragments and derivatives. Given that it would be undesirable to reduce any of these parameters, a novel peptide linker with the amino acid sequence (X)₃Cys(X)₃, wherein “X” can be any amino acid can be employed to generate a unique and effective antibody conjugate platform for linkage of antibodies, fragments, and derivatives thereof to delivery vehicles including but not limited to liposomes, microparticles, and/or nanoparticles. As set forth herein, in some embodiments an antibody, fragment, or derivative thereof that is desired to conjugation to a delivery vehicle can be modified to include the amino acid sequence (X)₃Cys(X)₃, in some embodiments at or near the C-terminus of the antibody, fragment, or derivative’s heavy chain. As a result, a modified antibody, fragment, or derivative thereof that includes a (X)₃Cys(X)₃ extension of the carboxy terminus of heavy chain Fc (called Fc-Linkered) ca be produced.

For example, as disclosed herein a unique cysteine (Cys) residue in the Fc-linkered sequence of the hole chain of both Farletuzumab and LK26 antibodies has been produced. The knob chain of both Farletuzumab and LK26 antibodies is exactly similar to hole chain except for the presence of this unique cysteine residue. With the newly engineered cysteine residue in Fc-linkered hole chain, unlike the THIOMAB-siRNA conjugate platform described in Cuellar et al., 2015, any chemical-linker can be easily covalently linked (e.g., in a site specific, plug-play manner) without requiring the reduction of the antibody, fragment, or derivative using maleimide chemistry. In some embodiments and as demonstrated herein, addition of an Fc-linkered sequence with the unique cysteine does not affect the binding affinity, the biological activity, or the Fc effector functions of these antibodies, fragments, and derivatives (see the discussion in the EXAMPLES below).

By way of example and not limitation, an antibody or a fragment or derivative thereof can be conjugated to a delivery vehicle using a non-cysteine linker (e.g., a peptide encoded by SEQ ID NO: 97 having an amino acid sequence comprising, consisting essentially of, or consisting of an amino acid sequence of SEQ ID NO: 98) or a cysteine-containing peptide linker (e.g., a peptide encoded by SEQ ID NO: 99 having an amino acid sequence comprising, consisting essentially of, or consisting of an amino acid sequence of SEQ ID NO: 100). Any antibody or fragment or derivative thereof can be so modified, including but not limited to a heavy chain of antibody LK26 (SEQ ID NO: 93), a light chain of antibody LK26 (SEQ ID NO: 94), a heavy chain of antibody Farletuzumab (SEQ ID NO: 95), or a light chain of antibody Farletuzumab (SEQ ID NO: 96). The modified antibody or a fragment or derivative thereof can then be conjugated to a delivery vehicle (e.g., a liposome, a microparticle, and/or a nanoparticle) using techniques disclosed herein and/or those known in the art.

### II.B. Formulations

The compositions of the presently disclosed subject matter can be administered in any formulation or route that would be expected to deliver the compositions to the subjects and/or target sites present therein.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject. For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

### II. C. Routes of Administration

By way of example and not limitation, suitable methods for administering a composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intratumoral injection, intranasal delivery, hyper-velocity injection/bombardment. Where applicable, continuous infusion can enhance drug accumulation at a target site (see e.g., U.S. Pat. No. 6,180,082, which is incorporated herein by reference in its entirety). In some embodiments, a composition comprising a nanoparticle and/or an exosome is administered intratumorally.

Thus, exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, intratumoral, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated (e.g., delivered directly to the tumor and/or the cancer to be treated).

### II.D. Dose

An effective dose of a composition of the presently disclosed subject matter is administered to a subject in need thereof. An “effective amount” or a “therapeutic amount” is an amount of a composition sufficient to produce a measurable response. Exemplary responses include biologically or clinically relevant responses in subjects such as but not limited to an improvement in a symptom. Actual dosage levels of the compositions of the presently disclosed subject matter can be varied so as to administer an amount of the composition that is effective to achieve the desired response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore an “effective amount” can vary. However, using the methods described herein, one skilled in the art can readily assess the potency and efficacy of a composition of the presently disclosed subject matter and adjust the regimen accordingly.

As such, after review of the instant disclosure, one of ordinary skill in the art can tailor the dosages to an individual subject, taking into account the particular formulation, method of administration to be used with the composition, and particular disease, disorder, and/or condition treated or biologically relevant outcome desired. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

By way of example and not limitation, in some embodiments the dosage of the inhibitor of TRIM37 activity is sufficient to sensitize a tumor and/or a cancer to sensitize the tumor and/or the cancer to a chemotherapeutic activity of an anti-tumor and/or anti-cancer therapeutic agent, which in some embodiments can be a chemotherapeutic agent. By way of example and not limitation, the compositions of the presently disclosed subject matter can be employed to treat TNBC, and as such, the anti-tumor and/or anti-cancer therapeutic agent can comprise one or more of an anthracycline including but not limited to Adriamycin, an alkylating agent such as Cytoxan, a taxane such as paclitaxel (including conjugated paclitaxel such as Abraxane) or docetaxel, capecitabine, gemcitabine, eribulin, 5-fluorouracil (5FU), or any combination thereof. Other non-chemotherapeutic anti-tumor and/or anti-cancer therapeutic agents include immunotherapy agents such as but not limited to immune checkpoint inhibitors. Exemplary immune checkpoint inhibitors include cytotoxic T-lymphocyte antigen 4 (CTLA4) inhibitors such as but not limited to anti-CTLA4 antibodies, programmed cell death 1 (PD-1) inhibitors such as but not limited to anti-PD-1 antibodies, and programmed cell death ligand 1 (PD-L1) inhibitors such as anti-PD-L1 antibodies. Particular immune checkpoint inhibitors can be selected from the group consisting of Ipilimumab, Tremelimumab, Nivolumab, Pidilizumab, Pembrolizumab, AMP514, AUNP12, BMS-936559/MDX-1105, Atezolizumab, MPDL3280A, RG7446, RO5541267, MEDI4736, Avelumab, and Durvalumab.

### III. Methods

In some embodiments, the presently disclosed subject matter relates to the use of the presently disclosed compositions in preventive and/or treatment methods. Thus, in some embodiments the presently disclosed subject matter relates to methods for sensitizing a tumor, a cancer, and/or a cell present in and/or derived therefrom in a subject to a therapeutic agent. In some embodiments, the methods comprise administering to the subject a composition comprising an effective amount of an inhibitor of TRIM37 activity.

As used herein, the phrases “effective amount” and “therapeutically effective amount” are used interchangeably and refer to an amount of a compound or composition sufficient to produce a desired effect, such as but not limited to alleviating symptoms of a condition, disease, or disorder. In some embodiments, an effective amount is an amount of a compound or composition that sensitizes a cancer in a subject to a therapeutic agent, which in some embodiments refers to an amount that, when administered to a subject or otherwise brought into contact with a tumor, cancer, or a cell present in and/or otherwise derived therefrom, including but not limited to a metastatic cell derived therefrom, results in the tumor, cancer, or the cell present in and/or otherwise derived therefrom becoming more sensitive to a particular therapeutic agent.

As used herein, the term “sensitizes” and grammatical variants thereof refers to a set of circumstances whereby a tumor, cancer, and/or a cell present in and/or otherwise derived therefrom that has been contacted with the compound and/or composition (also referred to herein as a “sensitizing compound and/or composition”) either dies, senesces, or grows more slowly that it would have had it not been contacted with the compound and/or composition. In some embodiments, the growth of (in some embodiments, the growth rate of) the tumor, cancer, and/or a cell present in and/or otherwise derived therefrom is reduced (in some embodiments, reduced to zero) as a result of the contacting, which would not have occurred in the absence of the contacting.

In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

Thus, in some embodiments the presently disclosed subject matter relates to administering a TRIM37 inhibitor to a subject with a tumor and/or a cancer in order to sensitize the tumor and/or the cancer to a therapeutic agent. In some embodiments, the therapeutic agent is a chemotherapeutic agent.

In some embodiments, the presently disclosed methods can further comprise administering the therapeutic agent (e.g., the chemotherapeutic agent) to the subject. Thus, in some embodiments the presently disclosed subject matter also provides methods for treating tumors and/or cancers in subjects in need thereof. In some embodiments, the presently disclosed methods comprise, consist essentially of, or consist of administering to the subject a first composition comprising an effective amount of an inhibitor of TRIM37 activity and a second composition comprising an effective amount of an anti-cancer therapeutic agent. In some embodiments, the first and the second compositions are components of the same composition, and in some embodiments the first and the second compositions are separate compositions. In some embodiments, the first and second compositions are co-administered, and in some embodiments the compositions are administered separately. In some embodiments, the first composition comprising the effective amount of an inhibitor of TRIM37 activity is administered prior to administering the second composition, wherein the administering of the first composition is at a time and in an amount to sensitize a tumor and/or a cancer, and/or a cell thereof to the anti-cancer therapeutic activity of the second composition. In some embodiments, the cancer is triple negative breast cancer (TNBC).

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Cell lines and cell culture. MDA-MB-231-luc-D3H2LN-BMD2b (231-2b; provided by Takahiro Ochiya, National Cancer Center Research Institute, Japan), MDA MB 231, MDA MB 468 and HCC1806 (provided by Michael J. Lee, University of Massachusetts Medical School) cells were maintained in RPMI medium supplemented with 10% fetal bovine serum (FBS, Invitrogen) at 37° C. and 5% CO₂. MCF10A (provided by Michael R. Green, University of Massachusetts Medical School), MCF10AT (provided by Michael R. Green, University of Massachusetts Medical School) and MCF10A TP53-/- (provided by David Weber and Michele Vitolo) cells were maintained in DMEM/F12 supplemented with 5% horse serum (Invitrogen), EGF (Peprotech), hydrocortisone (Sigma), cholera toxin (Sigma), insulin (GIBCO) and penicillin/streptomycin (Life Technologies). MCF7 (National Cancer Institute (NCI)), MDA MB 468 (provided by Michael J. Lee, University of Massachusetts Medical School), HCC1806 (provided by Michael J. Lee, University of Massachusetts Medical School) and HCC1806RR (provided by Sophia Ran, Southern Illinois University School of Medicine) cells were maintained in DMEM supplemented with non-essential amino acids (NEAA) and 10% fetal bovine serum (FBS). MCF10A, MCF7, MCF10AT, HCC1806RR (provided by Sophia Ran) and TP53^{-/-} MCF10A (provided by David Weber and Michele Vitolo) were cultured as described previously (Weiss et al., 2010; Volk-Draper et al., 2012; Bhatnagar et al., 2014). Cells cultured at the same time were pooled together and then seeded after counting in a 6-well or 10-cm dish. Cells were then subjected, in a random order, to treatment with a control or test different biologics, which included shRNA, sgRNA, vectors, and small molecule inhibitors. Cells were routinely tested for mycoplasma using PlasmoTest kit from (Invivogen).

Animal Care. NOD. Cg-Prkdc^{scid} Il2rg^{tm1Wjl}SzJ and Balb/cJ were obtained from the Jackson Laboratory. The mice were housed in a specific-pathogen-free facility accredited by the American Association of Laboratory Animal Care. All animal studies were approved (#4112 and #4222) by the Institutional Animal Care and Use Committee of the University of Virginia School of Medicine.

RNA interference. For stable shRNA knockdowns, cells were seeded in a six-well plate to 60-80% confluency and subsequently transduced with 200-500 µl lentiviral particles expressing shRNAs (obtained from Open Biosystems/Thermo Scientific through the UMMS RNAi Core Facility, listed in Table 6) in a total volume of 2 ml of appropriate media supplemented with ~6-10 µg/ml polybrene. Media was replaced after overnight incubation and cells were subjected to puromycin selection (2 µg/ml) for at least 3 days as described previously (Bhatnagar et al., 2014).

Quantitative RT-PCR (qRT-PCR). Total RNA was isolated and reverse transcribed using Superscript II Reverse Transcriptase (Invitrogen). Quantitative real-time PCR was performed as described previously (Bhatnagar et al., 2014) using primers listed in Table 5. Gene expression was normalized to Gapdh. Controls lacking reverse transcriptase were carried out in parallel to rule out the possibility of DNA contamination. Each sample was analyzed at least two independent times and the results from at least three different biological triplicates are presented.

TRIM37 and p53 overexpression. A TRIM37 cDNA clone (Origene) was sub-cloned into the vector pcDNA3.1(-) (Sigma) using Not1 and BamH1 sites, and verified by full-length sequencing. TRIM37 expression vector was linearized with PciI and transfected into cells using either 4D Amaxa nucleofection system (Lonza) or Effectene reagent (Qiagen). A p53 cDNA clone (provided by Marty Mayo, University of Virginia School of Medicine) was transfected into cells using Effectene reagent (Qiagen). After 24 hrs., cell medium was replaced by appropriate selection markers and stable cells were selected.

Comet assay. Cells were treated with 1 µM of Dox, and harvested at 24 hrs. Neutral comet assays with ethidium bromide staining (Invitrogen) were performed as described previously (Zhang et al., 2016). The quantitation of tail DNA was done using OpenComet software (Gyori et al., 2014).

DSB repair efficiency assay. NHEJ and HR assays were designed and performed as described previously (Bennardo et al., 2008; Pierce et al., 1999). MDA-MB-468 cells were stably transfected with pimEJ5GFP for the NHEJ assay or pDRGFP for the HR assay. Stable cell lines were infected with control or TRIM37 shRNA lentivirus, followed with transfection with pCBASceI (Richardson et al., 1998). After 24 hrs., cells were analyzed for GFP expression by flow cytometry and normalized to non-silencer (NS) control for each biological replicate.

Colony formation assay. Cells were plated in 6-well plates after indicated treatment and cultured until visible colonies were observed. Cells were treated with chemotherapeutic drugs as indicated at the following concentrations: 0.1-1 µM doxorubicin (Cayman Chemical), 0.1-1 µM Daunorubicin (Cayman Chemical), 250-500 µM temozolomide (Cayman Chemical), 0.5-25 µM etoposide (Cayman Chemical) and 1-10 µM cisplatin (Cayman Chemical) for 24 hrs. Colonies were fixed (100% methanol, 37° C.) and stained with 0.1% crystal violet dissolved in 20% methanol/80% PBS. Cells were imaged using a Chemidoc (BioRad) and colonies were counted.

Caspase 3 activity assay. The Caspase 3 assay was performed using Caspase-3 assay kit (BD Pharmingen) according to the manufacturer’s instructions.

Directed-ChIP assays. ChIP assays were performed as described previously (Bhatnagar et al., 2014) using cell extracts prepared 1 day after Cas9 transduction or inhibitor treatment, and antibodies against BMI1 (Abcam), H2Aub (Cell Signaling Technology), TRIM37 (Abcam), STAT1 (Cell Signaling Technology), STAT3 (Cell Signaling Technology), E2F1 (Cell Signaling Technology), and as a negative control, IgG. The sequences of primers used for amplifying ChIP products are listed in Table 5. Each ChIP experiment was carried out at least three independent times and the results from at least three biological experiments, with technical duplicates, are shown.

Sucrose gradient fractionation. Sucrose gradient sedimentation analysis was performed as described previously (Bhatnagar et al., 2014). Briefly, 10-40% gradients were formed by layering 200 µl NEB1 buffer containing 10%, 20%, 30% or 40% sucrose in a 13 x 51 mm centrifuge tube (Beckman) and allowed to equilibrate at room temperature for 2 hrs. Gradients were chilled, loaded with 500 mg HCC1806 extract (adjusted to a volume of 200 µl) or 200 µl molecular weight markers (Sigma MW-GF-1000), and centrifuged in a Beckman SW 55 Ti rotor at 214,000 g for 14 hrs. Twenty-five fractions of 200 µl were collected. For the markers, 20 µl of each fraction was electrophoresed and Coomassie stained. For the gradient fractions, 20 µl of fractions was analyzed by immunoblotting using TRIM37 (Abcam), KU80 (Cell Signaling Technology), KU70 (Cell Signaling Technology), RAD51C (Cell Signaling Technology), RAD50 (Cell Signaling Technology), MRE11 (Cell Signaling Technology) and NBS1 (Cell Signaling Technology).

Immunoblotting. Cell extracts were prepared by lysing cell pellet in RIPA buffer supplemented with 1 mM sodium ortho-vanadate and 10 mM PMSF. To prepare protein extract from mouse tissue, it was homogenized in lysis buffer (1% SDS, 1 mM sodium ortho-vanadate, 10 mM Tris pH 7.4 and protease inhibitor). Immunoblots were probed using antibodies against TRIM37 (Abcam), PARP (Cell Signaling Technology), p21 (Cell Signaling Technology), γ-ph-H2AX (Cell Signaling Technology), RAD51C (Invitrogen), ph-ATM (Cell Signaling Technology), ph-STAT1 (Cell Signaling Technology), ph-STAT3 (Cell Signaling Technology), ph-STAT5 (Cell Signaling Technology), BIM (Abcam), FOLR1 (R&D) and α-tubulin (Invitrogen).

Co-immunoprecipitation. MDA MB 468 nuclear extract (~1 mg) was incubated with either TRIM37 (Abcam), XRCC5 (Cell Signaling Technology), KU80 (Invitrogen), KU70 (Invitrogen), NBS1 (Invitrogen), or RAD51C (Invitrogen) antibody at 4° C. for 24 hrs. in the presence of ethidium bromide (100 µg/ml). Immune complexes were captured on protein A/G-Agarose (GenDEPOT), washed three times in NEB1 buffer, and eluted by boiling for 10 min in SDS sample buffer. Immunoprecipitated proteins were analyzed by immunoblotting as described earlier. Input lanes represent ~1-5% of extract loaded in the immunoprecipitation lanes.

CRISPR/Cas9 targeting. The Gapdh sgRNAs (Sanjana et al., 2014) were cloned in pLentiCrispr v2 plasmid (Addgene) and packaged into virus as recommended by the manufacturer. Cells were seeded in a 10 cm-well plate to ~60-80% confluency and subsequently transduced with 1000-1500 µl lentiviral particles expressing sgRNAs in 6 ml of appropriate media, supplemented with 10 mg/ml polybrene as generally described in Bhatnagar et al., 2014. Cells were harvested after 24 hours for ChIP analysis.

Chemical inhibitor treatment. MDA-MB-468 cells were treated for 24 hrs with 10 µM Dox and small molecule inhibitors at the following concentrations: 10 µM KU000553 (Cayman Chemical), 50 µM AG490 (Cayman Chemical), and 40 µM HLM006474 (Sigma-Aldrich) for 24 hrs.

MTT cell proliferation assay. MTT was performed using MTT Cell Proliferation Assay Kit (Trevigen) according to the manufacturer’s instructions.

Anoikis assay. 3 × 10⁵ cells/ml cells were suspended in recommended media containing 0.5% methylcellulose (Sigma) and plated on poly-HEMA (Sigma)-coated 6-well plates. Where indicated cells were cultured in poly-HEMA for 1-5 days prior to seeding for colony formation assay.

Recombinant Antibody Cloning. Coding sequences for Farletuzumab (anti-human FOLR1; commercially available from Morphotek, Inc., Exton, Pennsylvania, United States of America) and LK26 (anti-mouse FOLR1; commercially available from Abcam, Cambridge, Massachusetts, United States of America) antibodies were cloned, engineered, expressed, and purified as described previously (Shivange et al., 2018). The detailed amino acid sequences of the recombinant antibodies (Farletuzumab and LK26) were published along with the described engineering and generation of Knob-Hole antibodies (Shivange et al., 2018). To generate a unique and effective antibody conjugate platform for siRNA or nanoparticles linkage, we have engineered a novel linker sequence ((X)₃Cys(X)₃ amino acid sequence), which is in continuation of the carboxy terminal of heavy chain Fc (called Fc-Linkered). In particular, we have engineered a unique cysteine (Cys) residue in the Fc-linkered sequence (examples presented below) of the hole chain of both Farletuzumab and LK26 antibodies. The knob chain of both Farletuzumab and LK26 antibodies is exactly similar to hole chain except for the presence of this unique cysteine residue. With the newly engineered cysteine residue in Fc-linkered hole chain, unlike the THIOMAB-siRNA conjugate platform described in Cuellar et al., 2015, any chemical-linker can be easily covalently linked (site specific, plug-play manner) without the reduction of antibody using maleimide chemistry. Importantly, we have further characterized that addition of Fc-linkered sequence with unique cysteine does not affect binding affinity, activity and Fc effector functions of these antibodies as disclosed herein.

Recombinant Antibody Expression. Free style CHO—S cells (Invitrogen) were cultured and maintained according to supplier’s recommendations (Life technologies). A ratio of 2:1 (light chain, V_{L}: heavy chain, V_{H}) DNA was transfected using 1 mg/ml polyethylenimine (PEI) and cultured at 37° C. After 24 hrs., transfected cells were cultured at 32° C. for additional 9 days. Cells were fed every 2^{nd} day with 1:1 ratio of Tryptone feed and CHO Feed B. After 10 days, supernatant was harvested and antibodies were purified using HiTrap MabSelect SuRe (GE Healthcare) protein-A columns in Shivange et al., 2018. An Autodesk Inventor Professional 2020 was used to draw the design of antibody.

Antibody Purification. Transfected cultures were harvested after 10 days and filtered through 0.2-mm PES membrane filters (Millipore Express Plus). Cleaning-in-place (CIP) was performed for each column using 0.2 M NaOH wash (20 min.). Following cleaning, columns were washed 3 times with Binding buffer (20 mM sodium phosphate, 0.15 M NaCl, pH7.2). Filtered supernatant containing recombinant antibodies or antigens were passed through the columns at 4° C. Prior to elution in 0.1 M sodium citrate pH 3.0-3.6, the columns were washed 3 times with binding buffer, pH 7.0. The pH of eluted antibodies was immediately neutralized using 3 M sodium acetate, pH 9.0. After protein measurements at 280 nm, antibodies were dialyzed in PBS using Slide-A-Lyzer 3.5 K (Thermo Scientific). Antibodies were run on gel filtration columns (next section) to analyze the percent monomers. Whenever necessary a second step size exclusion chromatography was performed.

Size exclusion chromatography. The percent monomer of purified antibodies was determined by size exclusion chromatography as described previously (Shivange et al., 2018).

Binding studies by ELISA. Binding specificity and affinity of described Farletuzumab was determined by ELISA using the recombinant FOLR1 as described previously (Shivange et al., 2018). Briefly, recombinant FOLR1 protein was coated on 96-well plate by incubation overnight at 4° C. Different amounts of nanoparticles and free antibody were incubated in the coated plates and binding was detected by two-component peroxidase substrate kit (BD Biosciences).

Nanoparticle synthesis and packaging. Cholesterol, 1,2-dioleoyl-3-trimethylammonium-propane chloride salt (DOTAP), 1,2-dioleoyl-sn-glycero-3-phosphate (DOPA), and 1,2-distearoryl-sn-glycero-3-phosphoethanolamine-N-[maleimide-(polyethyleneglycol-2000)] ammonium salt (DSPE-PEG2000-Maleimide) were purchased from Avanti Polar Lipids, Inc. (Alabaster). All other chemicals were purchased from Sigma-Aldrich. TRIM37 and control antisense oligonucleotide sequences of primers used for targeting TRIM37 were synthesized by IDT (#198892132, #198892133).

TRIM37-ASO and control-ASO loaded bilayer nanoparticles (smart NPs and control NPs) were prepared as previously described with modification (see Chen et al., 2017; Lou et al., 2018). Briefly, 150 mM CaCl₂ with 0.5 mM ASO were dispersed in Cyclohexane/Igepal CO-520 (70:30 v/v) solution to form water-in-oil reverse micro-emulsion (Solution A). The phosphate phase was prepared by mixing 1.5 µM NaHPO₄ (pH=9.0) and 6.9 µM DOPA in Cyclohexane/Igepal CO-520 (70:30 v:v) solution (Solution B). CaP core was prepared by mixing Solution A and B with continuous stirring. Micro-emulsion was mixed with ethanol and centrifuged at 9,000 g. The pellet was dissolved in chloroform for lipid coating. For assembly of outer leaflet, CaP core was mixed with 0.5 µM DOTAP/Cholesterol (1:1), and 0.15 µM DSPE-PEG-2000-Maleimide. The residual lipid was dispersed in Tris-HCl buffer (5 mM, pH 7.4), and was gently sonicated for 5 min. CaP-bilipids nanoparticles were mixed with 0.56 µM engineered Farletuzumab solutions and incubated overnight at 4° C. Finally, CaP-bilipids nanoparticles were sterile filtered (0.22 µm) and stored at 4° C. for subsequent experiments. An Autodesk Inventor Professional 2020 was used to draw the design of smart nanoparticle.

Particle Characterization. The particle size was determined by dynamic light scattering (DLS) and the zeta potential of NPs was determined using a Zetasizer ZS (Malvern). The morphology of NPs was characterized by transmission electron microscope (JEOL JEM 1400 instrument, JEOL Ltd., Japan) at a voltage of 120 kV using CLSM (Zeiss LSM510 instrument, Carl Zeiss) at the Electron Microscopy Core, University of Virginia School of Medicine. Briefly, nanoparticles were dissolved in 0.01 M PBS (pH 7.4) and were negatively stained with 1% phosphotungstic acid (Bailey et al.) as described previously (Chen et al., 2017; Lou et al., 2018). A dialysis method was used to measure the release kinetics of ASO-loaded nanoparticles. In brief, 5 mg of drug-loaded NPs was dissolved in PBS with 0.1% (v/v) Tween 80, and dialyzed against 5 mL of that same buffer using ready-to-use dialysis tubes (MWCO 12,000-14,000; Fisher Scientific) under continuous stirring at 37° C. At predetermined time points, 10 µL aliquot was removed for quantification and replaced with fresh buffer. The concentration of the drug was determined by Nanodrop (Thermo Scientific).

FOLR1-overexpression cellular target and uptake. The protocols for cellular uptake assays were performed according to the previously published work (Shivange et al., 2018). HCC1806RR cells with either MCF7 or MCF10A were seeded onto an 8 well-chamber slide (Thermo Fisher Scientific Inc.) at a ratio of 1:1 with cell concentration of 30,000 per well in 100 µl of medium and cultured overnight at 37° C. The original medium was replaced with fresh medium containing 8.3x 10⁶ nanoparticles. Cells were incubated for indicated times and imaged for nanoparticle uptake by Zeiss Axio Observer 200 inverted microscope (Zeiss).

Immunofluorescence. Cells were stained with an anti-phospho yH2AX primary antibody (1:100, Santa Cruz Biotechnology). Cells were visualized on a Zeiss Axio Observer Live-Cell microscope and images were adjusted for contrast and brightness using AxioVision Software. For quantification, 100-500 cells in at least 10 different fields from biological and technical replicates were counted and scored for positive signal.

NSG tumor xenograft studies. All animal procedures were conducted under the accordance of the University of Virginia IACUC with approved protocol (4112 and 4222). For different cell lines, weight and aged matched female NSG mice were injected subcutaneously with 2×10⁶ TNBC cells in their right flank with indicated cell line in matrigel. 2×10⁶ HCC1806RR or 231-2b were injected in 100 µl volume. For Dox-induced TRIM37 upregulation studies in tumor, mice bearing ~200 mm³ HCC1806 tumors weight matched animals were randomly assigned into groups and injected with 2 mg/kg Dox. Tumors were harvested 24 hrs. following Dox treatment. For smart nanoparticles efficiency in vivo, mice bearing ~200 mm³ 231-2b tumors weight matched animals were randomly assigned into groups and injected with control or smart nanoparticles. At the endpoint, RNA and protein lysates were prepared from isolated tumors and analyzed by western blotting.

Patient sample analysis. Patients with triple negative breast cancers treated between 2001 and 2009 at Saint Louis hospital (Paris), by neoadjuvant chemotherapy regimen and with available pre and post-chemotherapy frozen tumor tissues were selected. Frozen tumor tissue sections were provided by the biological resource center of St Louis Hospital (agreement # DC 2009-929), following the Ethics and Legal national French rules for the patients’ information and consent (ANAES, HAS and INCA). Total RNAs were extracted using phenol/chloroform method (Chomczynski and Sacchi, 2006). The p53 status was determined by the FASAY yeast functional assay, as previously reported (Flaman et al., 1995; Lehmann-Che et al., 2010).

Bioluminescent imaging (BLI150 mg/kg luciferin (Perkin Elmer Inc.) was administered to mice intraperitoneally. Mice were imaged in ventral and dorsal position using Xenogen IVIS spectrum in vivo imaging system (Perkin Elmer Inc.). Luciferase signal in gross tissues was measured immediately after whole body imaging at the termination of the experiment as described previously (see Shivange et al., 2018).

Spontaneous metastatic tumor studies. All animal procedures were conducted under the accordance of the University of Virginia IACUC with approved protocol (4112 and 4222). 2×10⁶ 4T1 or 231-2b cells were injected into the inguinal mammary fat pad or right flank of 6 to 8-week-old female Balb/cJ or NSG mice and tumor growth was monitored thrice weekly, and tumor growth was calculated using the formula (length X width²)/2 as described in Bhatnagar et al., 2015). Where indicated, primary tumors were resected (in some instances when they reached ~500 mm³ in tumor size) and animals were allowed to develop lung metastases. Mice bearing TNBC tumors were weight matched and randomly assigned into groups that received 1.2 mg/kg smart or control nanoparticles at the indicated times. The lung metastases in the animals were monitored by BLI. At the termination of the experiment, animals were euthanized and indicated tissues were harvested and processed for histological examination and immunohistochemical staining or for qRT-PCR analysis. Metastatic burden was calculated either as the number of visible metastatic lesion in each organ or as the relative luminescence signal from gross organ tissue.

Experimental metastasis in vivo. 3 × 10⁵ 231-2b cells were inoculated directly into the left cardiac ventricle as described previously (Jenkins et al., 2005). Metastatic growth was monitored using BLI. Lungs, liver, femurs and brains were harvested post-mortem and processed for histological examination and gross analysis.

AST/ALT assays. To examine the hepatoxicity of the smart nanoparticle treatment, serum was isolated from blood samples collected from the treated mice at the termination of tumor studies. Samples were assessed for aspartate aminotransferase (Stephens et al.) and alanine aminotransferase (ALT) levels using Liquid AST (SGOT) reagent set (Pointe Scientific) and EnzyChrom Alanine Transaminase Assay Kit (Bioassay Systems) as per manufacturer’s instruction.

Hematoxylin and eosin and Ki67 staining. Animals were either perfused with 10% neutral buffered formalin or organs were collected and then fixed in 10% neutral buffered formalin and embedded in paraffin. H&E staining was performed by Research Histology Core and Ki67 was performed by Biorepository and Tissue Research Facility at University of Virginia School of Medicine (National Cancer Institute P30 UVA Cancer Center Grant).

Bioinformatic analysis. cBioPortal for cancer genomics (Cerami et al., 2012; Gao et al., 2013) was used to obtain TCGA expression z-scores for genes in METABRIC breast cancer samples (Pereira et al., 2016). FireBrowse (Broad Institute) was used to obtain normal breast tissue expression z-scores. Cooccurrence and mutual exclusivity analysis was performed with cBioPortal. Hazard ratios were assessed with Cox proportional hazard model for which TNBC and non-TNBC patients were stratified into high and low TRIM37 as third and first quartile as described previously (Spruance et al., 2004). For Kaplan-Meier analysis, the p-value was calculated with log-rank test. For boxplot analysis, TRIM37-regulated genes were stratified according to TRIM37 expression using z score thresholds (z>0.5, z<-0.5). For TRIM37 expression analysis according to ethnicity, data from GTEx (breast tissue samples), TCGA (normal adjacent breast tissue) and UNC (GSE111601) were combined and p-values were calculated by t-test. GTEx was used to obtain eQTLs for TRIM37.

RNA-seq analysis. 231-2b cells were transfected with control or TRIM37-ASO twice and RNA was isolated with RNeasy Mini Kit (Qiagen) at day 7 post-transfection. RNA-seq was performed by Novogene genome Sequencing Company using Illlumina Novoseq platform with paired-end 150 bp sequencing strategy. RNA-Seq data was aligned to the human genome assembly GRCm38/hg38 using STAR software package. Using DESeq2 R package, differential expression of genes in control and TRIM37-ASO cells was determined with a significant criterion p_{adj}<0.05. GSEA (Subramanian et al., 2005) was used for gene set enrichment analysis for all genes with FPKM>1. GeneTrail2 (Stockel et al., 2016) was used for analysis of enriched KEGG pathways. MA plot and heatmap of gene expression clusters were drawn using R.

Statistical analysis. All experiments were performed at least in triplicate and the results presented are the mean of at least three different biological replicates. The comparisons between the two groups were done by unpaired t-test; comparisons between multiple treatment groups were done by one-way or two-way ANOVA with indicated multiple comparisons post hoc tests. The enrichment of genes positively correlating with TRIM37 were calculated with two-tailed Fisher’s exact test. The distributions in correlation between GO terms was calculated by Kolmogorov-Smirnov test. All statistical analyses were performed using R/Bioconductor (version 2.15.2).

### EXAMPLE 1

### TRIM37 Associates With Double Strand Break (DSB) Repair Machinery in TNBC and Promotes TNBC Cell Survival Under Chemotherapy

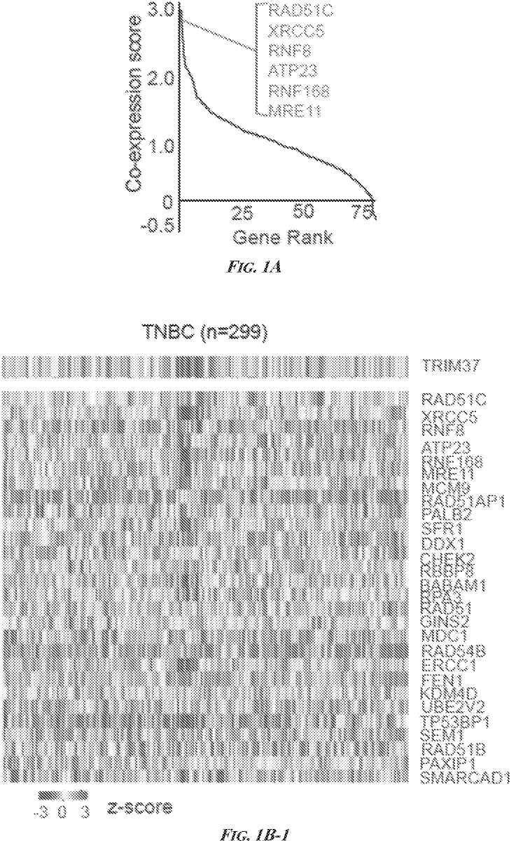

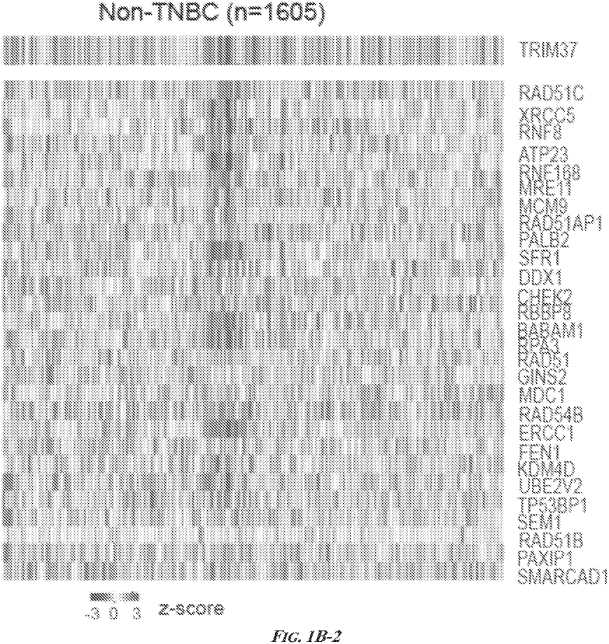

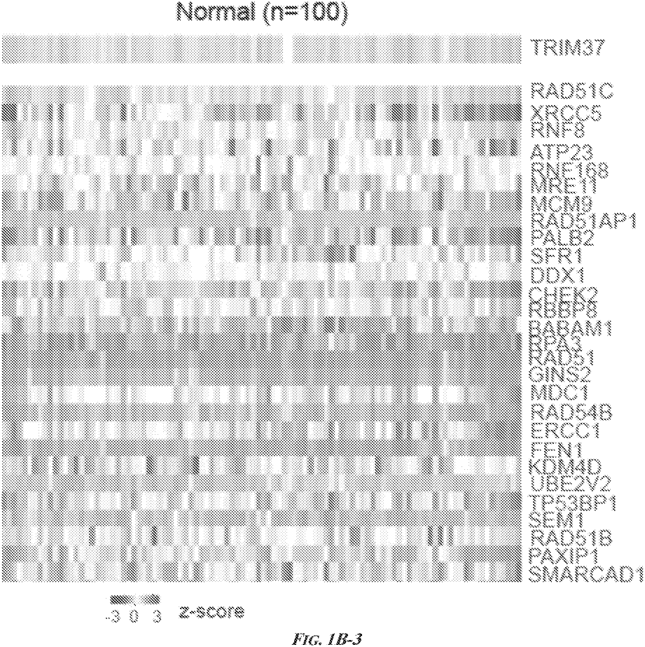

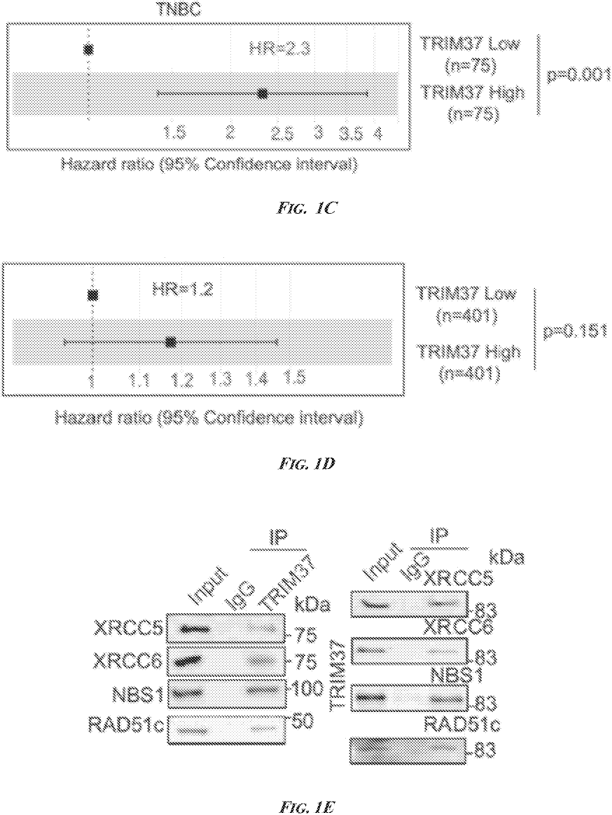

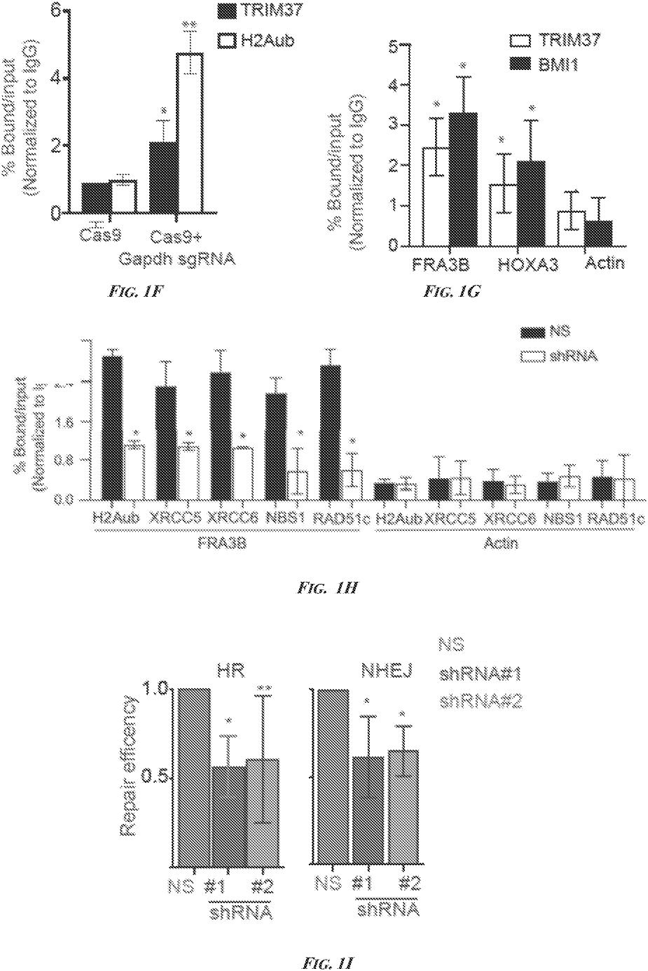

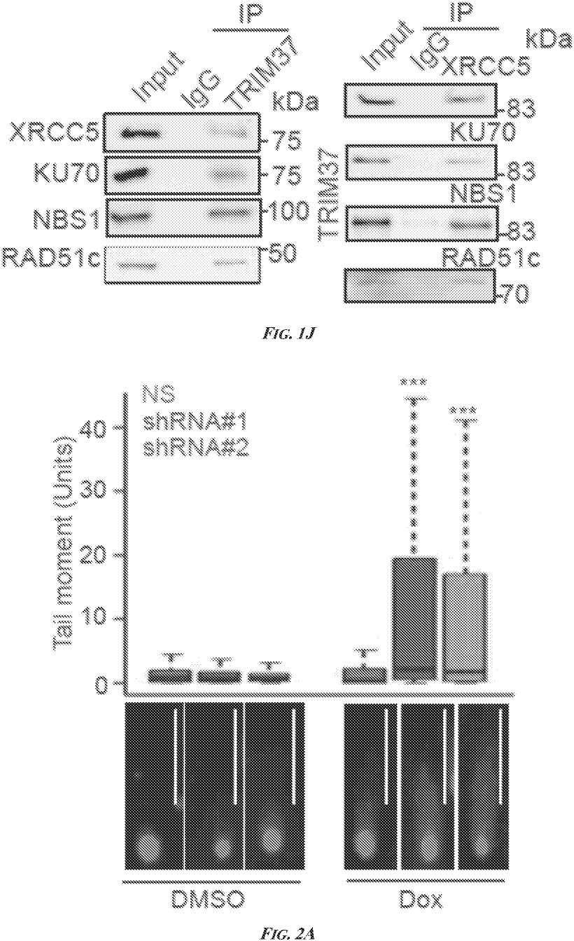

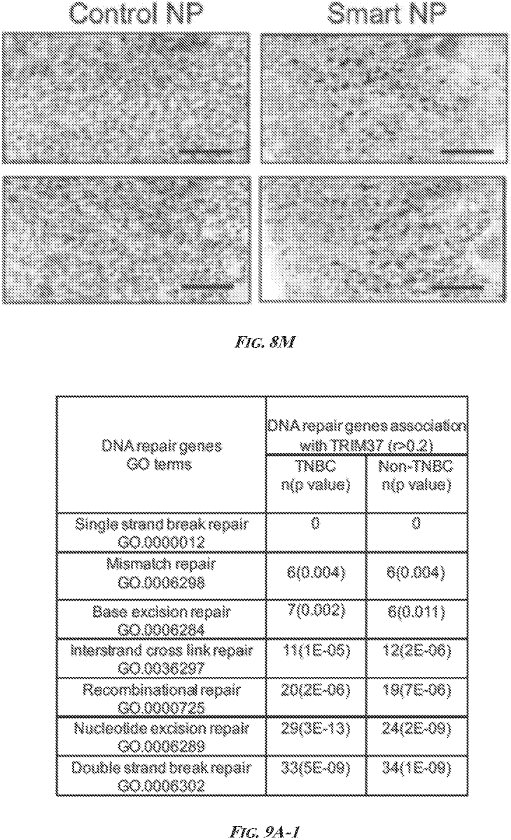

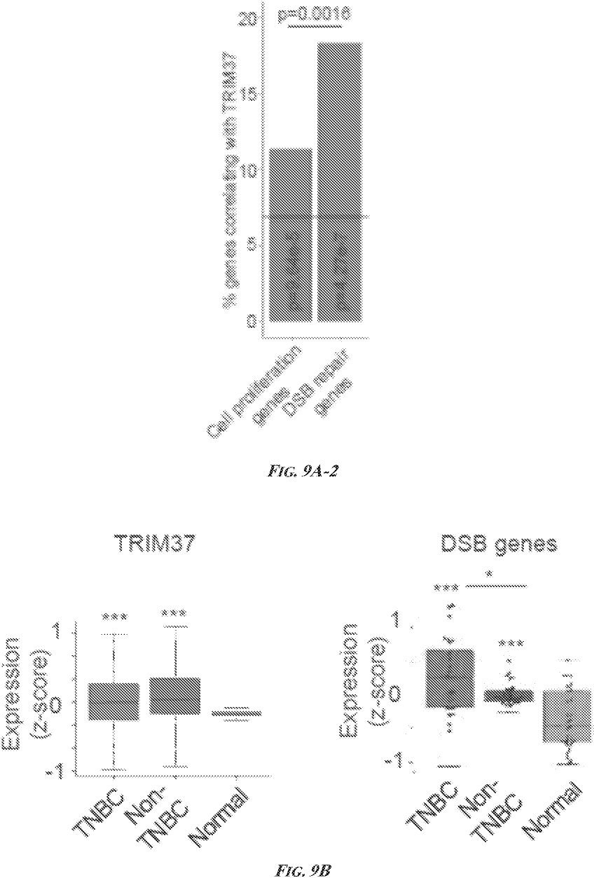

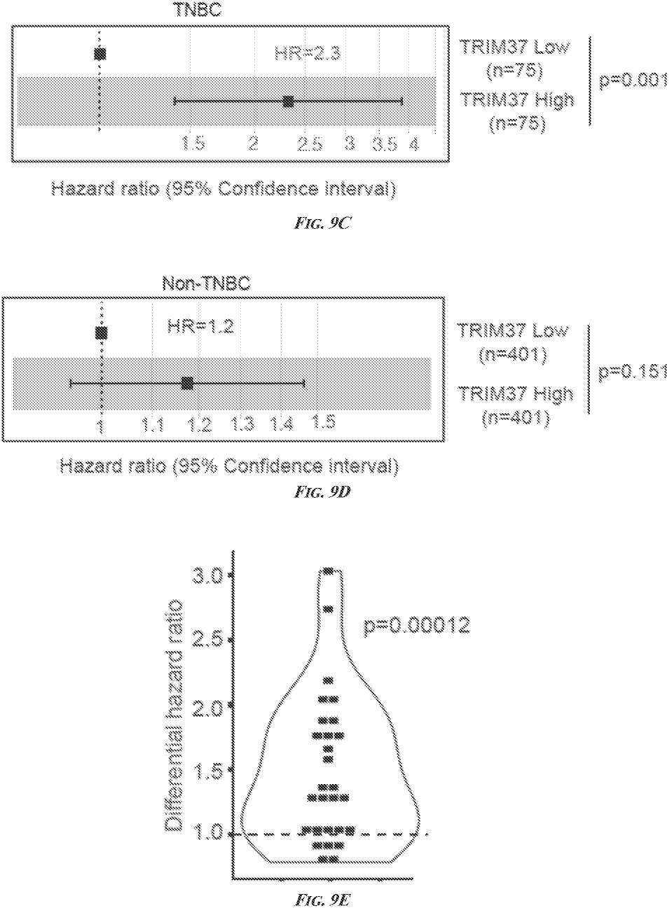

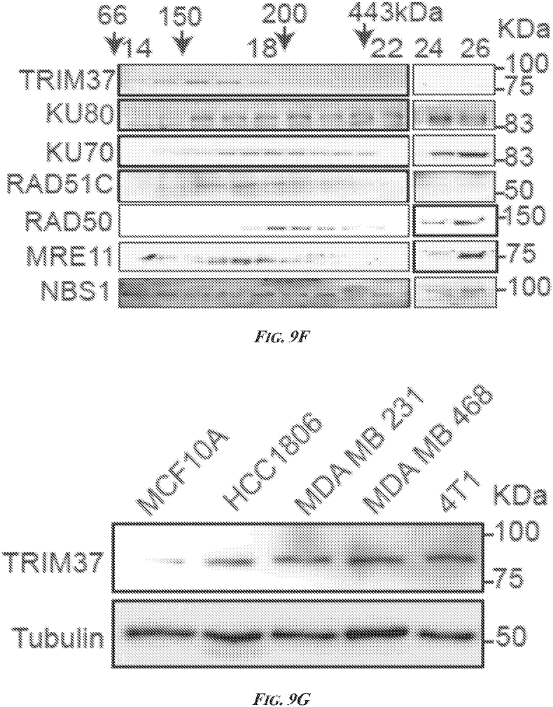

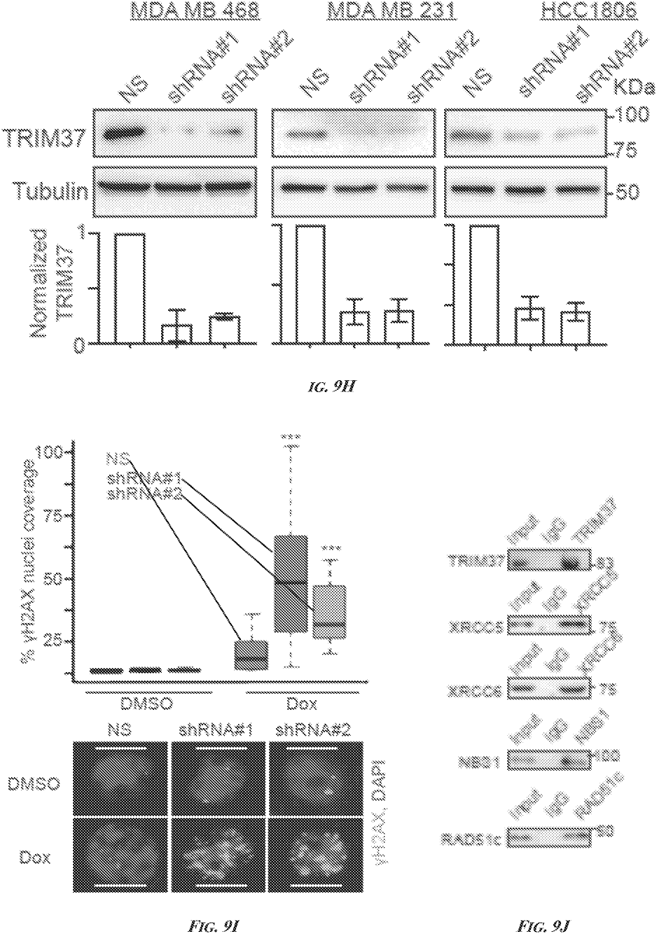

Several observations provided a rationale to explore the potential role of TRIM37 in promoting resistance to chemotherapy in cancer cells, including resistance to chemotherapeutic drugs that induce high level of DNA damage. TRIM37 catalyzes mono-ubiquitination of H2A (Bhatnagar et al., 2014), which is also an epigenetic signature enriched at the DNA damage sites in cancer cells (Kakarougkas et al., 2014; Ui et al., 2015)(9). Our analysis of the Molecular Taxonomy of Breast Cancer International Consortium (METABRIC) cohorts further revealed aberrant expression of DNA repair genes, ~90% positively and ~10% negatively correlated, in breast cancer patients stratified according to TRIM37 expression (Pearson’s coefficient > 0.2; FIGS. 1A and 9A-1; Table 4). Top-ranked twenty-eight double strand break (DSB) repair genes, which positively correlated with TRIM37, were significantly upregulated in TNBC (n=299) compared to non-TNBC patients (n=1605) or normal tissue (n=100, FIGS. 1B-1 to 1B-3 and 9B). Notably, a Cox proportional-hazards regression analysis revealed that the hazard ratio was ~2.3-fold higher for TNBC patients with high TRIM37 compared to low TRIM37 expression (FIG. 9C), linking TRIM37 to a greater risk of death in TNBC patients. In contrast, no significant association between TRIM37 expression and survival in non-TNBC patients was observed (FIG. 9D). Likewise, higher expression of top-ranked TRIM37-associated DSB genes (~80%) predicted worse overall-survival in TNBC patients as determined by HR>~1 (FIG. 9E).

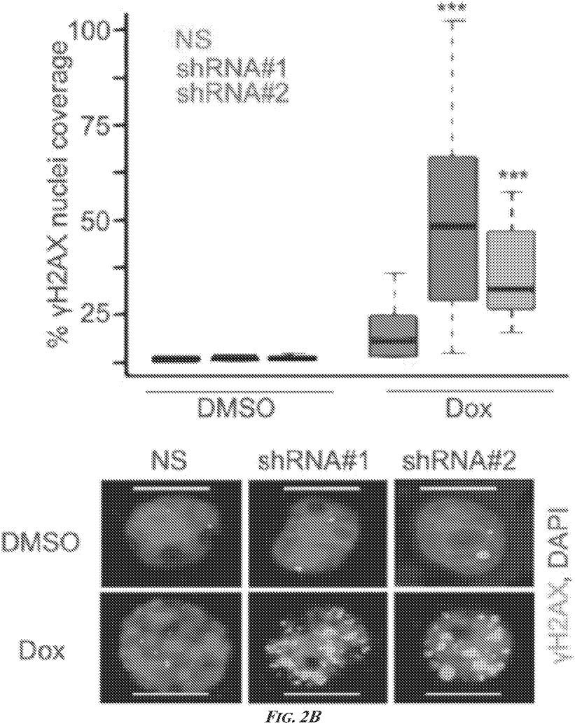

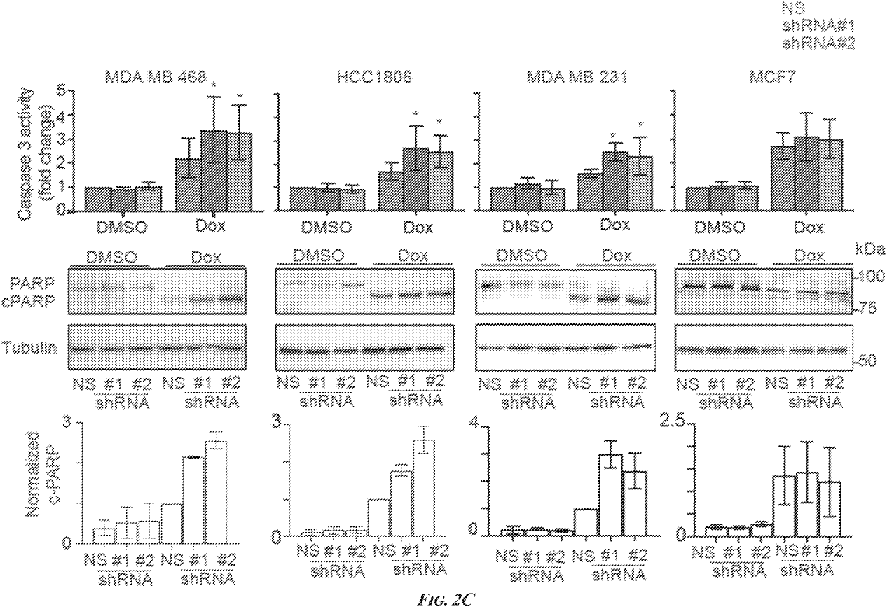

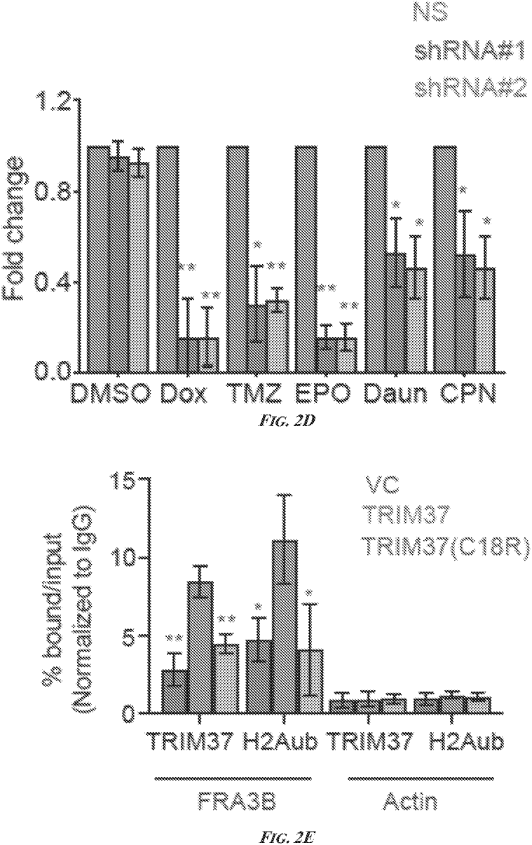

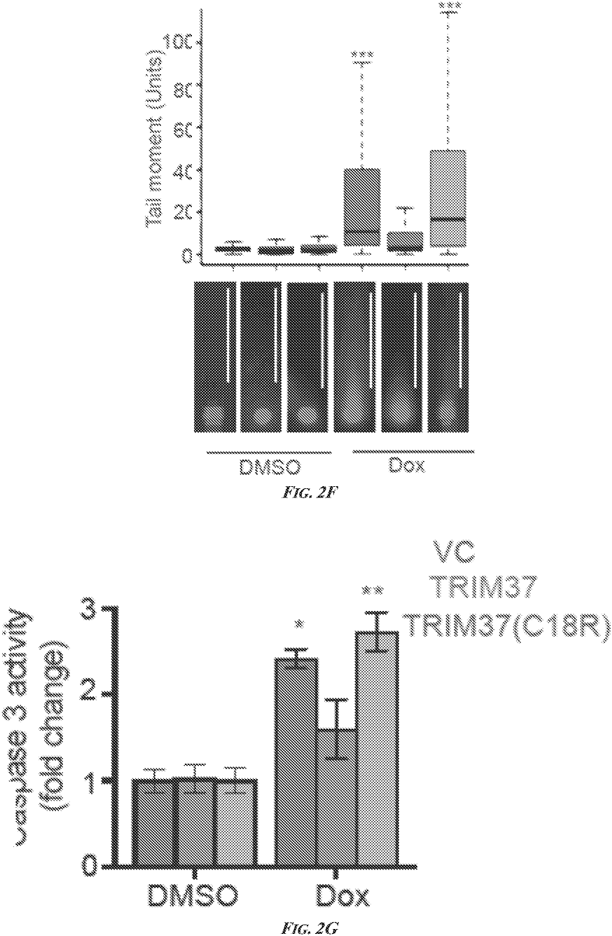

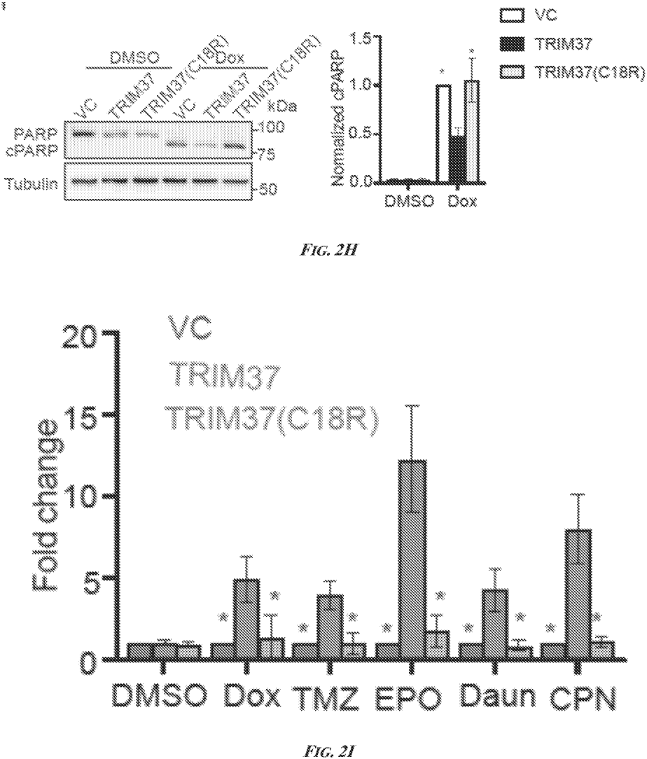

TRIM37 also catalyzes a chromatin modification enriched at transcriptionally repressed gene promoters (Zhou et al., 2008) as well as at DNA damage sites (Shanbhag et al., 2010). Furthermore, our analysis of the METABRIC cohorts revealed TRIM37 association with DNA repair genes (FIG. 1A; Table 4), which was significantly stronger than genes involved in cellular proliferation (FIG. 9A-2; Table 4). Double strand break (DSB) repair is one of the major pathways to repair damaged DNA in cancer cells and its kinetics predicts resistance to therapy (Lord & Ashworth, 2012; Goldstein & Kastan, 2015). We therefore limited the analysis to repair proteins that participate in homologous recombination (HR) and non-homologous end-joining repair pathways (NHEJ), the two subtypes of DSB repair. RAD51C, XRCC5 (Ku80), RNF8, XRCC6BP1 (Ku70 binding protein), RNF168 and MRE11 were among the DSB genes whose expression most strongly associated with TRIM37 (FIG. 1A). RAD51-associated proteins generally promote HR repair (Ciccia & Elledge, 2010). XRCC5 and XRCC6BP1 are involved in NHEJ repair pathway (Ciccia & Elledge, 2010). MRE11 forms complex with NBS1 and RAD50 to regulate response at DSB (Willims et al., 2007). RNF8 and RNF168 are E3 ligases that promote binding of DSB genes (Ciccia & Elledge, 2010). Additionally, TRIM37 also correlated with the overexpression of a family of other DSB factors, including RAD51AP1, SFR1, DDX1, RAD51, ERCC1 and CHEK2. Together, these findings identified 28 DSB genes that significantly correlated with TRIM37 in breast cancer patients (Pearson’s coefficient > 0.2; Table 4).

The analysis of METABRIC cohorts stratified by breast cancer subtypes revealed statistically significant correlation between TRIM37 and DSB genes in TNBC patients but not in non-TNBC patients or histologically normal breast tissue adjacent to tumor (NAT; FIG. 1B and FIG. 9B). As shown in FIG. 1C, the hazard ratio (HR) was ~2.3-fold higher for TNBC patients with high-compared to low-TRIM37 expression, linking TRIM37 to poor prognosis in TNBC patients. In contrast, no significant association between TRIM37 levels and survival was observed in non-TNBC patients (FIG. 1D). Likewise, higher expression of a subset of DSB genes analyzed predicted poor overall-survival in TNBC patients as determined by HR>~1 (FIG. 9E).

We next performed a series of functional experiments to determine the molecular mechanisms underlying the biological activity of TRIM37 in DSB repair using several human TNBC cell lines (HCC1806, MDA MB 468 and MDA MB 231; FIG. 9G). We first asked whether TRIM37 was physically associated with DSB genes. To test this idea, HCC1806 whole cell extract was fractionated by sucrose gradient sedimentation, and individual fractions were analyzed for TRIM37 and a representative subset of DSB proteins identified in FIG. 1A. Results shown in FIG. 9F demonstrated that TRIM37 co-sediments with XRCC5, XRCC6, RAD51c, MRE11, and NBS1. Physical interactions between TRIM37 and DSB proteins could be confirmed by co-immunoprecipitation (FIG. 1E and FIG. 9J) and immunofluorescence analysis (FIG. 9K) in MDA MB 468 cells treated with doxorubicin (Dox), a first-line chemotherapeutic agent. No significant changes in the expression of DSB genes were observed in HCC1806 cells expressing TRIM37-specific short hairpin RNA (shRNA), excluding TRIM37-mediated transcriptional regulation of these DSB genes (FIGS. 9L-9M; Table 5).

Prompted by these findings, we interrogated TRIM37 recruitment to the site of DNA damage and DSB using two independent experimental systems. We first enzymatically-induced DSB at Gapdh in MDA MB 468 cells using sequence-specific guide RNA (sgRNA) to promote Cas9-nuclease binding and analyzed TRIM37 recruitment by chromatin. Chromatin immunoprecipitation (ChIP) assay confirmed TRIM37 as well as H2Aub enrichment at the DSB in Gapdh (FIG. 1F). Next, we analyzed recruitment of TRIM37 and BMI1, a component of the polycomb complex that participates in DSB repair (Ismail et al., 2010), to the endogenous fragile site, FRA3B. Consistently, ChIP analysis confirmed TRIM37 and BMI1 binding to FRA3B following Dox treatment (FIG. 1G), a first-line chemotherapeutic agent (Cancer Genome Atlas Network, 2012; Foulkes et al., 2010). Both BMI1 and TRIM37 were bound to HOXA3, a paradigmatic site for regulation by polycomb complex (FIG. 1G). In marked contrast to the control cells, knockdown of TRIM37 substantially decreased H2Aub enrichment as well as DSB proteins binding to FRA3B in Dox-treated MDA MB 468 cells (FIG. 1H). As expected, repair efficiencies of NHEJ and HR pathways were significantly reduced in TRIM37-knockdown cells compared to control cells (FIG. 1I). Together, these results demonstrate that TRIM37 interacts with DSB repair factors and functionally contributes to the repair of therapy-induced DNA damage.

To explore the molecular mechanisms underlying the biological activity of TRIM37, HCC1806 whole cell extract was fractionated by sucrose gradient sedimentation, and individual fractions were analyzed by immunoblotting for TRIM37 and a representative set of DSB proteins. Results shown in FIG. 9F demonstrated that TRIM37 co-sediments with the following DSB proteins: KU80, KU70, RAD51c, RAD50, MRE11, and NBS1. Physical interactions between TRIM37 and DSB proteins could be demonstrated by co-immunoprecipitation (FIG. 1J).

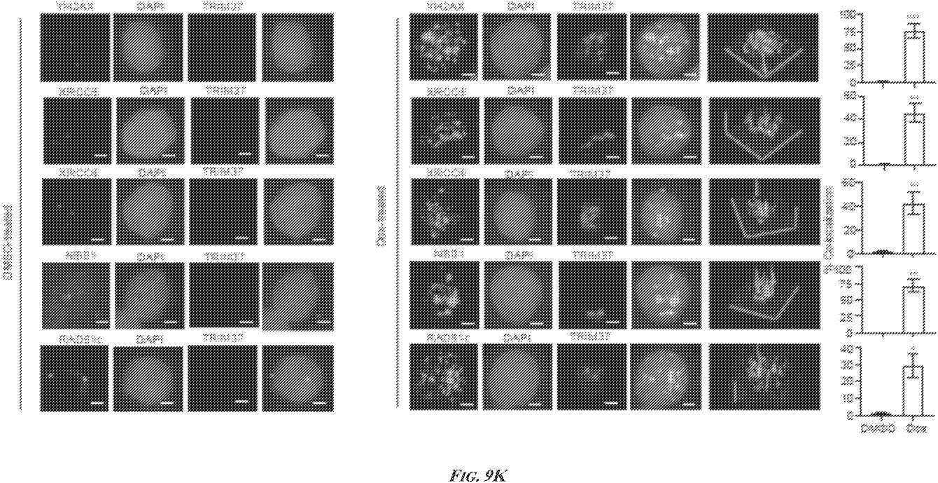

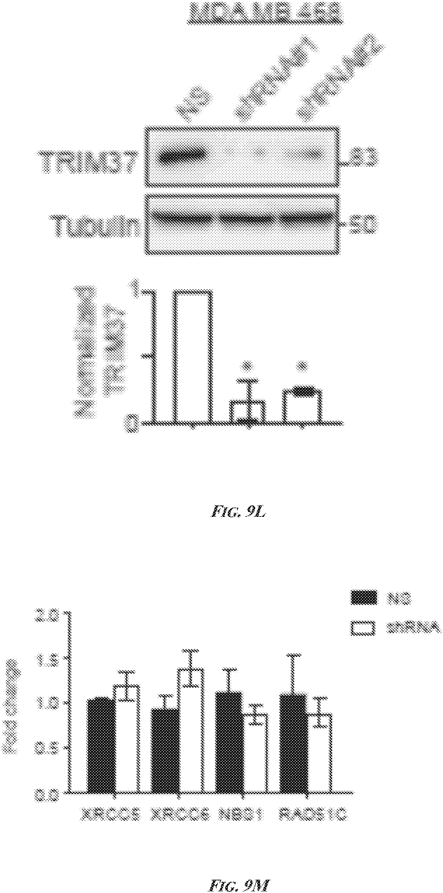

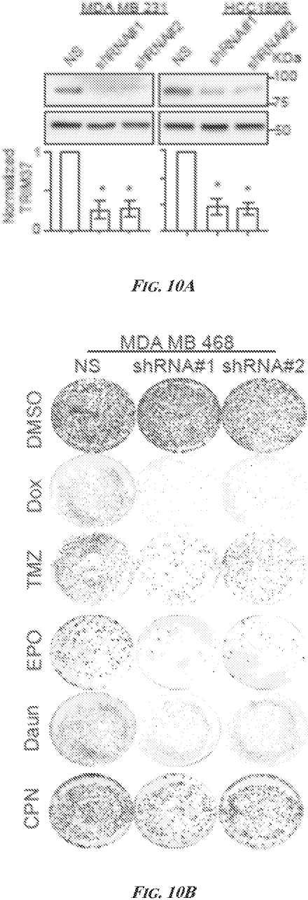

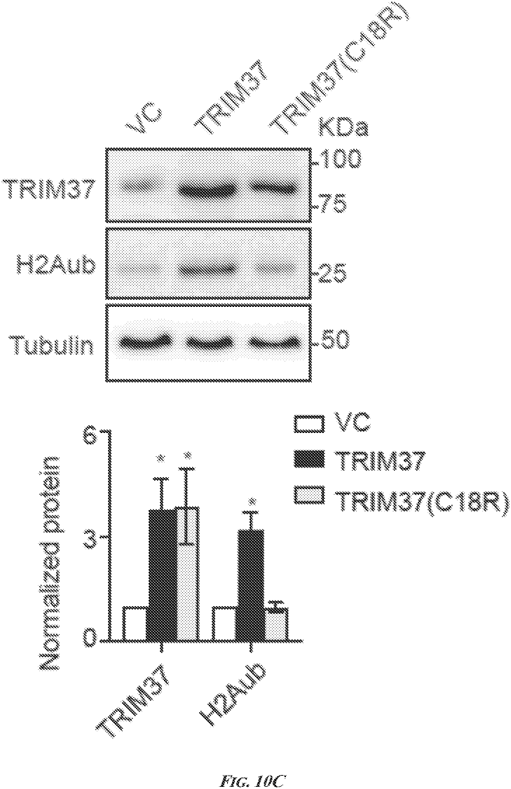

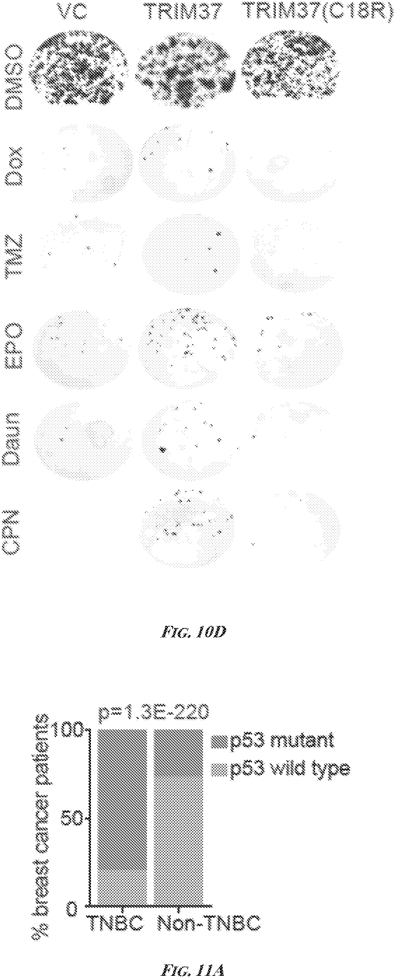

Given the association of TRIM37 with DSB genes, we directly examined the impact of TRIM37 on the extent of chemotherapy-induced DNA damage. After short hairpin RNA (shRNA)-mediated knockdown of TRIM37 in MDA MB 468 cells, in which TRIM37 is highly expressed (FIGS. 9G-9H; Table 5), we observed significantly higher Dox-induced DNA damage than in control cells as indicated by ~6-fold increase in median tail moment (FIG. 2A) and ~5-fold increase in the median nuclear coverage of phosphorylated histone H2AX (y-H2AX), a marker for DNA damage (FIG. 9I). We next analyzed the relationship between the therapy-induced DNA damage and cell death. TNBC cells transduced with TRIM37 shRNA showed significant increase in Caspase 3 activity and PARP cleavage (FIG. 2C), hallmarks of cell death (Green, 2019). As a control, we treated MCF7 (a wild type TP53 and hormone positive breast cancer cell line) with Dox, which did not augment PARP cleavage or Caspase 3 activity (FIG. 2C). Consistently, TRIM37-knockdown cells showed ~5-fold decrease in the colony formation compared to the control TNBC cells following treatment with Dox (FIGS. 2D and 10B). As anticipated, treatment of TRIM37-knockdown cells with temozolomide, etoposide, daunorubicin, or cisplatin also substantially decreased cell survival relative to the control cells (FIGS. 2D and 10B). We also found that TRIM37-knockdown in MDA MB 468 cells reduced the repair efficiency of Dox-induced damage by non-homologous end-joining and homologous recombination pathways, the two subtypes of DSB repair (FIG. 1I).

We next asked whether TRIM37 function in chemoresistance is dependent on its catalytic activity. To address, we ectopically expressed either wild type TRIM37 (TRIM37) or a TRIM37 derivative bearing a point mutation in a conserved cysteine residue in the RING finger motif (TRIM37(C18R)), which interferes with the catalytic activity in MCF10AT, a premalignant K-RAS transformed triple negative breast cell line (Bhatnagar et al., 2014; FIG. 10C). A directed-ChIP analysis showed that TRIM37 and H2Aub were significantly enriched at FRA3B in TRIM37 over-expressing MCF10AT cells relative to vector control (VC) following Dox treatment (FIG. 2E). In marked contrast, ectopic expression of TRIM37(C18R) inhibited the H2Aub enrichment at FRA3B in the Dox-treated MCF10AT cells (FIG. 2E). Furthermore, a comet assay (FIG. 2A), Caspase 3 activity (FIG. 2G), and PARP cleavage (FIG. 2H) confirmed that TRIM37, but not TRIM37(C18R), expression reduced chemotherapy-induced DNA damage. As anticipated, a colony formation assay confirmed that wild type TRIM37 expression in MCF10AT promoted survival following chemotherapy, which was not observed in VC or TRIM37(C18R) expressing MCF10AT cells (FIGS. 2I and 10D). Collectively, we concluded that TRIM37 catalyzed H2A mono-ubiquitination contributes to cancer cell survival by reducing chemotherapy-induced DNA damage in TNBC.

### EXAMPLE 2

### TRIM37 Catalyzed H2Aub Is Required for Its Function in Chemoresistance

We next directly examined the impact of TRIM37-knockdown on chemotherapy-induced DNA damage and clonogenic growth. Knockdown of TRIM37 resulted in ~6-fold increase in median tail length in comet assay (FIG. 2A and FIGS. 9L, 10A) and ~5-fold increase in the median nuclear coverage of phosphorylated histone H2AX (y-H2AX, FIG. 2B) in Dox-treated cells. TRIM37-knockdown in TNBC cell lines also markedly increased Caspase 3 activity and PARP cleavage (FIG. 2C), hallmarks of cell death. By contrast, Dox-treated MCF7, a hormone receptor positive breast cancer cell line, did not augment PARP cleavage or Caspase 3 activity (FIG. 2C), suggesting TRIM37 function in chemoresistance was limited to TNBC cells. As expected, knockdown of TRIM37 sensitized MDA MB 468 cells to chemotherapeutic stress without affecting proliferation as indicated by substantially decreased clonogenic growth relative to the control cells (FIGS. 2D and 10B).

Given ubiquitin is critical for DSB factor recruitment to damaged DNA (Cohn & D′Andrea, 2008), we next asked whether TRIM37-catalyzed H2Aub is required for its function in chemoresistance. To test this idea, we ectopically expressed either wild type TRIM37 (TRIM37) or catalytically-dead TRIM37 (TRIM37(C18R)) in MCF10AT, a premalignant K-RAS transformed triple negative breast cell line (FIG. 10C). While Dox-treatment induced significant enrichment of H2Aub at FRA3B in TRIM37 over-expressing cells, TRIM37(C18R) failed to promote H2Aub enrichment at FRA3B (FIG. 2E). Consequentially, Dox-treatment of TRIM37(C18R)-expressing cells caused substantially longer comet tails (FIG. 2F) as well as increased Caspase 3 activity (FIG. 2G) and PARP cleavage (FIG. 2H) relative to TRIM37-expressing cells. Finally, substantially fewer colonies were observed for cells expressing TRIM37(C18R) compared to TRIM37 following chemotherapeutic stress (FIG. 2I and FIG. 10D).

### EXAMPLE 3

### TRIM37 Promotes Chemoresistance in the Absence of Functional P53

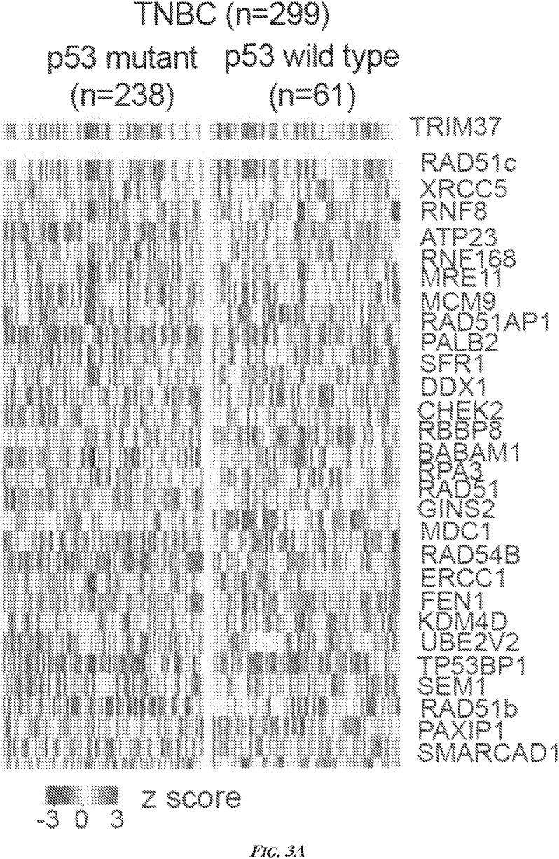

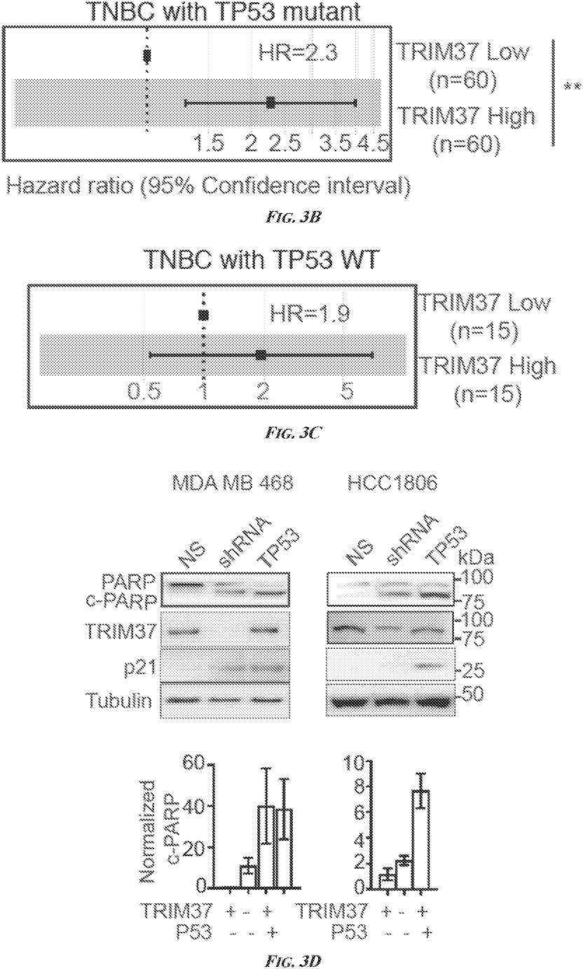

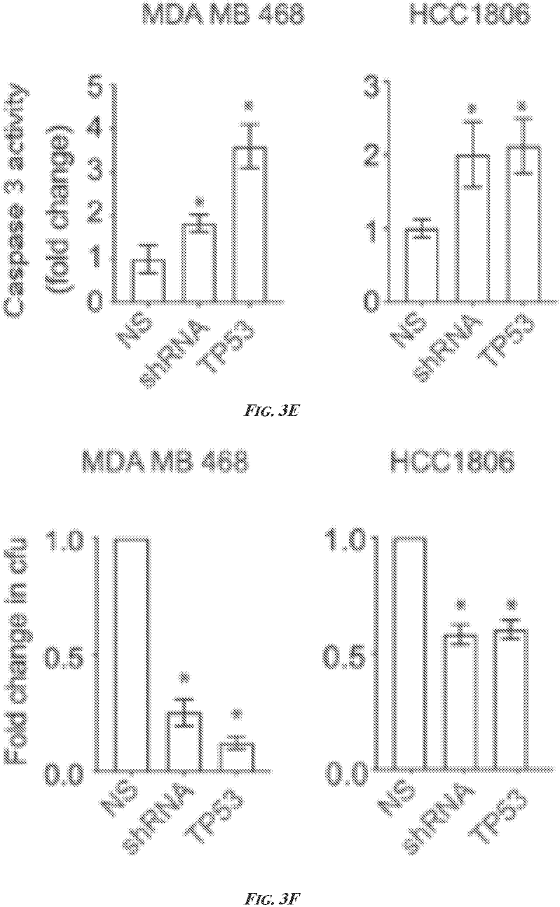

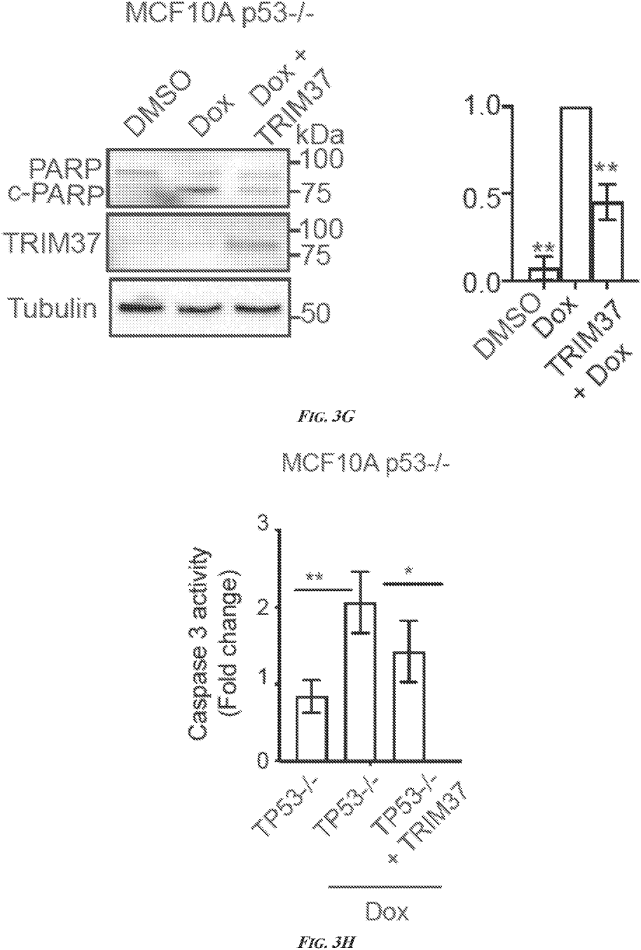

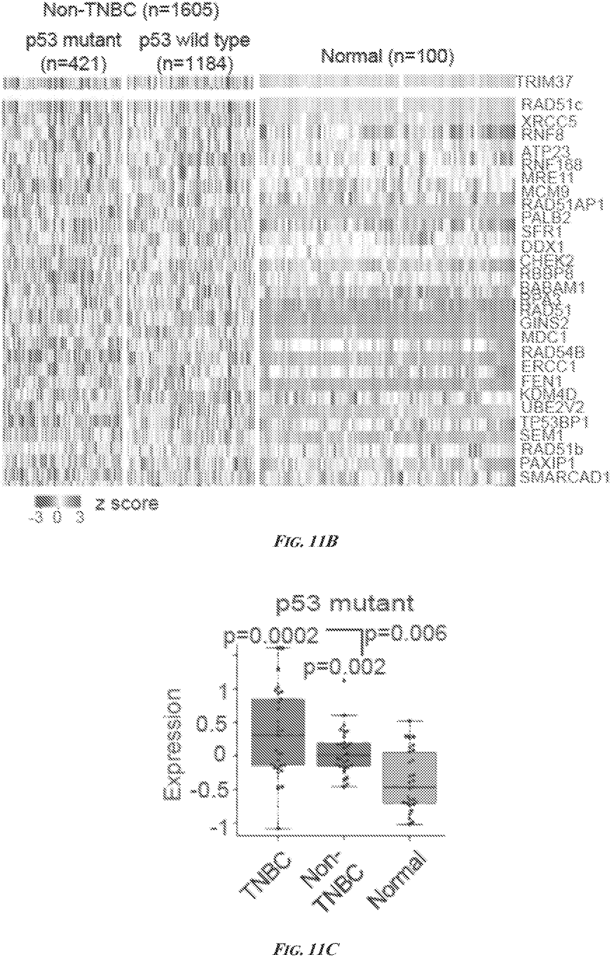

Wild type TP53 represents a barrier to chemoresistance by altering DSB repair responses, activating checkpoints and the stress responses (Kastenhuber & Lowe, 2017). Strikingly, TNBC tumors frequently harbor disrupting TP53 mutations (FIG. 11A), which primarily cause the loss of its wild type function (Gasco et al., 2003). Surprisingly, analysis of representative TRIM37-associated DSB genes showed a striking correlation between TRIM37 and DSB genes expression in TNBC patients carrying mutant TP53 but not in wild type TP53 (FIG. 3A and FIGS. 3B-3D). Consistently, TP53 mutant, but not wild type, TNBC patients with high-TRIM37 were at ~2.3-fold higher risk of death relative to low-TRIM37 (FIGS. 3B-3C).

To investigate the relationship between p53 and TRIM37, we transiently expressed wild type p53 in MDA MB 468 (carries transcriptionally inactive p53 R273H) and HCC1806 (carries p53 T256Kfs*90 deletion) cells. For each cell line, p53-reconstituted TNBC cells showed significantly higher PARP cleavage (FIG. 3D) and Caspase 3 activity (FIG. 3E) compared to the control cells following Dox-treatment. A clonogenic assay confirmed that p53 over-expression sensitized MDA MB 468 (~4-fold) and HCC1806 (~2-fold) cells to Dox despite high levels of TRIM37 (FIG. 3F and FIG. 11E). Reciprocally, ectopic expression of TRIM37 in genetically ablated p53 null (p53-/-) MCF10A cells (Weiss et al., 2010) substantially reduced PARP cleavage, and Caspase 3 activity relative to empty vector (FIGS. 3G-3H). As expected, TRIM37-expressing p53-/- MCF10A showed an ~4-fold increase in colony formation in comparison to control cells (FIG. 3I and FIG. 11F). In summary, consistent with previous results for MDM2, KRAS, and ARID1 A (Mina et al., 2017, we find that TRIM37 requires loss of p53 to drive the chemoresistant phenotype in TNBC cells.

### EXAMPLE 4

### Chemotherapy Amplifies a TRIM37 Survival Axis in TNBC

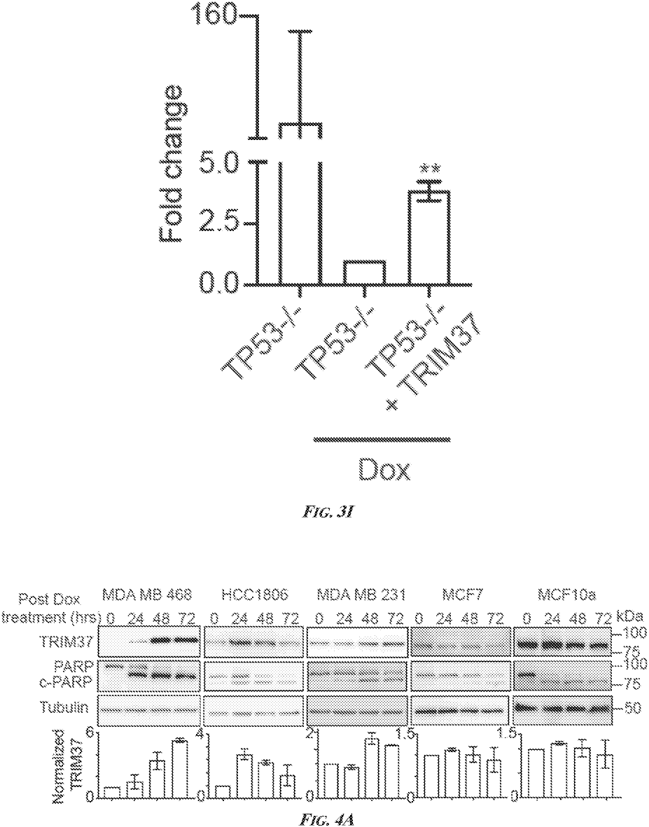

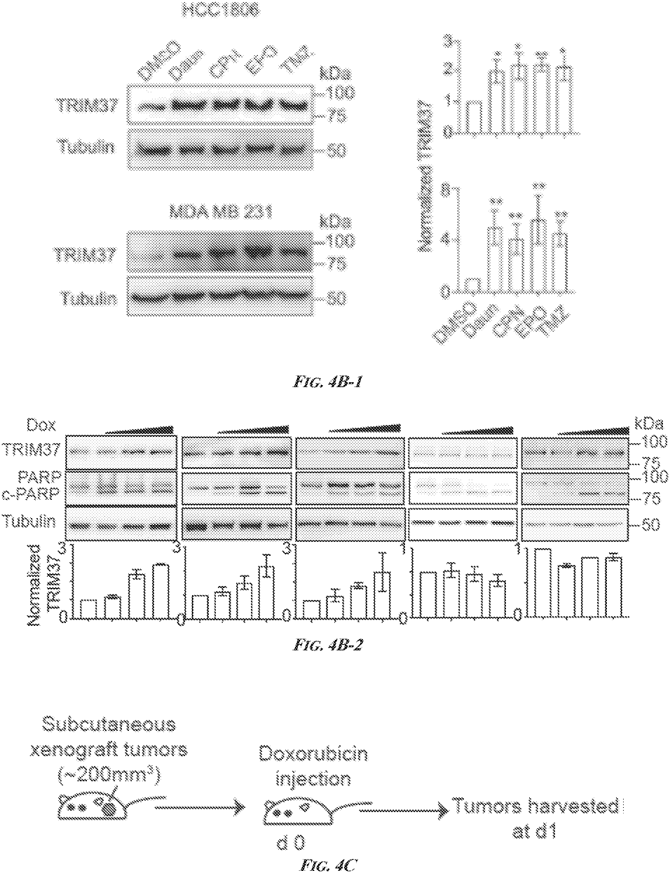

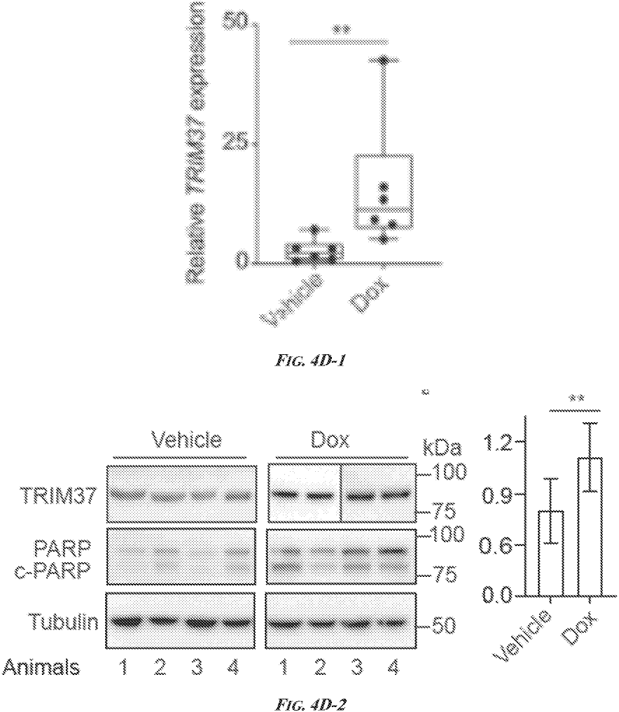

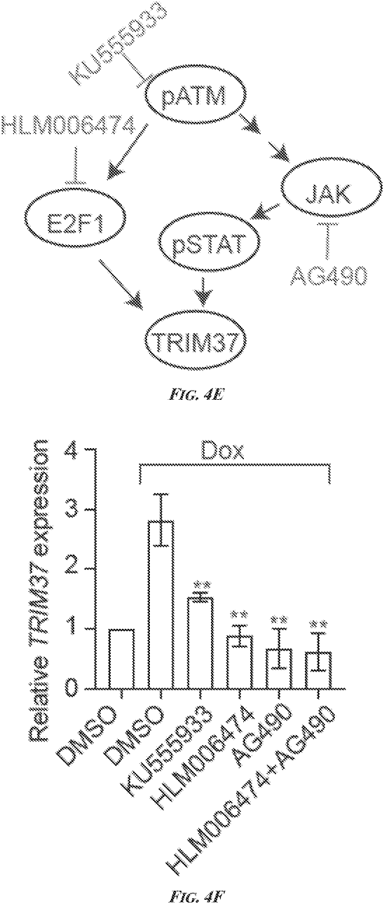

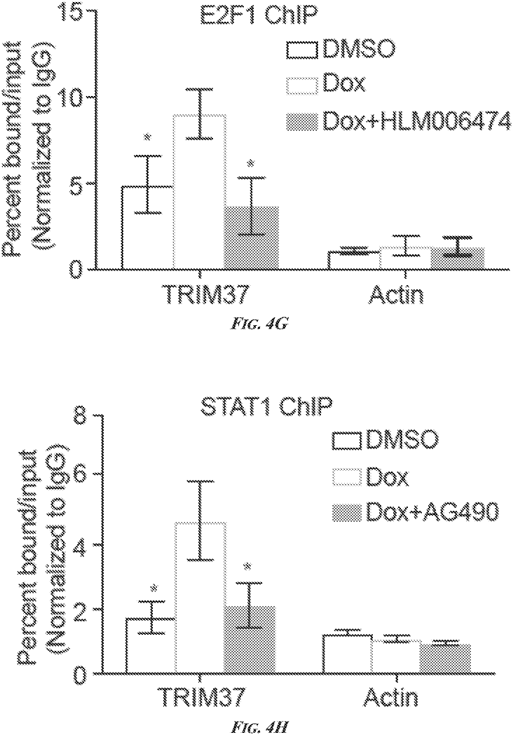

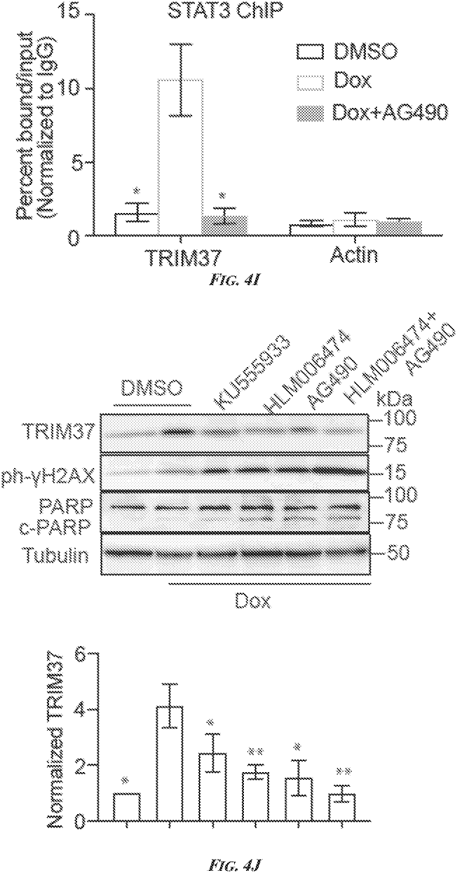

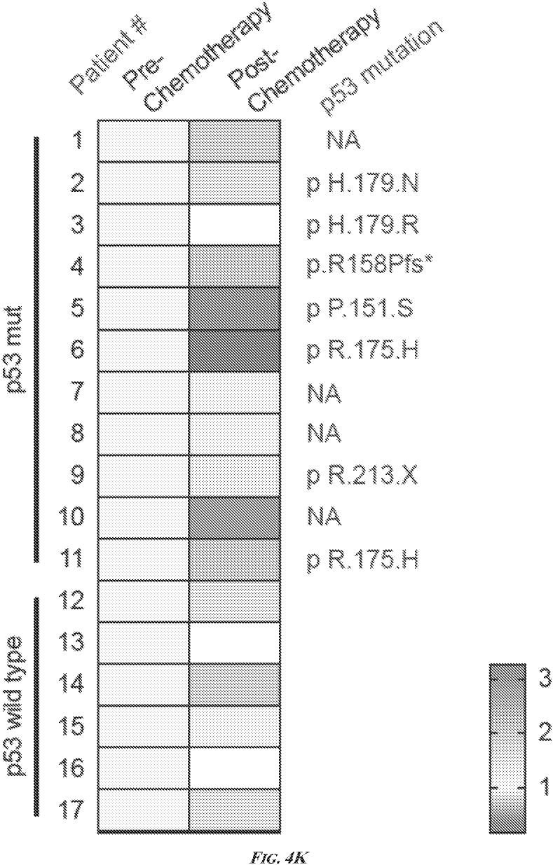

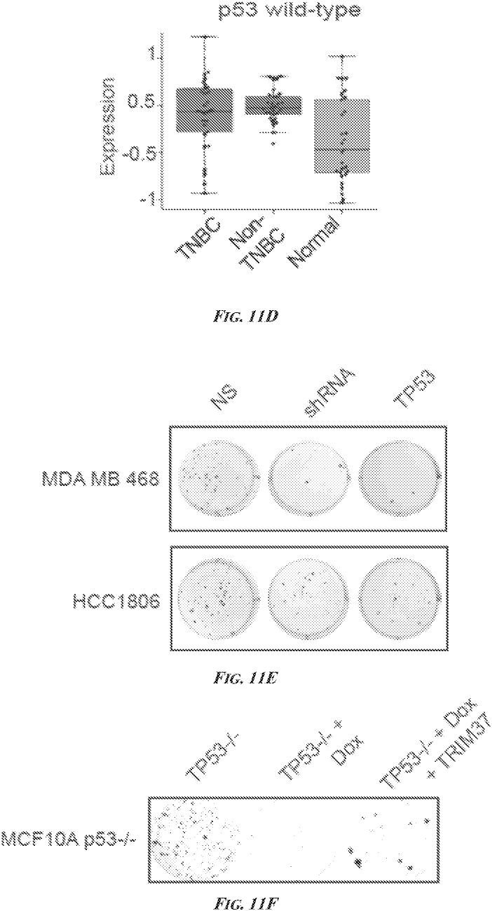

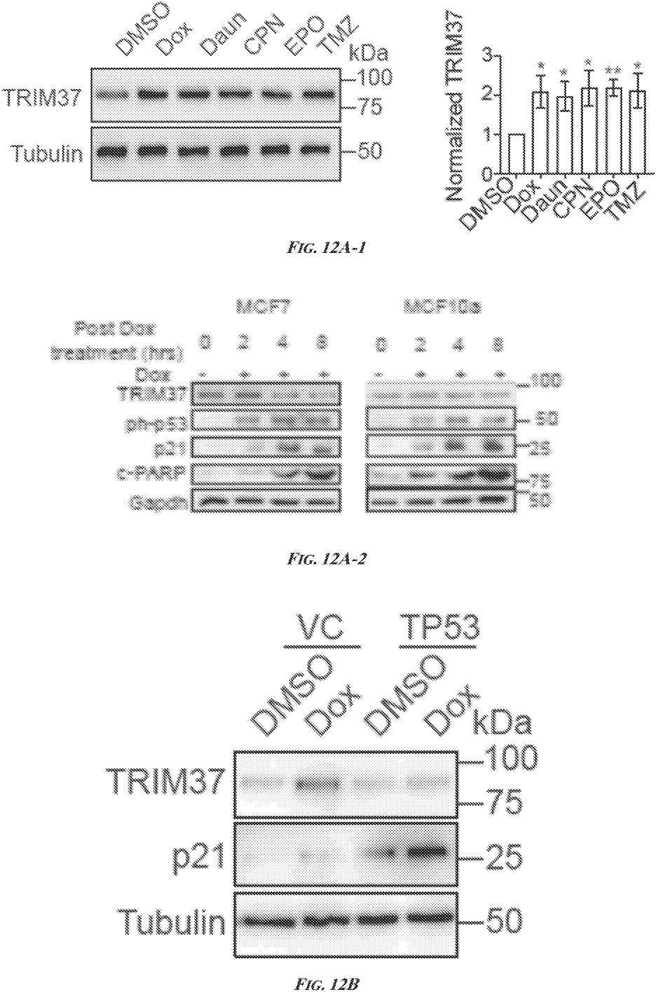

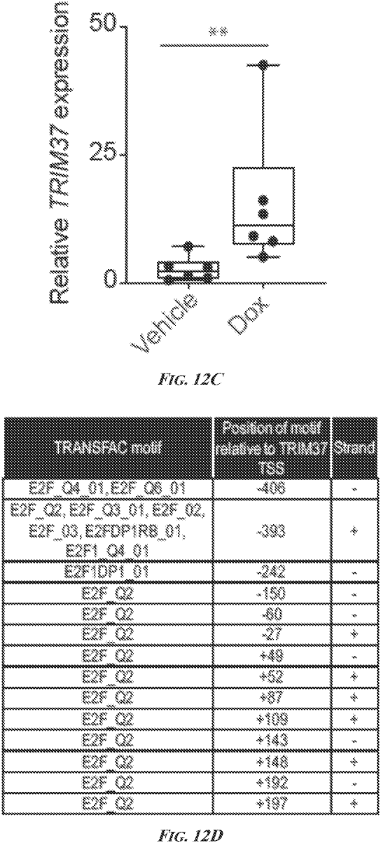

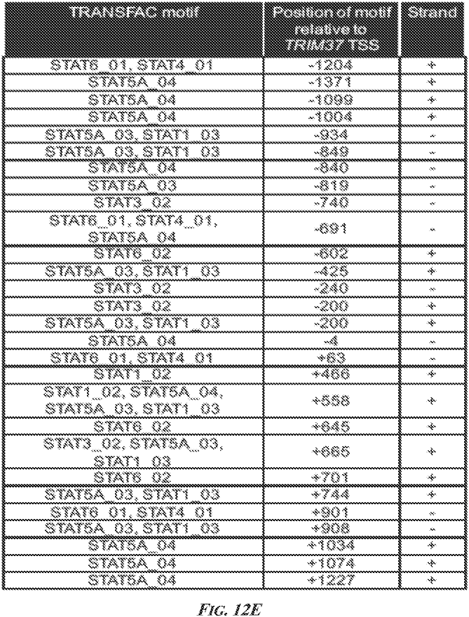

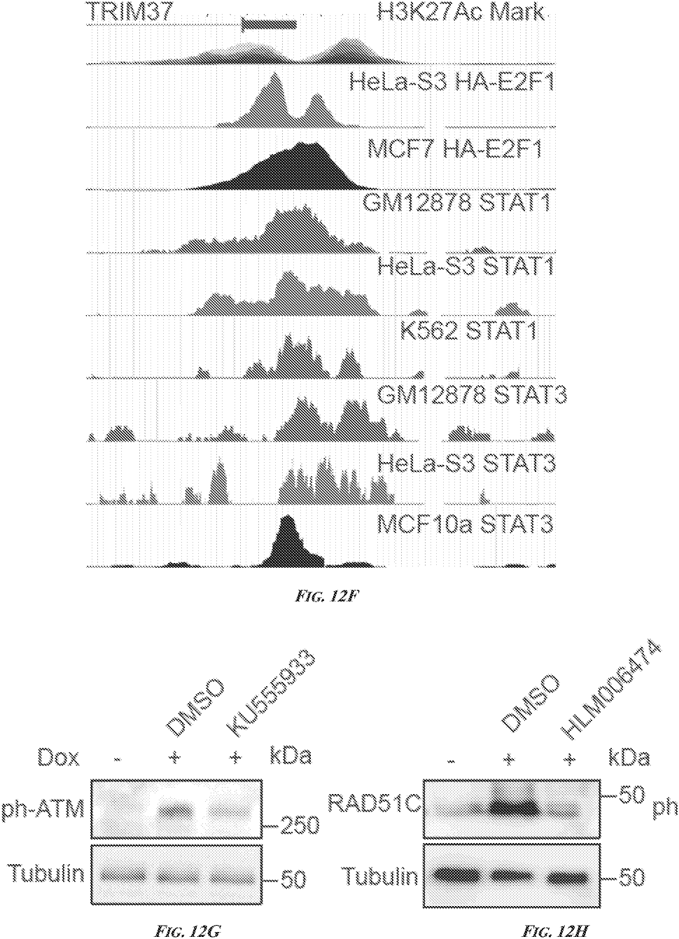

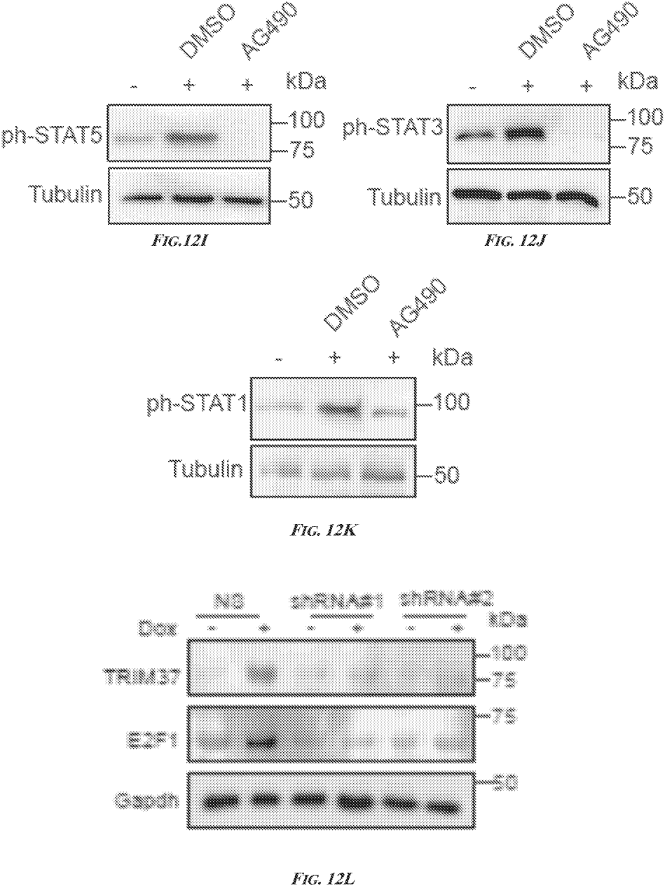

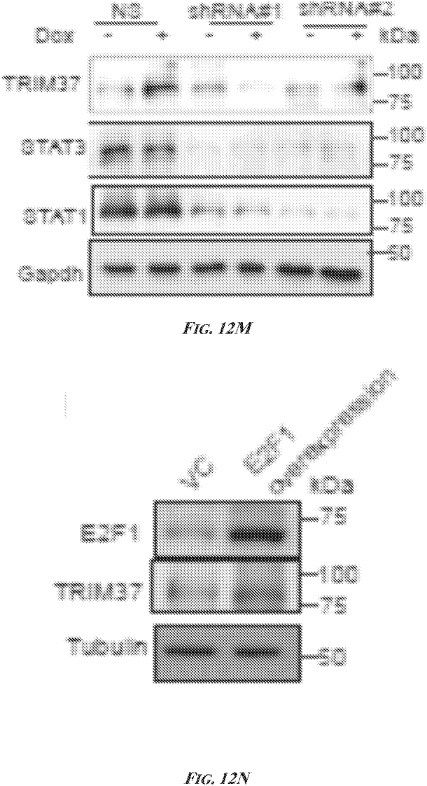

Despite continuous chemotherapy, TNBC tumors metastasize at a relatively higher rate than other breast cancer subtypes. This led us to hypothesize that TRIM37 overexpression could favor metastasis by selecting for highly aggressive chemoresistant TNBC cells. Consistently, a dose- and time-dependent upregulation of TRIM37 was observed in MDA MB 468, HCC1806 and MDA MB 231 cells treated with doxorubicin (FIGS. 4A-4B). Similarly, TNBC cells treated with chemotherapeutic drugs daunorubicin, cisplatin, etoposide, and temozolomide increased TRIM37 expression post-treatment relative to DMSO (FIG. 12A-1). In contrast, no increase in TRIM37 expression was observed in MCF7 or MCF10A cells (FIGS. 4A-4B). A p53-reconstituted MDA MB 468 cells also obliterated Dox-induced TRIM37 upregulation (FIG. 12B). To extend our results in vivo, we next treated mice bearing HCC1806RR subcutaneous tumors with Dox (FIG. 4C). Doxorubicin treatment but not DMSO upregulated TRIM37 in tumors (FIG. 4D), which was predominantly transcriptional (FIG. 12C).

We next explored the molecular mechanism underlying chemotherapy-induced TRIM37 upregulation. Most TNBC patients receive chemotherapy, which is effective in early stages of the disease but ~30-50% patients develop resistance (Pal et al., 2011). While the exact mechanisms of chemoresistance remain to be understood, chemotherapeutic drugs often induce genomic and transcriptomic reprogramming of resistant signatures (Kim et al., 2018), including alterations in DNA repair capacity (O′Connor, 2015). Previous studies have suggested that accumulation of such changes accompany selection and expansion of resistant TNBC cells (Navin, 2014). We therefore analyzed the expression of TRIM37 in MDA MB 468, HCC1806 and MDA MB 231 cells following chemotherapy. Surprisingly, we found that TRIM37 is upregulated in all the three TNBC cell lines tested in a time-dependent manner (FIG. 4A). In contrast, no significant increase in TRIM37 was observed in p53 wild type MCF7 or MCF10A, an immortalized breast epithelial cell (FIG. 4A). The analysis of TRIM37 upregulation kinetics in MCF7 and MCF10a revealed quick and robust p53 activation following Dox treatment (FIG. 12A-2), supporting our previous findings that p53 overrides TRIM37 function in chemoresistance (FIGS. 3A-3I). Indeed, ectopic expression of p53 in MDA MB 468 cells obliterated Dox-induced TRIM37 upregulation (FIG. 12B). As expected, TNBC cells treated with additional chemotherapeutic drugs also increased TRIM37 expression post-treatment (FIG. 4B). Finally, increased TRIM37 level in xenograft tumors following dox-treatment revealed therapy-induced transcriptional upregulation of TRIM37 in vivo (FIGS. 4C - 4D).

We next sought to determine the mechanistic basis for chemotherapy-induced burst in TRIM37 levels in TNBC tumors. A previous study identified TRIM37 association with ataxia-telangiectasia-mutated (ATM) kinase, a DNA damage sensor (Wu et al., 2018). Moreover, TRIM37 promoter harbors regulatory elements for STAT and E2F1, downstream effectors of ataxia-telangiectasia-mutated (ATM kinase (FIGS. 12C-12F). We therefore investigated the potential role of ATM signaling in the transcriptional regulation of TRIM37 by utilizing small molecule inhibitors of either ATM (KU000553) or its downstream effectors, E2F1 (HLM006474) and JAK (AG490) to evaluate ATM-dependent TRIM37 upregulation in TNBC (FIG. 4E and FIGS. 12G-12K). JAK is a tyrosine kinase that phosphorylates STAT. qRT-PCR analysis revealed that pharmacological inhibition of ATM or its downstream effectors blocks TRIM37 upregulation following Dox-treatment (FIG. 4F). Similarly, E2F1- and STAT ⅓-knockdown abolished Dox-induced TRIM37 upregulation (FIGS. 12L-12M), whereas their overexpression significantly increased TRIM37 levels (FIGS. 4M-4N). ChIP analysis confirmed Dox-induced STAT1, STAT3, and E2F1 recruitment to TRIM37, which substantially decreased following pharmacological inhibition of JAK or E2F1 activation (FIGS. 4G-4I). Consequently, inhibition of ATM signaling in MDA MB 468 cells induced significantly higher DNA damage and cell death relative to control cells as determined by γ-H2AX and PARP cleavage (FIG. 4J).

Finally, to clinically validate chemotherapy-induced burst in TRIM37, we analyzed TRIM37 expression in a panel of matched pre- and post-neoadjuvant chemotherapy-treated tumor biopsies from TNBC patients (see also Methods). qRT-PCR analysis showed that chemotherapy treatment increased TRIM37 expression in ~82% of TNBC tumors carrying mutation in TP53 (n=11, FIG. 4K). Consistent with TNBC cellular models, no significant change in TRIM37 was observed in TP53-wild type TNBC tumors (n=6; FIG. 4K). Collectively, our results show that chemotherapeutic stress increases TRIM37 expression in TNBC tumors in an ATM-dependent manner.

### EXAMPLE 5

### TRIM37 Confers Anoikis Resistance to Promote to Promote Metastatic Potential in TNBC

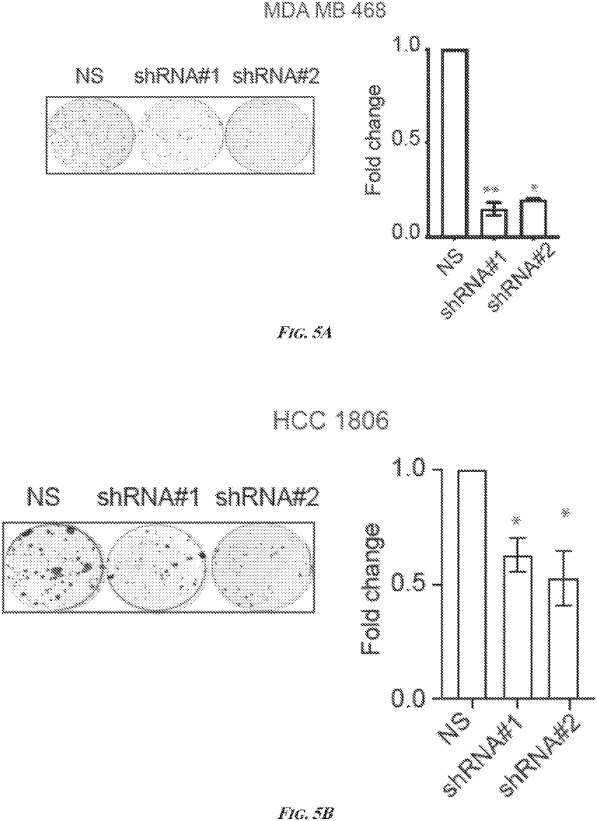

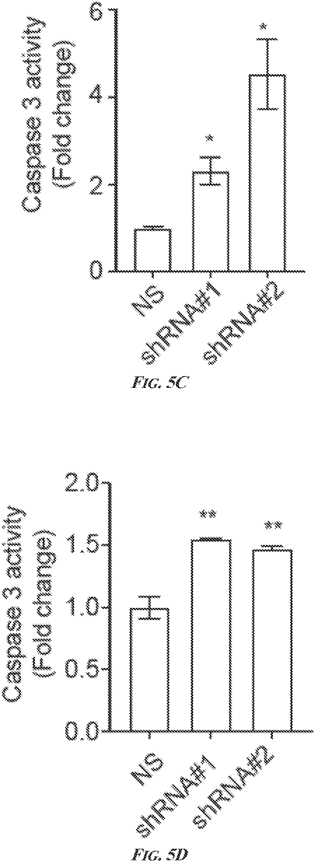

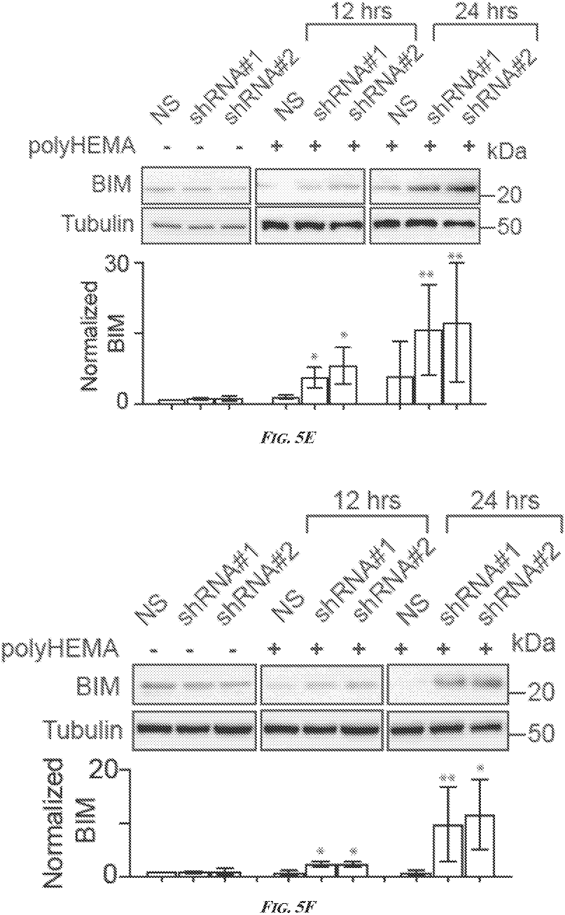

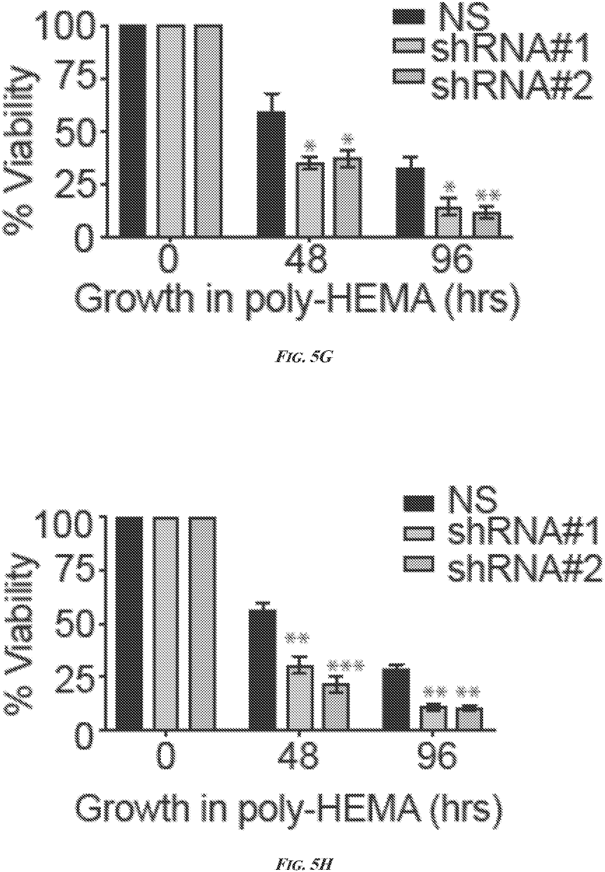

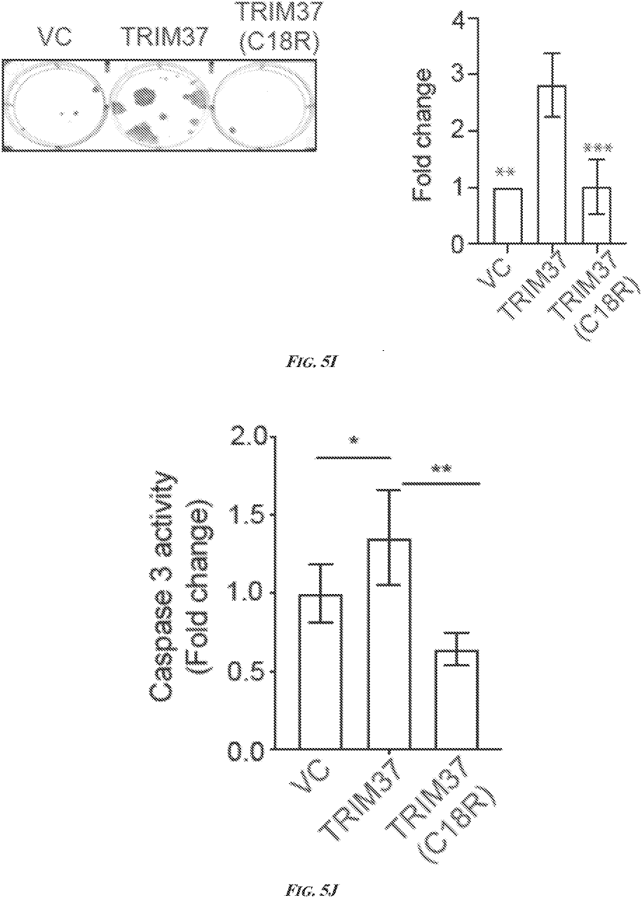

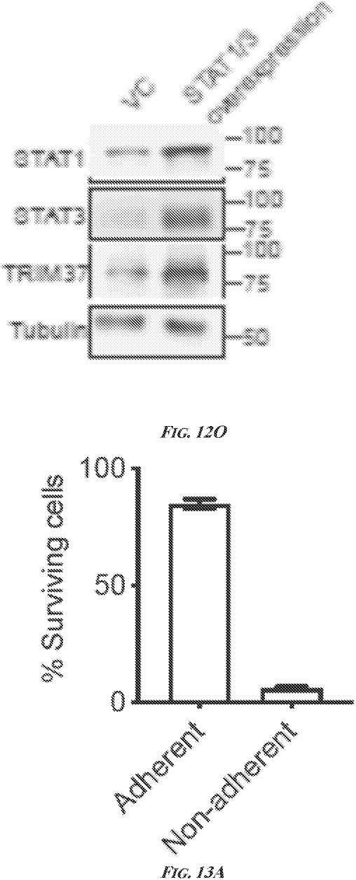

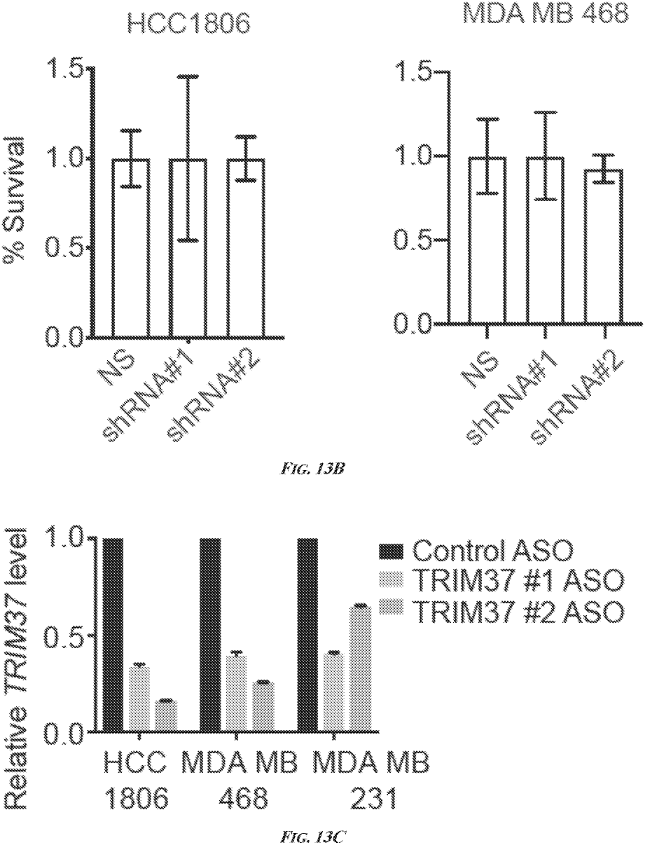

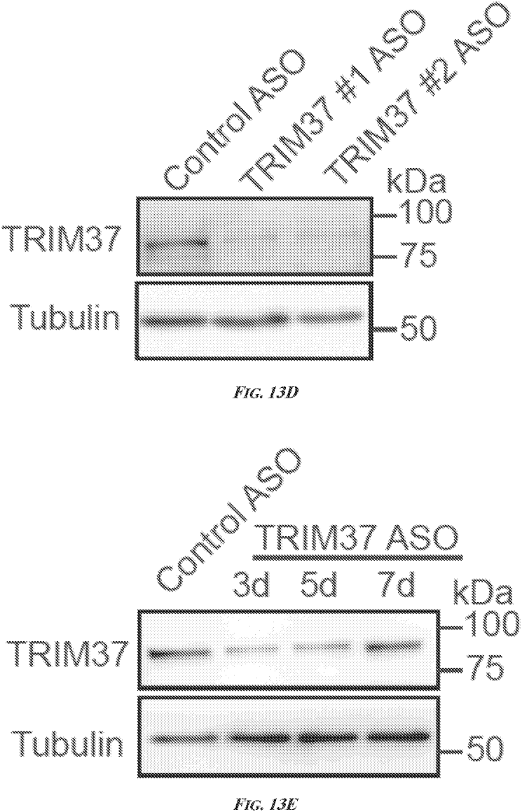

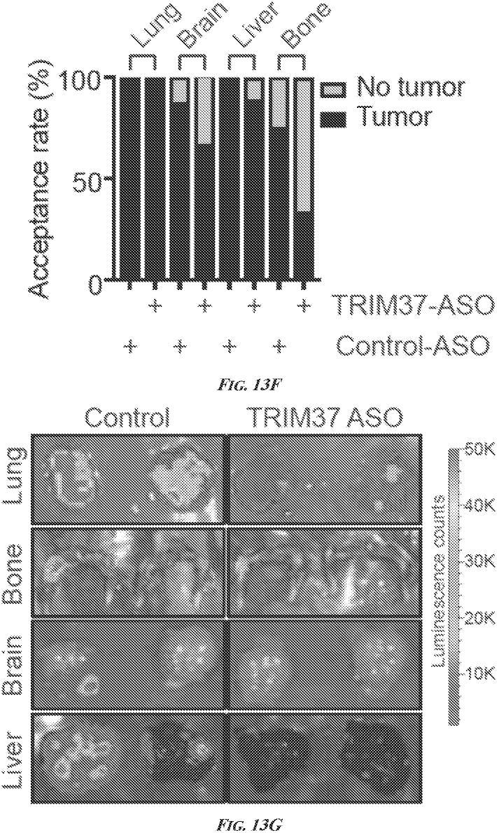

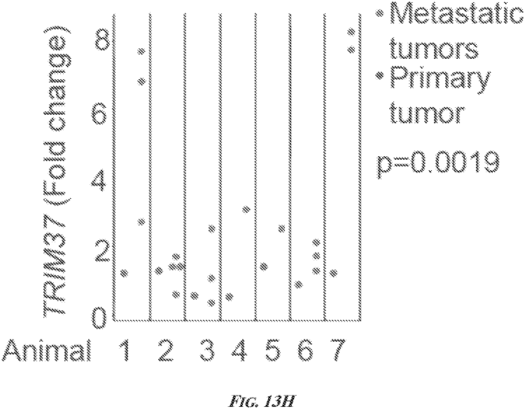

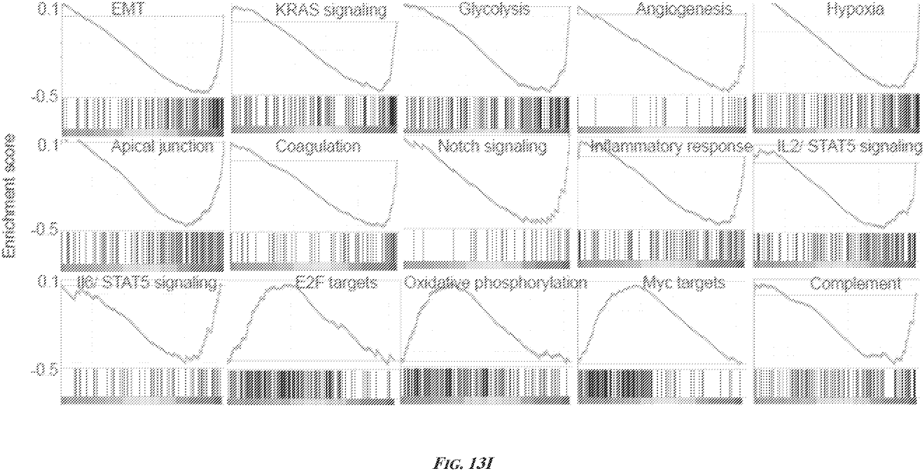

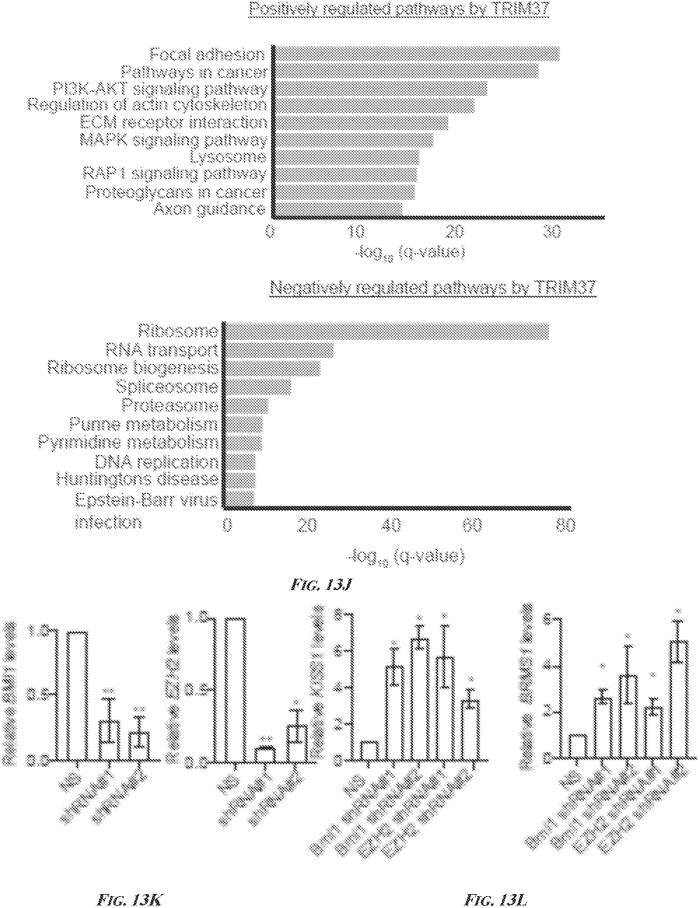

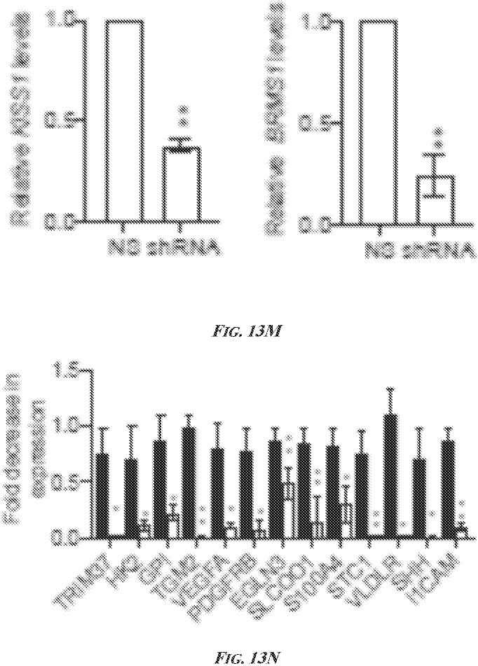

In addition to being chemoresistant, TNBC tumors are also highly aggressive, demonstrating high metastatic potential. We therefore asked whether TRIM37 contributes to the acquisition of anoikis resistance, a prerequisite for metastasis. We therefore examined anoikis in control and TRIM37-knockdown TNBC cells induced due to growth in the suspension culture. Colony formation assay (FIGS. 5A-5B) and caspase 3 assay (FIGS. 5C-5D) demonstrated that TRIM37-knockdown sensitized TNBC cells to detachment-induced anoikis, whereas non-silencer (NS) TNBC cells remained viable. In contrast, MCF10A cells showed ~90% decrease in cell viability in non-adherent conditions (FIG. 13A). No significant differences between NS and TRIM37-knockdown TNBC cells were observed when cultured in adherent conditions (FIG. 13B). Next, we examined whether TRIM37 attenuates the activity of the pro-apoptotic factor BIM, a downstream effector in the BAK/BAX-dependent apoptotic pathway (Woods et al., 2007). Immunoblot analysis and viability assay confirmed that BIM levels were significantly upregulated in TRIM37-knockdown TNBC cells (FIGS. 5E-5F), which showed decreased viability compared to control cells post-detachment (FIGS. 5G-5H). Reciprocally, the overexpression of wild type TRIM37, but not TRIM37(C18R), rendered MCF10AT cells resistant to anoikis (FIG. 5I). Quantitation of the apoptotic effector Caspase 3 showed significantly reduced activity in TRIM37-expressing MCF10AT cells relative to TRIM37(C18R)-expressing MCF10AT cells post-detachment (FIG. 5J). Consistently, decreased BIM expression (FIG. 5K) and increased viability (FIG. 5L) was observed in TRIM37-expressing cells relative to control or TRIM37(C18R)-expressing MCF10AT cells post-detachment, confirming that TRIM37 induces the anoikis resistance in TNBC cells, which is required for survival in circulation.

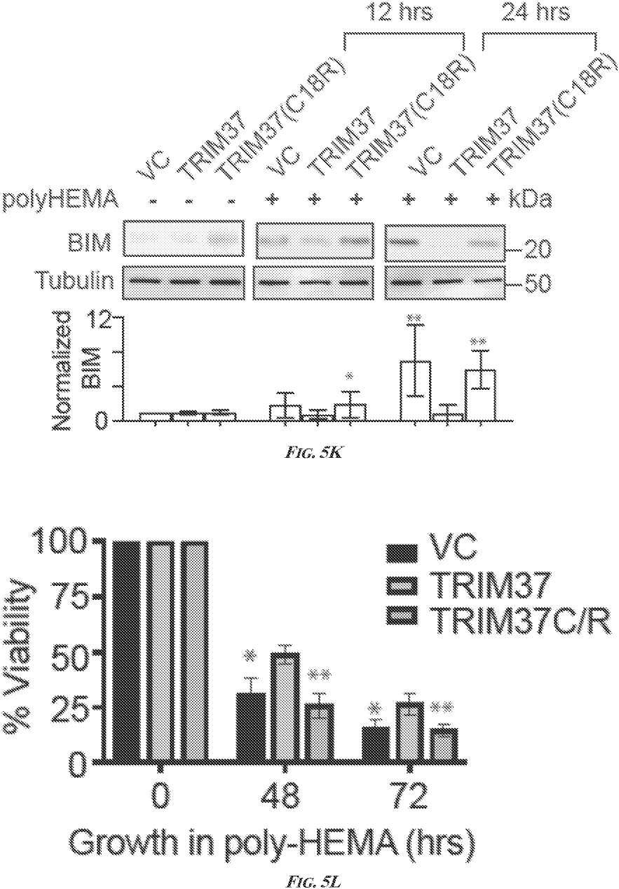

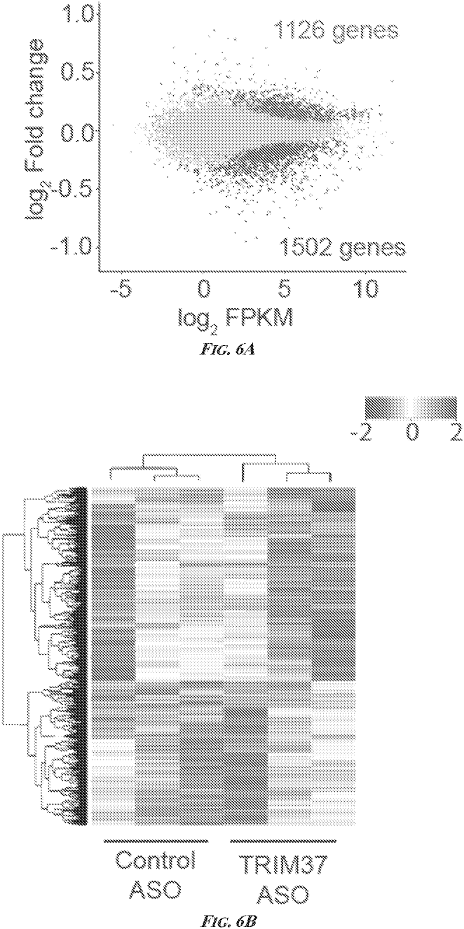

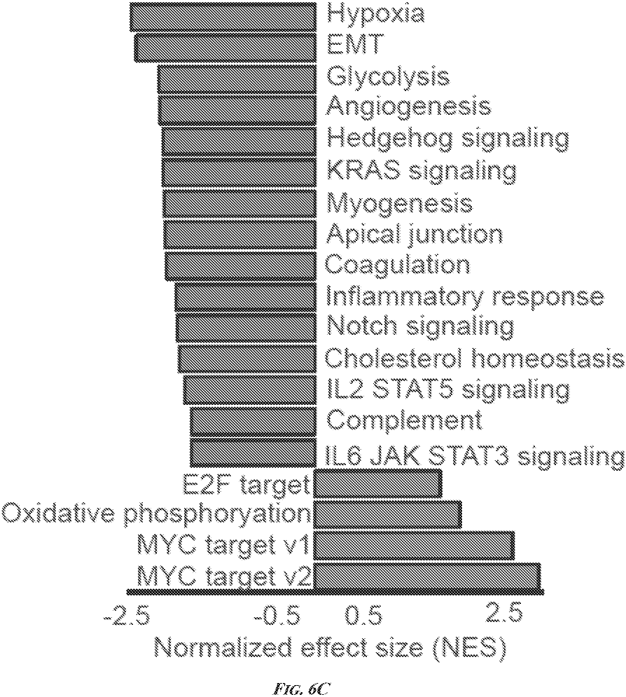

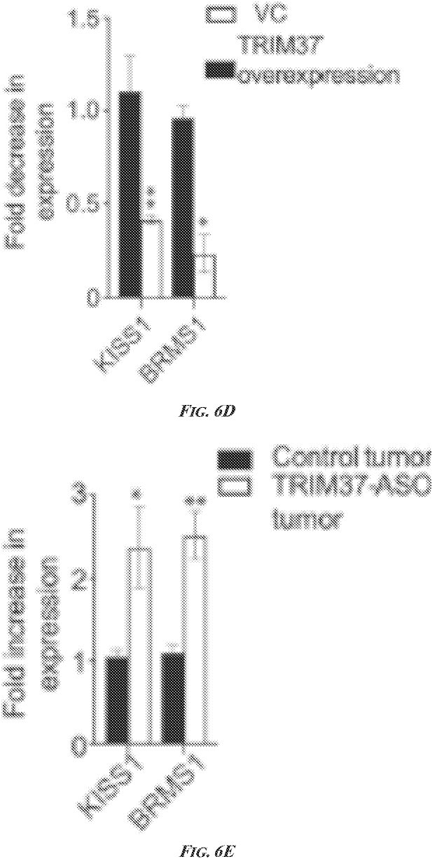

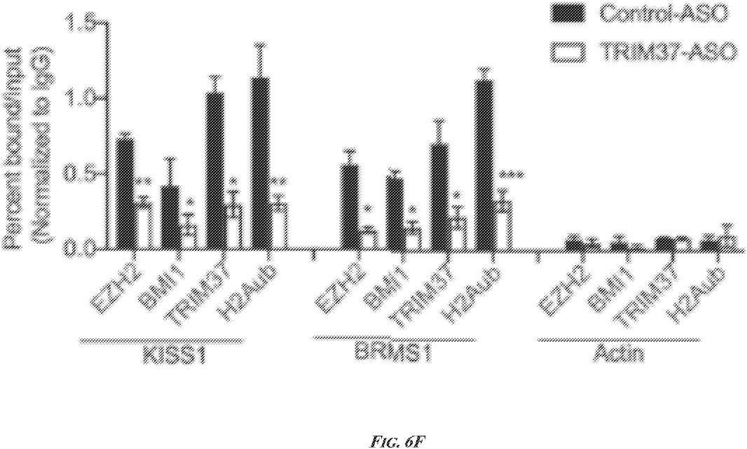

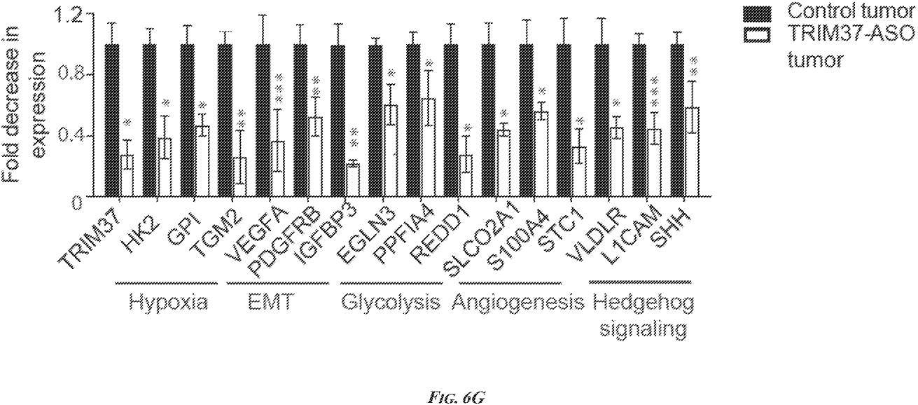

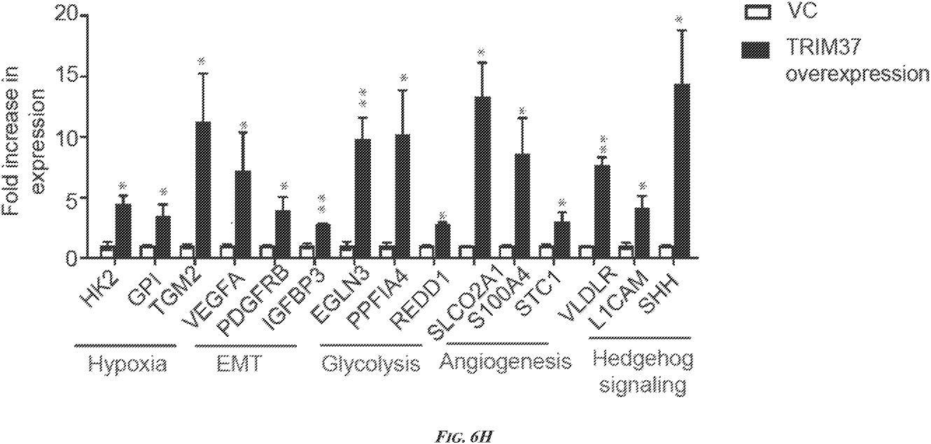

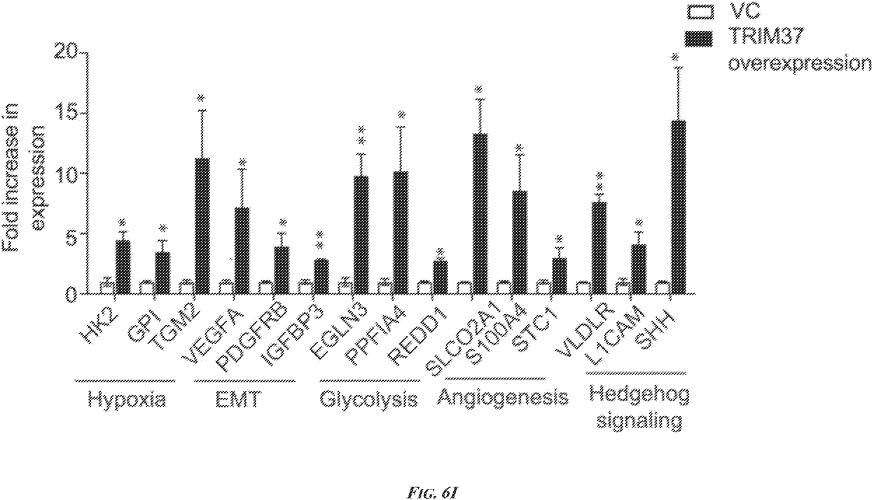

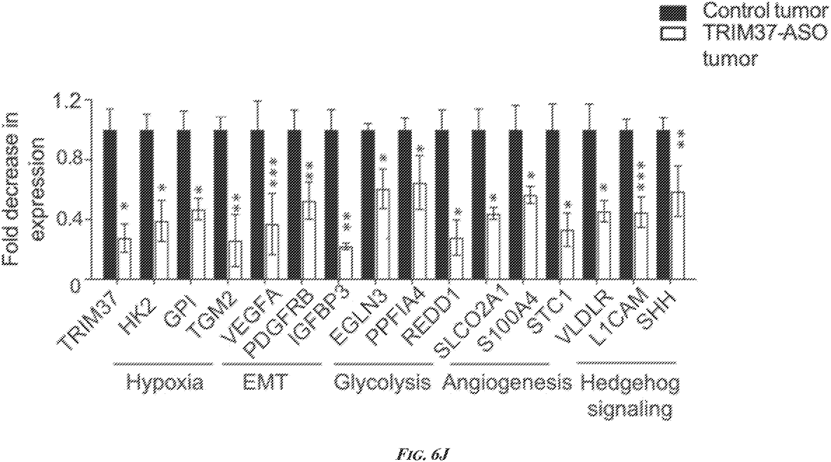

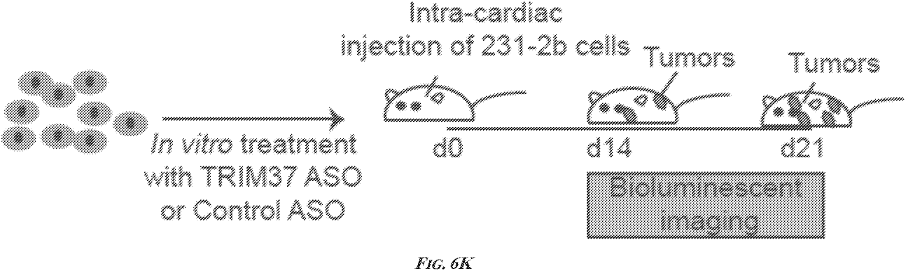

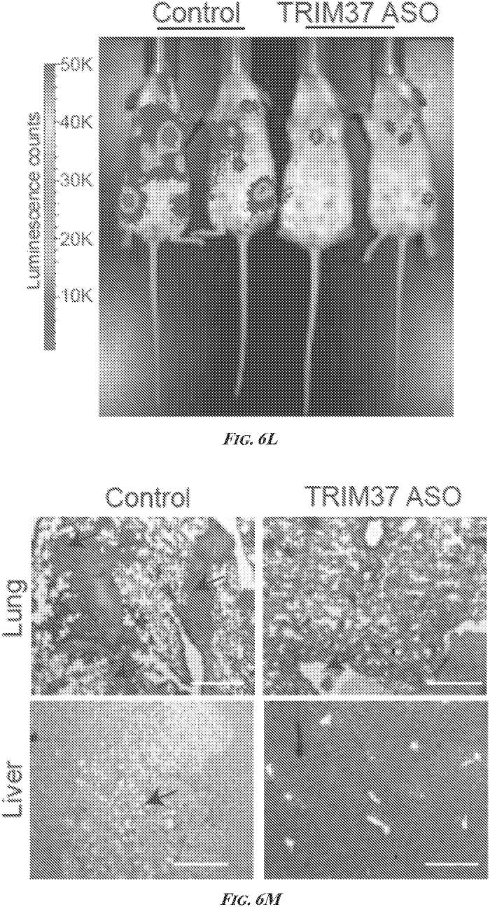

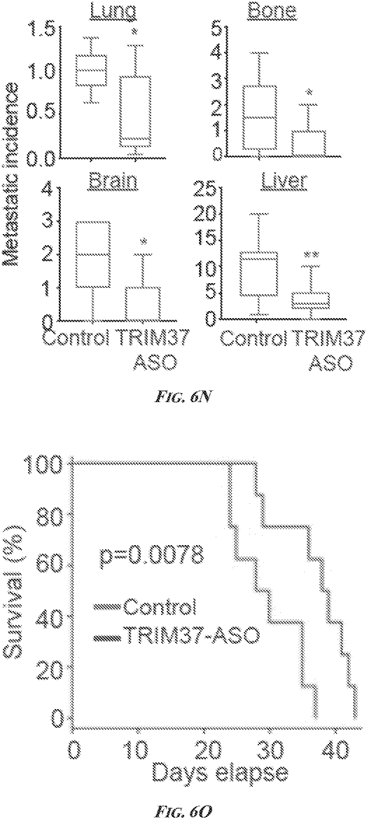

We next asked whether TRIM37 expression alters metastatic seeding and growth in an experimental metastasis model using a brain metastatic derivative of human mammary TNBC and luciferase expressing cells, MDA-MB-231-D3H2LN-2b, hereafter referred to as 231-2b (Jenkins et al., 2005; Minn et al., 2005; Tominaga et al., 2015). To knockdown TRIM37, we used TRIM37-specific antisense oligonucleotides (TRIM37-ASO), which have higher metabolic stability, as well as penetrate the blood-brain-barrier. TRIM37-ASO knockdown efficacy was confirmed in a panel of TNBC cells at the RNA level by qRT-PCR (FIG. 13C), and at the protein level by immunoblotting (FIGS. 13D-13E). We injected 2×10⁴ control or TRIM37-knockdown 231-2b cells into the left cardiac ventricle of female NOD scid gamma (NSG) mice and monitored in vivo metastasis capability by noninvasive bioluminescent imaging (BLI) after intraperitoneal injection of luciferase (FIG. 6K and FIG. 13F). Mice injected with 231-2b control tumors developed tumors in sites comparable to human breast cancer metastases, such as the brain, lung, liver, lymph nodes, and bone (FIG. 6L). In contrast, mice injected with TRIM37-knockdown 231-2b cells showed dramatic reduction in metastatic burden (FIG. 6L). The distant tumor growth of the control and TRIM37-knockdown 231-2b cells was confirmed by histological analysis postmortem (FIG. 13G) as well as by hematoxylin and eosin staining of tumor tissue sections (FIG. 6M). TRIM37-knockdown reduced the tumor burden in lungs by ~2-fold, bone by ~2-fold, brain by ~3-fold, and liver by ~3-fold compared to the control animals 21-days after TNBC cell injection (FIG. 6N). We also found that TRIM37 inhibition lengthened the survival of mice by ~7-days (FIG. 6O). Furthermore, our analysis of previously published comparative expression profiling of PDX mammary fat pad primary and corresponding lung metastatic tumors (Echeverria et al., 2018) revealed higher TRIM37 expression in metastatic tumors compared to corresponding matched primary tumors (FIG. 13H). Together, in vivo results confirm our in vitro findings that TRIM37 increases the survival of TNBC cells in circulation, promotes TNBC proliferation, and metastatic growth.

### EXAMPLE 6

### TRIM37 Remodels Transcriptional Program Favoring TNBC Metastasis

While the emergence of chemoresistance is closely related to metastasis, the ability of a cancer cell to survive and proliferate under continuous standard chemotherapy does not warrant metastasis. Our analysis of previously published expression profiling of PDX mammary fat pad primary and corresponding lung metastatic tumors (Echeverria et al., 2018) revealed higher TRIM37 in metastatic lesions (FIG. 13H). These results indicated that higher TRIM37 levels are maintained throughout the metastatic transition.

TRIM37 in association with polycomb complex alters gene expression to promote tumorigenesis (Bhatnagar et al., 2014). To test whether TRIM37 causes transcriptional misregulation of genes involved in metastasis, we knocked down TRIM37 in MDA-MB-231-D3H2LN-2b (Tominaga et al., 2015), hereafter referred to as 231-2b, using TRIM37-specific ASO (TRIM37-ASO; FIG. 13D) and performed transcriptomic analysis. Of the ~2,600 genes whose expression differed significantly between TRIM37-knockdown and control cells (GSE136617; FIGS. 6A-6B), ~71 tumor and metastases suppressors, such as KISS1 and BRMS1, were significantly down-regulated by TRIM37 (Table 6). As robust metastatic suppressors, KISS1 and BRMS1 reciprocally correlate with increased tumor recurrence, metastatic foci and reduced disease-free survival (Kodura & Souchelnytskyi, 2015; Ulasov et al., 2019). The anti-metastatic function is mediated by altered gene expression through cell signaling pathways (Cicek et al., 2005; Wu et al., 2019) as well as transcriptional regulation (Hurst et al., 2008; Cicek et al., 2009). Additionally, gene set enrichment analysis (GSEA) revealed that TRIM37-knockdown downregulates hypoxia, EMT transition, glycolysis, angiogenesis, inflammatory, and immune response-related genes in TNBC cells, indicating TRIM37-dependent activation of a pro-metastatic transcriptional program (FIG. 6C and FIG. 13I). Likewise, KEGG pathway analysis identified TRIM37 target genes that associated with focal adhesion, pathways in cancer, actin cytoskeleton, ECM interaction, and signaling pathways (FIG. 13J).

To validate the RNA-seq results, we analyzed expression of representative genes in p53^{-/-} MCF10A cells ectopically expressing TRIM37. Expression of KISS1 and BRMS1 was significantly lower in cells ectopically expressing TRIM37 compared with empty vector (FIG. 6D). Conversely, TRIM37-knockdown tumors expressed KISS1 and BRMS1 at significantly higher levels relative to control xenograft tumors (FIG. 6E). To investigate the mechanism by which TRIM37 regulate KISS1 and BRMS1, we analyzed binding of polycomb complex components, BMI1 and EZH2, to BRMS1 and KISS1 by directed-ChIP assays. Both the gene promoters were enriched for BMI1 and EZH2 which was diminished after TRIM37 knockdown (FIG. 6F). These gene promoters were also enriched for H2Aub, which was reduced after TRIM37 knockdown (FIG. 6F). As expected, knockdown of BMI1 and EZH2 resulted in increased expression of these genes (FIGS. 13K and 13L).

Our results raised the possibility that TRIM37-mediated repression of metastases suppressors induces transcriptional program favoring metastasis. To test this idea, we analyzed a representative set of 15 genes in the top GSEA categories based on statistical analysis and their known biological functions in multiple steps of metastasis (Tables 7A-7E). For all 15 genes analyzed, knockdown of BRMS1 or KISS1 resulted in their increased expression (FIGS. 6G and 6H, and FIG. 13M). Consistently, ectopic expression of TRIM37 in p53^{-/-} MCF10A cells significantly increased expression of all the TRIM37 target genes compared with empty vector (FIG. 6I), indicating reciprocal relationship between TRIM37 and metastases suppressors. To validate RNA-seq results in vivo, a subset of TRIM37 target genes were analyzed in TRIM37-knockdown tumors. As expected, all the 15 genes analyzed were significantly reduced in TRIM37-knockdown tumors relative to control tumors (FIG. 6J). Moreover, knockdown of TRIM37 also decreased expression of TRIM37 target genes in MCF7 cells (FIG. 13N).

To investigate directly the potential function of TRIM37 in metastasis, we compared the in vivo propensity of control and TRIM37-knockdown cells in NSG mice (FIGS. 6K and 6L). Knockdown of TRIM37 showed dramatic reduction in the metastatic burden in comparison to control tumors that developed in sites comparable to human breast cancer metastases, such as the brain, lung, liver, lymph nodes, and bone (FIG. 6L and FIGS. 13F and 13G). TRIM37-knockdown reduced the metastatic tumor burden in lungs by ~2-fold, bone by ~2-fold, brain by ~3-fold, and liver by ~3-fold compared to the control animals 21-days after TNBC cell injection (FIG. 6N). Further, histological analysis of tumors confirmed the distant tumor growth of the control and TRIM37-knockdown 231-2b cells in lung and liver (FIG. 13O). Finally, TRIM37-knockdown in 231-2b resulted in a modest but significant improvement of post-injection survival (FIG. 6O). Collectively, these results demonstrate that TRIM37 overexpression enforces transcriptional program in TNBC tumors that promotes metastatic progression.

### EXAMPLE 7

### Design, Construction, and Characterization of Molecularly Targeted Nanoparticles to Deliver TRIM37-ASO In Vivo

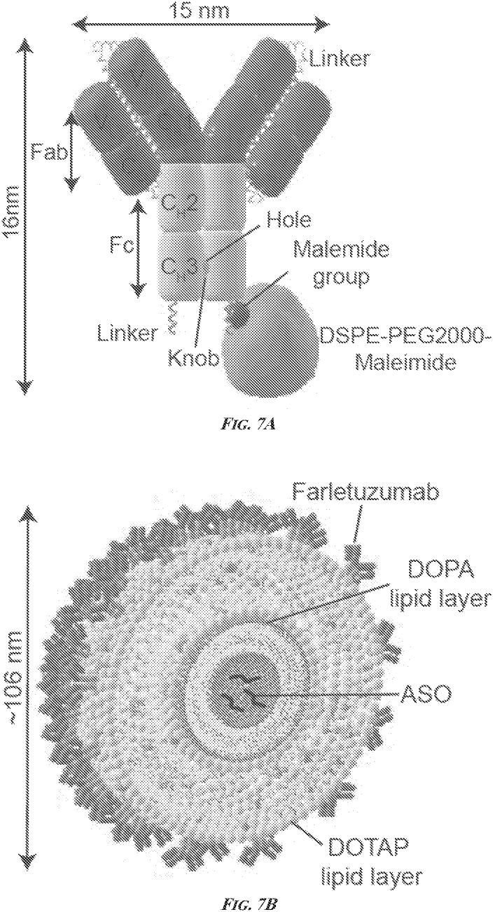

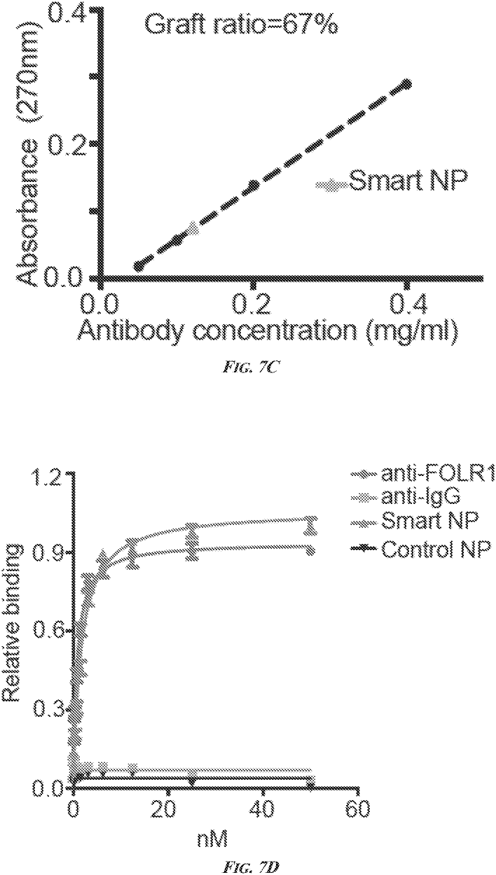

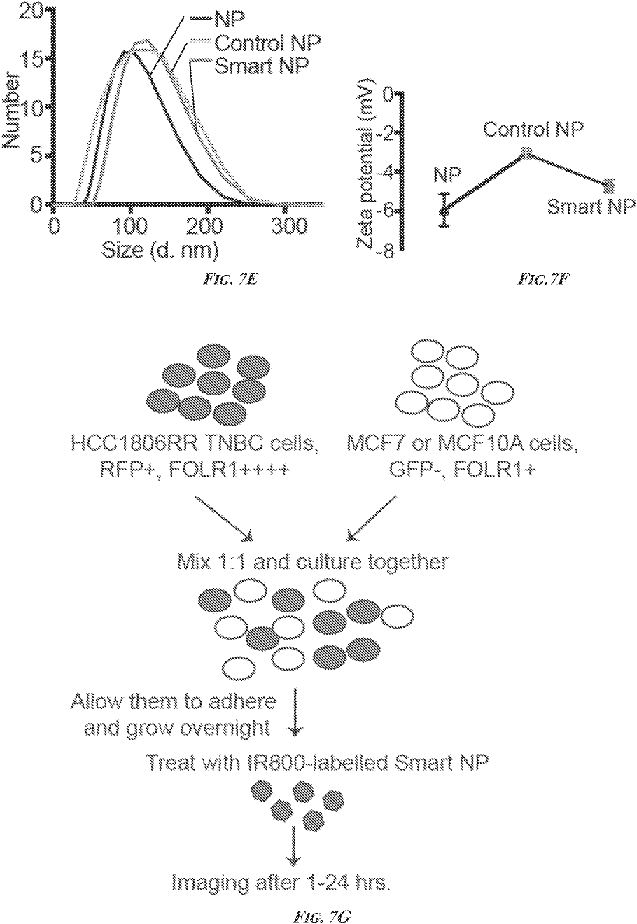

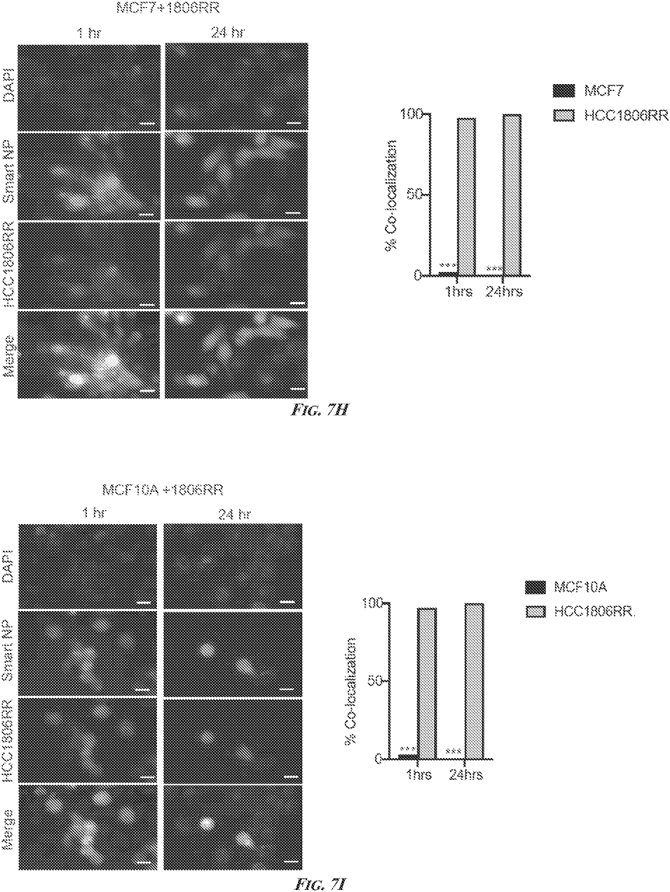

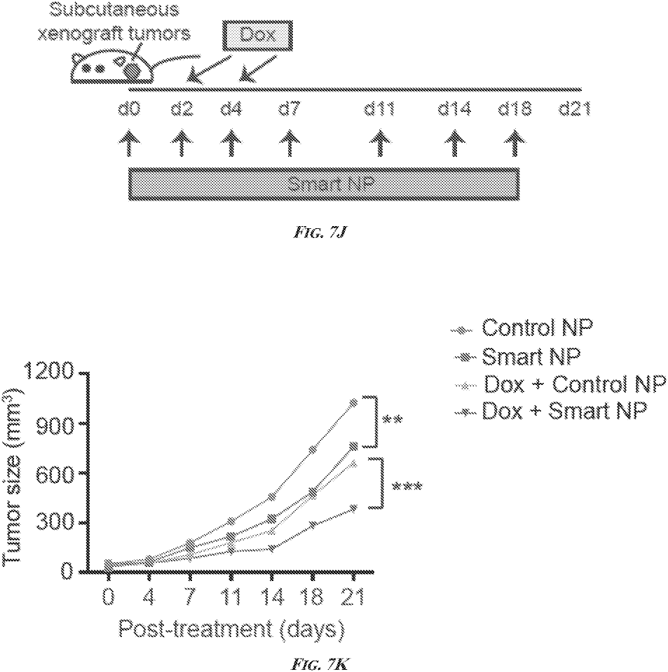

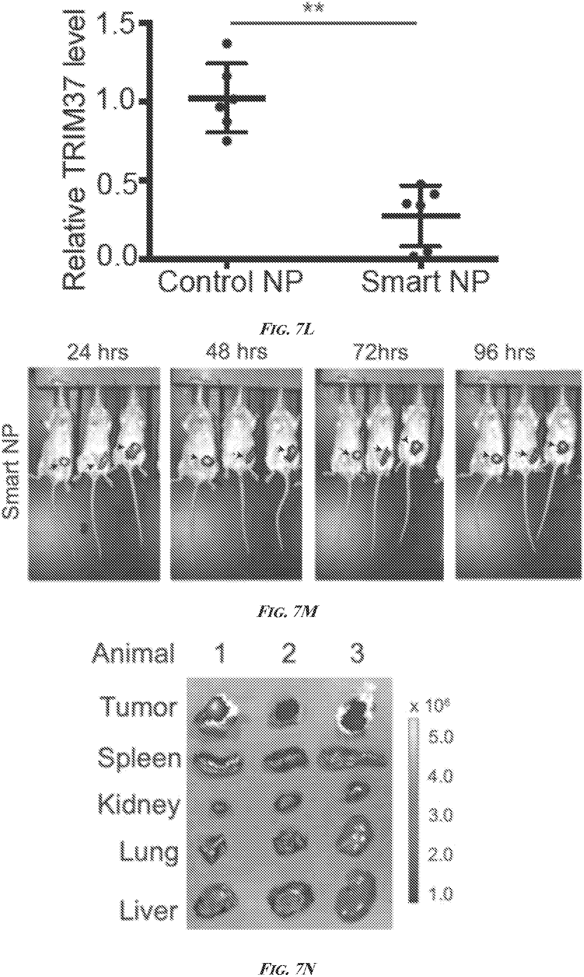

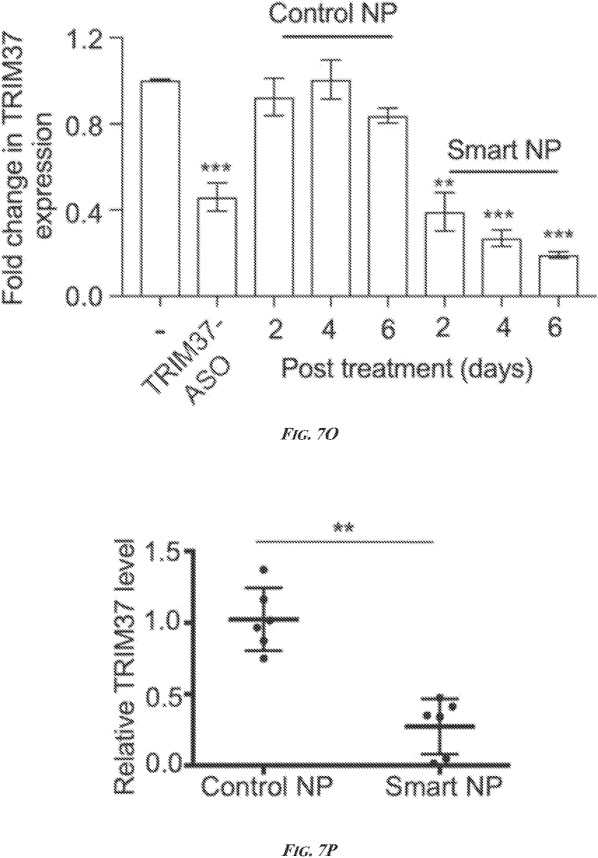

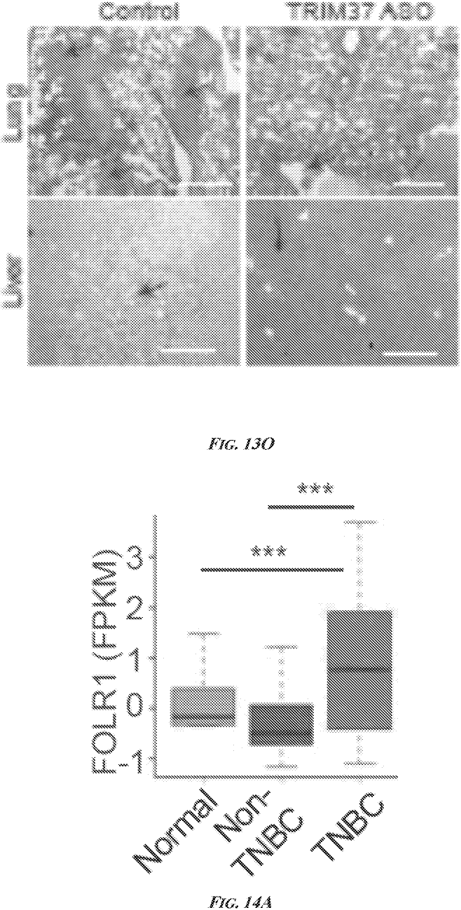

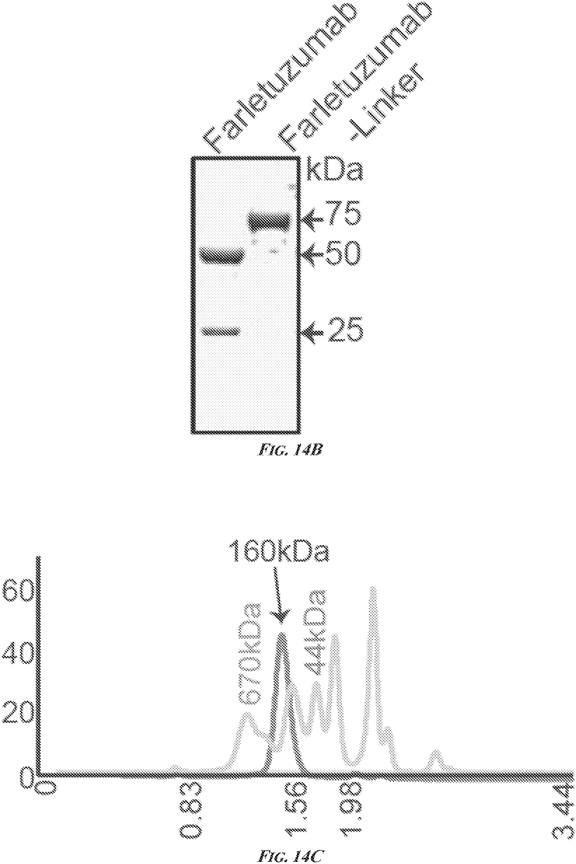

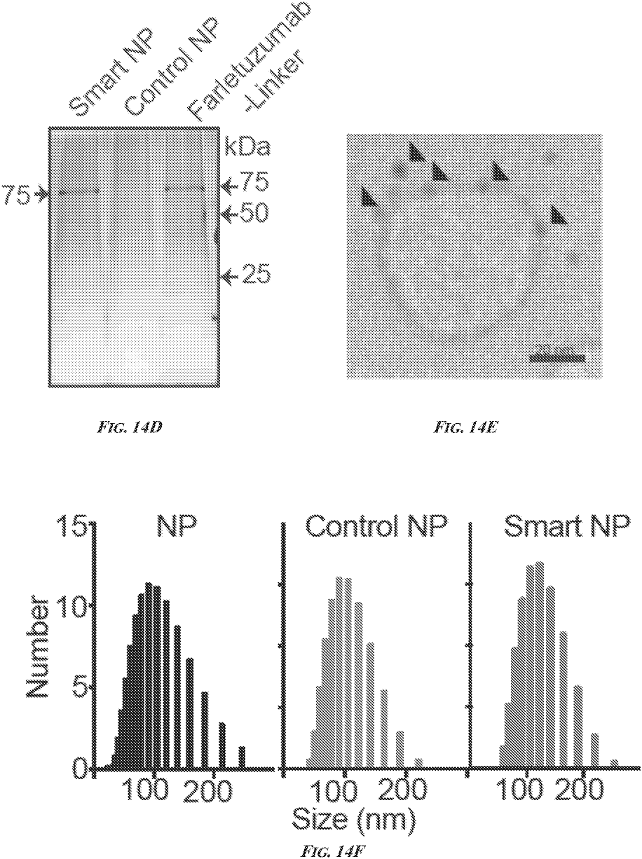

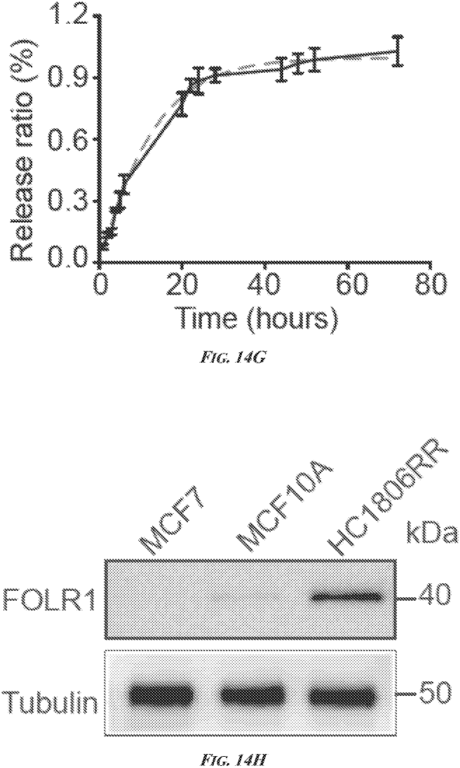

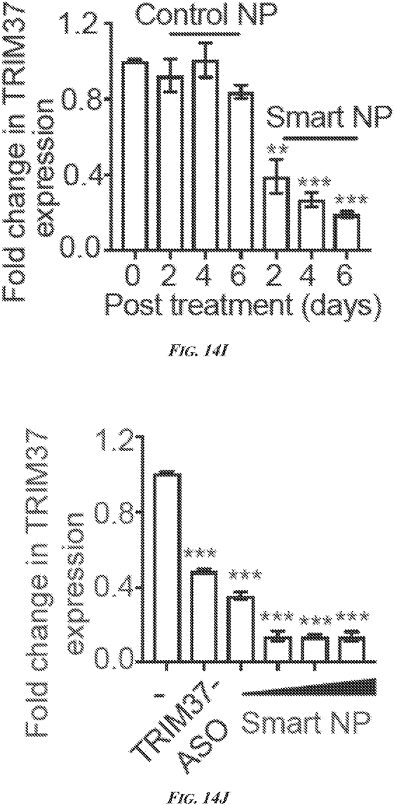

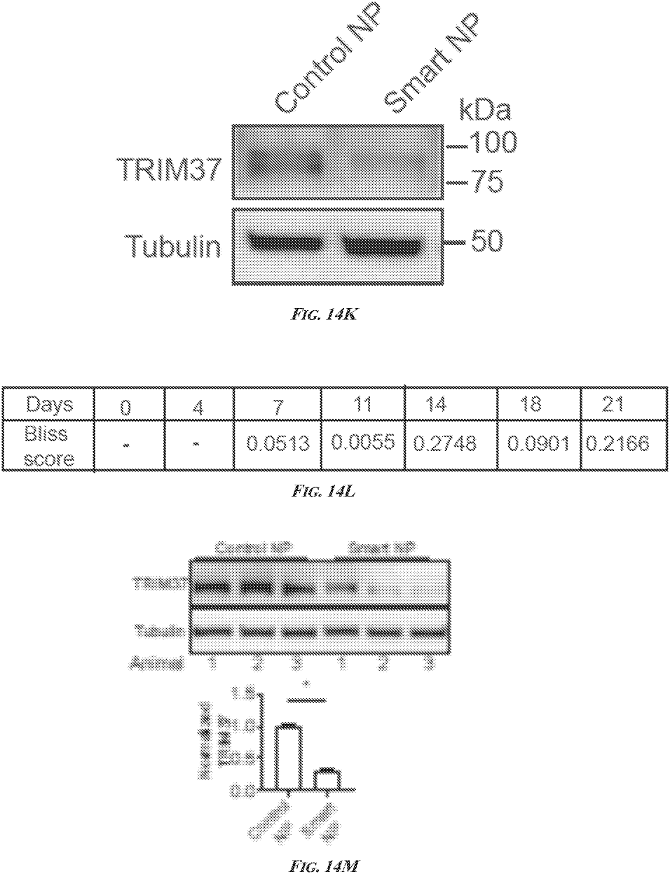

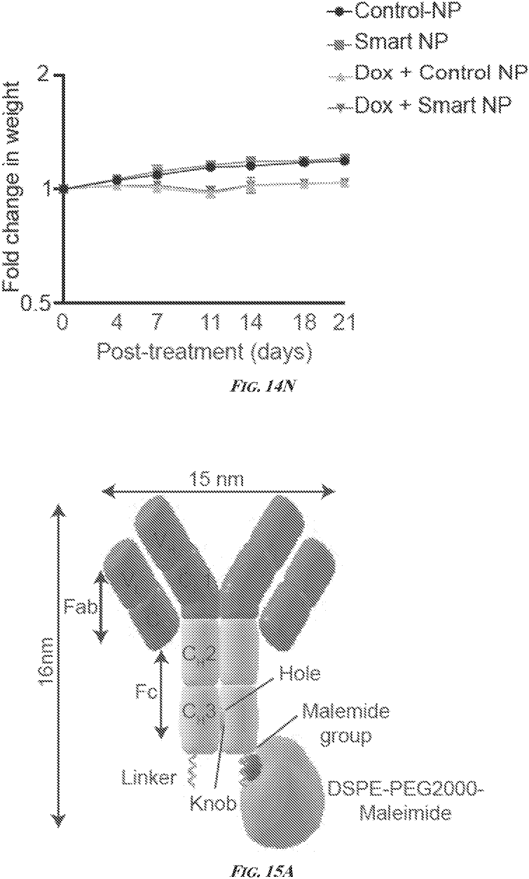

We show that TRIM37 alters chromatin modification to resist chemotherapy and enforces changes in gene expression to favor metastatic transition. These results formed the underlying rationale for targeting TRIM37 as a therapeutic strategy for treating TNBC. To systematically assess the therapeutic effectiveness of inhibiting TRIM37, we engineered liposome-based nanoparticles comprising DOPA, DOTAP, and DSPE-PEG2000-Maleimide with TRIM37-ASO encapsulated in the core. We functionalized the nanoparticles by incorporating an investigational FOLR1 antibody (Farletuzumab). To this end, we site-specifically, and covalently conjugated DSPE-PEG2000-Maleimide to a cysteine-containing Fc-linkered sequence at the C-terminus of a knob heavy chain in Farletuzumab (FIG. 7A and FIGS. 14B-14D). TCGA data analysis showed higher expression of FOLR1 in TNBC relative to non-TNBC and normal tissues (FIG. 14A). For convenience, these mono-disperse complexes are referred to as “smart nanoparticles” (FIG. 7B and FIG. 14E). FIG. 7C confirmed that ~67% of the maleimide groups present on the outer surface of the smart nanoparticles were functionalized with Farletuzumab. No significant differences in the binding affinity of Fc-linkered and monomeric Farletuzumab were observed (FIG. 7C). As a control, we generated and characterized Farletuzumab-conjugated nanoparticles with control-ASO as a payload; hereafter, referred to as “control nanoparticles”. As expected, the diameter of 106 ± 21 nm (FIG. 7E), and the overall charge of -4.72 ± 0.30 mV (FIG. 7F) were not significantly different between smart and control nanoparticles.

Because higher levels of FOLR1 associate with advanced metastatic stage and recurrence in TNBC (Ginter et al., 2017), we rationalized that smart nanoparticles would preferentially deliver TRIM37-ASO to TNBC cells. To test this idea, HCC1806RR (Volk-Draper et al., 2012) was co-cultured with either MCF7 or MCF10A cells (FIG. 7G). Immunoblot analysis confirmed that FOLR1 is expressed at significantly higher levels in HCC1806RR compared to MCF7 or MCF10A cells (FIG. 14F). Interestingly, we observed ~100% uptake of IR800-labeled nanoparticles by TNBC cells compared to MCF7 or MCF10A cells post-mixing (FIGS. 7H-7I).

Negative stain transmission electron microscopy of the smart nanoparticles revealed a mono-disperse population (FIG. 14E). The diameter of the nanoparticles, tested by Zetasizer, was 106 ± 21 nm (FIGS. 7E and 14F), and the overall charge of the nanoparticles was -4.72 ± 0.30 mV (FIG. 7F), which was not significantly different from empty or control nanoparticles. As shown in FIG. 14G, smart nanoparticles demonstrate sustained TRIM37-ASO release over a period of 48 hours with a biphasic release pattern. Because western blot analysis confirmed higher expression of FOLR1 in HCC1806RR cells compared to MCF7 and MCF10A cells (FIG. 14H), we evaluated TRIM37-ASO delivery to TNBC cells by smart nanoparticles. To this end, HCC1806RR cells stably expressing red fluorescent protein (RFP) were co-cultured with either MCF7 or MCF10A cells (FIG. 7G). We observed ~100% uptake of nanoparticles by TNBC cells post-mixing and no significant co-localization of nanoparticles by MCF7 or MCF10A cells was observed (FIGS. 7H-7I). Markedly, TRIM37 expression in MDA MB 231 cells treated with smart nanoparticles was reduced by ~80% in a time-dependent manner relative to control nanoparticles, and compared to TRIM37-ASO, showed ~50% more reduction in TRIM37 expression (FIGS. 14I-14K), which can be attributed to the higher stability of ASO in nanoparticles.

To investigate the outcome of TRIM37 targeting in clinical settings, we challenged 231-2b-derived xenografts with smart nanoparticles in combination with chemotherapy. When tumors reached the size of ~50 mm³, animals received two doses of either 2 mg/kg Dox or vehicle (FIG. 7J). Furthermore, within each group, animals were randomly administered 1.2 mg/kg (~4×10⁷nanoparticles) of smart or control nanoparticles intra-tumor every three days for 18 days (FIG. 7J). As anticipated, tumor growth was significantly attenuated in the smart nanoparticles and Dox-treated animals while the control-nanoparticle treated tumors continued to grow (FIG. 7K). Most strikingly, the combination of TRIM37 inhibition with Dox showed the greatest reduction in 231-2b tumors, indicating a therapeutic synergism as indicated by Bliss score (FIGS. 7K and 14L). qRT-PCR and immunoblot analysis confirmed TRIM37 inhibition in smart nanoparticle-treated tumors compared to control nanoparticle-treated tumors (FIGS. 7L and 14M). No significant changes were observed in the body weight of treated mice over the duration of the experiment (FIG. 14N). Collectively, our results demonstrate that smart nanoparticles selectively deliver TRIM37-ASO to the TNBC cells and effectively reduce xenograft tumor growth.

Finally, to test the TNBC cells selectivity of smart nanoparticles in vivo, we injected smart nanoparticles into the 231-2b xenograft-bearing mice. As expected, the smart nanoparticles selectively accumulated in the xenograft tumors within 24 hrs. and remained localized to the tumor up to 96 hrs. (FIG. 7M). The accumulation of smart nanoparticles in xenograft tumors was confirmed by the detailed tissue distribution using mice necropsies (FIG. 7N).

Next, we evaluated the biological activity of the smart nanoparticles, which demonstrated sustained TRIM37-ASO release over a period of 48 hours with a biphasic release pattern (FIG. 14H). Treatment of MDA MB 231 cells with smart nanoparticles decreased TRIM37 expression by ~80% in a time-dependent manner relative to control nanoparticles (FIG. 7O). To investigate smart nanoparticles-mediated TRIM37 targeting in vivo, we challenged 231-2b-derived xenografts with smart nanoparticles. qRT-PCR and immunoblot analysis confirmed TRIM37 inhibition in smart nanoparticle-treated tumors compared to control nanoparticle-treated tumors (FIG. 7P and FIG. 14M). Together, these results confirmed that smart nanoparticles-mediated delivery of TRIM37-ASO effectively decrease TRIM37 expression in TNBC tumors.

### EXAMPLE 8

### Targeting TRIM37 to Prevent Metastasis in TNBC

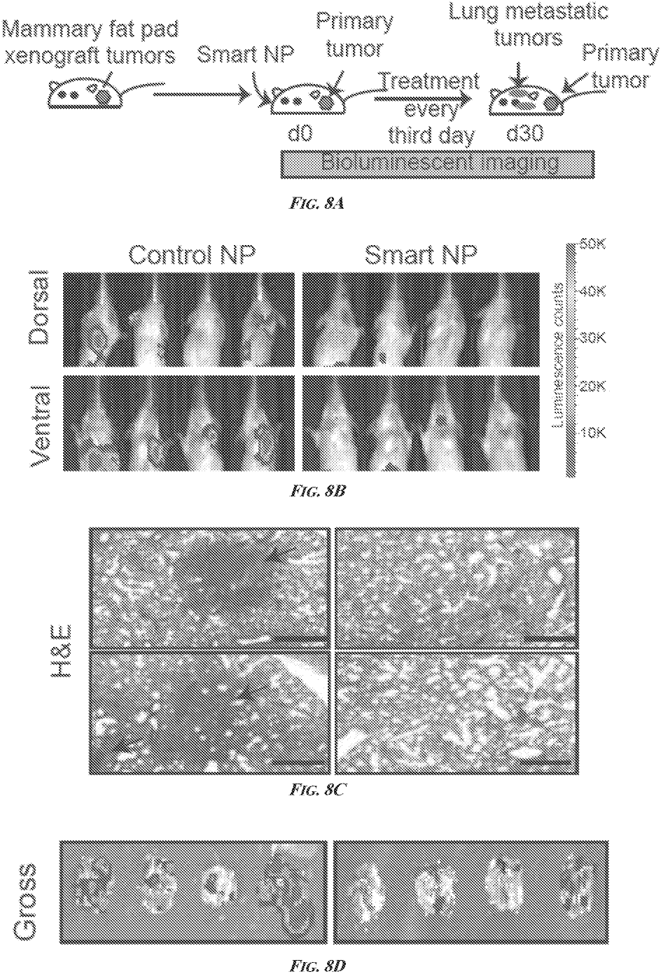

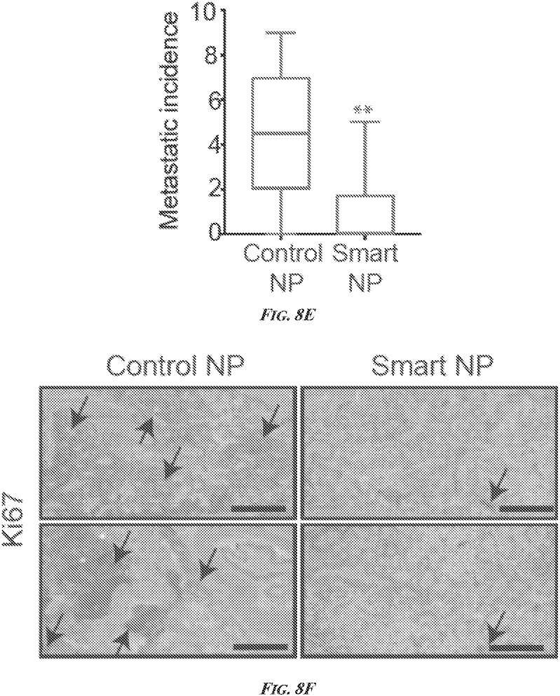

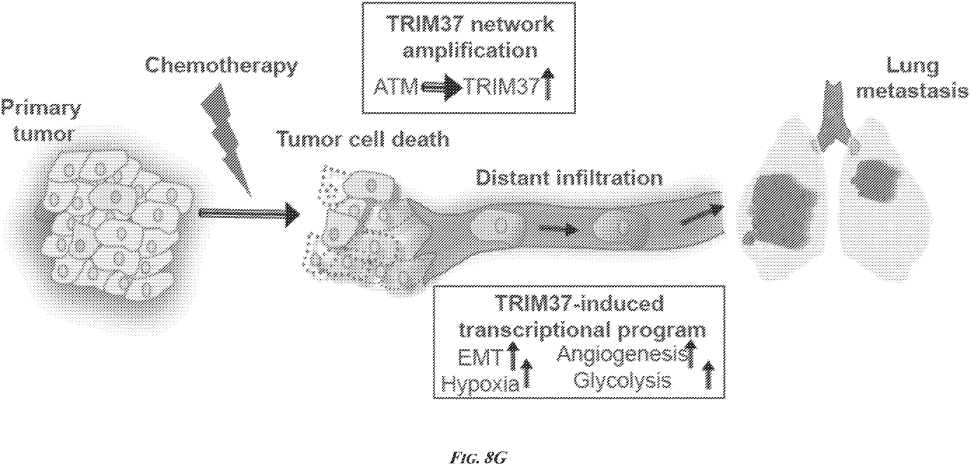

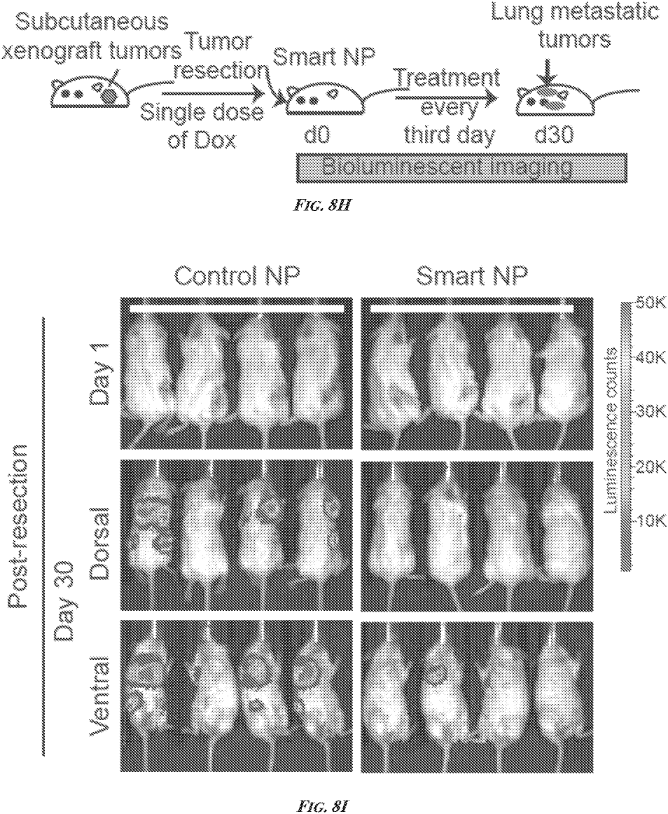

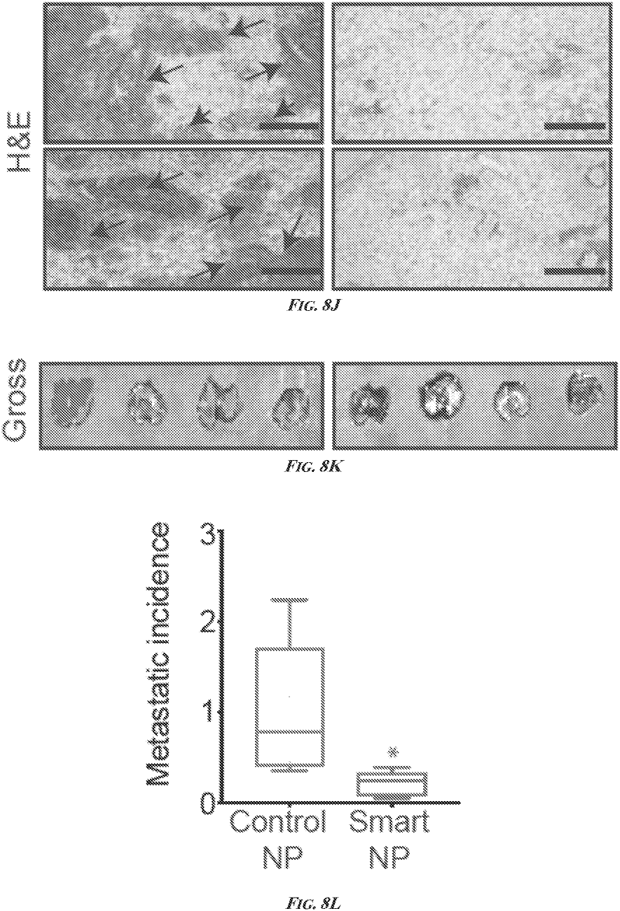

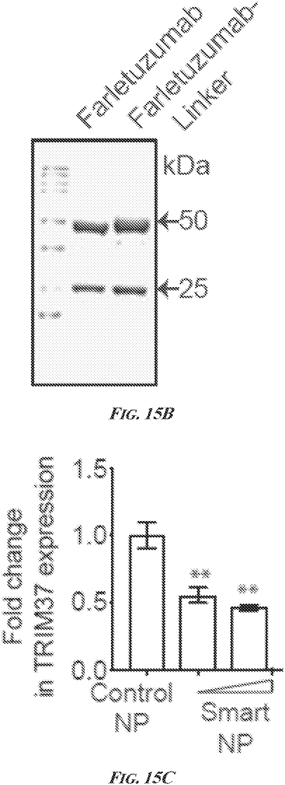

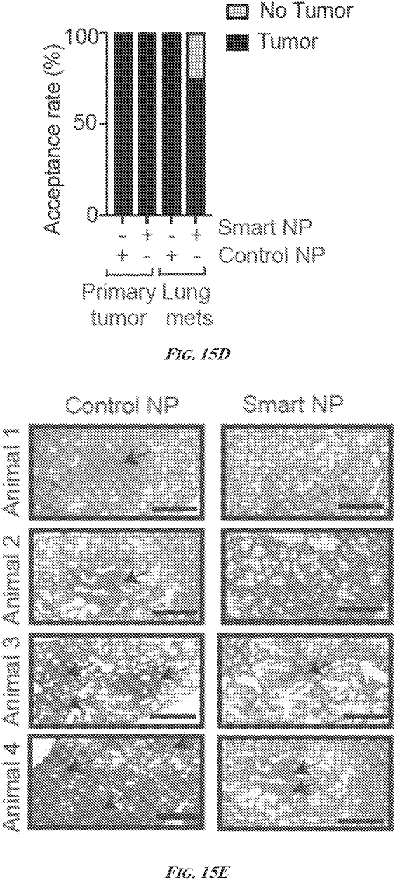

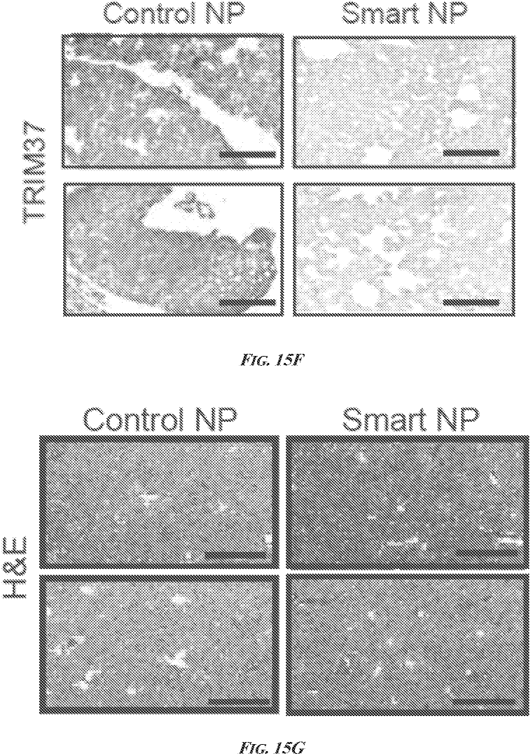

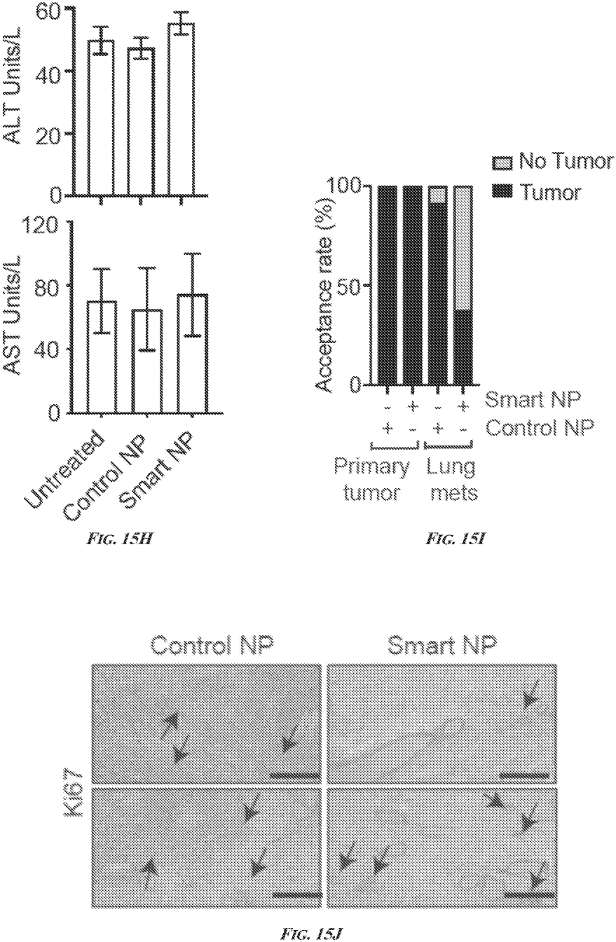

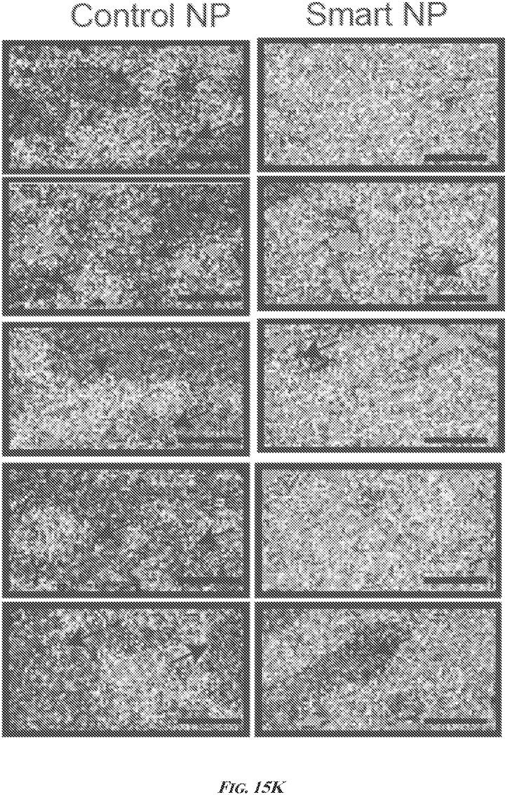

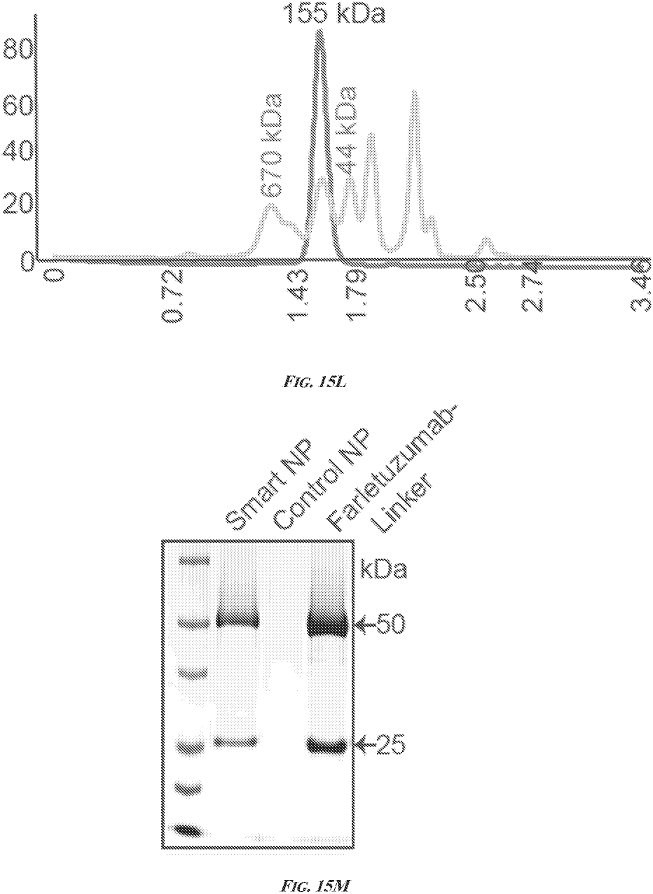

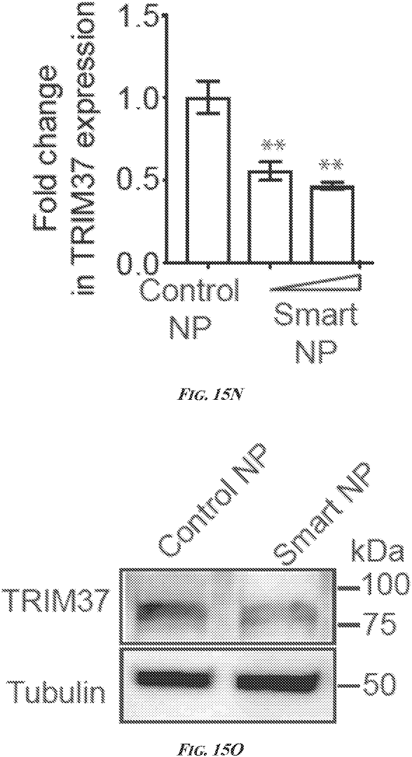

Next, we asked whether TRIM37 targeting will reduce metastatic lesions in syngeneic spontaneous metastasis murine model. To this end, Balb/c mice bearing mammary fat pad tumors derived from murine 4T1 cells were administered 1.2 mg/kg of control or smart nanoparticles (FIG. 8A). To inhibit TRIM37, we utilized smart nanoparticles conjugated with murine cross-reactive anti-FOLR1 (FIGS. 15A - 15C). While ~90% of the control nanoparticle treated mice developed overt lung metastasis, smart nanoparticles-treated animals showed a dramatic decrease in lung metastasis, with ~60% of smart nanoparticles-treated animals showing no detectable lung metastases (FIG. 8B and FIG. 15D). Animals were sacrificed at day 30 post-treatment due to the moribund condition of control nanoparticles-treated mice, which correlated with more rapid tumor growth at the primary and metastatic sites. The lung metastases were confirmed by H&E staining (FIG. 8C and FIG. 15E), the gross lung tissue isolated post-mortem (FIGS. 8D - 8E), and a high proliferative index as determined by Ki67 staining (FIG. 8F). Furthermore, TRIM37 was significantly reduced in the metastatic lesions isolated from the smart nanoparticle-treated animals compared to control animals (FIG. 15F). No significant changes in liver histology (FIG. 8G) or serum AST and ALT levels (FIG. 8H) between the control and smart nanoparticles-treated animals indicated a lack of hepatotoxicity.

Primary tumors are routinely treated with a combination of surgery and chemotherapy. Our findings revealed that TRIM37 augments the metastatic potential of TNBC tumors by promoting survival under chemotherapeutic stress, and by inducing metastatic effectors (summarized in FIG. 8G). These results raised the possibility that combining TRIM37 inhibition with chemotherapy will simultaneously increase chemotherapy efficacy and prevent metastatic progression of TNBC to increase the overall survival in TNBC patients.

To test this idea in a clinically relevant setting, we generated primary tumors by subcutaneously implanting 231-2b lung-tropic cells in female NSG mice (FIG. 8H). Animals were treated with either control or smart nanoparticles intranasally in combination with a single dose of 2 mg/kg of Dox intraperitoneally post-tumor resection. Significantly, animals treated with control nanoparticles developed lung metastases, whereas smart nanoparticles treatment dramatically reduced metastatic burden in the lungs (FIG. 8I and FIG. 15I). H&E-stained lung sections (FIG. 8J) and luciferase signals from gross lung tissues revealed ~5-fold decrease in metastatic growth in animals treated with smart nanoparticles compared to control nanoparticle treated animals (FIGS. 8K - 8L and FIG. 15K). Tumors from smart nanoparticles and Dox-treated animals showed decreased tumor growth as indicated by significantly higher staining for Caspase 3 (FIG. 8M) and lower Ki67 staining (FIG. 15J) in lung metastatic tumors in comparison to the control tumors.

To inhibit TRIM37 in murine cells, we generated and characterized smart nanoparticles conjugated with murine cross-reactive anti-FOLR1 (FIG. 15L). A murine cross-reactive smart nanoparticle was tested and confirmed for inhibiting TRIM37 in 4T1 cells (FIGS. 15M-15O).

### Discussion of the Examples

This study identifies a new TRIM37-driven epigenetic network, which is amplified by chemotherapeutic drugs, as a unifying mechanism that drives chemoresistant and metastatic phenotype in TNBC tumors. The results are relevant to ~80% of TNBC patients that lack functional p53 and rely on systemic chemotherapeutic treatments due to the unavailability of any targeted TNBC therapy.

With the therapeutic spectrum limited to systemic chemotherapy, the majority of treated TNBC patients resist, relapse, and develop distant metastatic tumors, with a median survival of only 6-13 months (Garrido-Castro et al., 2019). Chemoresistance, in general, is accompanied with extensive genetic and epigenetic alterations. However, whether selection of the clonal cancer cells or new mutations drive chemoresistant phenotype remain to be resolved (Navin, 2014). A recent genomic and phenotypic evolution profiling of TNBC tumors identified both pre-existing resistant genotypes as well as transcriptional reprogramming of resistant signatures in TNBC tumors (Kim et al., 2018). Our findings show that pre-existing higher levels of TRIM37 in TNBC tumors promote resistance to chemotherapeutic stress and thus, increase survival of TNBC cells. On the other hand, chemotherapy triggers ATM signaling to transcriptionally upregulate TRIM37, which could further select for aggressive TNBC cells. In summary, TRIM37-positive TNBC tumors are protected and thrive under continued chemotherapy to cause aggressive metastatic disease.

A major hurdle in finding cure for aggressive TNBC is the lack of known drivers of the metastatic transition, in part due to a lack of mechanistic insights in the development of chemoresistant, metastatic tumors. A genomics-driven discovery of recurring genetic mutations and epigenetic aberrations in the breast cancer genome has revealed tumorigenic drivers (Cancer Genome Atlas Network, 2012; Roy et al., 2014; Shen & Laird, 2013)(6,43). However, whether these drivers that were primarily identified in the primary tumors are maintained throughout the chemotherapy regimen and the multistep process of metastasis remains to be evaluated. An additional clinical challenge to validate findings from the genomic analyses is the limited availability of matched pre- and post-chemotherapy as well as primary and metastatic tumors. We used genomic and genetic approaches in relevant TNBC cellular, preclinical murine models and tumor biopsies to establish TRIM37 function in reducing therapy-induced DNA damage, increasing cancer cell survival, and causing transcriptional aberrations (FIG. 8G). The presently disclosed results thus provide a rationale to target such common molecular effectors, including epigenetic regulators, in combination with chemotherapy to prevent or significantly delay the metastatic progression in TNBC patients.

While targeted therapies are desperately needed to limit damage to healthy tissues, new delivery mechanisms for cancer cell-specific targeting are also required to reduce detrimental side effects in healthy tissues. Molecularly targeted nanoparticles represent one such mechanism and are being aggressively explored for developing new treatment designs. As such, there are four nanoparticle-based therapies in clinical trials – BIND-014 for NSCLC and prostate cancer (Sanna et al., 2014)(44), CALAA-01 for solid tumors (Zuckerman et al., 2014)(45), SEL-068 for nicotine addiction (Desai & Bergman, 2015)(46), and Yale BNP for skin cancer (Deng et al., 2015).

We utilized a clinically investigative monoclonal antibody, covalently conjugated to nanoparticles, which selectively delivers TRIM37-ASO into TNBC cells. The molecularly targeted nanoparticles offer a significant advantage over antibody-drug conjugates in terms of higher payload concentrations in tumor cells by enhancing retention times and permeability (Alqaraghuli et al., 2019; Rosenblum et al., 2018). A consideration for nanoparticle delivery designs is clearance from the mononuclear phagocytic system. This is relevant for therapies designed to treat metastatic TNBC because premature elimination from circulation will prevent uptake by circulating tumor cells, decrease their accumulation in cancer cells and minimize their therapeutic impact. To overcome these issues, we incorporated PEG into some embodiments our design, which enables steric stabilization of nanoparticles, and prevents interaction of the nanoparticles with immune cells (Suk et al., 201648). Functionalizing nanoparticles with “self” markers or homing molecules can further improve the systemic delivery of nanoparticles, which is one of the major hurdles to overcome in therapy development (Rosenblum et al., 2018). Despite limitations, several designs have reached early-phase clinical trials and include C225-ILS-Dox for high-grade gliomas (Piktel et al., 2016) and Erbitux-EDV_{Spac} for solid tumors (Richards et al., 2017).

Notably, the majority of single-agent therapies tested to date for metastatic TNBC achieved an unimpressive response rate of less than 20%, with minimal impact on patient survival (Jhan & Andrechek, 2017; Vidula & Bardia, 2017)(50). The effective and selective delivery of TRIM37-ASO by Farletuzumab-conjugated nanoparticles provides an opportunity to employ additional TNBC-enriched surface proteins using monoclonal or bi-specific antibody formats. Some of the targets that can be exploited include MUC1 protein, which is overexpressed in TNBC (Maeda et al., 2018), as well as Trop-2 and TNBC-enriched metastatic regulators, such as VEGFR2 (Ramucirumab), an angiogenic factor (Zhu & Zhou, 2015).TNBC-enriched MUC1, Trop-2 and VEGFR2. Our results also support that molecularly targeted nanoparticles can deliver diverse payloads selectively to cancer cells. These payloads can include chemotherapeutic drugs, such as but not limited to an Irinotecan metabolite SN-38, and thereby, limit the toxicity to healthy tissues.

The majority of clinical deaths in TNBC patients are due to chemoresistance and aggressive metastases, with high prevalence in younger women of African ethnicity. While tumorigenic drivers are numerous and varied, the drivers of metastatic transition remain largely unknown. Here, we uncovered a molecular dependence of TNBC tumors on the TRIM37 network which enables tumor cells to resist chemotherapeutic as well as metastatic stress. TRIM37-directed histone H2A monoubiquitination enforce changes in DNA repair that rendered TP53-mutant TNBC cells resistant to chemotherapy. Chemotherapeutic drugs triggered a positive feedback loop via ATM/E2F1/STAT signaling, amplifying the TRIM37 network in chemoresistant cancer cells. High expression of TRIM37 induced transcriptomic changes characteristic of a metastatic phenotype, and inhibition of TRIM37 substantially reduced the in vivo propensity of TNBC cells. Selective delivery of TRIM37-specific antisense oligonucleotides using anti-folate receptor 1-conjugated nanoparticles in combination with chemotherapy suppressed lung metastasis in spontaneous metastatic murine models. Collectively, these findings establish TRIM37 as a clinically relevant target with opportunities for therapeutic intervention. TRIM37 drives aggressive TNBC biology by promoting resistance to chemotherapy and inducing a pro-metastatic transcriptional program; inhibition of TRIM37 increases chemotherapy efficacy and reduces metastasis risk in TNBC patients. The results presented herein identify a new driver of metastatic progression in TNBC patients and provide a mechanistic link between the two clinically linked phenotypes: chemoresistance and metastasis. Our findings also raise the possibility of clinically targeting TRIM37 to diminish the resistance to therapy, reduce the dissemination of cancer cells, and infiltration of distant sites. We demonstrate that our therapeutic design selectively inhibits TRIM37 and attenuates metastatic progression of TNBC tumors in vivo.

TABLE 4

List of DSB Repair Genes Expressed in Non-TNBC, TNBC, and Normal Tissue Obtained from METABRIC Patient Cohort

| double-strand break repair (GO:0097551) gene | correlation with TRIM37 expression in no-TNBC | correlation with TRIM37 expression in normal tissue | correlation with TRIM37 expression in TNBC |
|---|---|---|---|
| RAD51C | 0.6291 | 0.3906 | 0.5871 |
| XRCC5 | 0.3386 | 0.7137 | 0.4975 |
| RNF8 | 0.3435 | 0.5433 | 0.4101 |
| ATP23 | 0.3516 | -0.5021 | 0.3691 |
| RNF168 | 0.3332 | 0.4714 | 0.3620 |
| MRE11 | 0.1971 | 0.7280 | 0.3511 |
| MCM9 | 0.1992 | 0.6861 | 0.3443 |
| RAD51AP1 | 0.4171 | 0.7414 | 0.3251 |
| PALB2 | 0.2838 | 0.7722 | 0.3070 |
| SFR1 | 0.2476 | 0.3140 | 0.2778 |
| DDX1 | 0.1358 | -0.0432 | 0.2744 |
| CHEK2 | 0.2631 | 0.6345 | 0.2699 |
| RBBP8 | 0.0309 | 0.7174 | 0.2685 |
| BABAM1 | 0.3260 | -0.2498 | 0.2642 |
| RPA3 | 0.2983 | 0.2496 | 0.2436 |
| RAD51 | 0.3241 | 0.4984 | 0.2436 |
| GINS2 | 0.3310 | 0.5280 | 0.2433 |
| MDC1 | 0.2331 | 0.6868 | 0.2346 |
| RAD54B | 0.3478 | 0.7950 | 0.2304 |
| ERCC1 | 0.1512 | 0.2384 | 0.2270 |
| FEN1 | 0.3093 | 0.5267 | 0.2208 |
| KDM4D | 0.0287 | 0.4423 | 0.2204 |
| UBE2V2 | 0.3124 | 0.1830 | 0.2192 |
| TP53BP1 | 0.1086 | 0.7711 | 0.2190 |
| SEM1 | 0.1666 | -0.1445 | 0.2178 |
| RAD51B | -0.0580 | 0.4273 | 0.2047 |
| PAXIP1 | 0.1511 | 0.7616 | 0.2016 |
| SMARCAD1 | 0.1707 | 0.8273 | 0.2002 |
| WDR48 | 0.1515 | 0.6241 | 0.1913 |
| RECQL | 0.1816 | 0.1049 | 0.1887 |
| NSMCE2 | 0.2620 | 0.3291 | 0.1881 |
| BABAM2 | 0.1480 | NA | 0.1851 |
| RAD51D | 0.2129 | NA | 0.1768 |
| MLH1 | 0.1842 | 0.3756 | 0.1710 |
| MORF4L1 | 0.1347 | -0.1545 | 0.1691 |
| EXD2 | 0.2126 | 0.6826 | 0.1671 |
| SPIDR | 0.2274 | NA | 0.1653 |
| LIG3 | 0.0889 | 0.7026 | 0.1644 |
| TDP1 | 0.1317 | 0.8004 | 0.1620 |
| UBE2N | 0.1982 | 0.4196 | 0.1579 |
| CDCA5 | 0.3292 | 0.4843 | 0.1564 |
| MARF1 | -0.0662 | NA | 0.1533 |
| CDC45 | 0.2667 | 0.4907 | 0.1464 |
| HIST1H4A | 0.0504 | -0.1086 | 0.1349 |
| EXO1 | 0.2851 | 0.5899 | 0.1222 |
| FANCB | 0.1791 | 0.7374 | 0.1191 |
| TRIP 13 | 0.3094 | 0.6753 | 0.1150 |
| PRKDC | 0.1627 | 0.8001 | 0.1064 |
| H2AFX | 0.1147 | 0.2312 | 0.1016 |
| EYA3 | 0.0938 | 0.7717 | 0.0990 |
| RPA1 | -0.0534 | 0.5794 | 0.0923 |
| DCLRE1A | 0.1022 | 0.8545 | 0.0865 |
| CDC7 | 0.1800 | 0.6307 | 0.0842 |
| APTX | -0.0292 | 0.5640 | 0.0835 |
| RAD21 | 0.1801 | 0.6070 | 0.0810 |
| MSH2 | 0.0603 | 0.8249 | 0.0799 |
| HUS1 | -0.0037 | 0.5365 | 0.0774 |
| C14ORF39 | 0.0067 | NA | 0.0729 |
| VCP | 0.2176 | -0.0763 | 0.0643 |
| MEIOB | 0.0468 | NA | 0.0619 |
| UVRAG | 0.1129 | -0.4397 | 0.0619 |
| PSMD14 | 0.2422 | -0.0093 | 0.0609 |
| EME1 | 0.2602 | 0.5273 | 0.0605 |
| SUMO1 | 0.0202 | 0.2595 | 0.0580 |
| BRCA1 | 0.1746 | 0.7840 | 0.0527 |
| RMI1 | 0.1550 | 0.8215 | 0.0513 |
| RIF1 | 0.0756 | 0.6917 | 0.0448 |
| XRCC3 | 0.0745 | 0.4106 | 0.0433 |
| BLM | 0.1936 | 0.6329 | 0.0366 |
| WRN | -0.0629 | 0.7911 | 0.0343 |
| HERC2 | 0.0115 | 0.5073 | 0.0296 |
| AUNIP | 0.1557 | NA | 0.0286 |
| ATM | -0.0257 | 0.6764 | 0.0264 |
| SMC6 | 0.1061 | 0.6393 | 0.0257 |
| SWI5 | 0.0781 | NA | 0.0257 |
| DMC1 | 0.0232 | 0.5431 | 0.0248 |
| GINS4 | 0.1507 | 0.6908 | 0.0238 |
| HIST3H3 | 0.0001 | 0.1888 | 0.0212 |
| HELQ | 0.0571 | 0.5561 | 0.0187 |
| ERCC4 | 0.1254 | 0.6787 | 0.0181 |
| TENT4A | 0.0663 | NA | 0.0154 |
| RFWD3 | 0.1440 | 0.8249 | 0.0133 |
| NABP2 | 0.0080 | NA | 0.0118 |
| MMS22L | 0.1335 | NA | 0.0111 |
| HUS1B | 0.1011 | 0.1288 | 0.0046 |
| CCDC155 | -0.0064 | 0.2083 | 0.0046 |
| RAD21L1 | -0.0487 | 0.1917 | 0.0034 |
| GGN | -0.0539 | 0.3393 | 0.0023 |
| POLA1 | 0.1276 | 0.7820 | 0.0005 |
| APBB1 | 0.0163 | 0.1309 | -0.0086 |
| XRCC6 | 0.0688 | 0.3867 | -0.0094 |
| BARD1 | 0.0765 | 0.7242 | -0.0096 |
| GEN1 | 0.0513 | 0.7128 | -0.0168 |
| NBN | -0.0173 | 0.6545 | -0.0174 |
| SPO11 | -0.0622 | -0.0638 | -0.0182 |
| NSD2 | -0.0039 | NA | -0.0197 |
| ESCO2 | 0.0328 | 0.5833 | -0.0203 |
| EYA1 | 0.1111 | -0.4795 | -0.0244 |
| RAD50 | 0.0061 | 0.7363 | -0.0261 |
| TNKS1BP1 | -0.1415 | -0.0690 | -0.0262 |
| SFPQ | 0.0158 | 0.6328 | -0.0306 |
| LIG4 | -0.0119 | 0.5820 | -0.0308 |
| XRCC4 | 0.0254 | 0.3977 | -0.0370 |
| DNA2 | -0.0435 | 0.6865 | -0.0395 |
| MCMDC2 | 0.0282 | NA | -0.0406 |
| ABRAXAS 1 | 0.0389 | NA | -0.0492 |
| POLQ | 0.1653 | 0.5881 | -0.0499 |
| NSMCE1 | 0.0485 | 0.0808 | -0.0523 |
| CYREN | -0.0595 | NA | -0.0611 |
| PARP2 | 0.0281 | 0.6457 | -0.0658 |
| SMC5 | -0.0070 | 0.7221 | -0.0663 |
| MUS81 | -0.0572 | 0.3454 | -0.0724 |
| INO80 | 0.0775 | 0.6514 | -0.0729 |
| RNF138 | 0.0142 | 0.6070 | -0.0754 |
| XRCC1 | -0.1818 | 0.2806 | -0.0814 |
| PARP9 | 0.1738 | 0.4583 | -0.0862 |
| TONSL | 0.0906 | NA | -0.0896 |
| MAD2L2 | 0.0845 | 0.1317 | -0.0896 |
| ZFYVE26 | -0.0580 | 0.4675 | -0.0905 |
| MCM8 | 0.1293 | 0.7084 | -0.0925 |
| PAXX | -0.0619 | NA | -0.0989 |
| EME2 | -0.0091 | 0.5406 | -0.1080 |
| NIPBL | -0.1348 | 0.7405 | -0.1137 |
| POLL | -0.1567 | 0.2532 | -0.1173 |
| TDP2 | 0.0220 | 0.2346 | -0.1173 |
| BRCA2 | -0.0640 | 0.7886 | -0.1233 |
| ZBTB7A | -0.0575 | 0.1620 | -0.1246 |
| RAD54L | 0.0781 | 0.5221 | -0.1257 |
| RECQL4 | 0.0607 | 0.4551 | -0.1266 |
| SWSAP1 | -0.0938 | NA | -0.1360 |
| PIAS4 | -0.2332 | 0.4740 | -0.1365 |
| YY1 | -0.0425 | 0.6594 | -0.1384 |
| AP5S1 | 0.0803 | NA | -0.1469 |
| PRPF19 | -0.1964 | 0.4806 | -0.1475 |
| ACTR5 | 0.0562 | 0.5250 | -0.1610 |
| UIMC1 | -0.0819 | 0.4500 | -0.1622 |
| FBH1 | -0.2725 | NA | -0.1630 |
| SETX | -0.1091 | 0.6310 | -0.1736 |
| MTA1 | -0.2362 | 0.2928 | -0.1741 |
| RPA4 | -0.1269 | -0.2271 | -0.1794 |
| RPA2 | -0.1975 | 0.3176 | -0.1867 |
| POLN | -0.1922 | -0.1243 | -0.1897 |
| REC8 | 0.0044 | 0.4805 | -0.1989 |
| KAT5 | -0.1050 | 0.2994 | -0.2037 |
| NABP1 | -0.1075 | 0.0853 | -0.2116 |
| FAN1 | -0.1999 | 0.4578 | -0.2151 |
| RECQL5 | -0.0815 | 0.4626 | -0.2175 |
| CIB1 | -0.0817 | -0.0036 | -0.2239 |
| PARP3 | -0.1945 | 0.0830 | -0.2260 |
| SLX1A | -0.1037 | 0.0015 | -0.2809 |
| POLM | -0.1871 | 0.4051 | -0.3162 |
| NUCKS1 | -0.2819 | 0.7425 | -0.3784 |
| AP5Z1 | -0.1551 | 0.0216 | -0.3844 |

TABLE 5

List of Primers and shRNA used for qRT-PCR, ChIP, and Vector Construction

| GENE | Forward primer (5′ -> 3′) | Reverse primer (5′ -> 3′) |
|---|---|---|
| qRT-PCR | | |
| TRIM37 | AACAGAGCGTGGAGAGCATT | CTTCTGCCCAACGACAATTT |
| | SEQ ID NO: 13 | SEQ ID NO: 14 |
| GAPDH | TGCACCACCAACTGCTTAGC | GGCATGGACTGTGGTCATGAG |
| | SEQ ID NO: 15 | SEQ ID NO: 16 |
| NBS1 | ATGCACTCACCTTGTCATGG | ACGTCCACAAATGAGTGCAC |
| | SEQ ID NO: 17 | SEQ ID NO: 18 |
| RAD51C | CGAGCTTAGCAAAGAAGTTGGG | TGCTCAAGAAGTTCCAGTGC |
| | SEQ ID NO: 19 | SEQ ID NO: 20 |
| KU70 | TTTCAAGCCGTTGGTACTGC | ACAATGCTGCAACCTCCTTC |
| | SEQ ID NO: 21 | SEQ ID NO: 22 |
| KU80 | TGCACTAATCGTGAGCATGG | TTTTGCTGAATCGGCTGCTG |
| | SEQ ID NO: 23 | SEQ ID NO: 24 |
| HK2 | TGGACAAAAGGCTTCAAGGC | TTCACCACAGCAACCACATC |
| | SEQ ID NO: 25 | SEQ ID NO: 26 |
| GPI | TTCCAGAATTGCAGCAATG | AAGCTGAAGTGGTTGAAGCG |
| | SEQ ID NO: 27 | SEQ ID NO: 28 |
| TGM2 | AGATGTCAACCCCAAGTTCCTG | ATCGTTGCAGTTGACCATGC |
| | SEQ ID NO: 29 | SEQ ID NO: 30 |
| VEGFA | GGCAAAAACGAAAGCGCAAG | CAAATGCTTCTCCGCTCTGAG |
| | SEQ ID NO: 31 | SEQ ID NO: 32 |
| PDGFRB | TGAACGTGGTCAACCTGTTG | AGGAAGGTGTGTTTGTTGCG |
| | SEQ ID NO: 33 | SEQ ID NO: 34 |
| IGFBP3 | AAAAAGCAGTGTCGCCCTTC | ACTTGCTCTGCATGCTGTAG |
| | SEQ ID NO: 35 | SEQ ID NO: 36 |
| EGLN3 | TGCTGAAGAAAGGGCAGAAG | ACAAGGCCAGCAGATTTCAG |
| | SEQ ID NO: 37 | SEQ ID NO: 38 |
| PPFIA4 | ATTGGTGGAGTTGGGCAATG | TTACGACATGGCCAACAGTG |
| | SEQ ID NO: 39 | SEQ ID NO: 40 |
| REDD1 | AAGACACGGCTTACCTGGATG | AAGTGTTCATCCTCAGGGTCAC |
| | SEQ ID NO: 41 | SEQ ID NO: 42 |
| SLCO2A1 | ACATGATGGTTCTGCGTGTG | AGCGCATCAACAAGAACTGC |
| | SEQ ID NO: 43 | SEQ ID NO: 44 |
| S100A4 | AGAACTAAAGGAGCTGCTGACC | TGTTGCTGTCCAAGTTGCTC |
| | SEQ ID NO: 45 | SEQ ID NO: 46 |
| STC1 | ACAGCAAGCTGAATGTGTGC | ATCACATTCCAGCAGGCTTC |
| | SEQ ID NO: 47 | SEQ ID NO: 48 |
| VLDLR | TGACATTGCTGCCCAGAAC | ACCCAATCAACAGCAATGGC |
| | SEQ ID NO: 49 | SEQ ID NO: 50 |
| SHH | ACTCACCCCCAATTACAACCC | TGGCCAAAGCGTTCAACTTG |
| | SEQ ID NO: 51 | SEQ ID NO: 52 |
| L1CAM | TGGCTGCCAATGACCAAAA | AAGGTCACCCTGGAACCTTTC |
| | SEQ ID NO: 53 | SEQ ID NO: 54 |
| BMI1 | CGGGAGCTGCTTGGGTCAAGTG | CAGCCTAACACCCACCTTGGCG |
| | SEQ ID NO: 54 | SEQ ID NO: 55 |
| EZH2 | CAGGCACAGTGGCTCACGTCTG | GTTGGCCAGGCTGGTCTCGAAC |
| | SEQ ID NO: 56 | SEQ ID NO: 57 |
| KISS1 | TGGTTTCTTGGCAGCTACTG | ATTCTAGCTGCTGGCCTGTG |
| | SEQ ID NO: 58 | SEQ ID NO: 59 |
| BRMS1 | ATGGTGGGATGACAAACTGC | ATGTATGGGCCAGAAACCAGAG |
| | SEQ ID NO: 60 | SEQ ID NO: 61 |
| ChIP | | |
| GAPDH | CCCATGTTCGTCATGGGTGT | GGAGCGTGTCCATAGGGTG |
| | SEQ ID NO: 62 | SEQ ID NO: 63 |
| FRA3B | TGTTGGAATGTTAACTCTATCCCAT | ATATCTCATCAAGACCGCTGCA |
| | SEQ ID NO: 64 | SEQ ID NO: 65 |
| TRIM37 | ACTTAACTACGGGTGTGGCTC | CTGGCGTACTGGTGGAAAGC |
| | SEQ ID NO: 66 | SEQ ID NO: 67 |
| ACTIN | AGGGACTCTAGTGCCCGACAC | CCCACCTCCACCCTACCTGC |
| | SEQ ID NO: 68 | SEQ ID NO: 69 |
| BRMS1 | TAACTAGCTGTGTGGCCTTGAG | ATCTGAGCCTCCCAACAGTTC |
| | SEQ ID NO: 70 | SEQ ID NO: 72 |
| KISS1 | TCCCATTTCGTCCTCCTTGC | TCCTTCCTGCTTCCCTTCTTTC |
| | SEQ ID NO: 73 | SEQ ID NO: 74 |
| Cloning | | |
| TRIM37* Not I_F BamHI_R | TAAAGCGGCCGCCATGGATGAACAGAGCGTGGAGAG | CCGCGGATCCTTATCTTCCACTATTTTCATCTGTATTG |
| | SEQ ID NO: 75 | SEQ ID NO: 76 |

snRNAs

| | |
|---|---|
| Target Gene | shRNA Sequence and SEQ ID NO: |
| TRIM37 shRNA #1: | TTCACTGGTAAAGTCTGGTGG (SEQ ID NO: 77) |
| TRIM37 shRNA #2: | ATGACCTCCTTTAGCATTAGC (SEQ ID NO: 78) |
| KISS1 shRNA #1: | AAGGAGTTCCAGTTGTAGTTC (SEQ ID NO: 79) |
| KISS1 shRNA #2: | TTCCAGTTGTAGTTCGGCAGG (SEQ ID NO: 80) |
| BRMS1 shRNA #1: | TTTAGCTCCGAGAACTGCTTC (SEQ ID NO: 81) |
| BRMS1 shRNA #2: | AAGCATGTACACGATGTATGG (SEQ ID NO: 82) |
| E2F1 shRNA #1: | AAAGGGCCGAAAGTGCAGTTA (SEQ ID NO: 83) |
| E2F1 shRNA #2: | CAAAGTCACAGTCGAAGAGGT (SEQ ID NO: 84) |
| STAT1 shRNA #1: | ACTGTGTTCATCATACTGTCG (SEQ ID NO: 85) |
| STAT1 shRNA #2: | TTCTCGTCCTGATACTTTGGG (SEQ ID NO: 86) |
| STAT3 shRNA #1: | ATCTCCTGACCTTATGATCCG (SEQ ID NO: 87) |
| STAT3 shRNA #2: | AAGTGGCATGTGATTCTTTGC (SEQ ID NO: 88) |
| EZH2 shRNA #1: | GCAGCTGGTGAGAAGGCAATA (SEQ ID NO: 89) |
| EZH2 shRNA #2: | TGAAGCTAAGGCAGCTGTTT (SEQ ID NO: 90) |
| BMI1 shRNA #1: | TTCTCCAGGTATAAATGTAGG (SEQ ID NO: 91) |
| BMI1 shRNA #2: | TTCCGATCCAATCTGTTCTGG (SEQ ID NO: 92) |

*NotI recognition site in forward primer and BamHI recognition site in reverse primer are underlined.

TABLE 6

List of Tumor and Metastasis Suppressors With Decreased Expression that Significantly Correlates with Increased TRIM37 Levels in 231-2b Cells

| Gene | log2FC_TRIM ASOvsctr | P value | Type | Description |
|---|---|---|---|---|
| GADD45A | 0.53275 | 6.80E-33 | Coding | growth arrest and DNA-damage-inducible, alpha |
| ZFAS1 | 0.45623 | 3.81E-21 | Non-coding | ZNFX1 antisense RNA 1 |
| RND3 | 0.41362 | 3.96E-21 | Coding | Rho family GTPase 3 |
| DKK1 | 0.30062 | 1.59E-18 | Coding | dickkopf WNT signaling pathway inhibitor 1 |
| GADD45B | 0.44748 | 6.80E-17 | Coding | growth arrest and DNA-damage-inducible, beta |
| HSPD1 | 0.25578 | 4.86E-16 | Coding | heat shock 60 kDa protein 1 (chaperonin) |
| GPRC5A | 0.3634 | 8.03E-15 | Coding | G protein-coupled receptor, class C, group 5, member A |
| ATF3 | 0.6269 | 7.29E-12 | Coding | activating transcription factor 3 |
| EEF1A1 | 0.13683 | 3.03E-10 | Coding | eukaryotic translation elongation factor 1 alpha 1 |
| GTPBP4 | 0.24467 | 2.45E-09 | Coding | GTP binding protein 4 |
| CHD5 | 0.71725 | 2.57E-09 | Coding | chromodomain helicase DNA binding protein 5 |
| MYBBP1A | 0.23361 | 3.14E-09 | Coding | MYB binding protein (P160) 1a |
| BRMS1 | 0.27051 | 8.59E-09 | Coding | breast cancer metastasis suppressor 1 |
| PLK2 | 0.26072 | 3.81E-08 | Coding | polo-like kinase 2 |
| CITED2 | 0.30665 | 6.24E-08 | Coding | Cbp/p300-interacting transactivator, with Glu/Asp-rich carboxy-terminal domain, 2 |
| GADD45G | 0.6066 | 6.24E-08 | Coding | growth arrest and DNA-damage-inducible, gamma |
| NPM1 | 0.17477 | 9.75E-08 | Coding | nucleophosmin (nucleolar phosphoprotein B23, numatrin) |
| PEA 15 | 0.18199 | 1.52E-07 | Coding | phosphoprotein enriched in astrocytes 15 |
| KISS1 | 0.21693 | 2.52E-07 | Coding | KiSS-1 metastasis-suppressor |
| GADD45GIP1 | 0.18661 | 1.06E-06 | Coding | growth arrest and DNA-damage-inducible, gamma interacting protein 1 |
| RPL5 | 0.13739 | 1.28E-06 | Coding | ribosomal protein L5 |
| DUSP1 | 0.31096 | 2.78E-06 | Coding | dual specificity phosphatase 1 |
| LIMA1 | 0.17529 | 3.14E-06 | Coding | LIM domain and actin binding 1 |
| GAS5 | 0.29399 | 4.07E-06 | Non-coding | growth arrest-specific 5 (non-protein coding) |
| IL24 | 0.41026 | 4.87E-06 | Coding | interleukin 24 |
| GNB2L1 | 0.11663 | 6.97E-06 | Coding | guanine nucleotide binding protein (G protein), beta polypeptide 2-like 1 |
| H2AFX | 0.16459 | 2.79E-05 | Coding | H2A histone family, member X |
| MAD1L1 | 0.22216 | 3.35E-05 | Coding | MAD 1 mitotic arrest deficient-like 1 (yeast) |
| EIF3F | 0.13426 | 6.63E-05 | Coding | eukaryotic translation initiation factor 3, subunit F |
| NKX3-1 | 0.2434 | 0.000126 | Coding | NK3 homeobox 1 |
| SAFB | 0.14452 | 0.00018 | Coding | scaffold attachment factor B |
| BAX | 0.14457 | 0.000232 | Coding | BCL2-associated X protein |
| RPL11 | 0.12563 | 0.000244 | Coding | ribosomal protein L11 |
| TNFRSF12A | 0.12189 | 0.000304 | Coding | tumor necrosis factor receptor superfamily, member 12A |
| ABCG2 | 0.32505 | 0.000322 | Coding | ATP-binding cassette, sub-family G (WHITE), member 2 (Junior blood group) |
| NEDD4L | 0.13128 | 0.000395 | Coding | neural precursor cell expressed, developmentally down-regulated 4-like, E3 ubiquitin protein ligase |
| TCEB3 | 0.18575 | 0.000417 | Coding | transcription elongation factor B (SIII), polypeptide 3 (110 kDa, elongin A) |
| DNAJA3 | 0.17118 | 0.000436 | Coding | DnaJ (Hsp40) homolog, subfamily A, member 3 |
| EPHA2 | 0.12537 | 0.000599 | Coding | EPH receptor A2 |
| TBL2 | 0.1803 | 0.00067 | Coding | transducin (beta)-like 2 |
| NUPR1 | 0.38406 | 0.000801 | Coding | nuclear protein, transcriptional regulator, 1 |
| SAFB2 | 0.15687 | 0.000824 | Coding | scaffold attachment factor B2 |
| PHB | 0.14459 | 0.001116 | Coding | prohibitin |
| GLTSCR2 | 0.14222 | 0.001182 | Coding | glioma tumor suppressor candidate region gene 2 |
| RINT1 | 0.19116 | 0.001535 | Coding | RAD50 interactor 1 |
| CPNE7 | 0.35839 | 0.001785 | Coding | copine VII |
| HTATIP2 | 0.18554 | 0.001794 | Coding | HIV-1 Tat interactive protein 2, 30 kDa |
| PTPN2 | 0.14253 | 0.001806 | Coding | protein tyrosine phosphatase, non-receptor type 2 |
| LIN9 | 0.24433 | 0.001963 | Coding | lin-9 DREAM MuvB core complex component |
| BMP4 | 0.18333 | 0.002002 | Coding | bone morphogenetic protein 4 |
| PDCD5 | 0.1362 | 0.002312 | Coding | programmed cell death 5 |
| TUSC1 | 0.30849 | 0.002818 | Coding | tumor suppressor candidate 1 |
| SDHB | 0.15631 | 0.002872 | Coding | succinate dehydrogenase complex, subunit B, iron sulfur (Ip) |
| PRDM11 | 0.27797 | 0.00333 | Coding | PR domain containing 11 |
| UBIAD1 | 0.19626 | 0.003982 | Coding | UbiA prenyltransferase domain containing 1 |
| NBN | 0.11814 | 0.004003 | Coding | nibrin |
| SFN | 0.095784 | 0.004152 | Coding | stratifin |
| DKK3 | 0.32945 | 0.004189 | Coding | dickkopf WNT signaling pathway inhibitor 3 |
| GPX3 | 0.15911 | 0.004293 | Coding | glutathione peroxidase 3 |
| ARMC5 | 0.24559 | 0.005366 | Coding | armadillo repeat containing 5 |
| TSSC4 | 0.17644 | 0.005418 | Coding | tumor suppressing subtransferable candidate 4 |
| BANP | 0.20157 | 0.005528 | Coding | BTG3 associated nuclear protein |
| CHST10 | 0.18043 | 0.005906 | Coding | carbohydrate sulfotransferase 10 |
| NUP98 | 0.1193 | 0.005935 | Coding | nucleoporin 98 kDa |
| EZH2 | 0.12565 | 0.006543 | Coding | enhancer of zeste 2 polycomb repressive complex 2 subunit |
| EPHB3 | 0.2621 | 0.006579 | Coding | EPH receptor B3 |
| ARID 1A | 0.13247 | 0.007808 | Coding | AT rich interactive domain 1A (SWI-like) |
| SMARCB1 | 0.12006 | 0.008141 | Coding | SWI/SNF related, matrix associated, actin dependent regulator of chromatin, subfamily b, member 1 |
| AHRR | 0.27571 | 0.008228 | Coding | aryl-hydrocarbon receptor repressor |
| BEX2 | 0.29158 | 0.008319 | Coding | brain expressed X-linked 2 |
| G0S2 | 0.099913 | 0.008681 | Coding | G0/G1 switch 2 |
| VEGFA | -0.44827 | 8.40E-44 | Coding | vascular endothelial growth factor A |
| BNIP3L | -0.48742 | 2.21E-33 | Coding | BCL2/adenovirus E1B 19 kDa interacting protein 3-like |
| HTRA1 | -0.5306 | 2.93E-19 | Coding | HtrA serine peptidase 1 |
| TGFBR2 | -0.30163 | 7.70E-17 | Coding | transforming growth factor, beta receptor II (70/80 kDa) |
| TXNIP | -0.42747 | 1.68E-16 | Coding | thioredoxin interacting protein |
| SRPX | -0.42043 | 1.22E-15 | Coding | sushi-repeat containing protein, X-linked |
| ITGA5 | -0.37797 | 1.07E-14 | Coding | integrin, alpha 5 (fibronectin receptor, alpha polypeptide) |
| WNT5A | -0.33185 | 3.42E-14 | Coding | wingless-type MMTV integration site family, member 5A |
| NDRG1 | -0.66632 | 2.22E-13 | Coding | N-myc downstream regulated 1 |
| ITGAV | -0.27785 | 2.84E-13 | Coding | integrin, alpha V |
| BMPR2 | -0.27705 | 2.40E-12 | Coding | bone morphogenetic protein receptor, type II (serine/threonine kinase) |
| TIMP3 | -0.57728 | 2.41E-12 | Coding | TIMP metallopeptidase inhibitor 3 |
| LOX | -0.47609 | 3.58E-12 | Coding | lysyl oxidase |
| IGFBP4 | -0.23587 | 4.91E-12 | Coding | insulin-like growth factor binding protein 4 |
| CD82 | -0.29397 | 6.58E-12 | Coding | CD82 molecule |
| EGLN3 | -0.60858 | 1.04E-11 | Coding | egl-9 family hypoxia-inducible factor 3 |
| ERRFI1 | -0.5272 | 2.54E-11 | Coding | ERBB receptor feedback inhibitor 1 |
| ITGB1 | -0.18212 | 3.92E-11 | Coding | integrin, beta 1 (fibronectin receptor, beta polypeptide, antigen CD29 includes MDF2, MSK12) |
| ST5 | -0.51129 | 5.82E-11 | Coding | suppression of tumorigenicity 5 |
| CD44 | -0.18339 | 2.35E-10 | Coding | CD44 molecule (Indian blood group) |
| EHF | -0.41958 | 2.97E-10 | Coding | ets homologous factor |
| TGFB1 | -0.27115 | 3.97E-10 | Coding | transforming growth factor, beta 1 |
| TSC22D1 | -0.24698 | 4.45E-10 | Coding | TSC22 domain family, member 1 |
| MXI1 | -0.41475 | 6.62E-10 | Coding | MAX interactor 1, dimerization protein |
| YPEL3 | -0.5295 | 4.11E-09 | Coding | yippee-like 3 (Drosophila) |
| ETV6 | -0.37738 | 4.21E-09 | Coding | ets variant 6 |
| PAEP | -0.7122 | 4.85E-09 | Coding | progestagen-associated endometrial protein |
| IFI16 | -0.31104 | 5.02E-09 | Coding | interferon, gamma-inducible protein 16 |
| CMTM3 | -0.34468 | 5.23E-09 | Coding | CKLF-like MARVEL transmembrane domain containing 3 |
| GSN | -0.33964 | 6.44E-09 | Coding | gelsolin |
| TGFBI | -0.37669 | 9.65E-09 | Coding | transforming growth factor, beta-induced, 68 kDa |
| DUSP6 | -0.28537 | 3.33E-08 | Coding | dual specificity phosphatase 6 |
| JUP | -0.34035 | 7.60E-08 | Coding | junction plakoglobin |
| NOTCH3 | -0.51991 | 1.67E-07 | Coding | notch 3 |
| MARCKS | -0.17149 | 1.76E-07 | Coding | myristoylated alanine-rich protein kinase C substrate |
| HBP1 | -0.38012 | 3.16E-07 | Coding | HMG-box transcription factor 1 |
| RUNX2 | -0.22325 | 4.38E-07 | Coding | runt-related transcription factor 2 |
| GJB2 | -0.42352 | 5.05E-07 | Coding | gap junction protein, beta 2, 26 kDa |
| BHLHE41 | -0.38532 | 5.85E-07 | Coding | basic helix-loop-helix family, member e41 |
| AIM2 | -0.43646 | 2.27E-06 | Coding | absent in melanoma 2 |
| RBL2 | -0.30435 | 2.87E-06 | Coding | retinoblastoma-like 2 |
| KDM3A | -0.22985 | 3.44E-06 | Coding | lysine (K)-specific demethylase 3A |
| IGFBP3 | -0.421 | 3.88E-06 | Coding | insulin-like growth factor binding protein 3 |
| PDCD4 | -0.3061 | 4.87E-06 | Coding | programmed cell death 4 (neoplastic transformation inhibitor) |
| EMP1 | -0.21722 | 1.03E-05 | Coding | epithelial membrane protein 1 |
| SIK1 | -0.2196 | 1.05E-05 | Coding | salt-inducible kinase 1 |
| HTRA3 | -0.31734 | 1.53E-05 | Coding | HtrA serine peptidase 3 |
| ARL6IP5 | -0.24206 | 1.58E-05 | Coding | ADP-ribosylation factor-like 6 interacting protein 5 |
| S100A11 | -0.14287 | 2.00E-05 | Coding | S100 calcium binding protein A11 |
| EXT2 | -0.18193 | 2.47E-05 | Coding | exostosin glycosyltransferase 2 |
| THRA | -0.26456 | 2.97E-05 | Coding | thyroid hormone receptor, alpha |
| CDCP1 | -0.32161 | 3.82E-05 | Coding | CUB domain containing protein 1 |
| PRKAR1A | -0.13367 | 3.93E-05 | Coding | protein kinase, cAMP-dependent, regulatory, type I, alpha |
| RASSF4 | -0.47299 | 4.23E-05 | Coding | Ras association (RalGDS/AF-6) domain family member 4 |
| CTNND1 | -0.15019 | 4.50E-05 | Coding | catenin (cadherin-associated protein), delta 1 |
| RASSF2 | -0.32889 | 4.51E-05 | Coding | Ras association (RalGDS/AF-6) domain family member 2 |
| DUSP22 | -0.2385 | 5.32E-05 | Coding | dual specificity phosphatase 22 |
| RNF144A | -0.36403 | 5.34E-05 | Coding | ring finger protein 144A |
| NUMB | -0.1901 | 7.02E-05 | Coding | numb homolog (Drosophila) |
| SEMA3B | -0.36198 | 7.18E-05 | Coding | sema domain, immunoglobulin domain (Ig), short basic domain, secreted, (semaphorin) 3B |
| CREB3L1 | -0.2064 | 8.49E-05 | Coding | cAMP responsive element binding protein 3-like 1 |
| KLF6 | -0.15089 | 0.000114 | Coding | Kruppel-like factor 6 |
| TNFRSF10B | -0.21556 | 0.000118 | Coding | tumor necrosis factor receptor superfamily, member 10b |
| BCORL1 | -0.26188 | 0.000126 | Coding | BCL6 corepressor-like 1 |
| ZNF292 | -0.25512 | 0.000128 | Coding | zinc finger protein 292 |
| AKAP12 | -0.18457 | 0.000151 | Coding | A kinase (PRKA) anchor protein 12 |
| CDK2 | -0.24217 | 0.000158 | Coding | cyclin-dependent kinase 2 |
| ADARB1 | -0.25098 | 0.000171 | Coding | adenosine deaminase, RNA-specific, B1 |
| ARHGEF12 | -0.12594 | 0.000188 | Coding | Rho guanine nucleotide exchange factor (GEF) 12 |
| SIRT2 | -0.23896 | 0.000197 | Coding | sirtuin 2 |
| ADAMTS9 | -0.27396 | 0.000225 | Coding | ADAM metallopeptidase with thrombospondin type 1 motif, 9 |
| IGFBP7 | -0.27679 | 0.000273 | Coding | insulin-like growth factor binding protein 7 |
| RHOA | -0.099613 | 0.000282 | Coding | ras homolog family member A |
| SH2B3 | -0.16439 | 0.000351 | Coding | SH2B adaptor protein 3 |
| KRT19 | -0.18487 | 0.000353 | Coding | keratin 19, type I |
| EGLN1 | -0.18386 | 0.000405 | Coding | egl-9 family hypoxia-inducible factor 1 |
| YAP1 | -0.13769 | 0.000419 | Coding | Yes-associated protein 1 |
| PML | -0.17409 | 0.000439 | Coding | promyelocytic leukemia |
| CAPG | -0.13081 | 0.000441 | Coding | capping protein (actin filament), gelsolin-like |
| CDK6 | -0.16007 | 0.000457 | Coding | cyclin-dependent kinase 6 |
| ITGB3 | -0.32358 | 0.000466 | Coding | integrin, beta 3 (platelet glycoprotein IIIa, antigen CD61) |
| RBMS3 | -0.23494 | 0.000521 | Coding | RNA binding motif, single stranded interacting protein 3 |
| CIC | -0.16411 | 0.00054 | Coding | capicua transcriptional repressor |
| SH3GLB1 | -0.13392 | 0.0006 | Coding | SH3-domain GRB2-like endophilin B1 |
| TANK | -0.23978 | 0.000631 | Coding | TRAF family member-associated NFKB activator |
| VIM | -0.091068 | 0.000642 | Coding | vimentin |
| PDLIM4 | -0.17419 | 0.000864 | Coding | PDZ and LIM domain 4 |
| NPAS2 | -0.18775 | 0.00097 | Coding | neuronal PAS domain protein 2 |
| ZYX | -0.14056 | 0.000985 | Coding | zyxin |
| SMAD4 | -0.22662 | 0.001014 | Coding | SMAD family member 4 |
| RPS6KA2 | -0.21452 | 0.001086 | Coding | ribosomal protein S6 kinase, 90 kDa, polypeptide 2 |
| BMF | -0.23976 | 0.001305 | Coding | Bcl2 modifying factor |
| MAP3K4 | -0.212 | 0.001477 | Coding | mitogen-activated protein kinase kinase kinase 4 |
| SUFU | -0.20261 | 0.001516 | Coding | suppressor of fused homolog (Drosophila) |
| TCF4 | -0.32498 | 0.001806 | Coding | transcription factor 4 |
| PRDM1 | -0.32475 | 0.001964 | Coding | PR domain containing 1, with ZNF domain |
| PRKCDBP | -0.14412 | 0.001966 | Coding | protein kinase C, delta binding protein |
| RB1CC1 | -0.16351 | 0.002081 | Coding | RB1-inducible coiled-coil 1 |
| GLIPR1 | -0.21534 | 0.002217 | Coding | GLI pathogenesis-related 1 |
| DFNA5 | -0.18633 | 0.002439 | Coding | deafness, autosomal dominant 5 |
| PLCB3 | -0.14537 | 0.002487 | Coding | phospholipase C, beta 3 (phosphatidylinositol-specific) |
| PLD1 | -0.28486 | 0.003105 | Coding | phospholipase D1, phosphatidylcholine-specific |
| CDKN2C | -0.18476 | 0.003736 | Coding | cyclin-dependent kinase inhibitor 2C (p18, inhibits CDK4) |
| MADD | -0.13253 | 0.004181 | Coding | MAP-kinase activating death domain |
| NDRG4 | -0.29716 | 0.004234 | Coding | NDRG family member 4 |
| SEPTIN_4 | -0.34374 | 0.004518 | Coding | septin 4 |
| HECA | -0.23123 | 0.004528 | Coding | headcase homolog (Drosophila) |
| GANAB | -0.081916 | 0.004644 | Coding | glucosidase, alpha; neutral AB |
| SPRY4 | -0.17884 | 0.004948 | Coding | sprouty homolog 4 (Drosophila) |
| TMPRSS6 | -0.31868 | 0.00507 | Coding | transmembrane protease, serine 6 |
| CTNNBIP1 | -0.22412 | 0.005826 | Coding | catenin, beta interacting protein 1 |
| AFAP1L2 | -0.14131 | 0.005827 | Coding | actin filament associated protein 1-like 2 |
| PRKAA1 | -0.11359 | 0.005864 | Coding | protein kinase, AMP-activated, alpha 1 catalytic subunit |
| PLA2R1 | -0.29061 | 0.00598 | Coding | phospholipase A2 receptor 1, 180 kDa |
| BMP2 | -0.27979 | 0.006421 | Coding | bone morphogenetic protein 2 |
| TSC2 | -0.14377 | 0.006621 | Coding | tuberous sclerosis 2 |
| EZH1 | -0.1873 | 0.006723 | Coding | enhancer of zeste 1 polycomb repressive complex 2 subunit |
| PTPRJ | -0.15053 | 0.00735 | Coding | protein tyrosine phosphatase, receptor type, J |
| TP53INP1 | -0.22892 | 0.007455 | Coding | tumor protein p53 inducible nuclear protein 1 |
| PPARG | -0.15772 | 0.007531 | Coding | peroxisome proliferator-activated receptor gamma |
| PLA2G16 | -0.15599 | 0.007864 | Coding | phospholipase A2, group XVI |
| HIF1A | -0.084023 | 0.007995 | Coding | hypoxia inducible factor 1, alpha subunit (basic helix-loop-helix transcription factor) |
| HSP90B1 | -0.20385 | 0.008267 | Coding | heat shock protein 90 kDa beta (Grp94), member 1 |
| FAT1 | -0.13353 | 0.008362 | Coding | FAT atypical cadherin 1 |

TABLE 7A

Hypoxia Genes With Expression that Significantly Correlates with Increases in TRIM37 Levels in 231-2b Cells

| Gene Name | log2FC_TRIM_ASOvsctr | padj | GeneDescription |
|---|---|---|---|
| ALDOC | -0.67566 | 1.20E-09 | aldolase C fructose-bisphosphate |
| NDRG1 | -0.66632 | 3.07E-11 | N-myc downstream regulated 1 |
| DDIT4 | -0.65926 | 1.26E-18 | DNA-damage-inducible transcript 4 |
| PPFIA4 | -0.65733 | 1.13E-10 | protein tyrosine phosphatase receptor type f polypeptide (PTPRF) interacting protein (liprin) alpha 4 |
| ENO2 | -0.5959 | 3.33E-36 | enolase 2 (gamma neuronal) |
| CA12 | -0.53361 | 6.99E-12 | carbonic anhydrase XII |
| ERRFI1 | -0.5272 | 2.22E-09 | ERBB receptor feedback inhibitor 1 |
| BHLHE40 | -0.51511 | 1.14E-24 | basic helix-loop-helix family member e40 |
| CCNG2 | -0.50884 | 3.98E-16 | cyclin G2 |
| S100A4 | -0.49582 | 1.85E-06 | S100 calcium binding protein A4 |
| BNIP3L | -0.48742 | 2.38E-30 | BCL2/adenovirus E1B 19 kDa interacting protein 3-like |
| CXCR7 | -0.48467 | 0.0006958 | chemokine (C-X-C motif) receptor 7 |
| LOX | -0.47609 | 3.83E-10 | lysyl oxidase |
| PAM | -0.47225 | 3.72E-17 | peptidylglycine alpha-amidating monooxygenase |
| P4HA1 | -0.44941 | 4.99E-18 | prolyl 4-hydroxylase alpha polypeptide I |
| VEGFA | -0.44827 | 2.53E-40 | vascular endothelial growth factor A |
| STC1 | -0.43099 | 6.10E-24 | stanniocalcin 1 |
| IGFBP3 | -0.421 | 9.02E-05 | insulin-like growth factor binding protein 3 |
| SRPX | -0.42043 | 2.16E-13 | sushi-repeat containing protein X-linked |
| MXI1 | -0.41475 | 4.32E-08 | MAX interactor 1 dimerization protein |
| PGK1 | -0.41349 | 2.07E-24 | phosphoglycerate kinase 1 |
| COL5A1 | -0.41137 | 7.46E-06 | collagen type V alpha 1 |
| PDK1 | -0.40752 | 1.44E-06 | pyruvate dehydrogenase kinase isozyme 1 |
| SLC2A3 | -0.40102 | 1.80E-09 | solute carrier family 2 (facilitated glucose transporter) member 3 |
| KLHL24 | -0.40008 | 0.0014347 | kelch-like family member 24 |
| ANKZF1 | -0.38919 | 3.41E-07 | ankyrin repeat and zinc finger domain containing 1 |
| AK4 | -0.38455 | 8.80E-16 | adenylate kinase 4 |
| VLDLR | -0.38066 | 0.0002173 | very low density lipoprotein receptor |
| TGFBI | -0.37669 | 4.67E-07 | transforming growth factor beta-induced 68 kDa |
| RORA | -0.37387 | 1.23E-07 | RAR-related orphan receptor A |
| BRO1L | -0.35758 | 2.90E-20 | ERO1-like (S. cerevisiae) |
| PFKL | -0.3527 | 4.19E-14 | phosphofructokinase liver |
| KLF7 | -0.31404 | 0.0009368 | Kruppel-like factor 7 (ubiquitous) |
| GBE1 | -0.31391 | 1.06E-09 | glucan (14-alpha-) branching enzyme 1 |
| NFIL3 | -0.31122 | 0.0017932 | nuclear factor interleukin 3 regulated |
| PGF | -0.29579 | 8.15E-06 | placental growth factor |
| PDGFB | -0.26991 | 0.017173 | platelet-derived growth factor beta polypeptide |
| SDC3 | -0.25591 | 1.23E-07 | syndecan 3 |
| ZNF292 | -0.25512 | 0.0016948 | zinc finger protein 292 |
| TPBG | -0.24386 | 1.22E-05 | trophoblast glycoprotein |
| P4HA2 | -0.23751 | 0.000763 | prolyl 4-hydroxylase alpha polypeptide II |
| FAM162A | -0.23499 | 0.0051023 | family with sequence similarity 162 member A |
| SAP30 | -0.23413 | 0.012521 | Sin3A-associated protein 30 kDa |
| SLC2A1 | -0.2327 | 1.87E-10 | solute carrier family 2 (facilitated glucose transporter) member 1 |
| KDM3A | -0.22985 | 8.20E-05 | lysine (K)-specific demethylase 3A |
| LDHA | -0.22633 | 1.55E-14 | lactate dehydrogenase A |
| BTG1 | -0.20897 | 0.010386 | B-cell translocation gene 1 anti-proliferative |
| WSB1 | -0.20725 | 0.0061261 | WD repeat and SOCS box containing 1 |
| PFKP | -0.20681 | 1.67E-08 | phosphofructokinase platelet |
| SLC6A6 | -0.20287 | 7.94E-05 | solute carrier family 6 (neurotransmitter transporter taurine) member 6 |
| PIM1 | -0.20283 | 0.0033706 | pim-1 oncogene |
| PGM1 | -0.20168 | 2.27E-05 | phosphoglucomutase 1 |
| SCARB1 | -0.20167 | 0.0007171 | scavenger receptor class B member 1 |
| PFKFB3 | -0.19979 | 8.48E-05 | 6-phosphofructo-2-kinase/fructose-26-biphosphatase 3 |
| IDS | -0.19237 | 8.92E-05 | iduronate 2-sulfatase |
| AKAP12 | -0.18457 | 0.0019424 | A kinase (PRKA) anchor protein 12 |
| UGP2 | -0.18075 | 0.0012081 | UDP-glucose pyrophosphorylase 2 |
| KDELR3 | -0.17883 | 0.049979 | KDEL (Lys-Asp-Glu-Leu) endoplasmic reticulum protein retention receptor 3 |
| GAA | -0.17684 | 0.001063 | glucosidase alpha |
| KLF6 | -0.15089 | 0.0015355 | Kruppel-like factor 6 |
| ALDOA | -0.15014 | 1.94E-05 | aldolase A fructose-bisphosphate |
| PRKCDBP | -0.14412 | 0.01555 | protein kinase C delta binding protein |
| HK2 | -0.13992 | 0.010955 | hexokinase 2 |
| GPI | -0.13849 | 0.0008850 | glucose-6-phosphate isomerase |
| PRDX5 | -0.13205 | 0.047234 | peroxiredoxin 5 |
| TGM2 | -0.1265 | 0.0019221 | transglutaminase 2 (C polypeptide protein-glutamine-gamma-glutamyltransferase) |
| HK1 | -0.11376 | 0.014741 | hexokinase 1 |
| F3 | -0.11115 | 0.0021453 | coagulation factor III (thromboplastin tissue factor) |
| TPI1 | -0.10054 | 0.0014553 | triosephosphate isomerase 1 |
| ENO1 | -0.082787 | 0.01718 | enolase 1 (alpha) |
| NEDD4L | 0.13128 | 0.0042375 | neural precursor cell expressed developmentally down-regulated 4-like E3 ubiquitin protein ligase |
| MT1E | 0.13934 | 0.0042384 | metallothionein 1E |
| PGM2 | 0.14832 | 0.015262 | phosphoglucomutase 2 |
| IL6 | 0.16097 | 0.043979 | interleukin 6 (interferon beta 2) |
| CYR61 | 0.23994 | 2.38E-12 | cysteine-rich angiogenic inducer 61 |
| CITED2 | 0.30665 | 2.52E-06 | Cbp/p300-interacting transactivator with Glu/Asp-rich carboxy-terminal domain 2 |
| DUSP1 | 0.31096 | 6.90E-05 | dual specificity phosphatase 1 |
| ADORA2 B | 0.34829 | 1.19E-10 | adenosine A2b receptor |
| DDIT3 | 0.44592 | 0.002176 | DNA-damage-inducible transcript 3 |
| PPP1R15 A | 0.51236 | 9.48E-44 | protein phosphatase 1 regulatory subunit 15A |
| ATF3 | 0.6269 | 7.28E-10 | activating transcription factor 3 |

TABLE 7B

EMT Genes With Expression that Significantly Correlates with Increases in TRIM37 Levels in 231-2b Cells

| Gene Name | log2FC_TRIM_ASOvsctr | padj | GeneDescription |
|---|---|---|---|
| COL6A3 | -0.69302 | 9.17E-08 | collagen type VI alpha 3 |
| ENO2 | -0.5959 | 3.33E-36 | enolase 2 (gamma neuronal) |
| COL7A1 | -0.59267 | 1.43E-36 | collagen type VII alpha 1 |
| TIMP3 | -0.57728 | 2.69E-10 | TIMP metallopeptidase inhibitor 3 |
| COL6A2 | -0.5379 | 3.22E-10 | collagen type VI alpha 2 |
| HTRA1 | -0.5306 | 8.00E-17 | HtrA serine peptidase 1 |
| ITGA2 | -0.52835 | 1.35E-26 | integrin alpha 2 (CD49B alpha 2 subunit of VLA-2 receptor) |
| FN1 | -0.51554 | 4.53E-06 | fibronectin 1 |
| THBS2 | -0.50155 | 2.37E-25 | thrombospondin 2 |
| LAMC2 | -0.49914 | 1.21E-07 | laminin gamma 2 |
| SPP1 | -0.4797 | 7.91E-05 | secreted phosphoprotein 1 |
| LOX | -0.47609 | 3.83E-10 | lysyl oxidase |
| NT5E | -0.47508 | 2.11E-32 | 5′-nucleotidase ecto (CD73) |
| VEGFA | -0.44827 | 2.53E-40 | vascular endothelial growth factor A |
| PDGFRB | -0.44146 | 0.001646 | platelet-derived growth factor receptor beta polypeptide |
| PTHLH | -0.42564 | 0.00303 | parathyroid hormone-like hormone |
| IGFBP3 | -0.421 | 9.02E-05 | insulin-like growth factor binding protein 3 |
| COL5A1 | -0.41137 | 7.46E-06 | collagen type V alpha 1 |
| PMEPA1 | -0.38354 | 1.17E-06 | prostate transmembrane protein androgen induced 1 |
| ITGA5 | -0.37797 | 1.72E-12 | integrin alpha 5 (fibronectin receptor alpha polypeptide) |
| TGFBI | -0.37669 | 4.67E-07 | transforming growth factor beta-induced 68 kDa |
| BMP1 | -0.37103 | 5.69E-05 | bone morphogenetic protein 1 |
| WNT5A | -0.33185 | 5.10E-12 | wingless-type MMTV integration site family member 5A |
| LOXL2 | -0.3281 | 5.21E-10 | lysyl oxidase-like 2 |
| PLOD2 | -0.32559 | 2.02E-17 | procollagen-lysine 2-oxoglutarate 5-dioxygenase 2 |
| LUM | -0.32416 | 0.044914 | lumican |
| ITGB3 | -0.32358 | 0.004820 | integrin beta 3 (platelet glycoprotein IIIa antigen CD61) |
| COL8A2 | -0.31419 | 0.002617 | collagen type VIII alpha 2 |
| MYL9 | -0.29422 | 4.79E-09 | myosin light chain 9 regulatory |
| TNC | -0.29344 | 1.55E-08 | tenascin C |
| TNFRSF1 1B | -0.29287 | 6.47E-05 | tumor necrosis factor receptor superfamily member 11b |
| TIMP1 | -0.28947 | 3.99E-08 | TIMP metallopeptidase inhibitor 1 |
| MMP14 | -0.28663 | 4.19E-12 | matrix metallopeptidase 14 (membrane-inserted) |
| ITGAV | -0.27785 | 3.86E-11 | integrin alpha V |
| TGFB1 | -0.27115 | 2.78E-08 | transforming growth factor beta 1 |
| SCG2 | -0.26599 | 0.035037 | secretogranin II |
| LRP1 | -0.24806 | 0.001513 | low density lipoprotein receptor-related protein 1 |
| INHBA | -0.24341 | 6.08E-07 | inhibin beta A |
| ITGB5 | -0.24256 | 1.91E-05 | integrin beta 5 |
| LOXL1 | -0.24153 | 0.002724 | lysyl oxidase-like 1 |
| PLOD1 | -0.24114 | 1.90E-05 | procollagen-lysine 2-oxoglutarate 5-dioxygenase 1 |
| IGFBP4 | -0.23587 | 5.03E-10 | insulin-like growth factor binding protein 4 |
| CD59 | -0.23574 | 2.49E-07 | CD59 molecule complement regulatory protein |
| EDIL3 | -0.23262 | 2.20E-09 | EGF-like repeats and discoidin I-like domains 3 |
| COL4A2 | -0.23101 | 1.70E-06 | collagen type IV alpha 2 |
| SLC6A8 | -0.2305 | 0.018164 | solute carrier family 6 (neurotransmitter transporter creatine) member 8 |
| EMP3 | -0.216 | 3.51E-06 | epithelial membrane protein 3 |
| GLIPR1 | -0.21534 | 0.017097 | GLI pathogenesis-related 1 |
| SGCB | -0.21334 | 5.10E-05 | sarcoglycan beta (43 kDa dystrophin-associated glycoprotein) |
| TPM4 | -0.20635 | 4.11E-10 | tropomyosin 4 |
| WIPF1 | -0.20429 | 0.000835 | WAS/WASL interacting protein family member 1 |
| LAMA3 | -0.20066 | 0.007495 | laminin alpha 3 |
| COL4A1 | -0.19848 | 0.002191 | collagen type IV alpha 1 |
| PLOD3 | -0.19821 | 4.28E-05 | procollagen-lysine 2-oxoglutarate 5-dioxygenase 3 |
| LAMC1 | -0.1908 | 2.65E-05 | laminin gamma 1 (formerly LAMB2) |
| CD44 | -0.18339 | 1.73E-08 | CD44 molecule (Indian blood group) |
| ITGB1 | -0.18212 | 3.36E-09 | integrin beta 1 (fibronectin receptor beta polypeptide antigen CD29 includes MDF2 MSK12) |
| APLP1 | -0.17568 | 0.048209 | amyloid beta (A4) precursor-like protein 1 |
| PDLIM4 | -0.17419 | 0.008049 | PDZ and LIM domain 4 |
| CALD1 | -0.16218 | 0.000650 | caldesmon 1 |
| CXCL1 | -0.16003 | 0.026641 | chemokine (C-X-C motif) ligand 1 (melanoma growth stimulating activity alpha) |
| LGALS 1 | -0.14181 | 0.000257 | lectin galactoside-binding soluble 1 |
| LEPRE1 | -0.13158 | 0.01596 | leucine proline-enriched proteoglycan (leprecan) 1 |
| CAPG | -0.13081 | 0.004625 | capping protein (actin filament) gelsolin-like |
| TGM2 | -0.1265 | 0.001922 | transglutaminase 2 (C polypeptide protein-glutamine-gamma-glutamyltransferase) |
| VCAM1 | -0.12597 | 0.044467 | vascular cell adhesion molecule 1 |
| FERMT2 | -0.12003 | 0.024081 | fermitin family member 2 |
| VIM | -0.091068 | 0.006269 | vimentin |
| MCM7 | 0.10245 | 0.031231 | minichromosome maintenance complex component 7 |
| PVR | 0.12063 | 0.024021 | poliovirus receptor |
| TNFRSF 12A | 0.12189 | 0.003415 | tumor necrosis factor receptor superfamily member 12A |
| IL6 | 0.16097 | 0.043979 | interleukin 6 (interferon beta 2) |
| CYR61 | 0.23994 | 2.38E-12 | cysteine-rich angiogenic inducer 61 |
| DKK1 | 0.30062 | 3.99E-16 | dickkopf 1 homolog (Xenopus laevis) |
| MMP1 | 0.40575 | 5.38E-07 | matrix metallopeptidase 1 (interstitial collagenase) |
| GADD45B | 0.44748 | 1.51E-14 | growth arrest and DNA-damage-inducible beta |
| GADD45 A | 0.53275 | 6.83E-30 | growth arrest and DNA-damage-inducible alpha |
| LAMA1 | 0.61799 | 7.43E-12 | laminin alpha 1 |
| MMP3 | 0.79443 | 5.10E-10 | matrix metallopeptidase 3 (stromelysin 1 progelatinase) |

TABLE 7C

Glycolysis Genes With Expression that Significantly Correlates with Increases in TRIM37 Levels in 231-2b Cells

| Gene Name | log2FC_TRIM_ASOvsctr | padj | GeneDescription |
|---|---|---|---|
| DDIT4 | -0.65926 | 1.26E-18 | DNA-damage-inducible transcript 4 |
| PPFIA4 | -0.65733 | 1.13E-10 | protein tyrosine phosphatase receptor type f polypeptide (PTPRF) interacting protein (liprin) alpha 4 |
| EGLN3 | -0.60858 | 1.00E-09 | egl nine homolog 3 (C. elegans) |
| ENO2 | -0.5959 | 3.33E-36 | enolase 2 (gamma neuronal) |
| NT5E | -0.47508 | 2.11E-32 | 5′-nucleotidase ecto (CD73) |
| PAM | -0.47225 | 3.72E-17 | peptidylglycine alpha-amidating monooxygenase |
| P4HA1 | -0.44941 | 4.99E-18 | prolyl 4-hydroxylase alpha polypeptide I |
| VEGFA | -0.44827 | 2.53E-40 | vascular endothelial growth factor A |
| STC1 | -0.43099 | 6.10E-24 | stanniocalcin 1 |
| IGFBP3 | -0.421 | 9.02E-05 | insulin-like growth factor binding protein 3 |
| MXI1 | -0.41475 | 4.32E-08 | MAX interactor 1 dimerization protein |
| PGK1 | -0.41349 | 2.07E-24 | phosphoglycerate kinase 1 |
| COL5A1 | -0.41137 | 7.46E-06 | collagen type V alpha 1 |
| ANKZF1 | -0.38919 | 3.41E-07 | ankyrin repeat and zinc finger domain containing 1 |
| AK4 | -0.38455 | 8.80E-16 | adenylate kinase 4 |
| VLDLR | -0.38066 | 0.000217 | very low density lipoprotein receptor |
| TGFBI | -0.37669 | 4.67E-07 | transforming growth factor beta-induced 68 kDa |
| SPAG4 | -0.37501 | 0.000676 | sperm associated antigen 4 |
| AGRN | -0.36008 | 1.26E-18 | agrin |
| EROIL | -0.35758 | 2.90E-20 | ERO1-like (S. cerevisiae) |
| TGFA | -0.3567 | 2.09E-11 | transforming growth factor alpha |
| B3GAT1 | -0.33781 | 0.006887 | beta-13-glucuronyltransferase 1 (glucuronosyltransferase P) |
| PLOD2 | -0.32559 | 2.02E-17 | procollagen-lysine 2-oxoglutarate 5-dioxygenase 2 |
| SOX9 | -0.32151 | 1.35E-08 | SRY (sex determining region Y)-box 9 |
| ARTN | -0.32082 | 0.003973 | artemin |
| IDUA | -0.28703 | 0.041653 | iduronidase alpha-L- |
| SDC3 | -0.25591 | 1.23E-07 | syndecan 3 |
| ZNF292 | -0.25512 | 0.001695 | zinc finger protein 292 |
| TPBG | -0.24386 | 1.22E-05 | trophoblast glycoprotein |
| PLOD1 | -0.24114 | 1.90E-05 | procollagen-lysine 2-oxoglutarate 5-dioxygenase 1 |
| P4HA2 | -0.23751 | 0.000763 | prolyl 4-hydroxylase alpha polypeptide II |
| FAM162A | -0.23499 | 0.005102 | family with sequence similarity 162 member A |
| SAP30 | -0.23413 | 0.012521 | Sin3A-associated protein 30 kDa |
| SLC16A3 | -0.23034 | 3.24E-09 | solute carrier family 16 member 3 (monocarboxylic acid transporter 4) |
| LDHA | -0.22633 | 1.55E-14 | lactate dehydrogenase A |
| DSC2 | -0.22604 | 0.003113 | desmocollin 2 |
| GLCE | -0.21701 | 0.000619 | glucuronic acid epimerase |
| LHPP | -0.21176 | 0.048186 | phospholysine phosphohistidine inorganic pyrophosphate phosphatase |
| PFKP | -0.20681 | 1.67E-08 | phosphofructokinase platelet |
| FUT8 | -0.1967 | 7.61E-05 | fucosyltransferase 8 (alpha (16) fucosyltransferase) |
| IL13RA1 | -0.19289 | 0.011117 | interleukin 13 receptor alpha 1 |
| PC | -0.18653 | 0.009081 | pyruvate carboxylase |
| CD44 | -0.18339 | 1.73E-08 | CD44 molecule (Indian blood group) |
| EXT2 | -0.18193 | 0.000433 | exostosin glycosyltransferase 2 |
| UGP2 | -0.18075 | 0.001208 | UDP-glucose pyrophosphorylase 2 |
| MPI | -0.18014 | 0.040866 | mannose phosphate isomerase |
| KDELR3 | -0.17883 | 0.049979 | KDEL (Lys-Asp-Glu-Leu) endoplasmic reticulum protein retention receptor 3 |
| MED24 | -0.16139 | 0.003110 | mediator complex subunit 24 |
| ALDOA | -0.15014 | 1.94E-05 | aldolase A fructose-bisphosphate |
| HK2 | -0.13992 | 0.010955 | hexokinase 2 |
| PYGL | -0.12711 | 0.007204 | phosphorylase glycogen liver |
| PGAM1 | -0.12566 | 0.000462 | phosphoglycerate mutase 1 (brain) |
| TPI1 | -0.10054 | 0.001455 | triosephosphate isomerase 1 |
| ENO1 | -0.082787 | 0.01718 | enolase 1 (alpha) |
| TXN | 0.12214 | 0.006123 | thioredoxin |
| AURKA | 0.13924 | 0.006070 | aurora kinase A |
| ME2 | 0.14653 | 0.049199 | malic enzyme 2 NAD(+)-dependent mitochondrial |
| PGM2 | 0.14832 | 0.015262 | phosphoglucomutase 2 |
| PSMC4 | 0.14843 | 0.002443 | proteasome (prosome macropain) 26S subunit ATPase 4 |
| HAX1 | 0.17152 | 0.006409 | HCLS1 associated protein X-1 |
| RARS | 0.18705 | 0.000263 | arginyl-tRNA synthetase |
| FKBP4 | 0.19476 | 0.000493 | FK506 binding protein 4 59 kDa |
| GNPDA1 | 0.23125 | 0.006953 | glucosamine-6-phosphate deaminase 1 |
| CITED2 | 0.30665 | 2.52E-06 | Cbp/p300-interacting transactivator with Glu/Asp-rich carboxy-terminal domain 2 |
| ADORA2B | 0.34829 | 1.19E-10 | adenosine A2b receptor |
| CTH | 0.41375 | 0.000278 | cystathionase (cystathionine gamma-lyase) |

TABLE 7D

Angiogenesis Genes With Expression that Significantly Correlates with Increases in TRIM37 Levels in 231-2b Cells

| Gene Name | log2FC_TRIM_ASOvsctr | padj | GeneDescription |
|---|---|---|---|
| SLCO2A1 | -0.62764 | 5.98E-13 | solute carrier organic anion transporter family member 2A1 |
| S100A4 | -0.49582 | 1.85E-06 | S100 calcium binding protein A4 |
| SPP1 | -0.4797 | 7.91E-05 | secreted phosphoprotein 1 |
| VEGFA | -0.44827 | 2.53E-40 | vascular endothelial growth factor A |
| STC1 | -0.43099 | 6.10E-24 | stanniocalcin 1 |
| LUM | -0.32416 | 0.044914 | lumican |
| TIMP1 | -0.28947 | 3.99E-08 | TIMP metallopeptidase inhibitor 1 |
| ITGAV | -0.27785 | 3.86E-11 | integrin alpha V |
| PDGFA | -0.23259 | 0.019937 | platelet-derived growth factor alpha polypeptide |
| NRP1 | -0.20684 | 2.86E-07 | neuropilin 1 |
| FGFR1 | -0.16004 | 0.000685 | fibroblast growth factor receptor 1 |
| JAG1 | -0.12148 | 0.047841 | jagged 1 |
| OLR1 | 0.35333 | 0.024868 | oxidized low density lipoprotein (lectin-like) receptor 1 |

TABLE 7E

Hedgehog Signaling Genes With Expression that Significantly Correlates with Increases in TRIM37 Levels in 231-2b Cells

| Gene Name | log2FC_TRIM_ASOvsctr | padj | GeneDescription |
|---|---|---|---|
| L1CAM | -0.63661 | 5.03E-35 | L1 cell adhesion molecule |
| SHH | -0.5153 | 4.90E-06 | sonic hedgehog |
| VEGFA | -0.44827 | 2.53E-40 | vascular endothelial growth factor A |
| VLDLR | -0.38066 | 0.000217 | very low density lipoprotein receptor |
| GPR56 | -0.3416 | 1.74E-05 | G protein-coupled receptor 56 |
| SCG2 | -0.26599 | 0.035037 | secretogranin II |
| NRP2 | -0.24703 | 0.006104 | neuropilin 2 |
| DPYSL2 | -0.23274 | 4.97E-05 | dihydropyrimidinase-like 2 |
| CELSR1 | -0.21551 | 0.005120 | cadherin EGF LAG seven-pass G-type receptor 1 |
| NRP1 | -0.20684 | 2.86E-07 | neuropilin 1 |
| PML | -0.17409 | 0.004613 | promyelocytic leukemia |
| LDB1 | -0.16623 | 0.011516 | LIM domain binding 1 |
| CDK6 | -0.16007 | 0.004754 | cyclin-dependent kinase 6 |
| RASA1 | -0.15799 | 0.022928 | RAS p21 protein activator (GTPase activating protein) 1 |
| TLE3 | -0.14215 | 0.002694 | transducin-like enhancer of split 3 (E(sp1) homolog Drosophila) |
| CDK5R1 | 0.2134 | 0.045487 | cyclin-dependent kinase 5 regulatory subunit 1 (p35) |

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein), which are identified by numbers within parentheses and that correspond to the numbering set forth below, are incorporated herein by reference in their entireties to the extent that they supplement, explain, provide a background for, and/or teach methodology, techniques, and/or compositions employed herein.

The discussion of the references is intended merely to summarize the assertions made by their authors. No admission is made that any reference (or a portion of any reference) is relevant prior art. Applicants reserve the right to challenge the accuracy and pertinence of any cited reference.

Acharyya et al. (2012) Cell 150:165-78.

Alqaraghuli et al. (2019) Curr Pharm Biotechnol 20:1087-107.

Astete et al. (2006) J Biomater Sci Polymer Edn 17:247-289.

Bailey et al. (2018) Cell 173:371-385 e318.

Bass (2001) Nature 411:428-429.

Batlevi et al. (2016) Haematologica 101:968-975.

Bauer et al. (2007) Cancer 109:1721-1728.

Bennardo et al. (2008) PLoS Genet 4:e1000110.

Bertucci et al. (2019) Nature 569:560-564.

Betageri et al. (1993) Liposome Drug Delivery Systems:Technomic Publishing. Lancaster, Pennsylvania, United States of America.

Bhatnagar & Green (2015) Cell Cycle 14:1345-6.

Bhatnagar et al. (2014) Nature 516:116-20.

Bouaoun et al. (2016) Hum Mutat 37:865-876.

Calhoun et al. (1997) Clin Orthopaed Related Res 341:206-214.

Cancer Genome Atlas Network (2012) Nature 490:61-70.

Cerami et al. (2012) Cancer Discov 2:401-404.

Chen et al. (2017) Front Neurosci 11:476.

Cheng et al. (2019) Signal Transduct Target Ther 4:62.

Cheung et al. (2018) Clin Cancer Res 24:5098-5111.

Chomczynski & Sacchi (2006) Nat Protoc 1:581-585.

Ciccia & Elledge (2010) Mol Cell 40:179-204.

Cicek et al. (2005) Cancer Res 65:3586-95.

Cicek et al. (2009) Clin Exp Metastasis 26:229-37.

Cohn & D′Andrea (2008) Mol Cell 32:306-12.

Cortazar et al. (2014) Lancet 384:164-172.

Cuellar et al. (2015) Nucleic Acids Res 43:1189-1203.

de Faria et al. (2005) Macromol Symp 229:228-233.

De Mattos-Arruda et al. (2019) Cell Rep 27:2690-2708 e2610.

Deng et al. (2015) Nat Mater 14:1278-85.

Desai & Bergman (2015) Neuropsychopharmacology 40:2207-16.

Echeverria et al. (2018) Nat Commun 9:5079.

Elbashir et al. (2001) Nature 411:494-498.

Flaman et al. (1995) Proc Natl Acad Sci U S A 92:3963-3967.

Foulkes et al. (2010) N Engl J Med 363:1938-1948.

Gao et al. (2013) Sci Signal 6:pl1.

Garrido-Castro et al. (2019) Cancer Discov 9:176-198.

Gasco et al. (2003) Hum Mutat 21:301-306.

GENBANK® Accession Nos. NC_000017.11; NM_000802.3; NM_001005207.5; NM_001108288.1; NM_001110182.1; NM_001320987.3; NM_001320988.3; NM_001320989.3; NM_001363025.1; NM_015294.6; NM_016724.3; NM_016725.3; NM_0167829.3; NP_000793.1; NP_001005207.1; NP_001101758.1; NP_001103652.1; NP_001307916.1; NP_001307917.1; NP_001349954.1; NP_01307918.1; NP_056109.1; NP_057936.1; NP_057937.1; NP_057941.1; XM_011289104.3; XM_038677132.1; XP_01128746.1; XP_038533060.1.

Gibson et al. (2016) Nat Genet 48:848-855.

Ginter et al. (2017) Clin Breast Cancer 17:544-549.

Goldstein & Kastan (2015) Annu Rev Med 66:129-43.

Gonsalves et al. (1998) Biomaterials 19:1501-1505.

Green (2019) Cell 177:1094-1107.

Greenblatt et al. (2001) Cancer Res 61:4092-4097.

Gregoriadis (ed.) (1993) Liposome Technology. CRC Press, Boca Raton, Florida, United States of America.

Gyori et al. (2014) Redox Biol 2:457-465.

Hans et al. (2002) Solid State Mater Sci 6:319-327.

Holen et al. (2017) Dis Model Mech 10:359-371.

Holstege et al. (2009) Cancer Res 69:3625-3633.

Hu et al. (2009) Cancer Cell 15:9-20.

Hurst et al. (2008) J Biol Chem 283:7438-44.

Ismail et al. (2010) J Cell Biol 191:45-60.

Janoff (ed.) (1999) Liposomes: Rational Design. M. Dekker, New York, New York, United States of America.

Jenkins et al. (2005) Breast Cancer Res 7:R444-454.

Jhan & Andrechek (2017) Triple-negative breast cancer and the potential for targeted therapy. Pharmacogenomics 18:1595-1609.

Kakarougkas et al. (2014) Mol Cell 55:723-732.

Kastenhuber & Lowe (2017) Cell 170:1062-78.

Kim et al. (2018) Cell 173:879-93 e13.

Kirschbaum et al. (2014) Leuk Lymphoma 55:2301-2304.

Kodura & Souchelnytskyi (2015) Cancer Metastasis Rev 34:611-8.

Konner et al. (2010) Clin Cancer Res 16(21):5288-5295.

Kumar et al. (2004) Biomaterials 25:1771-1777.

Kwon et al. (2001) Colloids Surf A 182: 123-130.

Labat-Moleur et al. (1996) Gene Therapy 3:1010-1017.

Lambert et al. (2017) Cell 168:670-91.

Lamprecht et al. (1999) Intl J Pharmaceut 184:97-105.

Lasic & Martin (1995) Stealth Liposomes. CRC Press, Boca Raton, Florida, United States of America.

Laurencin et al. (2001) Biomaterials 22:1271-1277.

Lehmann-Che et al. (2010) Oncologist 15:246-252.

Liedtke et al. (2008) J Clin Oncol 26:1275-1281.

Lord & Ashworth (2012) Nature 481:287-94.

Lou et al. (2018) ACS Omega 3:9210-9.

Maeda et al. (2018) Cancer Res 78:205-215.

Mandal et al. (2002) Pharmaceut Res 19:1713-1719.

Mina et al. (2017) Cancer Cell 32:155-168 e156.

Minn et al. (2005) Nature 436:518-524.

Navin (2014) Cell Rep 6:417-9.

Nik-Zainal et al. (2016) Nature 534:47-54.

O′Connor (2015) Mol Cell 60:547-60.

Olivier et al. (2010) Cold Spring Harb Perspect Biol 2:a001008.

Ozdemir & Dotto (2017) Trends Cancer 3:181-197.

Pal et al. (2011) Breast Cancer Res Treat 125:627-36.

PCT International Patent Application Publication Nos. WO 99/07409; WO 99/32619; WO 2000/01846; WO 2000/44895; WO 2000/44914; WO 2001/36646; WO 2001/29058; WO 2012/119077; WO 2014/087863; WO 2019/099374.

Pereira et al. (2016) Nat Commun 7:11479.

Pfister & Prives (2017) Cold Spring Harb Perspect Med 7.

Pierce et al. (1999) Genes Dev 13:2633-2638.

Piktel et al. (2016) J Nanobiotechnology 14:39.

Richards et al. (2017) Chem Sci 8:63-77.

Richardson et al. (1998) Genes Dev 12:3831-3842.

Rosenblum et al. (2018) Nat Commun 9:1410.

Roy et al. (2014) Protein Cell 5:265-296.

Ryan & Bernstein (2012) Science 336:1513-4.

Sanjana et al. (2014) Nat Methods 11:783-784.

Sanna et al. (2014) Int J Nanomedicine 9:467-83.

Shah et al. (2012) Nature 486:395-399.

Shanbhag et al. (2010) Cell 141:970-81.

Shen & Laird (2013) Cell 153:38-55.

Shivange et al. (2018) Cancer Cell 34:331-45 e11.

Silver et al. (2010) J Clin Oncol 28:1145-1153.

Song et al. (1997) J Controlled Release 43:197-212.

Soppimath et al. (2001) J Controlled Release 70:1-20.

Spruance et al. (2004) Antimicrob Agents Chemother 48:2787-2792.

Stephens et al. (2012) Nature 486:400-4.

Stockel et al. (2016) Bioinformatics 32:1502-1508.

Stracquadanio et al. (2016) Nat Rev Cancer 16:251-265.

Subbiah et al. (2010) Curr Med Chem 17:4559-4577.

Subramanian et al. (2005) Proc Natl Acad Sci USA 102:15545-15550.

Suk et al. (2016) Adv Drug Deliv Rev 99:28-51.

Tominaga et al. (2015) Nat Commun 6:6716.

U.S. Pat. Application Publication Nos. 2011/0104069; 2013/0330279; 2018/0078657; 2019/0091280; 2020/0283537; 2020/0038452.

U.S. Pat. Nos. 4,235,871; 4,551,482; 5,011,634; 5,948,767; 6,056,938; 6,132,766; 6,180,082; 6,197,333; 6,200,598; 6,210,707; 6,217,886; 7,332,586; 7,901,711; 7,973,015; 8,137,697; 8,236,557; 8,268,962; 8,304,398; 8,361,979; 8,449,915; 8,663,599; 8,802,645; 9,080,170; 9,238,042; 9,598,703; 9,599,621; 9,738,891; 9,862,945; 9,972,015; 9,777,727; 10,030,894; 10,188,633; 10,590,420; 10,781,541.

Ui et al. (2015) Mol Cell 58:468-482.

Ulasov et al. (2019) Cancer Metastasis Rev 38:493-506.

Vidula & Bardia (2017) Oncotarget 8:106167-8.

Volk-Draper et al. (2012) Neoplasia 14:926-42.

Weiss et al. (2010) Oncogene 29:4715-24.

Williams et al. (2007) Biochem Cell Biol 85:509-20.

Woods et al. (2007) Cancer Res 67:10744-10752.

Wu et al. (2018) Cancer Res 78:6399-412.

Wu et al. (2019) Cancer Research 79:5550-5562.

Yates et al. (2017) Cancer Cell 32: 169-84 e7.

Yee & Raje (2018) Oncologist 23:516-517.

Zhang et al. (2016) Nat Struct Mol Biol 23:522-530.

Zhou et al. (2008) Mol Cell 29:69-80.

Zhu & Zhou (2015) Front Endocrinol (Lausanne) 6:159.

Zuckerman et al. (2014) Proc Natl Acad Sci USA 111:11449-54.

It will be understood that various details of the presently disclosed subject matter can be changed without departing from the scope of the presently disclosed subject matter. Furthermore, the foregoing description is for the purpose of illustration only, and not for the purpose of limitation.

1. A method of sensitizing a cancer in a subject to a therapeutic agent, the method comprising administering to the subject a composition comprising an effective amount of an inhibitor of TRIM37 activity.

2. (canceled)

3. (canceled)

4. (canceled)

5. The method of claim 1, wherein the inhibitor of TRIM37 activity is selected from the group consisting of an anti-sense oligonucleotide, a small molecule inhibitor, and a combination thereof.

6. The method of claim 1, wherein the composition comprises a nanoparticle, a targeting moiety, or a combination thereof.

7. (canceled)

8. The method of claim 6, wherein the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide(polyethyleneglycol-2000)] (DSPE-PEG2000-Maleimide), or any combination thereof.

9. The method of claim 6, wherein the targeting moiety comprise an anti-FOLR1 antibody.

10. The method of claim 9, wherein the antibody comprises a linker sequence through which the antibody is conjugated to the nanoparticle.

11. The method of claim 10, wherein the linker sequence comprises an Fc-linkered sequence containing a cysteine.

12. The method of claim 11, wherein the Fc-linkered sequence is present at the C-terminus of a heavy chain of the antibody.

13. A method of treating a cancer in a subject in need thereof, the method comprising administering to the subject (a) a composition comprising an effective amount of an inhibitor of TRIM37 activity; and (b) an anti-cancer therapeutic agent.

14. (canceled)

15. (canceled)

16. The method of claim 13, wherein the inhibitor of TRIM37 activity is selected from the group consisting of an anti-sense oligonucleotide, a small molecule inhibitor, and a combination thereof.

17. The method of claim 13, wherein the composition comprises a nanoparticle, a targeting moiety, or a combination thereof.

18. (canceled)

19. (canceled)

20. The method of claim 17, wherein the targeting moiety comprises an anti-FOLR1 antibody.

21. The method of claim 20, wherein the antibody comprises a linker sequence through which the antibody is conjugated to the nanoparticle.

22. The method of claim 21, wherein the linker sequence comprises an Fc-linkered sequence containing a cysteine.

23. The method of claim 22, wherein the Fc-linkered sequence is present at the C-terminus of a heavy chain of the antibody.

24. A purified and isolated antibody, or a fragment thereof comprising at least one paratope, comprising a linker sequence through which the antibody can be conjugated to a carrier, wherein the linker sequence comprises the amino acid sequence ((X)₃Cys(X)₃, wherein each X is independently any amino acid.

25. The purified and isolated antibody or the fragment thereof of claim 24, wherein the linker sequence comprises an Fc-linkered sequence containing a cysteine.

26. The purified and isolated antibody or the fragment thereof of claim 25, wherein the Fc-linkered sequence is present at the C-terminus of a heavy chain of the antibody.

27. The purified and isolated antibody or the fragment thereof of claim 24, wherein the carrier is a nanoparticle.

28. The purified and isolated antibody or the fragment thereof of claim 24 wherein the purified and isolated antibody or the fragment thereof specifically binds to a folate receptor.

29. The purified and isolated antibody or the fragment thereof of claim 24, wherein the carrier comprises an inhibitor of TRIM37 activity.

30. The purified and isolated antibody or the fragment thereof of claim 29, wherein the inhibitor of TRIM37 activity is selected from the group consisting of an anti-sense oligonucleotide, a small molecule inhibitor, and a combination thereof.

31. The purified and isolated antibody or the fragment thereof of claim 27, wherein the nanoparticle is liposome-based.

32. The purified and isolated antibody or the fragment thereof of claim 27, wherein the nanoparticle comprises a lipid bilayer comprising 1,2-dioleoyl-sn-glycero-3-phosphate (DOPA), 1,2-dioleoyl-3-trimethylammonium-propane (DOTAP), 1,2-distearoryl-sn-glycero-3-phosphoethanolamine-N-[maleimide(polyethyleneglycol-2000)] (DSPE-PEG2000-Maleimide), or any combination thereof.


SEQUENCE LISTING (100 sequences)

SEQ ID NO: 1 - DNA, 4489 bp, Homo sapiens
  CDS (420)..(3314)
gcagcttctctcgccgccgcgcccccagctgctctggttacggttggtgtttgcgaattctctgcggcaccttcttcgctgaactccaccgtcagttccataggcgcgcgcccgcgtcacgtgggccgaggttccggcgcggctgctggccggcggctgagccgggagaaaggagctgcggccaccgttcgccctacttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggagagcattgctgaggttttccgatgtttcatttgtatggagaaattgcgggatgcacgcctgtgtcctcattgctccaaactgtgttgtttcagctgtattaggcgctggctgacagagcagagagctcaatgtcctcattgccgtgctccactccagctacgagaactagtaaattgtcgttgggcagaagaagtaacacaacagcttgatactcttcaactctgcagtctcaccaaacatgaagaaaatgaaaaggacaaatgtgaaaatcaccatgaaaaacttagtgtattttgctggacttgtaagaagtgtatctgccatcagtgtgcactttggggaggaatgcatggcggacatacctttaaacctttggcagaaatttatgagcaacacgtcactaaagtgaatgaagaggtagccaaacttcgtcggcgtctcatggaactgatcagcttagttcaagaagtggaaaggaatgtagaagctgtaagaaatgcaaaagatgagcgtgttcgggaaattaggaatgcagtggagatgatgattgcacggttagacacacagctgaagaataagcttataacactgatgggtcagaagacatctctaacccaagaaacagagcttttggaatccttacttcaggaggtggagcaccagttgcggtcttgtagtaagagtgagttgatatctaagagctcagagatccttatgatgtttcagcaagttcatcggaagcccatggcatcttttgttaccactcctgttccaccagactttaccagtgaattagtgccatcttacgattcagctacttttgttttagagaatttcagcactttgcgtcagagagcagatcctgtttacagtccacctcttcaagtttcaggactttgctggaggttaaaagtttacccagatggaaatggagttgtgcgaggttactacttatctgtgtttctggagctctcagctggcttgcctgaaacttctaaatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtttcaggtacgttcaccaactttctttcaaaaatcccgggaccagcattggtacattactcagttggaagctgcacagactagttatatccaacaaataaacaaccttaaagagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatcacgagctttcagatggagatctggatctggatcttgtttatgaggatgaagtaaatcagctcgatggcagcagttcctctgctagttccacagcaacaagtaatacagaagaaaatgatattgatgaagaaactatgtctggagaaaatgatgtggaatataacaacatggaattagaagagggagaactcatggaagatgcagctgctgcaggacccgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactccacaaataagaagagtaattcgcccaagccagctcgatccagtgtagcaggtagtctatcacttcgaagagcagtggaccctggagaaaatagtcgttcaaagggagactgtcagactctgtctgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttgggggctaatgctaaaggaggtcatctggaaggactgcagatgactgatttggaaaataattctgaaactggagagttacagcctgtactacctgaaggagcttcagctgcccctgaagaaggaatgagtagcgacagtgacattgaatgtgacactgagaatgaggagcaggaagagcataccagtgtgggcgggtttcacgactccttcatggtcatgacacagcccccggatgaagatacacattccagttttcctgatggtgaacaaataggccctgaagatctcagcttcaatacagatgaaaatagtggaaggtaattgccaaatcaagagaactgacttgcaagctaccttgaccctgaattttgctgtagttggtgctcaaatttgtcatcagtcagataatcagatttggtcttatttcttcattatctcgacctgaaatagtaatttggaaactgttggaaggtggcacagtttagtctaagacagcagtagtacatgggaaaaacagtatgggaagagttctttgtaatgtaaggaaataacaatgtagttctctattaatttagcaaatttgtacattcacaaaaggcagtttgtctactacagcagaaggctggttaactgccagaaaatgtacctccaggccctgcatgccgtcagtaacccgcccggcattggtgctctactgtctttggctagagcttagttgtgtttaaataatcatctttatatttggggttttaattacagttccattagtgcctgtagattagtgaacagaaaattgctttggaagagattctgccctgtagacactatgtgaataactgaagtaacactagactgaatctcctttttggagtatgtatcttctctcacttgttcaagtacaggcacactgttcaaccgcatggtatctttctgttgtgtgacttctacaaatgtaattttaaatgaaattaagttaacatggattcattacgttcctggccctgtagacacgtgtaagattatttaaaattctttcatttttttctgcctcttactatacgactgtagtgcaacaaatattttaaagcccccttttcttctttattttcattagttgtacattgatttcagtgtcaacacatttaaagattcattcatgttgcacagtggcttacatgaacgtgaaactgtgatataaggttttctttcatactcataattagcccaaaacagttgccaaactttgccattgtgctcctgcatttgtgtttgagctgctatatatttgtggaaattacactgaaagttgactaagagactattgaaaaagcatgaataattaaatatacatgtgagagacatctcatctgctgtattttacttagtgaatattgttcactcttccgtgtctgatgtcttgctgaatgctgtgactcatagtttacttttgttcaaaatagtttgcactttttgttaataaaatcaacttgagaaaa

SEQ ID NO: 2 - protein, 964 aa, Homo sapiens
MDEQSVESIAEVFRCFICMEKLRDARLCPHCSKLCCFSCIRRWLTEQRAQCPHCRAPLQLRELVNCRWAEEVTQQLDTLQLCSLTKHEENEKDKCENHHEKLSVFCWTCKKCICHQCALWGGMHGGHTFKPLAEIYEQHVTKVNEEVAKLRRRLMELISLVQEVERNVEAVRNAKDERVREIRNAVEMMIARLDTQLKNKLITLMGQKTSLTQETELLESLLQEVEHQLRSCSKSELISKSSEILMMFQQVHRKPMASFVTTPVPPDFTSELVPSYDSATFVLENFSTLRQRADPVYSPPLQVSGLCWRLKVYPDGNGVVRGYYLSVFLELSAGLPETSKYEYRVEMVHQSCNDPTKNIIREFASDFEVGECWGYNRFFRLDLLANEGYLNPQNDTVILRFQVRSPTFFQKSRDQHWYITQLEAAQTSYIQQINNLKERLTIELSRTQKSRDLSPPDNHLSPQNDDALETRAKKSACSDMLLEGGPTTASVREAKEDEEDEEKIQNEDYHHELSDGDLDLDLVYEDEVNQLDGSSSSASSTATSNTEENDIDEETMSGENDVEYNNMELEEGELMEDAAAAGPAGSSHGYVGSSSRISRRTHLCSAATSSLLDIDPLILIHLLDLKDRSSIENLWGLQPRPPASLLQPTASYSRKDKDQRKQQAMWRVPSDLKMLKRLKTQMAEVRCMKTDVKNTLSEIKSSSAASGDMQTSLFSADQAALAACGTENSGRLQDLGMELLAKSSVANCYIRNSTNKKSNSPKPARSSVAGSLSLRRAVDPGENSRSKGDCQTLSEGSPGSSQSGSRHSSPRALIHGSIGDILPKTEDRQCKALDSDAVVVAVFSGLPAVEKRRKMVTLGANAKGGHLEGLQMTDLENNSETGELQPVLPEGASAAPEEGMSSDSDIECDTENEEQEEHTSVGGFHDSFMVMTQPPDEDTHSSFPDGEQIGPEDLSFNTDENSGR

SEQ ID NO: 3 - DNA, 3583 bp, Homo sapiens
  CDS (420)..(3314)
gcagcttctctcgccgccgcgcccccagctgctctggttacggttggtgtttgcgaattctctgcggcaccttcttcgctgaactccaccgtcagttccataggcgcgcgcccgcgtcacgtgggccgaggttccggcgcggctgctggccggcggctgagccgggagaaaggagctgcggccaccgttcgccctacttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggagagcattgctgaggttttccgatgtttcatttgtatggagaaattgcgggatgcacgcctgtgtcctcattgctccaaactgtgttgtttcagctgtattaggcgctggctgacagagcagagagctcaatgtcctcattgccgtgctccactccagctacgagaactagtaaattgtcgttgggcagaagaagtaacacaacagcttgatactcttcaactctgcagtctcaccaaacatgaagaaaatgaaaaggacaaatgtgaaaatcaccatgaaaaacttagtgtattttgctggacttgtaagaagtgtatctgccatcagtgtgcactttggggaggaatgcatggcggacatacctttaaacctttggcagaaatttatgagcaacacgtcactaaagtgaatgaagaggtagccaaacttcgtcggcgtctcatggaactgatcagcttagttcaagaagtggaaaggaatgtagaagctgtaagaaatgcaaaagatgagcgtgttcgggaaattaggaatgcagtggagatgatgattgcacggttagacacacagctgaagaataagcttataacactgatgggtcagaagacatctctaacccaagaaacagagcttttggaatccttacttcaggaggtggagcaccagttgcggtcttgtagtaagagtgagttgatatctaagagctcagagatccttatgatgtttcagcaagttcatcggaagcccatggcatcttttgttaccactcctgttccaccagactttaccagtgaattagtgccatcttacgattcagctacttttgttttagagaatttcagcactttgcgtcagagagcagatcctgtttacagtccacctcttcaagtttcaggactttgctggaggttaaaagtttacccagatggaaatggagttgtgcgaggttactacttatctgtgtttctggagctctcagctggcttgcctgaaacttctaaatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtttcaggtacgttcaccaactttctttcaaaaatcccgggaccagcattggtacattactcagttggaagctgcacagactagttatatccaacaaataaacaaccttaaagagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatcacgagctttcagatggagatctggatctggatcttgtttatgaggatgaagtaaatcagctcgatggcagcagttcctctgctagttccacagcaacaagtaatacagaagaaaatgatattgatgaagaaactatgtctggagaaaatgatgtggaatataacaacatggaattagaagagggagaactcatggaagatgcagctgctgcaggacccgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactccacaaataagaagagtaattcgcccaagccagctcgatccagtgtagcaggtagtctatcacttcgaagagcagtggaccctggagaaaatagtcgttcaaagggagactgtcagactctgtctgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttgggggctaatgctaaaggaggtcatctggaaggactgcagatgactgatttggaaaataattctgaaactggagagttacagcctgtactacctgaaggagcttcagctgcccctgaagaaggaatgagtagcgacagtgacattgaatgtgacactgagaatgaggagcaggaagagcataccagtgtgggcgggtttcacgactccttcatggtcatgacacagcccccggatgaagatacacattccagttttcctgatggtgaacaaataggccctgaagatctcagcttcaatacagatgaaaatagtggaagataatttgatttgaaactgacactgcacctgatgggttaacaagatctaggcttcagaaggtgacagatatgagtgaggaccatgtgtggggcaaagcctcagaatgatgaaaaggttccggcactatagttggggccatgttgactccttttcaaccatttgtcacagacgtgagaagaagaaatgacttcaaaatcaagagaaaacaaatactgaaagtctctacttacatccaaattttaaaaaataaaatctgtagattaacaatctga

SEQ ID NO: 4 - protein, 964 aa, Homo sapiens; identical to SEQ ID NO: 2

SEQ ID NO: 5 - DNA, 4387 bp, Homo sapiens
  CDS (420)..(3212)
gcagcttctctcgccgccgcgcccccagctgctctggttacggttggtgtttgcgaattctctgcggcaccttcttcgctgaactccaccgtcagttccataggcgcgcgcccgcgtcacgtgggccgaggttccggcgcggctgctggccggcggctgagccgggagaaaggagctgcggccaccgttcgccctacttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggagcgctggctgacagagcagagagctcaatgtcctcattgccgtgctccactccagctacgagaactagtaaattgtcgttgggcagaagaagtaacacaacagcttgatactcttcaactctgcagtctcaccaaacatgaagaaaatgaaaaggacaaatgtgaaaatcaccatgaaaaacttagtgtattttgctggacttgtaagaagtgtatctgccatcagtgtgcactttggggaggaatgcatggcggacatacctttaaacctttggcagaaatttatgagcaacacgtcactaaagtgaatgaagaggtagccaaacttcgtcggcgtctcatggaactgatcagcttagttcaagaagtggaaaggaatgtagaagctgtaagaaatgcaaaagatgagcgtgttcgggaaattaggaatgcagtggagatgatgattgcacggttagacacacagctgaagaataagcttataacactgatgggtcagaagacatctctaacccaagaaacagagcttttggaatccttacttcaggaggtggagcaccagttgcggtcttgtagtaagagtgagttgatatctaagagctcagagatccttatgatgtttcagcaagttcatcggaagcccatggcatcttttgttaccactcctgttccaccagactttaccagtgaattagtgccatcttacgattcagctacttttgttttagagaatttcagcactttgcgtcagagagcagatcctgtttacagtccacctcttcaagtttcaggactttgctggaggttaaaagtttacccagatggaaatggagttgtgcgaggttactacttatctgtgtttctggagctctcagctggcttgcctgaaacttctaaatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtttcaggtacgttcaccaactttctttcaaaaatcccgggaccagcattggtacattactcagttggaagctgcacagactagttatatccaacaaataaacaaccttaaagagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatcacgagctttcagatggagatctggatctggatcttgtttatgaggatgaagtaaatcagctcgatggcagcagttcctctgctagttccacagcaacaagtaatacagaagaaaatgatattgatgaagaaactatgtctggagaaaatgatgtggaatataacaacatggaattagaagagggagaactcatggaagatgcagctgctgcaggacccgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactccacaaataagaagagtaattcgcccaagccagctcgatccagtgtagcaggtagtctatcacttcgaagagcagtggaccctggagaaaatagtcgttcaaagggagactgtcagactctgtctgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttgggggctaatgctaaaggaggtcatctggaaggactgcagatgactgatttggaaaataattctgaaactggagagttacagcctgtactacctgaaggagcttcagctgcccctgaagaaggaatgagtagcgacagtgacattgaatgtgacactgagaatgaggagcaggaagagcataccagtgtgggcgggtttcacgactccttcatggtcatgacacagcccccggatgaagatacacattccagttttcctgatggtgaacaaataggccctgaagatctcagcttcaatacagatgaaaatagtggaaggtaattgccaaatcaagagaactgacttgcaagctaccttgaccctgaattttgctgtagttggtgctcaaatttgtcatcagtcagataatcagatttggtcttatttcttcattatctcgacctgaaatagtaatttggaaactgttggaaggtggcacagtttagtctaagacagcagtagtacatgggaaaaacagtatgggaagagttctttgtaatgtaaggaaataacaatgtagttctctattaatttagcaaatttgtacattcacaaaaggcagtttgtctactacagcagaaggctggttaactgccagaaaatgtacctccaggccctgcatgccgtcagtaacccgcccggcattggtgctctactgtctttggctagagcttagttgtgtttaaataatcatctttatatttggggttttaattacagttccattagtgcctgtagattagtgaacagaaaattgctttggaagagattctgccctgtagacactatgtgaataactgaagtaacactagactgaatctcctttttggagtatgtatcttctctcacttgttcaagtacaggcacactgttcaaccgcatggtatctttctgttgtgtgacttctacaaatgtaattttaaatgaaattaagttaacatggattcattacgttcctggccctgtagacacgtgtaagattatttaaaattctttcatttttttctgcctcttactatacgactgtagtgcaacaaatattttaaagcccccttttcttctttattttcattagttgtacattgatttcagtgtcaacacatttaaagattcattcatgttgcacagtggcttacatgaacgtgaaactgtgatataaggttttctttcatactcataattagcccaaaacagttgccaaactttgccattgtgctcctgcatttgtgtttgagctgctatatatttgtggaaattacactgaaagttgactaagagactattgaaaaagcatgaataattaaatatacatgtgagagacatctcatctgctgtattttacttagtgaatattgttcactcttccgtgtctgatgtcttgctgaatgctgtgactcatagtttacttttgttcaaaatagtttgcactttttgttaataaaatcaacttgagaaaa

SEQ ID NO: 6 - protein, 930 aa, Homo sapiens
MDEQSVERWLTEQRAQCPHCRAPLQLRELVNCRWAEEVTQQLDTLQLCSLTKHEENEKDKCENHHEKLSVFCWTCKKCICHQCALWGGMHGGHTFKPLAEIYEQHVTKVNEEVAKLRRRLMELISLVQEVERNVEAVRNAKDERVREIRNAVEMMIARLDTQLKNKLITLMGQKTSLTQETELLESLLQEVEHQLRSCSKSELISKSSEILMMFQQVHRKPMASFVTTPVPPDFTSELVPSYDSATFVLENFSTLRQRADPVYSPPLQVSGLCWRLKVYPDGNGVVRGYYLSVFLELSAGLPETSKYEYRVEMVHQSCNDPTKNIIREFASDFEVGECWGYNRFFRLDLLANEGYLNPQNDTVILRFQVRSPTFFQKSRDQHWYITQLEAAQTSYIQQINNLKERLTIELSRTQKSRDLSPPDNHLSPQNDDALETRAKKSACSDMLLEGGPTTASVREAKEDEEDEEKIQNEDYHHELSDGDLDLDLVYEDEVNQLDGSSSSASSTATSNTEENDIDEETMSGENDVEYNNMELEEGELMEDAAAAGPAGSSHGYVGSSSRISRRTHLCSAATSSLLDIDPLILIHLLDLKDRSSIENLWGLQPRPPASLLQPTASYSRKDKDQRKQQAMWRVPSDLKMLKRLKTQMAEVRCMKTDVKNTLSEIKSSSAASGDMQTSLFSADQAALAACGTENSGRLQDLGMELLAKSSVANCYIRNSTNKKSNSPKPARSSVAGSLSLRRAVDPGENSRSKGDCQTLSEGSPGSSQSGSRHSSPRALIHGSIGDILPKTEDRQCKALDSDAVVVAVFSGLPAVEKRRKMVTLGANAKGGHLEGLQMTDLENNSETGELQPVLPEGASAAPEEGMSSDSDIECDTENEEQEEHTSVGGFHDSFMVMTQPPDEDTHSSFPDGEQIGPEDLSFNTDENSGR

SEQ ID NO: 7 - DNA, 4371 bp, Homo sapiens
  CDS (420)..(3197)
gcagcttctctcgccgccgcgcccccagctgctctggttacggttggtgtttgcgaattctctgcggcaccttcttcgctgaactccaccgtcagttccataggcgcgcgcccgcgtcacgtgggccgaggttccggcgcggctgctggccggcggctgagccgggagaaaggagctgcggccaccgttcgccctacttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggagagcattgctgaggttttccgatgtttcatttgtatggagaaattgcgggatgcacgcctgtgtcctcattgctccaaactgtgttgtttcagctgtattaggcgctggctgacagagcagagagctcaatgtcctcattgccgtgctccactccagctacgagaactagtaaattgtcgttgggcagaagaagtaacacaacagcttgatactcttcaactctgcagtctcaccaaacatgaagaaaatgaaaaggacaaatgtgaaaatcaccatgaaaaacttagtgtattttgctggacttgtaagaagtgtatctgccatcagtgtgcactttggggaggaatgcatggcggacatacctttaaacctttggcagaaatttatgagcaacacgtcactaaagtgaatgaagaggtagccaaacttcgtcggcgtctcatggaactgatcagcttagttcaagaagtggaaaggaatgtagaagctgtaagaaatgcaaaagatgagcgtgttcgggaaattaggaatgcagtggagatgatgattgcacggttagacacacagctgaagaataagcttataacactgatgggtcagaagacatctctaacccaagaaacagagcttttggaatccttacttcaggaggtggagcaccagttgcggtcttgtagtaagagtgagttgatatctaagagctcagagatccttatgatgtttcagcaagttcatcggaagcccatggcatcttttgttaccactcctgttccaccagactttaccagtgaattagtgccatcttacgattcagctacttttgttttagagaatttcagcactttgcgtcagagagcagatcctgtttacagtccacctcttcaagtttcaggactttgctggaggttaaaagtttacccagatggaaatggagttgtgcgaggttactacttatctgtgtttctggagctctcagctggcttgcctgaaacttctaaatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtttcaggtacgttcaccaactttctttcaaaaatcccgggaccagcattggtacattactcagttggaagctgcacagactagttatatccaacaaataaacaaccttaaagagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatcacgagctttcagatggagatctggatctggatcttgtttatgaggatgaagtaaatcagctcgatggcagcagttcctctgctagttccacagcaacaagtaatacagaagaaaatgatattgatgaagaaactatgtctggagaaaatgatgtggaatataacaacatggaattagaagagggagaactcatggaagatgcagctgctgcaggacccgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactccacaaataagaagagtaattcgcccaagccagctcgatccagtgtagcaggtagtctatcacttcgaagagcagtggaccctggagaaaatagtcgttcaaagggagactgtcagactctgtctgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttgggggctaatgctaaaggaggtcatctggaaggactgcagatgactgatttggaaaataattctgaaactggagagttacagcctgtactacctgaaggagcttcagctgcccctgaagaagatacacattccagttttcctgatggtgaacaaataggccctgaagatctcagcttcaatacagatgaaaatagtggaaggtaattgccaaatcaagagaactgacttgcaagctaccttgaccctgaattttgctgtagttggtgctcaaatttgtcatcagtcagataatcagatttggtcttatttcttcattatctcgacctgaaatagtaatttggaaactgttggaaggtggcacagtttagtctaagacagcagtagtacatgggaaaaacagtatgggaagagttctttgtaatgtaaggaaataacaatgtagttctctattaatttagcaaatttgtacattcacaaaaggcagtttgtctactacagcagaaggctggttaactgccagaaaatgtacctccaggccctgcatgccgtcagtaacccgcccggcattggtgctctactgtctttggctagagcttagttgtgtttaaataatcatctttatatttggggttttaattacagttccattagtgcctgtagattagtgaacagaaaattgctttggaagagattctgccctgtagacactatgtgaataactgaagtaacactagactgaatctcctttttggagtatgtatcttctctcacttgttcaagtacaggcacactgttcaaccgcatggtatctttctgttgtgtgacttctacaaatgtaattttaaatgaaattaagttaacatggattcattacgttcctggccctgtagacacgtgtaagattatttaaaattctttcatttttttctgcctcttactatacgactgtagtgcaacaaatattttaaagcccccttttcttctttattttcattagttgtacattgatttcagtgtcaacacatttaaagattcattcatgttgcacagtggcttacatgaacgtgaaactgtgatataaggttttctttcatactcataattagcccaaaacagttgccaaactttgccattgtgctcctgcatttgtgtttgagctgctatatatttgtggaaattacactgaaagttgactaagagactattgaaaaagcatgaataattaaatatacatgtgagagacatctcatctgctgtattttacttagtgaatattgttcactcttccgtgtctgatgtcttgctgaatgctgtgactcatagtttacttttgttcaaaatagtttgcactttttgttaataaaatcaacttgagaaa

SEQ ID NO: 8 - protein, 925 aa, Homo sapiens
MDEQSVESIAEVFRCFICMEKLRDARLCPHCSKLCCFSCIRRWLTEQRAQCPHCRAPLQLRELVNCRWAEEVTQQLDTLQLCSLTKHEENEKDKCENHHEKLSVFCWTCKKCICHQCALWGGMHGGHTFKPLAEIYEQHVTKVNEEVAKLRRRLMELISLVQEVERNVEAVRNAKDERVREIRNAVEMMIARLDTQLKNKLITLMGQKTSLTQETELLESLLQEVEHQLRSCSKSELISKSSEILMMFQQVHRKPMASFVTTPVPPDFTSELVPSYDSATFVLENFSTLRQRADPVYSPPLQVSGLCWRLKVYPDGNGVVRGYYLSVFLELSAGLPETSKYEYRVEMVHQSCNDPTKNIIREFASDFEVGECWGYNRFFRLDLLANEGYLNPQNDTVILRFQVRSPTFFQKSRDQHWYITQLEAAQTSYIQQINNLKERLTIELSRTQKSRDLSPPDNHLSPQNDDALETRAKKSACSDMLLEGGPTTASVREAKEDEEDEEKIQNEDYHHELSDGDLDLDLVYEDEVNQLDGSSSSASSTATSNTEENDIDEETMSGENDVEYNNMELEEGELMEDAAAAGPAGSSHGYVGSSSRISRRTHLCSAATSSLLDIDPLILIHLLDLKDRSSIENLWGLQPRPPASLLQPTASYSRKDKDQRKQQAMWRVPSDLKMLKRLKTQMAEVRCMKTDVKNTLSEIKSSSAASGDMQTSLFSADQAALAACGTENSGRLQDLGMELLAKSSVANCYIRNSTNKKSNSPKPARSSVAGSLSLRRAVDPGENSRSKGDCQTLSEGSPGSSQSGSRHSSPRALIHGSIGDILPKTEDRQCKALDSDAVVVAVFSGLPAVEKRRKMVTLGANAKGGHLEGLQMTDLENNSETGELQPVLPEGASAAPEEDTHSSFPDGEQIGPEDLSFNTDENSGR

SEQ ID NO: 9 - DNA, 3466 bp, Homo sapiens
  CDS (420)..(3197)
gcagcttctctcgccgccgcgcccccagctgctctggttacggttggtgtttgcgaattctctgcggcaccttcttcgctgaactccaccgtcagttccataggcgcgcgcccgcgtcacgtgggccgaggttccggcgcggctgctggccggcggctgagccgggagaaaggagctgcggccaccgttcgccctacttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggagagcattgctgaggttttccgatgtttcatttgtatggagaaattgcgggatgcacgcctgtgtcctcattgctccaaactgtgttgtttcagctgtattaggcgctggctgacagagcagagagctcaatgtcctcattgccgtgctccactccagctacgagaactagtaaattgtcgttgggcagaagaagtaacacaacagcttgatactcttcaactctgcagtctcaccaaacatgaagaaaatgaaaaggacaaatgtgaaaatcaccatgaaaaacttagtgtattttgctggacttgtaagaagtgtatctgccatcagtgtgcactttggggaggaatgcatggcggacatacctttaaacctttggcagaaatttatgagcaacacgtcactaaagtgaatgaagaggtagccaaacttcgtcggcgtctcatggaactgatcagcttagttcaagaagtggaaaggaatgtagaagctgtaagaaatgcaaaagatgagcgtgttcgggaaattaggaatgcagtggagatgatgattgcacggttagacacacagctgaagaataagcttataacactgatgggtcagaagacatctctaacccaagaaacagagcttttggaatccttacttcaggaggtggagcaccagttgcggtcttgtagtaagagtgagttgatatctaagagctcagagatccttatgatgtttcagcaagttcatcggaagcccatggcatcttttgttaccactcctgttccaccagactttaccagtgaattagtgccatcttacgattcagctacttttgttttagagaatttcagcactttgcgtcagagagcagatcctgtttacagtccacctcttcaagtttcaggactttgctggaggttaaaagtttacccagatggaaatggagttgtgcgaggttactacttatctgtgtttctggagctctcagctggcttgcctgaaacttctaaatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtttcaggtacgttcaccaactttctttcaaaaatcccgggaccagcattggtacattactcagttggaagctgcacagactagttatatccaacaaataaacaaccttaaagagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatcacgagctttcagatggagatctggatctggatcttgtttatgaggatgaagtaaatcagctcgatggcagcagttcctctgctagttccacagcaacaagtaatacagaagaaaatgatattgatgaagaaactatgtctggagaaaatgatgtggaatataacaacatggaattagaagagggagaactcatggaagatgcagctgctgcaggacccgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactccacaaataagaagagtaattcgcccaagccagctcgatccagtgtagcaggtagtctatcacttcgaagagcagtggaccctggagaaaatagtcgttcaaagggagactgtcagactctgtctgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttgggggctaatgctaaaggaggtcatctggaaggactgcagatgactgatttggaaaataattctgaaactggagagttacagcctgtactacctgaaggagcttcagctgcccctgaagaagatacacattccagttttcctgatggtgaacaaataggccctgaagatctcagcttcaatacagatgaaaatagtggaagataatttgatttgaaactgacactgcacctgatgggttaacaagatctaggcttcagaaggtgacagatatgagtgaggaccatgtgtggggcaaagcctcagaatgatgaaaaggttccggcactatagttggggccatgttgactccttttcaaccatttgtcacagacgtgagaagaagaaatgacttcaaaatcaagagaaaacaaatactgaaagtctctacttacatccaaattttaaaaaataaaatctgtagattaacaatctga

SEQ ID NO: 10 - protein, 925 aa, Homo sapiens; identical to SEQ ID NO: 8

SEQ ID NO: 11 - DNA, 108679 bp, Homo sapiens
ttttctcaagttgattttattaacaaaaagtgcaaactattttgaacaaaagtaaactatgagtcacagcattcagcaagacatcagacacggaagagtgaacaatattcactaagtaaaatacagcagatgagatgtctctcacatgtatatttaattattcatgctttttcaatagtctcttagtcaactttcagtgtaatttccacaaatatatagcagctcaaacacaaatgcaggagcacaatggcaaagtttggcaactgttttgggctaattatgagtatgaaagaaaaccttatatcacagtttcacgttcatgtaagccactgtgcaacatgaatgaatctttaaatgtgttgacactgaaatcaatgtacaactaatgaaaataaagaagaaaagggggctttaaaatatttgttgcactacagtcgtatagtaagaggcagaaaaaaatgaaagaattttaaataatcttacacgtgtctacagggccaggaacgtaatgaatccatgttaacttaatttcatttaaaattacatttgtagaagtcacacaacagaaagataccatgcggttgaacagtgtgcctgtacttgaacaagtgagagaagatacatactccaaaaaggagattcagtctagtgttacttcagttattcacatagtgtctacagggcagaatctcttccaaagcaattttctgttcactaatctacaggcactaatggaactgtaattaaaaccccaaatataaagatgattatttaaacacaactaagctctagccaaagacagtagagcaccaatgccgggcgggttactgacggcatgcagggcctggaggtacattttctggcagttaaccagccttctgctgtagtagacaaactgccttttgtgaatgtacaaatttgctaaattaatagagaactacattgttatttccttacattacaaagaactcttcccatactgtttttcccatgtactactgctgtcttagactaaactgtgccaccttccaacagtttccaaattactatttcaggtcgagataatgaagaaataagaccaaatctgattatctgactgatgacaaatttgagcaccaactacagcaaaattcagggtcaaggtagcttgcaagtcagttctcttgatttggcaattaccttccactattttcatctgtattgaagctgagatcttcagggcctatttgttcaccatcaggaaaactggaatgtgtatctatgattaaaaaataaataaaaaatttaaaatttaaaagcatataacaagggcagtattttccttcccaagtctttaaataatcttaccaaatgtgttatttaagtgataaagatgtgctgggttgtgaacaaatctctgtcttcagagaatttgtgatctgagtcaggtataagcaaacacaaaggaaaaataaaggagaaaaaaacaaaatgtgtaatagacacccacaattaaaagatgacaggaattgtctcattttgtcctcctggggacaatctggggacagcttaaaaagggaacaagaagcaatggtgtatattctcaagattcaagaccttataaacataaaacatatttcaaagccaattaaactacgcattcaagtatcactacaaaaacccaccagcactccaaataacttgcacaaaatttggatgcttattttagaagcagaggaagttactaaagatgccttaatacttacaaatcatcaaatgcttggttctgtattgaaaaagataccagagcaccaggaaaatatgcaaaatataaagcagaaaatggcataggcttaacaatgttgtcactccaggatatcttgcagaggaatgttcagattgattattaaggcctctgggtaactttaaatgtgtatttctctaacttgacagtatatccttttactgattaatctgtattccagaaaactgcttgacagcagaatacttatggtatatccacatgatagaatattaggttgccataaaaaatgaagtttatgaaattttaaataacatacaaatatatttaagtgaaaaaagcagaataaaaaactatttataaaatatttaggtagggctggcatggtagctcacacctgtaaccccagtactttgggaggccaaggtgggcagatcacctgaggtcaggagttcgagaccaggctggccaacatggtgaaaccctgtctctactaaaaattacaaaaactagctggatgtggtggcacatgcctgtagtcccagctattcaggaggctgaggtgggagaattgcttgaacccaggaggcagaagttgcagtgagctgagattgtgccactgcacttcagcttgggagacagagcaagactctgtctaaaaaaaaaagaagaagaaatgcaatgcaaataagacagaagaaaacaagggaaaatattaatagtaggtttctcttgtttttttcctaataacttttttgggggtaggagggaataaaaattatttcataatctgcatgtatttctaagttatttttattttaaaaggttaaagttatcttacagtaggctagaaaaggaaaaaaaacccaaacatctgaaatctccccagggattggcaatcatttcctatgaagggtaaatatttaggcattgtaggccacaaatggactctcttgaaaattcttctttgggctgggcacggtggctcacgcctgtaataccaggactttgggaggccaaggcaggtggatcacgaggtcaagagttcgagaccagcctggccaacatggtgaaactccatctctactaaaaatacaaaaacattagccaggcgtggtggtgtatacctgtaatcccagctactcaggaggctgaggcaggagaatcgcttgaacccaggaggcggaggttgcagtgagccaagatcacgccacagcactccatcctgggtgacagaacaagactccatctcggggcaaaaaaaaaaaaaaagaaaaaaaaaacccacaaagaaaattcttctttgtttcttttgcttggtttttaaagactctttacaaagtaaaaaccattctcaggtcacagactatacaaaagcaggttgtggaccccgtctctgcccagtaagtagcactcattaggtgcaaccaagccaaatattactggctgtgtttttttagtactcaccataatagttaaccaaagaatgttgtctcacaaatgacaataattaaaaaaaaaaaaaaagaagtagaagcagaagaagcaaaagcagtagagcggaaaaagtagaagtagcagcagcagaagaagaagaaagagaagcggcagcggtggttttagtaatctgtgtaaaatgacatcatgtgctgcaccttcatccgggggctgtgtcatgaccatgaaggagtcgtgaaacccgcccacactggtatgctcttcctgctcctcattctcagtgtcacattcaatgtcactgtcgctactcattcctggcaggaaggaaggagaacatgtttctgaaaagaaaccactgtaagtaaaaggaaacagaaatctccgtatctgctaaactgagattcctattgaattttacatggaatgccaagaattcaacaaactaaacactaacaaaacaaagacaaaagtaaccgcacaaacctcgattctctttagctatttagagtggttttatttctgttctcaaagtttaaaattgccctttccatagtacctgaaagttattatttgaaaccagcattaattttattatactgataatacttttttccaaatcaaacactgtttttttttcagtcacaagagataacaggaactcttttaatcctgattcattatagacataattatattccctagcctaaaacataaatataaatgtgttatacaaatccacatggtgtatgaatattagaaattctggcaattctaataatagtttgtattctaattgggcatctaatttttttgtgtatgtcggttgatttgtatttatttttattttttgagagacagggtctcgctctgtcaccctaggctgggctgcagtggcctgatcatagctcactgcagcctcaaactcctaagctcaagggatacttccgccttagcctcccaagtagataggtccacaggtacatgacaccatgccctgctaatttaaaaattttttggagggacagggtcttgctataatatgttgcccaggctggtcttgaaatcctgggcttgagaagagatcctcccactttagcctcttacagtgctgggattacaggtcactgtgcctggccacatcagttgatttttaaaagaataattctataaaaagtatttgtttcagcaagatagcaatcaagaaaacctgaaaatctttctgctacaaatccttacaaatagcaagataaaatataaatgtaaaacattattttaatgaacaagcacattaaaaggcagaaaggaaatccacagtgtcagaaacaaagaaagataaggaactttcattagtgggtcagggaccacagttgccctggagagcttcctgtgaggatttttaaacactgccttgggatagaaaaatgaaccccccacagaaagggaattaaaagtgagactaaaactccaccccctgcataatgttaaaatttttgtttgttttcttgagacagggtctcactctgttacccaggctggagtacagtggtgcaatcatggcttaccacagccttgacctcccagggctcagatgatcctcccacctcagcctcccaacaggcctgtgccatcacacctggctaatttttgtaatttgttagtagacatgggatttcgccatgttgcccagactggtcttcaactcctgagctgaagtgattcacccgctttggcctcctaaagtggtggcattacaggcatgagccaccatgcctggccaatgctaaaattcctattgaaagaacagactagaaaaatgtctgcctactggcataggaaaacagcaaggaattttgtctgtttctactgggctctgaagagggggaaaaaagtcttacctcaaaattcaaagtaagatttatgtgcctaatttcggtttgggatttaaggttgaggttcaaattttaagaaattaacataaaaattgttcaggagctagtgttatttgaataaagcaaaagtaaaatcctctctggaaggaaggttctaggtacaatggagcaagcacattccactctatcttttccactgaaagtagcaataaaacctggagagatgggtagtgtagttatttaaggatattcatagttatttactttgaaaagtaaatagaaacaggcatactgatgaagactgaatttgaatgacagcaaaactggcagattaaaacaagacccagagtcttattaaaaaaaaaaaaaaaaacctaatgaaaatgttcaggatacaaccccaaattattcagcatatgaaaaaccaggaaagacaaccaacaaacatcaatgatgagatgacacagatgtctggaattatttaagggctttaaagctagataaaatgctcatcagccaggcctggtggcacatgcctgtagtcccagctacttgaaaggctgaggcaggaggatcgcttgagcccaggagtttgggactagcctgggcaacttagcaaagccccatctctataaaaaaaaaaaaaaaaaaaaaaaatttaaaaattagccaggcacacattggtgtgcacctgtaattgtcctagctactagggaggctgaggagggaggcccacttgagcccaggagttcaagcctgcagtgagctatgattgtaccactgtactccagcctaggtaacagaacacaagaccctgagggacagaggaaggaaggaagaattaagaatgaaggggataaagggactggtctaaattgtgtcaaatagaatgccatagagatagcaaaaataacaaaggagagacaatagataaaaggaaaaaagttgaaaattttgtagaattataaaaaacattctgaattaaatacaaataaaacatatcaaaatttgtgggatgtgactatagcagtgcttaaaaggaaaggtatgacattatttatattagaagagaaaatatctcaaatcaataatctaaactactactttaagaaactagaaaaaggaaattaaactcaaagcaagtaaaaggaaggaaataataaaaaacataataaaatcagcagaaaaacaaaaaaaccacaatgaaacaaaatggtgtttctttgaaaagatcgataaagttaaaaaagcctctaggtagattcactaacaaaaagagagcaaacaaaatttccaatatcaggaatgaaagaggacatcaactacaggttctataaacagtaaaatataataaaatgctatcagtaactgtatgctcatgaaaccaaaaacagatgaaatgagcaaattctttcctaggtacaaactaccaaaggttactcaaggaaaaatcgatgggtattttaactcttaaagaaataaaatctgctctagtttaaaaagcttccaacaaaggcaactgtggccccacatggcttcagtggtacactcttccttttctctttttaagaagagtttggttagcattatttcagccttaaatatttagaaatcgtctccatcttcagcaaaacaaaaaaacaaaaaaacaaactttcctgttctaaaagagtgaagagtttacaacaattttatgaagtaggggaaaaagaaaaagagcagagacggttaaagaggccaaaactagctagttctgtgaagcaccacctccaccttgtggcccttcatggaactgttgctgaaaacactaaaacacactgagttgaacttcaaggcacaaatttgacctcttattagtagtgtttctgaattcccgacaaataccaaccttcaggattcaaaaacaatgaattggaattctctggaattccccaaatctacattattcttttaaaaaagttattaaatacatttttcaaactctattcattacatgtgtatcttttttttttgagacggcgtctcactctgtcacccaggttggcgtgccactgcgtgatctcagcctactgcaacctccacctcccaggttcaagcaattctcctgtctcagcctcccaagtagctgggattacaggcatgtgccaccacacccagctaatttttttgtatttttagtagagatgtgtttcaccatactggccaagctggtctcgaactcctgaccttgtgatctacccgcttcggcctcccaaagtgctgggattataggtgtgagccaccgcatccagcctacatgtgtatctttaaaacaaatcatttaattagtatttgtaagagctgaggcctatagtgaatattcaaatgctagactgcatgtgggtattactgggtagtatgtgaataatgtcacgtttagaactcagcaaggtttacagtactatgcagaatcacatgttaataaaaatgccaatataaggattttgcttcaaaatctttcttggaacaccaatcaatttttattaagtataccatgtgtaaactcattattatttacttagcagcagacagggccatgaacacacataaaacggcgatatttataatgaggactgatttgtaatcacttctttccaaggagctcctgttgaccttaacctttgattccaggtatcagtatttccagtttttactttcaacttgccctaagtagattccttcaaatcagtaatgtttctgtgttgttaaaattaattctaataagacaattagaataatgttttccatttattaaacttcttgactaatttttttcagaattcaaaaaaaacacacatttcccaagaaatgctaggaagaaattcctgcatgaaaacgcctggtcttttatcaagcactaagatctggaagtttcctacctttgtgaatgtaaagaaaaagacaaatggctggacgtcaatgatagcaattacaagatactattaattgtaaggagcttcagagattgttaacgcaaaagaaatctgcatcttattactctgtcaatcatctaccatattttttgagcacctactatgtatgtgctcagtaaagctaacttgagttataactcccttaaatcaagttgctcgaatttcagatgctatggaaaatacattaaccacctggtacagatgacactttatattctagtcaccaacaatttatctgcctttctcctttcgaagaggaccctgatattgttcaggtattttcccttaccacatatccatgtggaacgagttggtgagggagaccaactaactggacttggctggctgggtgcgcggtggctcatgcctataatcccagcactttgggtggccaaggcaggcagattgcccgaggtcaggagttcgagaccagcctggctaacatggcaaaaccccgtctctactaaaaatacaaaaattagccgggcatggcagcagatgcctgtaatcccagctacttgggaggctgaggcaggagaatcgcttgaacctgggaggtggaggtaacagtgagtcaagattgcaccactgcactccagcctgggcgacagagcaacactctgtctccaaaaaataaataaataaataaataaataactggacttggccactttccttgacagtggctgacgagatagaagaggaagttgttgaggtgcttagagaaaggttgttgctggagatatatagaataaatcaatcctcttccactcccagaatgctgttctgactctgatgtctagaactgcagcaaacatcttaccgccctgagaagagctaagcctctcactcagaatagcagggcaaaagataaaacaaccccacccccaccctccaacacacacacacacacacacacacacacacacacacacacacacacactaaaaccaccccacccccacccacccacacacacacacacgttcctgatgacactgagcagctgaattttctaatcctgaaaactgctctaccacaggtgtaccttcttattgagagatttgttaatgtaaaagagttaattttcctttttgttcaagtcagttaatttttcgatgaattttgttaaaggcatcttaataattaccctaaatgatcagcaatggcaggccatcaggctttgtattcctcttaaagaggccaccagcaaggtttttaacaggcatgccaaggcttttccccagatggtgttttgagtacccctgatttaactgaagggggtatgtgctacttcagcaagaagtcagagagcaaagcctttagttttacaaagaacaagaaataaactggcctacctacagttgtgcaggtcatcaattgaaagaaaaaataagtcaaccttttcagcatcagatcattaaaccagcgactaatacaaccaaaggttgtcccatatggttccagtaaaaaggaaaaggcctatttatgttcatatctaagaaaaaagttatttttaaactgatgcttaagagcaattaagagacactttagtagtcccaaattcccagccagtaccacaaacaacgccttaaagtcctccagacaaccttctctttgagaaagagagagaaccccacagtgatatgacaccgaatgtcttctttttgtattcagctaaaataaagagtcttgtttttcctaagcatggatacaagacaaatccattcaactgtctactaaggcttataggtatataagggtttcctgagaacttgctcttgctgatgatagaataatactggcaacagtgagcaaggccgcactctcctcgagaaacaaagctgattacaggaggcacagggcttcctttgtctcctgtactaccttctctcagcctcatcagttagtctccttctcacagtctatcctgggactctggaggtaaacaagctgttcagataccaacaaattacaagggtgtacttctgtgtccctgttgatttgcacattcacagcaatttttcttattgagttctaggtgctatttatagcacaacaccccacatataaagataaaatgtgttactgaataacactgaccatttaactctaaatcctacatgaataactccattctttcaaagacttggaaaatgtaccgatcttcactatggccaagcaaatgcctttcttcagtttaggacaagctgtcaattagctttaaataagaaatgaaactggtgtcaaaaagttccagtgttttctgtgctgaccagccattttatatataaaaattagtgctgagataatgtaaattgataaattggaggctgaaattaagtccaaatcgggcggccgggtgtggtggctcatgtctgtaacccagcactttgggaagctgaggcagaaggatcatttgagcccaggagtttaagaccagtctgagcatacagcaagaccaagtctctataaaaaattaaaaataaagaagtccaaatcatttcacctaaccatgcttcttaccaattttcctgcctctagttccacatatgctattcccacccccaaatccattctccagaatgcagctagagtagaaatgttaaagtgtcacttctaacttagatcctattcaaatgacaaagttcaaattctagctgggacattccacgtaactcttcatgatctggcctctggtctgtctcccttctggcctttcaccagtcttcatctggctaactcctactcatcccttaggatgatctttccaagggtggattagggactaggttagcagagagaggcgacagttaagagcactggctctaggttcaaattctagctcttccaagttcttttttttcttttcttcttttttttgagacagagtctcgctctgtcacccaggctggagtgcaatggccacaatctcagctcattgcaacctctgcctccttggttcaagtgattctcctgcctcagcctctcgagtagctgggattataggagtgtgccaccatgcctggctgatttttctatttttagtagagatggggtttcaccatgttggccaggctggtctcaaactcctgacctcaggtgatctgcccacctcggccttcccaaagtgctgggattacaggcattagccaacatgcccaaccttccaatttcttttctttttttttttttttttttgagacgaagtttcactcttgttgcccaggctggagcgcaatggtgcgatctcagctcactgtaacctccgcctcccgggttcaagcgattctcctgcctcagcctcccaagtagatgggattacaggtgcccgccaccacactcagctaatttttgtatatttagtaaagatggggtttcaccatgttgaccaggctggtcttgaacttctgaactcaggtgatccaccctccttggcctcccaaagtgttgggactacaggcatgagccactgcgcccggccctggccttccaatttctaaccctgtgactttgggtaaggtaaattacatctccaggcctcagcttactcacctgtaaaactaggttaacaaaagcaactaccatgtagagttattctgagaataacagaatactggtaaagtctttaacacagtaagaacttagtattactattatttttattacgtatctcatttttgagctgcctataaaccttatcacagcactcatcgtcctatattacaactggctattagtctcccctgacacctaacaattggttttcaactgctctccttgccgccctcctaccaataccaccacacatacctagcatacctgagagctacaacttatttctattaataactgtggcccactctggcaagtgattctcaaggagtggtcacctctggttgagaattatttagttcaactattggttcttgactggggatgattttgcctcccaggaaacatgtggcaatgactagagacatttttggttgttgtaaccggagggaaacactactggtatctagtggttaacagctgaggatgctgctaaacattccacagtgcagagaacagtccctgtaaaataaagaattatgtggcctaaatggcaatggtgcaaaggtggagaaagtctgaactagatagtaagttcaggaattttatcactggcatcaaatgcaacatccggtaaattagaagcatttcataaatgttgcatgaaaaaaatgagcttgttattactgtcatttgagatagagtctcactctgtcgtccaggctggagtgcagtggcgtgatctcagctcactgcaacctctgcctcccagttcaagcaattctcctgcctcagcctcccaagtagctgggattacaggtgtctgctaccacatctggctaatatttctatatttagtagagatgaggtgtcactatgtttgccaggctggtctcgaactcctgacttcaagtgatccgcctgcctcagcctcccaaagtactgggattacaggcatgagccactgcgcttggtggaagaaaaatgagcttgtaattccagcatattctgtagtaatttaagctactacaatccagtatgtatggagacacatagaaagtaactattccccggccggccttggtggctcacgcctgtaatcctagcactttgggaggctgaggcgagtggttcacgaggtcaggagttcaagaccagcctggccaacacagtgaaatcccgtctcttactaaaaatacaaaaattagccaggtgtgttggcaagcgcctgtaatcccagctatgcagaggctgaggcaggagaatcgcttgaacccgggagttggaggctgcagtgagccaagactgtgccactgcactccagactgggcaacagagctagactccgtctcggggaaaaaaaaaaaacaacagaaaaagaaagtattccccacaggcccattctataaagatcttcaatcataatatttctttcttgatgttttaacaataaacacatcatttacaaaaatcttttctatttaatctcttctactgaaataatgctgcccatcacagaagcaccaattaagaaaaccaaaatatttagtgttctttaactgagaaacctgaaaatgccaccacctacacaccctcttcttagggcagctgagtagatgctggcacctgtatgtatgctttgagctacaattcatcaatcattgaaattcaaatgagtgcagaggtggaaggtggtggagtttcactaagaaatgaaaaaattacaaggccaaatatcattaagagagttgacattcttttctaccaatttataaaggacacagcaagggcaatacacatagccttgttctaagcacctctgttgccccaaattgcaaaatgtcttctatgcttttagataaacaataatgaacttggaagaacatgcaaaagaaacactagaaagtacagctgagaacaagctgaagtcagtcatcatacaggatttaaaaaatggtcaaaacttggaatgcttctttaagaagaataaagcaatctacagacactaattaagactgactttaaaaaatacttaacatttactattctaattagttgttctagggcatatcttccccattagattgtgaacatatctaattcaactttgtattaataaatctagcaaaaattcgatgcctggtatgtaggtgccttaacatatttattgaccgaacagatgaactatatgcagattttttccccatacaaatgcctgttctagtaaggcctgtccttttatatagtaacttacccattttgccttcctctgtatttggtatcttagcaaatcaaattcagtggcacaaggaatgttgtgttcagatcaagcatgtcgaatagcagatctcagtaacatttccattaaaccaaccactccctatccctatcactaccaccagcagcagcagcaccaccaccaccaccaccaccaccaccacccaccacccaccaccaaaaaagggacagaattctcatttcctgcttacttataagtttatcattccttaccttcttcaggggcagctgaagctccttcaggtagtacaggctgtaactctccagtttcagaattattttccaaatcagtcatctgcagtccttccagatgacctcctttagcattagccctcaaagaagaaaacaacttgatatttctctataactagtgacacaataatctatattttctttttgaactgagcaccaaactaagtagggtacgtgctctggtgcttaatactaaatttcaaattttatctgaaaatatttcactaaggtcctggcacagtggcttatgcctgtaatcccagcactttgggaggccaaggtgggtggatcacctgaggtaggagttcaagaccagcctggccaagatggtgaaaccccatctccactaaaaatacaaaaattagccagatatggtggtgggcgcctgtaatcccagctactcgggaggctgaggcagagaatttcttgaacccgggacgtggaggttgcagtgagccgaggttgcaccattgcactccagcctgggcaacagagtgagactctgtctcaaaaaaaaaaaaaatttttttttcactaaatattcacaaaaaaatgttatgaaatggtggattacatcaatgttggtatcctggttgtgatattgtactacagttttgcaaaatgttatcattaggggagaaaaatacacaggatctctctgcattatttcttacaactgaatgtgaacctataattagctcaataaaattttaacttataaataccaatagaagttttaaataatatgttctaatagggaaagacttttgagatatattattattgctattatttgagatagagtctcactctgtcacccaggctagagtgcagtgatgtgatctcagctcactgcaaccgctgcctcctgggctcaagcgattctcctgcctcagcctcccaagtagctgggattacaggtgtccagcaccacgcccggcaatttttgtactttgtttttagtagagacagtgtttcaccatgatggccaggctggctcgaactcctgatctcaagtgatctgcccacctcagcttcccaaagtgctggggttacaggtgtgagccaccgtgcctggcctgagagatattattgaatgagaaaataaagtacatcttctgtattaaaaaagctacatagtcacagttccttaaatatacacaaaaactgagaagacacagcacacatgaaaaattaataacagcatgaatatgtgtgggcagggggttggaaaactaggtcatgggagttaaggatgggaaatttttcacttattcttttctcctttcattttgaaacacttatcattagtatttaatatttatttttatttaaaaaataaacagaggcaaggtcttgctatgttgcctagggtgttctcaaactcctgggctcaggccatcttcccacctcagcctccaaaagtgctgggattacaggtgtgtgccactgtgcccagcctttcattagtatttaaactttttgcttacactactttaaatcaggcaggaaaaaaaaagacatgagctggacagcacctacatgaaccaggctggcagataaaatcaactgcaaaatgaggatcaagctgcacacacttgcacaggattttacactctagaaaaaaaaaaactaagttcatgtatatttaattgtggtgactaaaggcagataaaaatatctccatagcaaataaaatataagagctagttgcactttaatttgaagaaatgtataaaacatcaggttaggtagtacgtagggaagacaagataatctccagtgattttaacacctttctactactgaaacttgagccagaaagctaaaccaccagctgatcttgtagttttggtaatgagccagaccttgactatataaaattaagtaaacttaaagggtgttaatctttctccaatttaggtaaaattcttttcagggtctccaaaatagaactatttcattgtttaaatttagaaataggtttaatatccataaaatagataagcagatggatattaatggctaatcatctgccacatccatagccaatatccataatcactctatcttttgaatagatggaagtttaattgccatgccggggatttgagtttatgtctcctaggggagagataaacgtgttaactactatatatctttttgggaaattccaactataagttttgaggatactttgataacaggagagaagaaagatgagataaaagcagaaaagcagtgttaaagttgctgtaagtgctttgtttcatacaaaatatatataaatgcctgtgacacataatatagcccaacactaacatctttgacaagtgtaggatagactgaaaaaaaaaaaaaaggctgaccgcccccctaccgccaacacatacaaatttacaaggcagagttcagataaggttacgtatcattgtgaaaatgagctctaacatatttaaactaaacttccggtactatttcatcccattaaaaaaagtactatctcttgagaccatggtgaaaccccatctctactaaaaataccaaaaaaaaaaaaaaatcagctgggcgtggtggtgggcgcctgtagtcctggctacttggagaggctgaggtaggagaatggcatgaacctgggaggcggagcgtgcagtgagctgagatcatgccactgcactccagcctgggcaacagagcgagactccatctcaaaaaaaaaaaaagtactatctcatcccattaaaaaaaaaaaagtaaaatctctgatactttaaaaaagttaaaatttaggccaggtgcagtggctcacgcctgtaatcccaacactctgggaggctgaggtgggcgtatcacttgaggtctcaggagttcaagaccagcctggccaacatggcaaaacctcgtctctactaaaaatacaaaaattagcagggtgtggtggcgggcacctgtaatcccagctacttgggaggctgaggcaggagaatcacttaaaccggggaggcagaggttgcagagagctgagatcacgccactgcactccagcctgggtgacagagcgaggctctgttaaaaaaaataaaaaaaattaaaaaagttaaaatttagtgcaataaatgtattttctgtccactaaaaaattaacaaggtagaaattaatttgttcatcatgatgcatacttaaagttccaaacaaagctattagctattataccattacatatacaaaacaacttaatttaccccaaggtgaccattttcctccttttctcaaccgcaggcaagccactgaaaactgcaaccacaacagcatctgaatccaaagctttacactgccggtcttcagtttttggcagaatatcaccgatactgccatgtatcaaggctcggggagaactgtgcctgctcccagactgagagcttcctggggagccttcaaaaaaaggaagatggaatacaaaaattagctgggcatggtggtgtgcgcctgtaatcccagctacttgggaggttgaaacacaaggatcacttgaacctgggaggcggaggttgcagtgagctgagattgtgctacactccagcctggacaacagagcaagactctgtctccaaaaaataaaataaaataaaataaaaggagggataatcagaaaaaaggcaaataaacatgaaaggtctctctcagttacattagaaaatctcactatatatatcattttaaaactacataaaaacttcaaaaataggccaggtgtggtggctcatgcctgtaatcctggcactctgggaggctaaggcggatggatcacctgaggtcaggagttcaagaccagcctggccaatgtggtgaaaccccatctctactacaaatacaaaaattagccaggcgtggtggcgtgcacttgtagtcccagttactcaggagactgaggcaggagaatcacttgaacacggcgggcggaggttgcggtgagccgagatcgcaccattgcactccagcctgggcaactagaggaaaaaactccatctcaaaaaaaaaaaaaaaaaaacttcaaaaataattttgaaacatatcccttgttttttatgccaagcatctaaaattaaggagtccaggccaagtgttgtggctcacacctgtaattttacactttgggaggctgagacaggaagattgtttgagcccaaaagttcaagaccagcctggacaatgtagcaaaaccctgtctcggcaaaaaaaaaaaaaaaaaaaaaaaaaaaaaaagccaggtatggctgcatgtgtctgtgtctgtggtcccagctacttgggaggctgaggtggaaggatcacttttgtctgggaggtcaaggctacagtgagctatgattgcaccactgcatccctagcctggaaaacagagcaagactctgtctcaggaaaacaacaaaaataaagaataaataaaattaaggtgtttagaatacatagtctaatggctgtattttgttaagggtctgttctgttgagatcaagaaactttccagccaggtgtggtggggctcattcctgtaatccgagcactttgggaggttgaggcaggcggattgcttgagctcaggagtttaagaccagcctgggcaacctgtctctacaaaaaatacaaaaattagctaggcgtagtggtgcacacctgtagtcccagctacttggtggggctgaggcaggaggatctcttaaaccaccggtcgaggccacggtgagccaagatcgcaccactgcactccagcctgggtgacaaagtgagaccctgtttcaaaaaaagaaactttcaaatccaactttagcatttttggtgcccttcaagatctaaagctccacgataacatcaatttcaggtaataaatatttaccccactaaaagtacattctgaatccctcaaatttaccttcagacagagtctgacagtctccctttgaacgactattttctccagggtccactgctcttcgaagtgatagactacctgctacactggatcgagctggcttgggcgaattactcttcttatttgtggctgcaaagacatttaagaacacctctgaataggctactacagcacaaaactcactatttagtctgtcagaaaaagttttaatcttaccttgcatggaagctgttctctctactgtctaaaaataaacaacattctgtagccccaattggtttagacttttgtcataaaacattcccatcttgttgcttgtagagcagaatcttttttttttttcccccagatggagtcttgctcttgttgcccaggctggagtgcaatggagcgatcttggctcactacaacctccacctcctgggttcaagcaattctcctgcctcagcctcccgagtagctcagattacaggcactcgccaccatgcccagctaatttttgtatttttagtagagacggggttttgtcatgttggccaggctgggtctcgaacacctgaactcaggtgatccacctgccgcggcctcccaaaatgctgggattacaggcatgggccaccgcacaccgcccgtaaaatcttaacttaaaagattatctgtgagctccttaagaaaaatcatacaaatttcaatgcagttaaactattaacaaatatataacccgtcaaacatttggaaattataaagtattattggattaaaaagaaaaatcaatgctgagttagaaacaaattacaattatggtacttgaaatcaaaatgtctgtggccaaaccagtagtgaaaagaaaatgtacggccttaaaatgaattaattagggaaaaaagggactatatgacctaatatgcatagattcaaaagctaggaagacaataaaaataagcaagcaagaatgctattgccagaaggcagggccctgcccttgaattcagcctgcagaactgtgagttaaataaatctcttttaaaaatacattttaaaaaaagaataaaaataaaattgtaaaaaacaataaaaataaaaaattcattaataggtaataaacaggtttagcatggttgcaggatataagacaaaataacttattgtatttctctgcactagcagacaatgtgagaatgaagttaagaaaacaatttcatttttaatagcataaaatacaattaaaatacttaggaatttatttaacaaaagaaatgtgagacttgtccactgaaaactaccacacattcttgaaagaaaatagagacaacagcaaagacatcctgtgttcatcactgattagaatacttaatactgttaacaaagcaatattcttaaaattgatctaccgattcaacacaatttctaaaataatagtagctgtgtttttttttttttgcagaaattgacaagctaatcctaaaatccatttgaaaatgtaagagacccacaatacccaaaacaatcttaaatggaacactcacttctgatttcaaaagttactataactaagtaatcaagactatgtggtactgccatatacacatatatatcatcaaaacagaattgagagtctagaaacaaacccttatatttatggtccactgatttttgacaaggatgccaagacaatgcaatgggcaaggaaaaaaagagggacaataagtgttgccaacggtatggaaaacctggagccctcatatactgctgctgggaatataaaatgatgcagctactttggaaaatagtctggcactggtagttcctcaaaaagttaaacacagttgccatgtaacccattaattccacttctaagcatatactcaaacaaaaccatatgtccaaacaaaaacttgtacatgaacgttcatagcagcactactgataatagccaaaaagtaaaaacaacccaaatatccatcaactaatgagtggataaacagtatgtagtacagccatacaataaaatatcatgcagacataaaaaggaatatatgccacaacatgaatgaaccttgaaagggctgtgttaagtgaaataagccagacatagaagacctcatatggtatgagtctatataaaaagtccaggctgggcgcagtggctcacatctgtaatcccagcactttgggaggctgaggtgggtggcgcatgaggtcaagagattgagaccaacctggccaacatggtgaaacctcgtctctactaaaaatacaaaagttagctgggcatggtggcgcacacctgtagtcccagctacttgggaggctgaggcaggagaatggcgtgaacccgggaagcggagcttgcagtgagccgagattgtgccactacgctccagcctgggtaacagggagagactccgtctcaaaaaaaaaaaagtccagaagaagaaaaatccatagggaaaaagtagattagtggttaccagaagatagtagaaggggaagatggggaacgacttttaatgggtttggagttctagggagagggttaatgaaaatgttcttagacttagataaatgttctagacttacgaaaaatgatgaccaatttctccactaaaaggataaatgaattattggttaaggaaaaaagaatgcattaacagtctgggtgcggtggctcatgccagtaatcccagcacttcaggaggccaaagtgggtggattacctgaggtcaggagttcgagaccagcctggccagcatggtgaaaccccatctctactaaaaatacaaaaattagcccggtgtggtggcacacacctgtaatcccagctactcaggaggctgaggtaggagaagtgcttgagcccaagagacggaggttgcagtgagccaagatcacgccactgcactccagcctggtggacagagcgagactctgtctcaaaaaaaaaaaaaaaaaaaaaaaaaaaaaagcattaacaaagacaaaagcagaaatataatgaaatggtaaaagacaatggatgaaataaatctaagaacttggtctttgaggagatgaacaaaattagcaaatcccttgagcaggtcaaatcaaaggagaaaaaaaaacagaagtaagcaataaaataagtgaaaaagaagttaacaacccatatgcacaagaagttttaaaaatttaagaaaatagaataaatgactgtatatcaataaatttaaagacccaaatataattagtttttcttggaaaactatagtaactgacttaagtagcagcagaagtatgagaaaactacatctgacaagagattaatgaccaggatctcaaacaactctataggaaaaaattcaataatctgattaaaaatgggtaaaaatctgaatagatatttcttaaaagacgacacacaaatagcaaacaggcatatgaaaaagtgctcaacatcactgatcatcagagaaatgcaaatcaaaactacaataagataccatctcatcccagttaaaatggcttttatccaaaacacaggcaataacaaatgctggtgaagatgtagagaaaaggcaactctcatacaccattggtgggaatgtaagttagtaaaaccactacggaaaacagtttggaggttcctcagaaaaactaaaaatagatctaccatatgatccagcaatcccactgctggttatatacccaaaagaaagcaaattagtatgtcaaagaggtatctgcattcccatgtttactacagcattattcacaataaccatggtttggaagcaacctaagatccacagacaaatggataaagaaaatgttaggttgggcgtggtagctcacgcccgtaatctcagcactttgtaagcccgactcaagtggatcatttgaggtcaggagtttgagaccagcctgaccaacaaggtgaaaccccatctctactaaaaaatacaaaaaaattagctgggcgtggtggcgcatgcctgtagtcgcagctagctgggaggccgaggcaggagaatgacttgaatccaggaggcagaggttgcagtgagccgagattgcaccactgcactccagcctgggcaacagagtgagactctatctcaaaaaacaaaaaaaaaatgtggtacatatacacatggaatactattcagccataaaaaaggaatgagatcctgtcatttgcaacaacatgggtggaactggagatcgttatgttaagtgaaatacatcaggcacagaaagacaaacttcacacattctcacttatttgtgggggctaaaaattaaaacaactgaactcaagataatacagcaatggttaccagaggctagggagtgtagtgggtggctggggggatggttaatgggtacaaaaatatagttagatagaatgaataagatctagtattcgttagtacaacagtgactacagtctacaataatttattgtacttctgaaaataactaaaagagtacaattggattgttttaacacaaaggataaatgcttgagatgatggatactccatttaccctgatgtgattattatgcattgtaagcgtgtatcaaaatatctcatatatcctataaatatatatacctacaatgtacccacaaaaattaaaaattaaaaaaattaaaaatatgaggaaaccaacaaatccaacagaaattaaaaaggctgttaaagttctaactctgagttactatactagttttatgaatgctttttattagacataaagtttaatacagcataggagcaaataacatatagttaaaataattttatagcatagaaaaggacaaaaacatctgaatttaatctgtaaaacaagcacagaattagaactaaagtgggttaaggatagtgtaatgaaatagatcgatttcacttatgactatgtagtcaaggatcctaagtaaaatattagcaaactgaatacagcagtgtttttttaaaaaaagtataggcccaaactaaataggatgcattccagtatttccagaatggtcaacattagaaaaataatataatttactttatgataacaaagtaatggaacaaaatagtaaatcattttgacaaatgtcaaaacatcatttgatataattctacacagcttcagtttaaaaagaaaagaaattctcgtagtaagaaaagaggcagtttggtattagggtgaagggcataggctctgaagttcaagtttttggctttgccaattaccaatgtgaaactagcaacattattcaatgcctgctgccttggtttcctcatctgtaaatctggaatgaaattgacttaccttacctcttagacttcttgttaagattaagtgagtcaatgcatgtgacttagaatagttagaataatgtctggcacacagttaacacttagtaaatgttagccattattggtattgttgttactaaacaggtttagtaacctttagttactaaatttctttagtaagaaatacagatgtacaataaaaatatgaaaagatattcggcctcacctgctattcaaggaaatggtatattaaaccaaatggtatattaaaaccaaaagtacagctttttgggtgaactttctcaaaaagcctttgttacatattttgcagatacagttgacgcttgaacagcacaggcttgttgtactgtacaggtccacttaatattcgaatttttttcaaccagagaaagattgaatatattactatactatactatagaatctattactataaatagaaggatgtaccttttaacttttttatttatttatttatttatttttgagacagtctcacttgtcacctaggctggagtgtggtggcgcaatcttggctcactgcaacctccacctcctgggttcaagcgattctagtggcttggcctcctgagtagctgggattacaggcatatgccatcatgcccaggtaatttttttgtatttttagtagacatggggtttctccatgttggccaggctggtctcgaactcctggcctcaagtaatctgcacgacttggcctcccaaagtgctgggattacaggcgtgagccattgcacacagcttttttaacttgatagaaacaggcctgtaatctttgggaggccaagacaggcagatcacgaggtcaggagatcaagaccatcctggctaacatggtgaaaccccgtctctactaaaaaatacaaaaaaattagccaggcgtggtggcaggcacctgtagtcccagctactcgggaggctgaggctggagaatagcatgaacccaggaggcggagcttgcagtaagccgagatagagctactgtaccctagcctgggtgacagagcaagactctgtctcaaaaaaaagaaacacaaaaacaaaaaagtacctattgaaacatacagcaaacctcaaatttaatgagaaaaaaactaaaggaattcctaaataaagttgaaaaattataaaaatcctaccactgttactaagaaactgttgaggtacctgcctagacaatgcaatacaaaaagtaagcagaaaatgaataaacaaattatgattatctgctgatgatataaatacttagaaaatcaaaataactaattgaaaaactgttaataaaacaaattcaccaggccaggtgtggtggctcacgcttgtaatcccagcactttgggaggccgaggtgggtggatcacctgaggtcaggagttcaagaccagcctgacgaatatgatgaaaccccgtctctactaaaaatacaaaaactagccgggcgtggtggcatgtgcctctaatcccagctacttgggaggctgacaaagaagaatcacttgaacccaggagggggaggttgcagtgagccgagatcgccccatcgcactccaggctgggcaacaagagtgaaattccgtatcaaaaaaaaaaaaaaaaaaaaaaaaattgccacattggttgaatacagaataaacataaaaatcaatatttttgtcttttatgtcagtaacaattagaaaatactatcagagaaaaagagattccactcacaacagctacctaagatataatacattgagaaatgaatttaacaagacatatatagaacttatatggaaaaaactgagagttctaaaaaaggatatgaatataacagatgacctgaataaatggagagaagtaccacattcctagacatgaatgtttgatgctgaaaagatctcaattgcccaaattagtctctctttttttaagagagagagagagactgttgcccagattggagtgcagtggcacgatcacggctcactgcacctcaaattcctgacctcaagtaatcctcccacctcagcctcccaagtagctggggttacaggtacataccaccatgactggcttatcaaaaaaatttatttttgtagagattaaggtcttgccatatgatgttgaactcctgggctcaagaagccatcctccctcatcagcatcccaaaatgctgggattaacaggcaggagccatggtgcccagcacccctaaactagtctttaaatgtaaagcaattccagtcaaaatactttggaggaggattaaaagtgcaagaaccaccaataacattttgaaaaccaagaacagaataaacatatcctatcatagtattacaaactataaaactacagtagttaaaaaaagtgttagtctggtagaggacatgacaaatactgggttaaacaataccaaattgcgtttttgtatgtccaaaacttgagtactggcaatttcagttgtttcgacctctatgagttcaaaaacagaaccatgtttgggaatctggtctataataaaggatggatttcatattgcatcatatttcaaaaaacatgtttcattcatttggggaaaaacatcattccatttacctcacacagcatcatataaaattaactttatatgaattaatgacctatatgtaaaaaagttagaaaacaaaaagtaccagaagaaattaagggttatttttattaacctgggagttaaaaagccttccaaataaaaataagacccatgaatattattgccatgaatattattattaattataatattattaatattcatgtcatgaatgttaattatattattaatattcatggatgaatattattaatacagttgtctaagataaaattttaaaactctgtgattagcaggaaaaaaaggtggcaatggggaagagacagttgtcccttggtatatgtgggggttggttccaggacccctgcgtatactaaaatcctcgactgaagtcccaccgtaagacctgcagaacccacctatacgaaagttggccctcctatatgcaggtttggcatcacccgaatactgtattttcaatctttctctggttgaaaaaaattcgaatattaagtggacctgtacagtacaacaagcctgtgctgttcaagggtcaactgtatctgcaaaatatgtaacaaaggctttttgagaaagtccacccaaaaagctgtacttttggttttaatataccatttggtttaatataccatttccttgaatagcaggtgaggccgaatatcttttcatatttttattgtacatctgtatttcttatgttaatggcctattcataacctttattcattctactaaattgtcttccttatattaagagaattctatatattttgctattatctcctcaccaacccatctttattggattattcacatcactatcaaacattaatacagccaattttaataaacaacacacacaaagagaacccagaaacaaaccctcgtctatatggtcaaatgatttcaacaatggtgctaaaaccattcaatgaggaaagaaaagtcttttcaacaaatagtgctgggaaaactggatatccatgggcaagcaagtcagacccttccctgacaccataaacaaaatttaattcttaatggatgaaagactaaaagtaagactaaaagcaagaactaaaactcttggaagaaaacaggagaaagcttcatgacactggattaggcaatgttttcttggctatgacaccaaaagcacagggaacaaaaggaaaaacacataaaatgggcttcttcaaattaaaaacttcaaatgttaaatgacacaatccacagagggaaaaggcaacccacacactgggagaaaacatctgcaaatcatatatgtgataaaagataaatatttaaaatatatacagaactcctaaaagtcaactacaaataaacaacccaacccaattttaaaatggatttaattcacagatgaaagacattggagaggacacaaaaaaggacacccctgccaacaagaaactattgaccccgcacctcctccagcttctacccacttcattgtgaaacttcttttaaagaagttgtgcatttgtgctctggtccctgacctcattcactcttctgctacactactccatgggaatagttcttaaaaaggccacttggcctcttgccttgacacttctcagtgatacttaacacacttaacacaggggtaaccactctacttcttgagatgcattatttttgatttttgagatattatcccttctagtttttctcctaccttaatgattgctctttcaatttcctttgctggcctctcctatttatcagatctttaaatgtttaagtgccacagagtcaaaaattttgcttctccattgaaactccctccaaacttgaccctattcagtcttctggatttaaataatataaaatactattgactcccaactgtttatatccagcattgacctctctctgagtttcaaagttgtgtaccattcacgtgtctatgacattttactaggatatctagcaggtgtctcaaattaaacatgtttaaaaaagaatacccaactttctgcccttacctcctcccaagccccataacctgcttcttatctgatttgcttctcaataaatgataccactgcctattaagttgcttaggccaaaacttaatcataattcttagtttctttctaaatagaactttcctaaaattctatttaggctttttcaactatggttctgagaaagaactcagccctgctgatctaagactggattaacttccctcctatgcatctagaatagaatagcacatccttaagagaactaagaaagatgggaacctcagtcaagaaaggctgctagactgtgaacaagcccctctatccattattgtaactccagaaaatgccatcatatttcacacaagataatgtaataggctgttactggtctcacaattcctgtcctcaacccttttcagacccttctccacacaacaagcaaagtcatctttgtaaagtgtaaatcagattataaacaccccttatttttattctctagaggcttcctccttacatcataataaaactcgaactcctttttgaggttttgcaagactctccctgatctaaattctgcctacctctctctgacttcgtctcctgctagtctcctctctctgctttctttgccatactggtcttccttcaatccttcaaatacgcaaaataaatcctgtgtcagtgactttgcagtttctgttctgtttacccagaacactgcttgccctagatctataaatagctgccaccttcctaaacttcaggtctcaggtcaagtgctgcctcctcacagagactttctctgactaccccaatagaacatgtctcatctgctcctcccatcccactcctcggttgtttccttcacagcaccttttacccactgaaatgatctttacttacttgtctacactttttaatgtctgattcccactactagaatacaagatctatgatgttaagaattttgtcttgtttattgccaaatacagagtaagcaatcaagaaatgttagttctattatatagtaaaaatatacggaatcaattttggatgaagaaaaaagtcacatgtaatttgtagtcttaactcactggaagagtggtatttaaatattattcttttgaaaaaaccagtcatctagtttcccaaataacgtgtggagaaatgacactgggaacatattacttacagtttcgtatgtaacaattggcaactgatgactttgccaggagttccattcccaaatcctgcaatctgccagagttttcagttccacatgcagccagagctgcctggtcagcagaaaaaaggcttgtctgcatgtctccagaagcagcactgctgctttttatttctgaaagtgtattctttacatcagttttcatacatcgaacttcggccatttgagttttgagtctttttagcatctttaaatcagagggcactcgccacattgcctgttgcttcctttggtctttatcttttcgagaatatgatgcttcagagaaattgacaagtcatgttaacataaacgttaatattaatgaaatcatttgtaccatgaatatatgcaaatttgatgtgtaaaataagctagcgtgctttacgtggtatcaacaataaaacatgcactaaattccattaatgaaaataaagataaagaatcctaccttattcacaccttcagagttagaatctgagtcacatcatactcaagttcgaattacacatttaattctaaaagtttaaaagctgccctactgccttgtgctttcttctttaaacttctccacttttctgaaactttctacagagcttttattatgtttataattgaaaaaaaagacaaagaaaaatctagaaaccaaatactttgtcaaatctgccatatataacattatattaaaaatacatatttctggccaggtgcagtggcttatgcctgtaatcccagcacttcaggaggctgaggtgagagaatcgcttgaggccagaagttcaagatcagccaggggagcatagtgacaccctatctgtacaaaaaaaaatttttttattagaacttccatttctgataattttctcagaactgtcagctttctgaaatacggtttgaatctaaccacttctaaaataaaacaatagctctccatgcctagtatgaataaattcctccggctagcatgcaagtcctttcatagaatggacccaacatgactctagttctacttaccactatatgtaccttaaaatctggttcaactagattacctgataccttttacagtactcctttacatggttaagaacatgaagttaatctgacaaatgttcactaaatatgtgttaaataaatacacagaaaaaaataaacatcaaagcccaagcaggccacgtgcagtggctcacgcctataatcccagcacactttgggaggccgaggcaggtggatcacttgagctcaggagttttgagacaaacctggccaacatggtgaaacctgtctctacaaaaaattgtagagcactccagcctgggcaacagagtgagactctgtctcaaaaacaaacaaacaatcaatcaaacaaaaaatgacccaagcaagccttttcaataaaaagaaagtcatatatttctgcacactagcttttattaacatcccttcactgaaaggtattttcatttggcttccatgagtattttttctcctttctaaaagctctttatctggctcatcttcctgacactcagtaaaaacagatattcatcaggattatgttcctggctctcttcttttaaaatattcttaaaactcctttgcaatctcacacccttggttttaattatccagtatcacctccagaaaaataactgtctgtattccagttcatttttttttttcatggagtttcactcgttgcccaggctggagtgcaatggcacgatctcggctcactgcaacctccgactcccaggtttaagccattctcctgcctcagcttcccaggtagctgggattacaggcacctgccacaatggccggctaattttttgtatttttagtagagacagggtttcaccatgttggctaggctggtctcgaactcctgacctcaggtgatccacccgcctcagcctccccaagtgttgagattacaggcgtgagccactgcgcccagccccagttcttaatttttagctgtctactggtcctttctaatttgatgtcacactgttatgcccaactcaagatgtttaaatccaaaacttgctttttctttctactattttccccgccctcattattgccagcattttatcacctaaatttgaaattcagatcatctcatttgtattgtttttacacagaaaactagttttacacagaaatcttactatttagtttgcatttttcccaatgatactgattattttcaaaaacacgattttacttgtctataaaatataccattgcatggttacactgtaatttattcagctataactgtattgttgaatatttttaaacatctgcttgttttctctttggaatatactcctcaaagtgaaattacaagtttaaaggatatagatatttttaaactactacaaaattgtccttcaaaaaggttaaaccaacttacatatctacctacagaatcagaacctgtctgtttcactgggcctttgcaaacaacacttaacatttaagtaaaactttatataaagcatacttggcgcacacataaaaggcaactgtttcataacctgaagatagtcttttaagctgaggatccagactttcaggaagtatatctggctcttaggaaagaagaagcctaaataatctgggctccatgtccctacaccagtagaagagctctatttctctgttatttacacagaggttctgccttggactttgattgaaaaatgagttatcttgccttaaaactacatcaaaatcagagttctaaatcattgatttgatgtatataatactattgtttaagaaggtagaaaaatatttggcatatatttttgaatatactgactatactttagaaagcattccaacaaaaatggtttttaaaatgtagcttttaaaggcaaatacttagttgtccaaaatgactctatccccgagagtttaaaaaagcccatatttgactatacaaatctccattttattatagtaacacaaatgtaatttctatttaacaaataacctttgcacagaaaacagtgctgccaactggcatacatttttaagctactgaacatcaaagaatgagtaagtcacctttttctacgttcaatttagatatagttccacttggaagtaagggaattcatgaagcctgaccaaaaaaagtccattcataaagaagataaccgtatttcatatggatcaaactgtcaggcaaaatctgagtagcaagtcacaaaaaaccttcttgcaggcatttttactttgtttttgcacaaatcacctttgaacggtaatttaaaaactaatttggttgacataaatgtcatttttatggtatacaatgaaaaacacagaaatacaccatgttccacacatagctgaaatacttaaatgaaaatccaagagttcttttagagaaccacagaaaaaaaggaaattatcattgttattattttacctgtgggctgcagaagtgaagcaggtgggcgaggctgtaagccccacaaattttctatactgctccggtccttaaggtccaacaaatgtattaaaattaatggatcaatgtctagtaaactactggtagcagcggagcataaatgtgttcttcttgatattctactactggaacccacataaccatggctactacctgcaaaagaggcgtagaaaatgatatatatatgcacaaacttagctatacttaaatgaaaaaatgaactggttaacattatatgaatacttgtctatggacctatctcttaggagttcaggttcatgataagaagcagaaagaaggaaccagggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccggcctgggcgacagagcgagactccgtctcaaaaaaaaaaaaaaaaaaaaaagaaagaaggaaccagacagttgtacattagcacagtgattgagtggtggcctcattagtacttagaattcagaagcaagcctcaaccccatcctgagccctagaaaaaaacagatgacaatgtgtacgatgtgcacaattggggggatgagaggtaagggtaaggagaaggagattatatcccatgaccacctggttttagataaaagaagacaaattcccatagtataacagattacaaagaattagacaccaaaatagaaatctcccccaagtctctggacagtaaaatgtataggtcatgacagtttggtagactttttctactttgattaaataatggggtacaatttgaagtttttggactgtcatgttaatgactgatgcacattacagacatctgtaggcagatattatgcctatacccctatattggactatgctctttaagccaagaaaagcatctgacttttctctagcacctagtataatgcataacaaataccagatatggaaatgttgcttaaaactgaccttcttcaataaagctgtttaaatatattttaaagagatagaacattttaaaacaatgctacacgcaggaaagtagataatataaaatattaaaaagtcaggttccaactacccatgagtatttcagagagatccagcaggatataacttaggaggaaagttccattgttcagaagcttcaagccaattaataatttatgcttttcacatcctcatgttctcactgtctcccacttctgcctcctcttctaaggattattatctcactgacagtttttcttcccagcctctgtattactgcaattcctattaaataacagaaaacgatttccccttcacattgaaaactgttctttgttgtgttttgagacacagtctcactgttgcccaggctggagtgcagcggcccgatctccactcacggcaacctccgcctcctgggttcaagcgattctcctgcctcagcctcccgtagtggggactacaggcatgcaccaccacacctgtattttcagtagatacggggtttcaccgtgttaggatggtctggatctcctaacctagtgatccgcccgcctcagcctcccaaagtgctgggattacaggtgtgagccaccgcacccagctgaaaactgtttttacctcttttcttttactaaggaattcccaaattgggttgtatgtcagtcagctgaaggagtttttaaaattacatcctagatacactgaacaagaatactggggtgagaagctgggtgcagtggctcacgcctgttatcccagcactttgggaggccaaggtgtgcagatcacctgaggtcaagagttagagaccagccttgccaacatggtgaaaccaaaatacaaaagaatagccaggtgtggtggcaggcacctgtaatcccagctactcgggaggctgaggcaggagaattgcttgaacctgggaggcagaggttacagtgagccaagatcacaccactgcactctagtctggttgacagagtgagactccatctcaaaaaaaaaaaaaggatacggaggtgagatccaggactttttcccaaaaagctactcagatcgttcttatgtatagttaagtccagtagcccagtgaatggaatctattgccctaatatctgattctcctttaaaaatgtaagttgctcattcttccatgttaaaggcacttacagtagagaaagcggtgtctcctgtttctcactgctttttcaggccattattctcactactgaacaaaaactgcttttttttttaatgtgggtatgacctttggctactcccctttcaaatgttcccagctttcttttcttttttgtttttttgagacggagtcttgcctcccaggttcaagcaattctcctgcctcagcctccctagtagctgggattacaggcatgcagcaccatgcctggctaattttgtatttttagtagagacggggtttctccatgttggttaggctggccttagaactcccgacctcaggtgattggcccgcctcagcctccgaaagtgctgggattacaggcaggagccatggtgcccggcctatgttcccacctttctacccactatcacatttccccacaggcatcaaagactttgggttggcactgatttcagtttctctctttccaagctattctgtgtgtctcatatataaatgtacatcccaatgaatgctcacaaagtgatcacacccagatgaagaaatatattatcagtaccccagaaccccctcctatgcctcctaaaattgctcactactcctactctacaaaaataaccttcatcctaacttaaattacagatgggttttgcctgttttgtatttgtataaaagtagtaacagagtatgctcttatgctcttcttctttggtcaacattattcatgagattcatctatatggctgtatatagaagtatcttattcattttctttttaaatatttttctttttttctttgagacaaagtcttgctttgtcacccaggctggagtacagtggcacaatcttggctcaccgcaacttccacctcccagtttcaaatgattcttgtgcctcagcctcccgagtagctggggttacaagcgtgcgccattgcaccaagctaattttttctactttcagtagaaatgaggtttcgccatgttggccaggctggtcttgaactcctggcctcaagtgatccacatgcctcagcctcccaaagtgctgggattagagacatgagccactgcctcattttcattattattgtgcagcatccatttatgaatatatccaatttatttctttattctattggaaaacaatttgggttgcttctagcttttggctattaagaataaagctatacagccaggcgcggtggttcacgcctgtaatcccagcactttcagaggccgaggcaggtggattacgaggtcaggagttcaagaccagcctggccacaatggtgaaaccgtctctactaaaaatacaaaaattagctggccgtggtggagggcacctgtaatcccagctactcgggaggctgaggcagaattgcttgaacccaggaggcagaggttgcagtgagctgagatcgtgccactgcattccagcttgggcaacagagcgagattcaaaaaaacaaaaaaacaaaaaaccaaaaaaacaaaaaaacaacaaaaaccatgcttctgaagttcacatagaaaaaaaacctcataaaaagacagacggatcggtgacaggttcttctccatttgtgccaagccatatggatcttgctaaaatgtcacacttatatacttgagttcattacagcatactgcctgtatgcattatcatgacactcaaggccctattatagtctagtctcaaactacctttccagaatattctacattttcacatccttcactttgttacaaattacataccgatatttaccatcacaaaatcttgtaatgatcagtgctggattttcatttaaaaattatgctcattcattcttctaacatctgtggtccaggtgcaatagctcacgcctataatcccaacactttgggaggctgaggcaggaggactaattgaggccaggagttggagaccagccagggcaagagagcaagactccgtctctataaaaaattagttgggcgtggtagcatctgcagacgtcttcttatgagaactgcttttagtttattcagttatttgaagttaaatgaaagacaatttatgttaaaaaaaatttgtgctaaaaaggttttaagtatcattctaatccagattattggctcattgaatccagtgagtaggaaaaatctagtttgtatttgctgggggtgtgtgtgtgtgtgtgtgtgtgtgtgtgtgtgtgtgcacgcgtgtgttttaagagacagggtcttgctctgtcacccaggctggagtacggtggcagaatcatggctcactgtaccctcgaactcctaggctcaagtgatcctacggccacagtctcccaagtagccaggactacagccagttgagtcaataaatatttcttgtttcaagctatctccttatttaatatggtctataaaataagattattaagtaaacatttaactttatacctagcttttatataagcccaatcatcccctttttaacgcataacttaacatcgtaattctttagatcacataactctttagacaattgtgtagttctaaaacattaaccaggccaggtgtggtagctcacatctgtaatcccagcactttgggaggctgaggcgggtggatcacttgaggtcaggagtttgagagcagcctggccagcatggtgaaaccccatctctactaaaaatacaaaaaagttagcttggaatggtggtgcatgcctgtagtcccaactacctgggaggctgaggtaagagaatggcttgaacccgggaggcagaggttgcagtgagccgagatcgtgccattgcactccagcctgggcaatacagcaaggctccatctcaaaaaacaaaacaaaacaaaacaaaaacaaaaaacacataaaccattcgaggcttgaaatggaccttcataataagcatattaaaaaaaagcatttccacactctgcccagtgtcaccccgatctagctttttcattgccttggtttaaaaagctttttattggccaggtgcagtggatcacacctgtaatcccagtactttgggaggccaaggagggcagatcacaagatcaggagatcgagaccatcctggctaacacggtgaaaccccatctctactaaaaatacaaaaaatcagccgggcgtggtggcgggcgcctgtagtcccagctattcgggaggctgaagcaggagaatggggtgaacctgggaggcggagcttgcagtgagccaagatcatgccactgcactccagcccgggcaacagagcaagactctgtctcaaaaaaaaaaaaaaaaaaaaaaaaaaaaaaaaggcttttttaatttttttattttagtatagttttaggtttacaggaaaggtaaaaatacagagagttcctgtacaacccagatccaatttccccaatgttaatacctttcattactatggtatatttgtcacaactaatgaaccaatactgatatattattaactaaatcctatactttattgagattttagtagttttcccctaatgttctttttctgttccagagtctcacctagagtactgaaatacatttagttgctcctgtctttttagactcttctggtctatgacagtctctcagactttccttatgtctgaaagtatgtcaaatattttgtagaatgttcctcaattaagggtatgattttttccctcatgattagattgtgtttgtgggtttttagggggaagatactacagatataaagtaccattgtcaacatatcaacatgacttatcacttacaatactgtcaacatgctatcaacatgacctatcacttacaatactaaatttaatcacctgggtaggtaatgtttgccaagtttctctacttcaaagttgtttcccccttttgaaactgtactattcagaagcaagtaactaagcacagcctacatttaaggggtagaagttatatttcatctccttgagaagggagtatttaaatgatttggaattcttttgtatgtacattgtcatattctagctaattgttcataaatatactcacccttaggttataaattacttagtgttgtatgttccaggtaggcatgatatttaacaaatttctgccaaatacataaatttcccaaagcatataataaaaataaactggttcattattatgtcttcaacatctagaatacagtgcctggattattacagaatattcagaattataacataggctccataagaacagggatatcttgttattcaatattgtatctctagatagtaaaactgtacacagctcaagtgctcagtaaatatttgatgaatatataattcaaaaatgtaaactcttctttaccctaagtgtatataaatgttatatacaagaatctctctaccacacagtttaagagaggaaagaatggacctaactaaaagaaaacacacaaggctgaaaagttagtcagtcagtaagacctaatgccataccagacaaggattaagtcacacgctccctatacttaaagaataaactacattctaactgcaacagaatttcttcttttttctagaagctaagcaagcactggcctcaagataagcaatattgaaacaattgcagcttactgactgccagatgctgactgactcactccctgttccacagccgtaactacagctttgactggacaagagactgatttcagtaactttctcctgataggagtcgccaaccatggactggttctggcccgtatacagaggctgagcattgagtgccttcgtgtccttggctttaccttttgacaacagggcctaattgtaatgcatttaaattgttcagtccccactccaaagtaaacatgaaatgcatgtaacatgcatgtttgtctactatgcatgcgtgtgtttcccttttcatgatgttcacagctcctcctatagcctgctgaatcgatttagttgtacttggccaacttattcagcatcaattcctatgttacccttccctcctgcaaagtgcctgcttttagtctttgccagaggccacacttcccagcctgtcaggatggccagtcttttttttttttttgagatggagtctcgctctgtcacccaggttggagtgcggtggcgcaatctcagctcactgcaagctccgcctcccaggttcacgacattctcctgcctcagcctcccgagtagctgggactacaggcgcctgccatcacacctggctaatttttgtatttttagtagagagggggttccatcatgttagccaggatggtctcaatctgctgacatcgtgatccacccacctcggcctcccaacgtgctgggattacaggtgtgagccaccacacccggcccaggatggccagtctttataagaattaaagctcttctctccaaattggtagatcttgtgattctttgaagttcctcctgactaaagaatgatacagaggaatgacagaaataaggctaaggaaacaaacaggaatccagacacaaaggaatttatgtgccatgttaacaagtttggactttatcctataataaatataatccttaaagagtttttaagtagagaaataacatgattagatattatttttcattatttttatttctatttttgacacagggtctcactctgttgcccaggctggagtacagtggtgctatctcagcttactgcaggctccgcttcctgggctcaggtgatcctcccacctcagcttcccaaatagctgggaccacaggcagcaatttagccaccacatctggctaatttttttgtatttttttgtagagacaaggtttcaccacgttgcccaggctggtctcaaactcctggactcaagcgatgcacccacatcagcctcccaaagtgctgagattacaggcgtgagtcaccaagactggtagagaagtaacatgattagatattaatcagaaagaaaaaaacaccctcattctgaatccatgctgtaaaaatacaatggaggggaacaataataaatacaaggaacccaggtgaaagacagaggcctagactagaaatggcaaaggagtgggagtaccaaatgaatttatgcacgatttaggacacagaaccaacagatcttggtaatacactaaaggtaaagttttagggagaggaagaagtaaagaatgtatagcattatcagtcacggtggctactgctgacaggcaggctggcagatgaaaagacagtaatttgtgtttgggaagtgctgaatcacacaccaagacatgggtttggaaactatggaaagacaaaggctaaagaaagaacttgaaaagtaatcagcatatagacagtaactgaaatacaggtgtggataagattacccaggattaaagtcaggagtagaaagaatatagagcttaggttggagttgaaatggtgaggactagtatttaagaaatctttagttatcccagcatattaagaatatgccaggccgggcgcggtggctcacgcctgtaatcccagcactttgggaggccgaggcgggtggatcatgaggtcaggagatcaagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccgacctgggcgacagagcgagactccgtctcaaaaaaaaaaaaagaatatgccagtgttcatactgaggagaacatgggtaaaacagaaacagtagaaagctaacttttaattactctaaccaagattcactacttaattaaatagatccgagaacacacaaaaaagtactcattatataagtcagttcttcccaggagaagcagtgttttagtttgggagaaaaatgctgggtccacaaacccaggacaatctggggttctagtctggttgtactaacaaccagctatatgaccaggtaactgaaaactataaaatgagctctttaagatccttcgtaagtctaagagtcaatgattccactgagatcattctatagtctagcaagatatacaaataagactttatatcagaaagaatagtataaaatatgagcatgggatacaaacaaatctggggattcaattactctcatttactaaattgttaaaagttggcaagttgctttaatttctctatgcatcagtgaatgtatcacacagaattttattaatataaggtacttaaagtgcttctcacagtgcctggtacataaaacatttttcaaatgttagctattgttttaaatatctttagtaataaactctaaaaaatccatattataagatataccacttccaacaccatgaaccatgtacaagcagtggctaccacctatttaaaatataatacagtcagagaagagcagagagaaaacagaatggcttggaggcagtggagtgacctcattacctgcgggtcctgcagcagctgcatcttccatgagttctccctcttctaattccatgttgttatattccacatcattttctccagacctaagaatatacaaaatccatcatttatatagagtgatacaataaacgttgtttttcacacctcaataaattttatgatatgcagatttttctgtgaaataagatatttctaaatacaaaaaggagaaagatttttgtcattttccaaatttaagtagcgttaatgttattgtttaaacaacctttaaaaaatatcggccgggcgcggtggctcatgcctgtaatcccagcactttgggaggctgaggcaggcggatcacgaggtcaggagatcgagaccatcctggctaacacggtgaaaccctgtctctactaaaaatacaaaaaattagctgggtgtggtggcaggcacctgtagtcccagctactcaggaggctgaatcaggagaatggcgtgaacccaggaggcaagcttgcagtgagccaagattgcacaactgcactccagcctgggcgacagagcgagactccatctcaaaaaaaaaaaaaaaatcagccagttcttcttagaaagctaagaaaaaaaggaatttaaaaaaaaaaaaaaaaaaatatatatatatatatatatatatatctcaaggccgggcgcagtgactcacgcctgaaatcccagaactttgggaggctaaggtgggaggatcacctgaggtcaggatttcgagaccagcctggccaacatggcaaaaccctgtctctcctaaaaatacaaaaattagctgggcgttgtggtggggacctgtaatcccagctcctcgggaggctgaggcagcagaatcggttgaacccgggaggtggaggttgtagtgagccaagattgcactactgcacttcagcctggacgacagagcgagactctgtctcaaggaggacaggacaggacaggaaaggaaaggaaaggaagattaaaaaaaaagaactgtgtcaggcaaaaaaatagaaataaatgaaacactaaaaaatctaagaaccactattagaagagattaatctctcctatttaattagtgttagaaacactaataaatacaagaaccactgttagaagaaattacagcttcagagaactggtagcttaatggcaataagcttaaaacactacatgtagtgttaggcttaaaacactacattttaacatccaaaaaaagcccaaattactgtatatgactaaataccatttttcaaaaataatatttcagcaaagagggtattacttcctactcggccatacaaagtctctcatgttaaaaaacaatcactattactttattttaaacaacatactacttgaaacagtaccagtcaatgacagtcttgggtatttaaggaaatctcaagataagcacccagcttgacaatgtaaggagctctgtaggtctgctctttagtcaaactggtgacaattattttaaaaacaaccacttaacgactctggaaatggtcccaagggcatacagcaaatgaagaaacatctactcaaaaaaatctataaaaactgggtaagacaagtgggcatctgcagtatttgaaccaagatcactccacttcctaacttggtgttacagaaattccactccagactggtgcagtgaagaacacaggcctcccacttcccgaacttccaagaggagggctatattcctgggagtggcaaggtatcagcatctctcattctgcccccagctacctattgctgatgctatagtacaggcacacacggccaagaggtggagactcccttctgctgagacccacttgtggactagatgctctacctggatgcagcatgatgagaatactggggcccagatcatccctgccccagcttattcataaggtggaggtcccaagcctgaaaaggaaagaactaaggctactgtccccaacatccagttagctcagcttctaaagtaagagaggtacacctttatccttactccaacttcaaagctttggctcagagattttgcctatggggagaagcagagacacatacaacactcagagaaacatacaacactattaagagcatcaacgaaatatagttggccttctttatctgtgggctctgcatctgtgcattcaaccaactgtggatcaaaaatattttttaagccctggtgtggtggttcatgcctgtaatcccagcactttgggaagccgaggctggagggacttcttgagcctaggagctccgggccagcctagaacatagtaaggcaacatagtgagaccctgtctctacaaaatattaaaaaattagccaggtgtgttggcatgcacctgtagtcctagctacttggaaaactgaggtgggaggatcacttgagcccaggaggtcgaggttgcagtgagctgtgatcacgcgaccactgcactccagcttgggtgacagggcaagaccccgtctcaaaaaataaagccggccgggtgctgtggctcacctgaggccaggagttcaaaaccagcctggtcaacattgcaaaaccctgtctctactaaaactacaaaaaattagtcgggcgtagtggcaggcgtgtataatcccagctactcaggaggctgaggcaggggaactgcttgaacccaggagacggaggctgcagtgagctgagattgtgccactgcactccagcctgggcgacaagagcgaaactccgtctcaaaaaaaaataaataaaacaaaacaaaataaagtcatgcgcagacattttccccttgtcattattctgtatacaatagagtataacaactacttacatagcatttgcattgtattaggtattataagtaatctagagatgatttaaaagtataggggaggatgtgcataggttatatgcaactactctgccattttatataagatacttgagcatcctcagattttagtatccaagggcggggtcctagaaccaataccgcaaggatactgagggacaactgtactagcaaacagaatgcagtaacatataaaaagaatcatgagccgagtgtgatgatgcacaccagtagtcctagctactatggaggctgaggcaggtggatcacttgaggccagagttgaaggttacagggagctataatcatgccaccatactccagcctggccacaaagccagattctcttaggaaaaaaaaaaaattgctgggtgccgtggctcatgcctgtaatcccagcactttgggaggccgaggcaggcagatcacaaggtcaggagattgagatcatcctggctaacacggtgaaaccccatctctattaaaaatacaaaacaaaattagccgggcaaggtggtgggcacctgtagtcccagctacttgggagactgaggcaggagaatggtgtgaatccagaaggcggagcttgcagtgagcggatattgcgccactgcactccagcctgggggacagagcgagactccgtttcaaataaaaaaaaaagaaaaaaaaaattacggctgggcgaagtggatcacacctgtaattccagcactttgggaggccaagacgggtggatcacgaggtcaggtgtttgagaccagcctggccaacgcagtgaaacctcgtctctactaaaaatacaaaaaattagctgggtgtggtggtgggtgcctgtaatcccagctacttgggaggctgaggcaggagaatcgcctgaacccgggaggcggaggttgcagtgagccgagatcatgccactgcacttaagcccgggtgacagtgcgaaactccgtcacaaaaaaaaaaaaaagaaaaaagaaaaaaaattacacactatgaccaagtgggatgtgtcccaggaatgcaagcttggtttaaaaatccaaaaatcgccaggcacagtggctcacacctgtaatcctagcactttgggaggctgaagtgggcagatcacctgaagtcaggagttcaagaccagcctggccaacatggtgaaaccccgtctccactaaaaatacaaaaaaatagccaggcgtggtggtgcacgcctgtaatccttgctactcgggaggctgaggtggaaggatcacttgaacccaggagatggaggttgcagtgagccaagatcgtgacactgcatgccagcctgggtgacagagcaagactccatctcaaaaaaaaaaaattatccacattaatgaaattcattagttcatcaattaacagggcagaaggaaaagctcttccttaaattgcaactaatgaatgagtaagaaataatggaattagaaaaacaccacttggcaatcaacacagtaacctttttcaggcaacactcatcaaagggtgtcaaaattcataggaaaaagtaggatgaaaaataggatatttacagtcccatacataagatacttattaattataaaggaaaaagtacaaattttggagtgaagaaacctggcggatgctacctcaactaagtgatcaaaattaaccacaagtaatgggacatactgacatcatgctccactctgtatgacgcagtgaaaaggatacattatcatttccgtagtatccctgccaaaagtgtgtaacctctatctaatcatgaggaaacttcagactaatccaaactgagggactttctacaaaataaactattctgtatttctcagaaagtattagtcatgaaaaacagtttttttttttttttgagacagagtctcaactgtcgcccaggctggagtgccgtggcgtgatcttagctcactgcaagctccgcctcccgggttcaccccattctcctgcctcagcctaccaagtagctgggactacaggcgcctgccaccacacctggctaatttcttttttgtatttttagtagagacggggtttcaccgtgttagccaggatggtctggatctcctgacctcgtgatccacccgcctcagcctcccagagtgctgggattacaggcgtgagccaccgtgcccagccgaaaaacaaatttttaaaaaagtaacccagattggccaggtgcggtggctcacgcctgtaatcccaacactttgggaggccgaggtggacggatcatgaggtcaggagatcaagaccaacctggctaacacggtgaaaccccgtctctactaaaaatacaaaaaattagctgggcgaggtggcgggcgcctgtagttctagctgctcgggaggctgaggcaggagaatggcgtaatccggaaggtggagcttgcagtgagctgagatcatgccactgcactccagcctgggcgacagagcgagactccgtctcaaaaaaaaaggaaaaaaaaaaaagtaacccagattgaagataactaaatcgatttaacaattaaatgcaacatgatcctgacaaaggacattactggcaaatttttagaaggttacagtattacatcaatgttaatttccagattttgataactattctatagttatgtaagaatatccttgtttttagaaaagtacacaatgagaagtatattcgatctcaagtgtttcagaaaaaattatatacagagaaatataaagcaaaatattcatagggaatgaatctgtgtgaaggaaacatgaaattctgaacttttcttaagtataaaataatttcaaaattcaaatttaaaagttaaaacacaaacaaaaattattggctaagagcccagagattagagagagaaaactgactattgattcaatctttcaaaaaagtgctgaatatccctacatttaagtgtgagttagttaatatgcttctaataattaccacttaaatgctttacagtagtaaagtgggaaactcacatagtttcttcatcaatatcattttcttctgtattacttgttgctgtggaactagcagaggaactgctgccatcgagctgatttacttcatcctcataaacaagatccagatccagatctccatctgaaagctcgtgctagatcaatgccaagaaaacaagacgtctattccaaatgtttctactcagttgatcaccccatccagcccataaaccaagcaccaaagaataatacagttttcaaaaatctattttctgctcctgtgcctcagctgctgagccctgtagagaaaaatcacacaaccacactgactgatatcattttactaacttatgcgtgccgccttcagctgggccctcagtgtgactggaatccttcacatattccacatcagcttcctcacagagtaacgtttcaaaccatcactcctcaattctacctaattttaccattatcaaggtcattaattaacactgtcacctatctgcacagaaaaaaaatacccactattaagcaaaaacttccccaacttttcagctgccttacctgcaaactccacccttcctttaactcagtttatcaaaggttaactgcttcacctgactgctccattccaaaccctattatctctagtttatcaaagcacttaatctgtgcattctctcgacccactgggtccttcattttaacttaaaactgaagtctctcatcttaaaaattactacaaaaaaaccaactgtctatgcatcccttcttatgtaaaaatatcaactacttcttgaactcttattctggattctgactgttgtttagctgggatagaacctggctagctaccgaatcctaaatcagtctacctacgtaagtaatgataaggcatgtactagtttcagtgaatgaaaatagaacaccttttttttttctttttctttgagacagagtcttgctctgtctcccaggctggagtgcagtggcacaatcttggctcactgcaacctccgcctcctaggttcaagtgattctcctacctcagcctcccaagtagctgggattacaggcaagcaccaccacacccagctaatttttgtatttttcgtagagacagggtttggccatgttggccaggatagtctcgaactcctgacctcatgtgatccacccaccttggcctcccaaagtgctgggattacaggtgtgagccaccgcacccggccagaacacatttttaaaatccacaatgtatggtagtttaggttttaaaatgtgtatcacggtttgttttatacaatgtattgtttttttcacaaccttgaaatgctttgattcaacaacttctgacttattaacatttacataagggcatacatacattactaatggaaatcaatggacaatatttttagcctactgttttagcacataatatgttaaaagccatgatgctactgtttttttttaatcaaacacaaaaatctaaaactggcctcattattacatgataatcttcattctgaatcttctcctcatcttcttcatcctctttggcctctcttacagaagctgtagtaggaccaccttcgagaagcatgtcagagcatgcagacttcttagctcgtgtctccagagcatcatcattttgggggctaagatggttatctggtggtgacaaatctcttgacttctgagttcgagacagctcaatagtaagtctcttttaaaacaagaaaagcacaaatattagccacagctcactggcacagtaaaagatgtctcaagtgaaaaaaaaatgcatccagatgtttctaaaaccattgctttatttattttttgagaatggtctcactctgttgcccaggctgaagtgcagtggtgccatcatagctcactgcaagccttgaactactgggctcaaacgatcctactgcctcagcctctcaagtggctgggactggaggtgtgtgccaccatgcttggctaatttcatttttatttatttttagagacggtatcttgctatgttgctcaagctggttttgaacccctgggctcaaatgttcctctggcctcggcctcccaacatgctgggattacaggaatgagccacctcacttggcaccatttcttaaaccatttctctgaatccctactgccttttatttataaaaaaaaattgatgacaaagataaactgccccatttatgctcatcctccttctttcatatccagcctaagctaaacatacaattagcaaacagtatagtgaatacaggcagttttaaatagcacatttaacatgaaaatctctgaaagcatgtatttattcacattttcctccctgacctattaacgactctcagcactccaaacctcctgccacaccctaacttcctaagggaaatgaaacattccttagtttaccactgtaaaataaaaattcatcaattaatcttccccagtgctcattcaacaatagtaagagttcaagatgggacaacttttaattgtgctatagcctagaatcctctaaatccaagataacttgtggaagagaaactaagtcactgacagaaacacaaacagcttacctcaaactacagccacactgttcaaaacaactttatgcaatgatggaaatgctctagatttgcagtaaccaatgcaagcacttgaaatgtggctactgcaactaaggaactaaatttttaattttaattaagctaaatttacagaccaggcacagtggctcacacctgtaatcccagcactttgggaggccaaggcaggcagactgcttgagcccaggagttcaagaccagcttgggcaacatggagaaacccatctccacaaaaacaaaaactagccatgacacatgctcgtagtcccagctacctgggaggctaaggtgggaagatcacccgagcttgggaagttgaagctgcagttagtggaaattgtgcctggcaacaagagtgagtccgacctgtctccaaaataaaaaaaaataaaaataaagtggcagggcgtggtggctcatgcctgtaatcccagcactttgggaggccgaggtgggcagatcacgaggtcaggacatcgagaccatcctggctaacactgtgaaaccctgtctctactaaaaatacaaaaaaattagcccggtgtggtggcaggcgcctgtagtcccagctactcaggaggctgaggcaggagaatggcgtgaacccaggaggcggagcttgcagtgagccgagattgcaccactgcactccagcctgggcaacagagcgagactccttctcaaaaaaaataaataaataaagtaaaataaaaatttacatggccgtatgtggctagtgtctactaaactggacactgcagaactggtaaggatggggaacaagataactagattttttttctaattacaaattacaaaattacaaatagcaataaaatatataaaatctaattacaaatataacaagccaaaaggacaataggaaacaccaaaacagaaatagatattttcttacctctttaaggttgtttatttgttggatataactagtctgtgcagcttccaactgagtaatgtaccaatgctggtcccgggatttttgaaagaaagttggtgaacgtacctgaaaccttaaaagaattgtgccacattaaaaaaaaaattcaaatagtaaaacattatcagtctagcactgaattctgcaatttgggttaacttgattataaggccaaaattcacataaacttagagctgctagtatttgttgtttatagatgtagtccacagattatgacacaaggttctgaaaagccagttgtcctcaaactacctttctaaagaaataacattgtacattgtagttaagcttcccaaactaacctactcaggcatatttattataataatgtaactaaaatacaatgatgatcatgagttgggaagaagagagccatatactatgcagtttcctcctataaggaggaagataactcttatccctttcttccctaaatgaggccccagcaaaatacaaagttttttttttttttgagacggagtctcgctctgtcgcccaggctggagtgcagtggcgggatctcggctcactgcaagctccacctcccgggttcacgccattctcctgcctcagcctcccaagtagctgggactacaggcgcccgccactacgcccggctaattttttgtagcaaaatacaaagttttaagtgctgctctttatatttctctattccatctcacgttcccgtatttcctgcccctcagaaaaaaaatcacttcctggctcaaccacccaggattgctttactatcccccaaggagttacatgctctaaataaactatatcctgaacccttcaagatagcaatcaaaactgcccttattacaatatggcattatttcagcttaggttaggaaaaaaagtactgcacctacataaaatcgtattaatatataaatgacagaatcttaatctatccagagttgttttaattaaaaaaaaaaaaaaaactattactaaagcaaacaaacctctacctttgttacatagcctaacgtgactatatttaaggtgaaaaaattgcagtgcatgtgtgaaggtgtttttgaagaaaggaaaaaccacaaggcaagactaatttggacatctacatctctctcattccttccagcccttttacccggctacacaatgttagctgttcatttttttctaggattttacaccttattgtaagaacctactgaccatttaacagttatctggcagtagatatctagttttagtgaagaatactaatgcaagaaaggacagaacataaacactgactataaaacaacagtcctatttaatgttgggatagtctttataggtcagaagtttttgggcatcattatatgttataactggatgatcaaaatgagaacttgagtattaaaaaaagagagagattggccgggcacagtggctcacacctgtaatcttagcactttgggaggccgaggtgggcagatcacctaaggtcaggagttcgagaccagcgtggccaacatggtgaaaccccgtctctactaaaagtacaaaaattagccgggtatggtggcgggcgcctgtaatcccagctactcaggaggctgaggcaggagaatcgcttgaatctgggaggcaaaggtggcagtgagccgagatcgcaccactgcactccagcctgggcaacagagcgaaactctgtctcaaaaaatcaaatcaaatcaaatcaaatcaaatcaaatcaaatcaaatcaagagattaaattacgttctgaacggaactgagtagagatgattcactcaactgagcagagacgattcacagtctaggaagtacccactggtaagacattaaagtaagaccgtctaaacactattattttctttgaatgatttaaaccactggttaactctgcacattagttctaatattttgccattaaaacaaaaccaagcagcaggtaaaatttcttaactttctgttttaatgaaagaatgaaacatacattcatagttatagaaaagccttaaataactttctaataaatacatacaaaattccaccagaatcttgaaataaaaggtttaaagtttgagcaatgtctcattaataggataatgtgaagccccctatccacaaacttcatagcaatttacttacggtgctactatagcattattttataataagagagtagtaaattctgtttttatctgccaatttcatcttgagttccctgtggacaaaaaccatgtctatatttttcttattagcaactcagtgtcaggcatatggtatagactcaatgttgcttaaaaaaaatgatgtcataacaggaatttgtttctgtactaataagtaacgctaataaggatgaaatcaaggctataagaaaatgggtcttttataggttggtaaaattgatgtttgaataattttgtaattctctctatggctgaagcatgaaatagcaataaattatttctcttggtgaatagttaaaccccaaaagtctgaaaagctatacgaagaaaactaaaataatccctcccctcaaaaaacatgattccaggcaccgtggcatgcacctgtaatcctacctacttgggaagttgagacaggaggatcacttgagcccgagaattctagactagactgggcaatatagcaagacactgactctaaacaaaacaaaacaataacaaaaacatatctttttccctacactgaaaatcatatcccttgttaaacttctttatacttccagaaaagctacggaaaacacataagagtaaatgctgatatgatagcaactcatctgtccagtatgctcaagaacaaagagctccgaataagggaaattcaggagtacattaaatcactctcaagaaaaaaatgtttaaaattgttgtgatgagaactttttaaactgttaaatactctgaaacttctcagttcagaacatattttaatcttttttaaatattcaagttcttattttgatcatcaaattataacatcatgtaatgatacccaaaaacctttttttgtttgttttttgtttttttaagatggagtttcactcttgtcgcccaggctggagtgcactggtgaaatctcggctcactgcaacatctgcctcccggactcaagcgattcaactgcctcagcttcccaagtagctgggattacaagtgcctgccaccatgcccagctaatttttgtatttttagtagagacagggtttcaccatgttgcccaggctgatcttgaactcctgacctcaggtgatccacccacctcagcctcccaaagtgctgggattacaggcgtgagccacggcacccagccaatgcccaaaaacttctgacctgtaaagactaccccacgttaaacagggctgttgtttttatttactttattattatttttgagatagggtcttgctatgtcgcccaggctggggtgcagtggcataatctcggttcactgcaacctccacctgccggattcaagcgattctcctgccttggcctcctgagtagctgggattacaggcgcgtgccaccacagcccggctaatttttgtatttttagtagagataggatttcaccatattggccaggctgctctcaaactcctgagcccaagtgatctgcctgcctcagcctcccaaagtgctgggattacaggcgtgagccaccacacctggctaaatagggctgttgtttgagtcagtatttacgttctatcctttcttgtaaatactacaccaacaatcaacaataaaaattgatggttttggccagacgtggtggctcatgcctataatcccagcactatgggaggccgaagtgggtggatcacctgaggtcgggagttagaaaccagcctgatcaacacggagaaaccccatctctactaaaagtacaaaattagccaggcatggtggcgcgtgcctgcaatcccatctactcgggaggctgaggcaggagaatcgcttgaacctgggaggcaaaggttgcagggagccaagatcgcgccattgcgctccagcctgggcaactagaaactctgtctcaaaaaaaaaaaaaaaaaaaaaaaaaaattgatggttttcgcaaataccacatgttctcacttattatttaagagctaaatgggccgggcgcagtggctcaggcctgtaatcccagcactttggaggctgaggcaggaggctcaagaggtcaggagatcgagaccatcctggctaacacggtggaaccccatctctactaaaaatacaaaaacaaaattagccagatgcagtggcaggcacctgtagtcccagctacttgggaggctgaggcaggagaatggtgtgaacccgggaggcagagcttgcagtgagccgatatgtcgcctctgggcgacagagggagactccatctcaaaaaaaaaaaaaaaaaaaaaaaagctaaatgatgagaactcaaggatataacgaggggtaacaacaacagacactggggcctacttgagggtggagggtgggaggagggagaggataagaaaaaataactattgggtactaggcttagtatctgggtgatgaaataatccgtacaccaaatcaccatgatgtgagtttacctgtataacaaacctacacatgtacccctgaacctaaaataaaagtttaagaaaaaagtagaaaattgctggttttcgtcaactttcatcttgactcttctacttttaatttgctgttattgacaaagaactgatagttgctttataaaattaagttgtaaaaccagtcactcaaatataatatgcactgtgaagtgaaaaaaatcagcactttatccctcactattcttagatttccaaatggtgaggtcactagaagttcaacagacaaatggaacattactatacttactggaagttgtagtttcccacccacccccttttttttttctttttaaaaagatggggggtctcataatgtttcctaagctggtctcaaactcctgggcccaagccgtcctcccatcttagcctccatagtagatggaaacaccggcgcaccactgcacccagcttatctttaatggaattgctaactagatgtcaaaaaaagtttgactttttgactacaaatttttgattattatccacttaaataaaacagttaagaaagtgataaggggccgggcacggtggctcacgcctgtaatcccagcactttgggaggccgaggcgggcggatcacgaggtcaggagatcgagaccatcctggctaaaacagtgaaaccccgtctctactaaaaatagtaaaaaattagccaggcgtggtggcgggcgcctgtagtcccagctactcgggaggctgaggcaggagaatggcgtgaacccgggaggcggagcttgtagtgagccgagatcgcgccagggcgacagagcgagactatgtctccaaaaaaaaaaaaaaaaaaaaaaaaaaaaaaaggtgataaggttatagttagtatgaatttcaatattcatctttgttaaccaaatgatctttgaaatatagttctgatgatattatttccccacaataaaaaccacaacatcaaatttttcctgttaccttaaaatcactgtatcattttgtggattcaagtatccttcatttgcgagtaagtccaaacggaaaaatctattatagccccagcattctccaacttcaaagtcagatgcaaattctcgaatgatattttttgtaggatcattacaggactggtgaaccatctctacacgatattcatatctaaaattgaaaaacagaccattactatacagttagtaaagtaagaataaaaaacaaactcatctaaacattaaggtcactaagtaattacctgagaagaaaccttattgatatacgcagttacatttcttttatcatttataaaaacagcagtattttggggtttttttatttatttaagatggagtttcactcttgttgcccaggctggagtgcaatggtgcaatttcagctcactgcatcctctgactccaggttcaagtgattctcctgcctcggcctacagagtagctgagattacaggcgtgtaccatgcttagctaattttgtatttttagtggagacgggggttcatgttggccaggctggtcttgaactcctgacctcaggtgatccacccaccttggcctcccagtgtgctgggattgcaggcgtgagccaccacacccggccagaaaaagagcagtatttttttgtttgcttttttgagatagagtctcactctgtagcccaggctagagggcagtggcggggtctcagctcactgcaacctctgccccccagttcaagtgattctgctgcctcagcctcccaagtagctgagattacaagtgcacaccaccatgcccagctaattttttgtatttttagtagagacagggtttcataatgttggacaggctagtatcaaactcttgacctcagatgatccaccctccttgacctcccaaagggctgggattacgggtgtgagccactgcgcccagccaaaacagcagtattttgaacgaaccaccaaaacatgaatggcaataggtccttttttcctcgtaaagatgtttaccgatatatcaggtataaaacaacacctcaaaaaataaataaattttgaataaacaagtaattcaaagttgcttttgttttactaatgcaatattttttccatatattcactacatatgtttcctcataaatattgttatttctattatcctttgaggtttatcaccttgcaattttaaaatataaattttgaaatactctactgtattttgccagtacaagtgataagaaagcaaattattgtgtattcagaaaatcaaaggctataaaatttcctaatagaaaataagtatgcagccataaaaaagaacgagattatgttttttgtgcaacatggatggatggagctggaggctattatcctcagcaaactaacacaggaacagaaaaccaaataccacgtgttctcacttatattgtgggagctaaatgatgagaacttatgaacacaaagaaggaaacaacagacactggggtatgcttgagggcagagggtgggaaaaaggagaggagcagaaaagataactattgggtactgggcttaattcctgggtgatgaaataatctgtacaatgaacccccatgacatgagtttacctatgtaacaaaccttcatacgtaacaccaaacctaaaataaaagttaaaaagaaaagaaaaaggaaaaagaaaataagtaagctggacacagtggctaacacctgtaatcccagcactttaagagaccgaggtgggcagatcgcctgagcccaggagtctgagaccagcctgggcaacatggcgaaaccccgtctccataaaaacaaaaaacaaaaaaattagctgggtacggtagtgcattcctgtagtcccagctacttgggagactgaggcaggaggatcacctgggcccaggaggttgaggctgcagtgagccataattgtgccactgtgctccagcctcagtgacacagcaagaccctgtctcaaaaagaaaagaagtatacattgggaaagccaatatattctaatttatatatccaatcttgtaaaattttcaggtttacaaaaagacaaagaagatagcctataaacttttgtgcattaatgcacattatcaagaaaatgaaaagaaaacctaccaaatgggagaaactatttgcaaatcatatgtctgataaaggtttagtatccaaaatatataaagaattcttgaccgggcacagtggcacacacctgtaatcccagcattttgggaggctgaggtgggcggatcacctgaggatacgagttcaagaccagcctggccaatatggtgaaaccccgtttctactaaaaatacaaaaattggctgggcgtggtggtgcatgcctgtaatcccagctattttgggaggctgaggcaggagaattgcttgaacctgggaggcagaggttgcagtgagctgagatcatgccactgtactccaagctgggtgataaagtgagactcaaaatacattaagtaaataaataaattgagacagggtctcactctgtcattcaggctggagtgcagtggtgcggacacagctcattgcagcctcgacttcctgggctcaagcaatcctcctgcctcagcctcctgagtagctgagacgtatgcagggtctcaccatattgcccaggctggtctcaaattcctggtctcaagcaatcctcctgccttggcctcccaaagtgctgggattacaggtatgggccagccagtctggcatattaacctttgactgaacacaagacactgtaaattttactctgttggatgttagatacttcagcaaccttataaattttcttgagcttccttctgggatgcagttaagttacttgaaaacaatttgatcctttcaggtctcacttttgttaggtgggtccagagcagtgttcagtctagggctaatttctgctatcgagacaaaaacttccttagtgttatacccaatcagtgacccatgaattataaatttctccaccgtggcaggttgcaatagacacagttcctggccccgtgtgaacaccagtcactcactattccctctaatcctgtcaaggagttctttccccaatctcaggtagtttcctcagacatatgccctgataagtactctgctgaatactcaaggggaacccattacggatctctagacttcagtctctgcagctttctcctctctggtattttgtccaatgcgctctagctgtattgtcctttctagactctcagcactatcccctcaactaagtctgccagattctccctcagtcccagctcctccctgtgttgcagcctgaaaactctctcaaggctgtaagctggagaaattgtagagttagtttgtgtttctcatctctcagggactactttcatttgttaccaccatgtccagtgtctagaaaatgttgtttcatgaggtctttttttttttttggttgttgcaggaaaatgggtagatctcatccctcttgcttcatgttagctgaaagcagacattccatactaaactaacagatttcatgtaacagagttaaaaaaaaaaatcattaatatgattccagatcccacattgtaaataacatttaacaacctaccacttgttcagtttggatgtaatatcaaagaagatccacaattatctaaaaccactttaaaaaatattcctccttgtcaaaaagtacatttgtggttctcagggactgagggaaacggggaatgggaagtgactgctaaggagtacagggattccttgtggagtaaatagaatgttataaaattagatgttggtgatggttgtagaactctgcagacagatttcatacatgtatttcaatcaaaatgatattttaaaaaaattgaatataaaagcagacatgagaatccagtagtcttcttctaagccagatagtaaagagatttctaaaaacgcaaagcaatacaagttttcttaaaaatattttgtttttagaaaaataccatcctatgatgccaagaacatattattctacatcttaacagacataccaatacagtaattaacaaaaataaaaattaacaaaaattgctagggggaaggtatgtaaatgcacattaaggttgttatttaattacacttcttacttagaagtttcaggcaagccagctgagagctccagaaacacagataagtagtaacctcgcacaactccatttccatcctaaaagaagaataatcagtttgagtgtaaaaaaattaagccacaataaaacaaaatgcagataatatctgatatctagattgagaagtacttctaagcctcaaagcaatggaagaaataattaaaaacaccaatagatttgagttcttaacaactttaaacttttgtgcattaaaaaaaaagtgaaactattaaggcacaaaaatgaagggaaatatcaacaaccacaacaaaggttaagagttttaacatatatagaactcttacaaatcaaccttaataaaaaaacaagaccctaatgggaaaaataatttcattagaaatgagtgaagcaaaaaaaaattttaaaccaaaaattaatttttaagaccaacatacaaaaaggtaataccctatgctgaaagtaattaaatataatttctatatgattttaatattagaattccatataagattctgtctagagattgagaagttgtttaaatctctactatagattttatttttataaagataattactccctccctttttgtttaatttggcaagactcttttaaaaaaagagaattacttcctatgtatttccctagtacttcaggtgtctatgttttaaaagaatcacatttattataactacttgtttaaacatctgttcccccaacgagacaatgaaggctgacaacagagatccttatacatctccagtgtctactgggatatgactacagagaggtacaagtactattaaccccttcatttttagtctactttgcttgaaaggacctgaggcaacttttgaggttcgacatgataatcactcagtagatatcaatgaataaatgactcatcagctaggtgaataaagccagacataaaagatgatatattattgtgtaatgccattatacaaaacttttctaaaaaaaaaaaaataatctgaaactacatattgaaagacttcaatgtgtaactgagaatactaacccaggagggctaaaatgtgtaatagtaaattactggactcaaaaagattatttagcatctaggcaatatcaccaaacatctttaaaggtaaagaaaattagtttattatcagacttttcctaaagaaaatggaattgcatattgaagatacccaatgaaagaaaatatgagccaaggattttatatccagcaatactagccttagaatataaaaggcaaaaactgttttcaacatatgagaactcagggaacattgtttccaataggccttccagcagaatgtattagagaacaaggtgtggacaaacaaacagttgtctaatatgggggaagaaggggaacagggaatgcggacagcatatgtaacaaaaaaaagaagagttaacagttctcagtaatcaaattagtatactacagaactctgaaagaaagaccttggtttcaaaatttattagaaatatgaaaaacttactgggtaaacttttaacctccagcaaagtcctgaaacttgaagaggtggactgtaaacaggatctgctctctgacgcaaagtgctaacgaaaaagaaaaccaaccaaatcccaagatcaaaactgttgtgaaatggcaacaggcaaaaagaaagaccaaccagtttctttgttacatacttgaaatcctagacaaataacaaggtgactgaacaggtcaaaattccactgtaaaagtttagatatgcttacatcctgaaataaagaaaatttaaagatactgaaatattcagcaatatcttttaagtttcaggggaaaaaaaaccctcaaaattgtgacatttaagggaaaaggaatgtatcaccaagtctcccaaaattaattaatgcttcaacaagcagtcttcaccattaaaaataaaacaaaacccagaatgtttcttcctcgcttctctgcagtataatcatcttttaatattgaagaacaagagtacactaaaccttaatacgtgtgggaaaaacctcaaagagactatgttttagccacaaaagtaaccctaagaaaagacaaattaaatgggaagagggataaattctgtgtggctttttttttttttgagatagagttttttcagacagagttttactcttgttgcccaggctggagcgcagtggcacgatctcagctcactgcaacctccacctcccggtttcaggcaattctcctgcctcagcctcccaagtagctgggattacaggcgcccaccaccacgcccagctaatttttgtatttttagtagagacggggtttaccatgttggccaggctggtcttgaactcctgacctcgatgatccacccgccttggcctcccaaagtgctgggattacaagcttaagccaccatgccctgcccagtgtggccttattttgatggcctttgcctattatttggagtttctctcctgaagaccttatagtccttgtttccacttccatgctgccctcaaagacaaattgctaagaacctttgcatgttacgatcttcacttctgatcttaagagaaaaagtagtaaaagctaacaaaattaacatacaactgacaaagcctacacatttcttacaataatggccccttatccacaattcttcactttgagctgagataaatggatctcaatggatttcttcccagcacttttatttctgaggataggtaagagggggctcatcaacctctaactgtccagccccccaccttagtcaaagctaagtgatctctcttatttaaaatggttcctcactttgggcagggcaccgtggctaatgcctataattccagcactttgggaggctgaggtgggagaattactggagcccaggagtttgagaccagcctaggaaacacagtgagaccccatctgtgtggtggcacacgcttgtagtcccagttacttgggaggctgaggtgggaggattgctgcagcccaggaggttgaggctgcagtgagcaatgatcacgccactgcactccagcctgggtgacagagcaaaacccagtctcaaaataaaaagaaaaggttccttttcccagactagagaagcagtcagtctaaagatacaatataatccagcacaaattatctctacatttctaataggaaacttattcttctaagacttctaaagtgagtgacacagttcactagattactgtcttaccaaagaatacctttctggctcttccttatatacacatacaaaaaaaccagaattgtcaaaaactcttacctgaaattctctaaaacaaaagtagctgaatcgtaagatggcactaattcactaaaaaaaaaaaggcaaaaaaaattatttagcttacatgtttaaaattccatttgcctaaaaaagccaagtccctcactagtatcttaaaaactttattaaactaaaatctaagaaatatttttaaatctttattgaacataacattaaatcaaatgaattcataaaaccatcaagtttgcaattaaattggagatacaaatactttataaaagttttttggctgggtgaggtggctcacccctataatcccagcactctgggaggccaaggtgggtggatcatctgaggtcgggagctcaagaccagcatggccaacatggtaaaaccccatctctgttaaaaatacacaaaaattacccagatgtggtggtgtgcacctgcaatcccagctacttgggaggctgaggcacgagaatcacttgaaccctagaggtggaggttgcggtgattcgagattgcactaccacacatctctattaaaaatacgcaaaaattacccaaatgtggtggtgtgcgcctgcaatcccagctacttgggaggctgaggcacgagaatcgcttgaaccccagatgcggaggttgcggtgaatcaagattgcactaccacactccagcgagggcgacagagccagactctgtctcagtcaaccaataaatcaataaataaatactttataaaagctttttttcctaaaagaaaaaaaggcaacacattatataattattaaactaagtacaagaaatacactttaaaaagtactcatggaaatgggaaaaagaaactgacgcttagggtgtgaatctgaattatgaaacagatataaaacatcctttaaaaaatgaaacaacagtcagtgataacacaattaatccccaaacttcctactctggaatggattatgggaaatattaacagacctcaaatactttgaaactatttactagcaaataaatctacacatttttataggatgaatacctcatcacctaaggggcaactgtgatggactagcacactcacactttcttggcaagtcaaaactacttcaagcaactttagtcccatccaatcttcttcagagagaactgtattataatcacagaatggactaaaagtcactttgctgctaactacctttaatcatgtggatggtttgtacactttcattagaacacctcaaaactcattaaaacactatactatgaaaaagactatcatacgatgttttaatagtgttataaatttgatatataaaattacaaatttcccaaaacaataaactgcaaaccatttcaccacatcttcagatatattttctaaagttcaattcatcataattttggttattatcaaaacaagataaaatatacatctatagcactgccacactatgatagtgtctaaagaaattagtggcttgcaatacaatatagacaaataattctttatttcaatgtaagtacgtatagctaaataggtcacgtcttttacacagtaagcagatgtactttgccctgtgattaactgtatagctactatccaaattgactatgtcaagaattcctatgatatccctagtcagttgttatccttcttagaagactcacattcctcatccccatatccttttgcctggttaacccttactatttcttcagataacagattagaagtcacatactcttggaggtttgctctaaggtcttcccattatatgatctcctagcactcaacatacacaaactccaattagcaggaccaatgcaattagtgcctaggaagcctttctggaaccataaacctagtaagagctctaagggagtcttcccagtatagccttcaaatttgacatgtttttgaaattttgtgcaactttcaatcaggtctctcatcaagtacagaatatcctccacaacatgcaaagactttaatcaatccccagttgcctgctaatctgcatattatttcttctgccatgtccagtgtaatttatttcctgtgttgttgttactgttatttctgctctaagtccagactatcattatcatattttattggccattaagggtcaaaagacttcagcagaacccaaattaaacaacaagagatgccattttcaactcagaatggtgaagtagaaagcttccaggctaaagagcataggttactaacactcatgaaccaagagagtacagtccagtttaccctagtctcacttgctaggagaaaatgaccacattcagagcacagaaaacaggtactgtgatatacttttacatatgtacattctcctagggaaaattttttggacacaaatagtataatgacaaattagtggtatggggcagcattaaacaggatacttacttcccctatcaccagttaccacattaattttattactgttccaataacctgtttttctaaatcataattataaacccaaagagctctgtattatgcttaatgctagcacttaacacatgcctaaaatatagctggcatatattcactgaatgaatgtttataaatacttgctggcagattcgtagagttggaaatatttgctcaaaaatttcttttgcgaaagaaattactttgctttttaaatttaaattttatttatttatttatttatttatttttttgagacgcagtctcgctctgtcacacaggctggagtgcagtggcgcgatctcggctcactgattttgcattctttatatgtatatacaaagctattgctactgctaggccatctaattccaccaccacatctccatattatgctcattcatttagagattatcttcaatcatactgatgttatcattctgtgtctgcagatgaggcactgctttatcttttcatagttttaaaattttctcctcctctcttagaagtgtagactgctctggttatgatgactggattcatctcatgatggtgtcagtgaatctttgtcttcacagaatatgggaaactaccctcaaaccatctctagcccaggaatctggaagtcactgatctgcaaatcttcagaccagtaacctcatcataacttcatcaacaaagggaaaaaagggagaaccacaagtatccatttgtttcttaaggaaataaactgctgagctatataaaaactggttctccttatcaacttgactatttctccctgaaacataaaactctctatgaactgacagctatactattgtatcttttattttttcttttctgcctcagcctccggagtagctgggattataggcatgcaccaccacacccggctaattttgtatttttagtagagatggggtttctccatgttggtcagactgctctggaactcctgacttcaggtgatccgcccgcctcggcctcccaaggtgctggggttacaggcatgagccaccgcgcccagcccacagtaaaatatttagttcctcttctcccatgcagcacctacaacaatactgggcacatagtctttaataaataacacttcactgatttagtgaccttttcttctccattgtggctccatcaccaattactaactgatagaactaaaggcaatagaaacaccactgctttgcatatctaaagtttcatgtgtaacttaatgtcacgaatgctgatctttcttatttggccctcttttcctgtatgttgcaactaataaaacaggtttctaataagaattaaattaaaagccaaagtttccaataaggtagtgagcattactggagaaagagagaaggccctgaaagtgattaattaacagactagcgagcattgttgcctgcctgatgagctgctgaaagtggtataagaatgggaatttacatacatcaatggttcccaaactttagtgcagtattattacttagagtgctcaagacgcatttctgaataccacctctaaagattctggggacattggcctgggaatttgcatttcagacaaatgccacacatagccctgacttaggtcatccatgcaccacactttgaattgaaaaatactaatttttatatatgacccttaggtcaaaatctgtttcattccacaggggatgaaatggttcagaaaacaaatcttcaaaactgaaatagctgaccacagtatcaatgaagcagcctagggattttggtgaacattcttcaaccaaatctacttaatgacagcaaatacatagtccttttaataatattcagtgctgtcattcagaaatgagatttaaacaaaaaagtcagtgctttaaaaagatatgaatattttatgaggttgttaatcaaaaagaaaggaagacttctactggtcaatgagaaccactggagttctagctgtcagaaaaactttgcccttctgtttataggactgtggcatctataacgagtaagaagggcttataacagtgattctcagtcaggggtgattttgtccccaaggacatttggcaatgtctggaaatatttttgtcacaactgcaaggtggtggggtggggagtgctactgggatactgctaaacatcttataaaacacaggaaagctcaccccaccccacaaataattattctgcttaaaatgtcaacaatgccaagattgaaaagccctggcttatatgatatactctagtacaatgaatgcggccaggcatggtggctcacacctataatcccagcactttgggaggtcgaggcaggcaaatcacttgaggtgaggagtttgagaccagcctggccaacatggagaaaccccgtctctactaaaaatacaaaacttagctgggcgtggtggtgcatgcctatattcccagctacccaggaggctgaggtgtgagaattgcttgaatccgggaggtggaggttgcagtgagccgagatcacgccactgcattccagcctgggtgacagagtgagatggagtataataataaataaataaaataataaattaaaaaaataaaaaataaaatagtacaatgaatgctctgtggtcatcagaggcatcacatattagaggaaaaagaaaaggcagctcaacatataaacatttttataacaaagactgttttctaaatgtgccacagccctcaccatggtaggaaaacttgatagtattccataagtcatctctctaagataaagtctgtcacaccaaaatgtttatctgaagcagtggcttcacccctcatcatagaataagctaaggagattttaaaagaacaaactgaaaatgtccatctgaaccctatgtcacaggctgaattgtgtctccccaaattcccatgttaaagccctaacccccaatgtgactgtatctatagccagggcctttaggaagataattaaggctaaatgagttgataatggtgggtctaatacaataaggcttgtgtcctcataaaaagagaaagagacaccagagactgatctctctccaagcaagaacagagaaaaggtcaggtgaagacacagaaagaaggcagctgtctgcaagctgaggcaaaaaagtgaattttctgacaccctgatcttggaattcttgtttctcgaactgtgagaaaatacatttctgttgtttaaaggcacccagtatatggtattttgttatagcagcccaagtagactaatacacacccaaagattttctttcaaatggcctggggtatggtatgacccagcactgattaattttcagccaaatcccaagtgattccaacatctagtcagaaaccaccagtctaacaccacagaatgattacaaacatgcttgaaggaatatgctgttcagaaaaattctaaggtttagcccaagatgaaaactaggatactttttgccataattatttcctctctctatcttttagtatccggagataacatggcgtcaccttcaaattcttttggcatttcatcagtttctgagttttgtgttaggagatatagttcaattcaaaaggtacactgatgaagctaatgtaatcaattatttttttctatactacggaagatggagctcttggaaaaaaaaatgatttctagttcattgtcattttctcttaagagtcaaaagtatcagtctgtgagacttttcaggagaactagatttgtatgtatacaacagagcttgccagaccactccattataaacaagaccttaaaaagctatggagtggccaagtgtggtggctcatgtctatgatccctagtaatttggggggtcaaggtgggaaaatcacttgagtccaggagtttgagaccagcctgggcaacaagtgagaccctatctataaaagaaaagagagagagagatggcattaaaaaaaaaaacattcttttgaaacaagtatatgcatttccacataaatttcaaatgaaaatgaaattaaaaactgtgtcatacctggtaaagtctggtggaacaggagtggtaacaaaagatgccatgggcttccgatgaacttgctgaaacatcataaggatctctgagctcttagatatcaactcactcttactacaagaccgcaactgtgtgaggaaaaaaattatctgaacaaacaaaattactattcactgccacctcaaaattcttaagaaattacaaattattctgattataaactgaaaaaaatgataaaaataactcaaagtgagcttgagaattaagaatgtacaaagaatctaggtttatttctataatattctaacagtaaagtcaatgaataagtatacttaatactaatgcatttttctctccttacattttaggataaatgaggttttaagcaagtacaaatatactttcaggctggtacagtgaagttcttatgaatgtattaatgaacaaaccggtaagttgaaagcttttttttttttttttttttgagacagagtcttgctctgttgcccaggctggagtgcaatggcgcgatctcggctcactgcaacctccgcctcctgggttcaagcgattctcctgcctcagcctcctgaatagctgggattacaggtgggtgccaccatacccaggtaatttttgtattttttagtagagacagggtttctccatgttggtcacactggtcttgaactcctgacctcatgatccacctgcctcggcctccccaagtgctgggattacatgcgtgagccaccgtgcccgaccaaggtgaaagcttttaaaagcaatttctcagattgtattctaggtgacagaatacaaaacgtacacacatacacccgtgtgtacacacacacacacgcactatctgatactaatttgggaaacactattttgggaaaaagtaacaaatctaacttcagtaacataaccatggtagataaaagataagactactaattttttcttcaaaaaaaccagtttgaagtaactcccaattctgccactgactaattacataactctgagtctgtttacatacaaaactaaaatagtaatatccaccactgcttacttcgtgaagtttgttataaggattatgaagctatagtgaaaaagctttgtaaactatctgttatgtgctgaattttgtgcccctttcctccataatttatatactgcaggctcaacctccagtacctcagtctatctggagataatctttaaagaggtgattatgttaaaatgaggccactggggtagggccttaatctaacatgactggtgtccttattaagaggaagagactctagggctgcacaaccacacagaaaagtccatgtgaggacacagtaagaaggcagtcatctacaggccaaggagagagacctcagaagaaacgaaacctgccaacacctgaatcttagacttccagcccctagaatactgagaaaacaaatttctattgtttaagtctcacagtcattggtattttgttatgacaggcctagcaagctaatatactatcaaatgccatatgaatgatattcataaacccacctacaggtgagtttatatcttttaaataacagttgcttaaggctcaccaaaaataacatatcatatggctgggcacaatagctcacacctgtaatcccaggactttgggaggctgaggcgggtggatcacttagggtgaggagttcaagacctcatgacctggctagttcaaggcctggccaacatggtgaaaccccatctctactaaaaatataacaattagctgggcgtggtggcacgtgcctgtagtcccagctactcaggaggctgaggcacaagaattgcttgaacccaggaggcggaggttacagtgagccaagatcacgccactgcactccagcgtgggcgacaaggcaagactctgtctcaaaaaaaaaaaaaagtatcatatatcatatgatatatatcaaagtttatagtcttgggttaccaagaataatgatatcatgtttgagctactgcatcaaccaatgacataatggtaagtggtaacagcagaaaacaacaaacagctgcatattttgtcattcatattattatgtgtcaggcattgcccaaggcattacggttttttgaacaatttatataaaatgatctaaagttatttgaactgtttgcttaagatttttaaggtatgctaaaaatcactgtgtatatgtacatacatacacacataatacaaaaaaatttaacttatattaattgaatgcttactgtgtatatccttccttgacaccatttcttccaaaagcaacttgctcccttcaacagaagtttattatcactatacatgtgtttccacaattactatgtgtgtacatacatccataaacaatgttctgaatgttttcaaactttacataaacactatcaggctatatcaccatgtagcctacatttttcagttaatattatttactttttttttttttgagacagggtctcgctctgtcgcccaggctggagtgcagtggcacgatctcggctcaccgcagcctctgcctcccgggttcaagcaattttcatgccttagcctcccgagtggctgggactccaagcatgtgccacctcgcccagctaagttttgtatttttagtagagacggggtttcaccatgttgcccaggctggtcttgaactcactcaagtgatctgcccaccttggcctctgaaagtactgagatcacaggcatgagccaccacgcccggcctatttctttacttttttaaacagatgggtcttgctatacttcccaggcgagcgccgtggctattcacaggtacaatcatagcacactgaagccttgaattcctgggctgaggcaatccttccacctcagcttcctgagtagctgggactacaggtgtacaccccatctcaatactgttttgttaaaaactaagacaatggcctaggcctacacatggtcaggatcatcaatatcaccaccttccacctccacatcttgtcccattgaaaggtcttcacgggtactagcacatatggatctacaatgctaacaatgccttcttctggaatatagtcaacgtttttttaaaaagtaggagtaaactctagaatatattctagaataatgaatggtatggtatagtaaatacataaaccagtaacacagttgtttattatgcttatcaagtattatataccatacataataatgtgttatatttttatacaactagcagtgcagtatgtgtgtttacactagcatcacaataaacacatgattaatgctttgtgctactacattacaatggctatgatgtcattagacatcactactaggcaacagaagtatttcagctccattttaatcttgtgggaccactgtcacatatgcagtctgtcattgaccaaaaggtcataatttgacacatgtttgtatatgggagatgtgtataggttatatgagaatactatgccattttatataagggacttgagtatctgcggattttggtatccattaaggtcctggggccaatgccctacagatatatatgtgtgtatgtaaaaagaaaaggaaaaaagaatggggataatgaagaaaaataagagtatttagtaaatatttatgtgctgaatcctacaagccaaattgtcaatcaagtacaaagataaaataaaaatattttcagacaatgcagtcaagaaaaatgtgttctgactacccttcctggaaagctactgagggaagtgtgatgttaaaacaaaacaagtaaaccaagaaagtggaaaccataggaatcaggaaacatgagattcaatacaagaaagatgtgaagggaattcccagaatgattaagataaaaatctcaggagacagctgtgtataaggcatagagagaaattacaaaaagcaaattccagacaagtgaatgtatcagatatttatgcaacactacatattttatttactcatttactatatgcttcatattcacccaccctaatctaaaatataaactctatgaggacagaattttaccattttgttcactgctgtatcaccaatatctacaatgatgcctgctatctatttaggacttaatatttcctcaataaataaaaatgctaaattttagagtaataatcgctacatgtaaagtaactggcaataatatctcaaagccaccacttaagtcacagtgtggggagattttaacctcttatacattcagatttctatggaaatagttctgttcttacaaagcagaatgtagttctaatgcagttcccaaacatgaagaaaaaacttggcaagaatcagctgattcctaatttcataaaaatctccattataacacaaaagatcaacagatcttttgatctcatacagataatctctgtcaaaaaaaaaacgtttataatatacagagaaggccgggcacggtggctcacgcctgtaatcccagcagcttgggaggctgaggcgggcagatcaccaggtcaggagatcgagaccatcccagctaacatggtgaaaccccgtctctactgaaaatacaaaaaaattagccgggcgtagtggtgggcgcctgtagtcccagctactcaggaggctgaggcaggagaatggtgtgaacccaggaggcggagtttacagtgagccgagatcgcgccactgcactccagcctgggcgacagagagagactccatctcaaaaaaaaaaaaaaaaaaaaacaactacagtatacagagaaaaaaggataaagactatttcattacatttaatatttcatcacctggtgctccacctcctgaagtaaggattccaaaagctctgtttcttgggttagagatgtcttctgacctgatgaaaaatagtgaatcatcaacacttgggttcattattggtttaagaatgaaattaacttccaatgaacatatttaataacaagaaatacacttcaagtaagcaaaaattcttcattacagctaccctaaaaattctcattcaactttaagaggctgaatattatagtagccttgaaaatggaacttcagaaccaattctaattagttttagacaagataccactgagatcaaattcatctaagaaacgaaatgtgcaatgacaatatactttaagcaaaaaatttacttttctagattttcatattcccttgggaagaagcaataacatgaatttcattcaatcagctattctgaaatcatttttcaccaaatttaactgtggcttaaaagagaatacaaagtgggccagatttttaggttaaatgtcagcaaaaggtctgtcctgtgaatatacaaggacctgtttaaaaaaaaaaatgcccacagaggtgaacagtctcaaaaagttggagacagaaaaggataaacaaattattactggccggggccatggctcatgcctgtaatcccagcactttgggaggccaaagcaagaggataacttgagcctaggtatttgagaccagcctaggcaacatggtgagacccagtctctacaaaaaagtaaaaaattagccaggagtggtggtgcatgcctgcagcccaatttactcaagaggctgaggcatgaggatcacttcagcccaagcggtcaaggctgcatgcagtcatgcaatgagccttgattgcaccactgcagtccttcctgggcaacagagtgagaccctgtctcaaattacaaacaaaaaaattataattacagaaattatccttatagaaaaggaagaaagtgctttatatgttttgaaacaaaaagaagataggtatgcttaccaaatcaggccattttctttgtaaacccaaaatttgaaaaactggaagtacatccatgaggacagcaaaagttttatcaatctgtagtgttatttgataaggaaaaacatttaataggtagaacttttatttattttatttatttattttttgagatggggtttcactcttgttgcccaggctagaatgcaatggcacgaactcagctcaccgcaacctttgcctcttgggttcaagtgattctcctgcctcagtctcctgagtagctgagattacaggcatgtgccaccatgcccggttaattttgtatttttagtagagatggggtttctccatgttggtcaggctggtctcaaactcccaacctcagatgatccacccgcctcggcctcccaaagtgctgggattacaggcatgagccaaggcgcccggcctttatttatttttatttatttttttgagacagggtctcattctgttgcccaggctggagtatagtggcgtgatctcggctcactgcaacctccacctctcaggctcaagcgatcctcccacctcagcctcctgagtagctgggactacaggaacacactatcacacctggctaatttttcttttttttttttttttggtagagatggggtttcaccacattgcccaggctgggcttaagagatccacctgcctcagcctagaacttttatttttgaaacaaaactcagaatttctaggttgcaaaaagagttaaaataaagtattagctagaatctaatattatcagacataatttctactataaccacatcccacaatcctaaaacattagcctatggacaacattacaatacataattaaacttaaaaatgagtggcagaggccgggggtggtggctcacgtctgtaatcctagcactttgggaggccaaggcagacagatcacaaggtcaggagtttgagaccagcctggccaatatggtaaaaccctgtctctactaaaaatacaaaaattagcagggcgtggtggcacgtgcctgtagtcccagctacttgggaggctgagatagaagaatctcttgaacctgggaagcagaggttgcagtgagccaagatcacgccactgcactccagcctgggcaacagaacgagactccatctcaaaaaaaaaaaaaaaaaaaaattagtggcagaggtcaggtgtggtggctcacgcctgttatcccagcacttcgggaggcggaggtgggtggatcacctgaggtcaggagctcaagactagcctggccaacatggcgaaaccccatctctactaaaaatacaaaaaattagctgggggtggtggcacgtgcctgtagtcccagctactcaggaggctgaggcaggagaattgcttgaacccaggaggcggagggtgcaatgagccgagatagtgccactgcactccagcctgaatgacagagcgagactccatctcaaataataataataataataataaattagtggcagatattatggtatgaattaaaaatctgaggcttataagactattctaaagataaactaaccctgaaaatatccttttccccacctcttatccttagaaagaatcctatttaatcataaaagctaccttggaaatcacaaattataatttttcataatgaaaaataatttgaaagccacaaaagcagggcaagttcagttattagaaagggaagttatttcacagttatacaggtagtaaaattaataaacttaagagatctgtactgtggtattatttgacttttagttgattcaattaaataaatgaagcaatgaacaaactcactcaagaaaaacaagaccacatgcaaatgtttatttggctacgactgaagatcacatcaacagtcaagatcccctcaattttaaagtcctaatacgtatcaacatccagtactctacatggcttaatgaacttaaagagatctcaaatattatctagagttctgaaaaactaagatcacctatatgtatgactgcaactgaggatctaagaacaattacgattttcatttacatgaaatatataagtggcattgtgaccagtacaatcaatacctacaggttttgtctaattgctccactgggtggatgaactgtgctttccaatctaactactagaagaatgattgtttaagactatttgctacttggtctgtgggaaaaaaactaaaaatcagatattaaatagtataggattataatagaaaagcaattcatgaaatgatatacaaatatacaaactcaaagatatttgcatctcatttttaaaaagttaaaaataaaatggataaaatcattattttggatgacacaaagacatggctaaaatcttggagaatcaagatagccaaaaaaaaaaaaaaaattctttgttcacaccttatttaaagtatatatatttcactttggactatgataaaaaatgcaaagctggtttttagtcagcaaaacactggaaggactaagatcaccaaaacaaatcaaacaaaacaaaccatagcattggattaaattcttacttaaaatttaagttcaataccaggctccacaatcaaagaagaatgaaggaatcttcgaaaagcaaaaaggtcagcaataaatataattaagcatatgtaaaatgaaccctactagaaatcaaaagctgagactttaaatctctaagaaaatgctggcaggggatgacgccgatgtttacaaatgtcagtttcttcatccccaatgaaaatggatggaaaggatatggaacattagtagaaggaattctgttagatttggagaaaaagctacctaacaatgatgcccactggccattaggattttcatccaaatggctatgaaaacttggattttcatccaaatggctatgaaaatttttctctcctatcatttctaaatacaagtctaacatttatctgtctaaattgtaacaacatggagttgcctaaaatctcaagattccttcctaggatgatcacccttattcttcaaagactgaatctcaaagaagctgaaagcaccaggtaccccagcagcatacataaacacttacccatcagtgttataagcttattcttcagctgtgtgtctaaccgtgcaatcatcatctccactgcattcctaatttcccgaacacgctcatcttttgcatttcttacagcttctacattcctttcctagaagataaagaggtaagaataattttaattgggtaaattttaaaaacagaagcaccacattataatattgccaagaatagataatatgaaaaaggtagttagaggaaggtcaaatggaaatgacccaacttgcttatatttcaaaatggcagacatgcaatcaaaaacacaaattttctgcataaagaatgttataaatgatcacttggttcttgattcctaaagaggtaaaacaaagaagacatctgcttttaatttaggatatggtccacaaacttcattttaagttaggaaatggtccataaacctcatttatgactgtcattctaacaacttgaaacaaaatgtattctaaaaacatgcttagtatttcattcttgggggcttatcacattttttcacagaaataacattataaatagctcactaaagctgtttgacctaaacaatatctaaacagtaatgttaaatattgcactttcaagtctgtggagttctaaatcaggacatcacaaatagttcattgcttagtaaaagaaactcacccaaaagctggggatccaaagagtgtataacttaatgaatgactgtaaaagtcaatactgattctgtttagaagtaaattattggggccaggcacagtggcttacacctgtaatcttagcactttgggaggccaaggcaggtggatcacgacgtcaggagtttgaaaccagcctggccaatatggtgaaaccctgtctctactaaaaatacaaaaattagctgggcgtggtggtgggcacctgtaatcccagctacttgggagggtgaggcaaaagaatcacttgagcccgggaggtggaggttgtagtgagctgagatcgcgccattacactccagcctgggcgacaagagtgaaactccatctcaaaaaaaagaaaaaataagttaattattgactctgtaacaaacagactttttaaattcactgattttctaattctgcataagtggtaattctctgactccaactgcttccttacaagccaggggctttgtatgcaaatttctcagatactctgaaatttatggaatttgactataggtacagcctatcaaatttgcaaatcttgaaaaccttgatttcaacatctgattaacttatattatataaatatattattatatattacagattaaaaacaaaataattatattttagtagtagtcttaccacttcttgaactaagctgatcagttccatgagacgccgacgaagtttggctacctcttcattcactttagtgacgtgttgctcataaatttctgccaaaggtttaaaggtatgtccgccatgctatttaaattagagtagctttagaacactttcacatatctcatctgcatttacattcctttgtaacactctatggacactaataaactggtaaatctctcaaatgaactttaccttattagctaatttaattttgttgaagcaggccagtgcccgcttaaaatcacttggtccaagtaggaactctttcaagattataaataaagggctttagcaaaatattcatttaactagaaattaacttattaaaaagaaccaagatctttaaccacagcccaaatcatctgccacaaccttatcagtcagtcactcttctctaaggcaaactagtcttcttccatgtcctcaaacttctttccaccaggactttacaaaaattactccctcttattaaaaaataattattagccaggggtggtggctcatgcctctaatcccagtactttgggacgctgaggagggcagatcacttgaactgaggaattcatgactggcctggccaacacggcaaaaacacgtctctactaaaatacaaaatgtagccacgcatgttggcgcacacctgtaatcccagctactcaagaggctgagacatgagaattgcttgaacccgggaggcagagcttacagtgagctgtgatcgggccactgtactccagcctgggtgacagagactctgtcccccccaaaaaataataattttttaataataaaaaccaaaaaaaaaaaaaaataaaaaaaaaaaaataataataataataataataatgctgggcatggtggctcacgcctgtaatcctagcactgtgggaggctgaggcgggtggatcacctgaggtcaggagttcgagaccagcctggccaacatggtgaaaccccatctctactaaaaaacacaaaaattagctgggcgtggtggcatgcgcctgtaatcccagctactcaggaggctgaggctggagaaccacttgaagccgggaggcggaggttggagtgagctgagatcaccccactgcactccagcctgggtgacagagtgagactctgtctcaaaaaaacaaaacaaaacaaataataataataattctccatcttccctctcccatcttggtttgtcttaatcctactcatcctttgcacttagctcagatttacaggatcagctgctatagttatgcatcctcatcagctctctgtatttattcctataatttatcaagccaattgagtatgtaattgtttgatatgttcatcaccaaatccctagtctttaccacagctgaatgaattagaaagacacttgaagaaaaagcccaggatacaaaattaacaaaagcaatgaacccattttccatcccacagagttactacagagacagaagactgatacaagttagcctcacaaatccagttgcattcagctcaaagaacaatttttatagctaactacctaaaaataattttgatttgtaaccagaattattaaattttaatataagaaacctcctataagttcttactagaattgtcttatttagccaaagtatcccattattgtctccaaaaaagatgtagatttttacatgttcactttaagtatagaaaaaaacatttctactgtctcttacaaaacctatgtatttaaagtattaaatgtgagaacttaattttttctagttagcacattaatattcaaattctagcagaaagctctgaattgagctctactcaaccaatctggcctgccactctctccgtaaaacatactgactgatgcccacagaatgatcaattcttatagcaactctagatacctacccacttccatttcacccataacttatttttcctaagaatctattgtgtttattctcaacctatttatgccaattgtacttattattgtaaggagatcatttaattaagtattcagaggctgggcgcggtggcttacccctgtaatcccagcactttgggaggccaagtcaggtggatcacctgaggtcagcagttcaagaccagcctgaccaacatggagaaaccccgtctctactaaaaatacaaaattagccaggcgtggtggtggcacatgcctgtaatcctagctactagggaggctgaggcaggagaatcacttgaacccgggaggcggaggttgcagtgagccgagatcccaccattgcactccagcctgggcaacaagagcgaaattctcaaaaaaaaaaaaaaagtattcagtaaacaacagaacacgactttaaaataaaaaagctgcttctaagaaagctaacttcaatgcttcaggctcaataatggtagactgtctttaaaaactgctcttgaattcaattggaaaaaataatctaaaaaataaaaaataattataagatctagataggcgtactcatgttgtttcacaagtgtctaagttcgtgctccattttaaaggaactaaaattggaaatgttggccagtacatctgatcaactgacctttaatgaaaaaagtgacctagatcctacaacgaaaagatctgcaaatgaatagaggatatgttttataggtgttacagattaaaatatttacataaggggtccatcaaaccacacaaacaagattcttctggaataaaagtacaatacctttaataaagcgtatagaaagcagatgcattttaactttcattctacgagagcataatgacactattttctatctgaataagtatttcagtgccttctagcctctaacttaaaaaaaatactgaatttgttctgctcaccattcctccccaaagtgcacactgatggcagatacacttcttacaagtccagcaaaatacactaagtttttcatggtgattttcacatctatacattatgaaaagaaaatgaagttataaattaaattggaacataatcacctccacaaattcttaagcttgggcaaacaggtcagttttaaatgattatatctcaagaaacacagtataaaatgtactccatggcttctgaaaaggaagctaaagaagtttatgcggacaattttttgtaaagtactgttttcattgatctggactttataatcacatgcgaaatacagatttgcttcttaaaaacataacacatattgcatcatttccacacagcactacagatgatatattctattgtactactacattttgggcatgtgcttaataatcatattgattatgcatctatacctacatgagcatgcacaagtacttttcctacatgtggtcaacgataacaataatcaagacacatgattaactgcacgatttaatactagaccctcatggaaaatgaacaactcaattttaaatgttcttttaaactggatattcacatgaaaaacaataaagttagactctgttatggattaactatgtcccccctcaaattcataggttaaagccttaacccccagtgtgaatgtatttgcagacagagcctttaaggaggtaattaaggttaagtaaggatcacaagggcaggacccaaatccaacaggactggtgtcctctataaagaggaggagacaccaggcatgcacgtgcacatagcaaagatcatgtgaggacagagctaggtggtggccatctataagtcaaggagaaagggaggcctcactagaaaccaacactgctagcatttaatcttggacttaacacctatcagaactgtgagaaaataaatttctattgtttaagctactcagttagcagtattgttatgaaagacctagcagactaatacagaccctcactttataccatatacacaaataaactacaggccgggtgcagcggtggctcatgcccgcaatcccagcactttaggaggccaaggcgggtggaacacttgaagccaggagtttgagactggcctggccaatatagtgaaactccttctctactagaaatacaaaaaattagctgggcgtggtggtgcactcctacaaccccagctactcagaaggctgaggcatgagaatcgcttgaacccgggaggcagaggttgcagtgagctgataatcacaccactgcactccagcctgtgcaacagagcaagactctgcctcaaaaaaaaaagaaaagaatgaaggtttaacatccagaatatataaataacacctgtaactcaacaaataaactttaaaaactctaatttaaaaatgagcaaaagatctccagtaagcatatgaaaagatgctcattactactaatcactagagaaatgtgaatcctaacctcataccatattaggatggttgctatcaaaaaaacaaaaaagagcaagtgttggccaggatgtggagaaactggaacccttgtgcactactggtgggactgtaaaatggtacagtcactatggaaaacagtatggagattcctcaaaaacttaaattaaaaatagaattaccacatgatctagcaattctacttctgattacataaccaaaagaactgaaagcagagtctcaaagacatgaacatccatagtcatagcagcattattcactacagccaaaaggtggaagcgacaccaattttcactgagagatgaatggacaaaaaaaatgtgacatatacatacaatataatattattaacccttaaaaaggtgggaaattctgacacatggatgaaccttgaggacattatgctaagtaaaataaaccagttacaaaaagagaggtactgtatggttccacttatatgcagtatctagagtactcaaacagatagaaacagaaagtacaatggtggctgccaggggctggtgcgaagaaggaatggggagttgtttaatgggtacagtttcagatttgcaagtttaaaaagttctggagattggctgaacatgtgaacatgcttaacataactgaactgtacattaaaaaatgattaagatggtaaattttatgtatattttaccataattaaaatgttttaagtattctcttgtttataaagtagacatactcttgcaatttttttttttttttttgagacggagtctagctctgtcatccaggctggaatgaagtggcgcgatcttggcccactacaacctccaccttctaggttcaagcaattcttgctgcctcagcctcccaagtagctgggattacaggcacatgccaccacgcccagctaatttttgtatttttaatacagacagggtttcaccatgttggccaggctggtctcgaactcctgaccttaggtgatccacccaccccggcctcccaaagtgctgggattacaagcatgagccactgtgcctggcccactcttgcatttttcaaaaagcatttcattgatgaaggacactgagcttagagaaaatacttacataagtttatattttatatttctaattcataagtaatcagaatataaaatgggaaatgaataagagttagaatacatcaggtactgtttggaagctcatttagcaagacttccaaaagaccaaatttataatatagcttttacaatatagaaaatttgccaaaactcctcttaattaacagcctgtattcacttgcagcaaggggaggccagtaggcatcaaacagcaagtaagaaagactagcaggttagcaaattgtagcagtctattccttttgagaggctctaatagaagaggcccaaaaagaggtaaaaggtctttccaaacagtaatacccaccacttcagaggaaaaacacctaagtgttcaccaaagaaatgtgccattgtatagaaaagaaattaaataattatttttggtgagacctctacaatgctacaagtgacagaattccttaaaagtccagttaaacttgaatagcaggtttttggttttgtttttgtttttttgggagatggagtctcgctcgttacccaggctggaatgcaatggcacgatctcggctcactgcaacttacgcctcctgggctcaagcgattctcctgcctcagccccccaagtagctgggattataggcgtccaccacgacgcccagctaatttttgtatttttagtagagacagggttctccatgttggccaggctggtcttgaactcctgacctcaggtaatccacccacctcggcttcccaaagtgctgagattataggcatgagccaccacacccgcccgaatagtaggttttaattctgattatgcattaatgtgttatactaccataattaagcctctttctttttttttttttttttctttttttaagagatgggttcttgctatgttacctaagccatcctctgggctcaagagatcctcctgcctgagcctcccaagtatctgggactacagatgcacaccaccacaccctgttctcttatttctatggttctatttcctcagtataatgaagaaacagataaatttttttctaatgacctgtgaggtttcaacctcttgctttaccatgttgtgactctacagaaagtgacaaggctttatttctaaacattcaggggtcctggaatctcacctaattcaagttattttattattgctggacagtgaagccttacaattaaccatgttgtagcttcttcatctataaaaaccaatcactgccacatcactctttcctttctttacctgctccccataacaactctccagatatgttcaaaattcttcaatcatggaacataagaccatttattcattcttgtcccttaaatgacttaacactgtattatcactgtgtccccattaaacagttaacagaaatcatgtctcatttacctttgtatgcctatcatctagcaaagtactttctgtgtggggggaggggagggcgagaggtatctgtgtatacatggcatgaaataactgttgttaaataaacacaataagaatatgttacttaacagtaacagcgcatgactgaaagaattaaaaacaagaaatataacactacttattcataaataactaaaaggcaactaccaatatagtgtcattaaacaatacaaaggcaaaatccattcaatattgaacaaagaaattgaggacatacttgtccttttcattttcttcatgtttggtgagactgcagagttgaagagtatcaagctgttgtgttacttcttctgcccaacgacaatttactagttctcgtagctggagtggagcactggggagaaaatccatacacatacactaattcaggaatttgagattattttataaaataaatacgtttctcaaaatagagagcacaataatactcataccataacactattttttttttttaagagatgagtctcactctatgacccagggtggaatgcagcggcatgcatgatcatggttcactgtagccttgacctcctaggctcaagcgatcctcctgcctcagcctcctgagtggctgggactacagcagcacaccatcaaacccagctaattttaaaattttttgtagagacgggaatcttgctatatttcccaggctggtcttgaactcctggcctccagtgatcctcccaaagtactaggattacaggtatgagccactacctggccccataatactattaaaactaaaaaaataaaaaataaaaaaaaagacaatgaaaaatattatctgtgacttattttcaccacattaacacgtcatttatgaaaagaaacaaaaagctaaacaaaaacatatgattcagaatatggactgaattctcaacttctgagtctaaagctgtactgttggcgtagtggctcgcgcctgtaatcccagcactttgggaggctgaggcaggtagattgcttgagcccaggtgttcgagaccagcctgggcaacatgatgaaaccctgtctctataaaaaattagccaagtgcaatggcatgtgcctacagtcccagctacccatgaagctgaggtaggaggaccacatgagccagggagattgaggctgcagtgagccaagatcatgcctctgcactccagcctggggaacagagtgagagcctgtctcaaaaacaaaacaaaacaaaacacaaatccacagctgtgctgtccaaaacgatagccactagccacatgtgactcttttaagttaaaattaaataaaaataaaattttggccaggtgcagtggctcacacctgtaatcccagcagtttgggaggccacagcgggtggatcacaaggtcaggagatcaagaccatcctggccaacatggtgaaacgctgtctctactaaaaatacaaaaattagctgggcgtggtggcacgtgcctgtaatcccagctacttgggaggatgaggcaggagaatcacttgaaccagggagtcagaggttgcagtgagccaagattgcgccactgcaccccagcctggcgaaagagtgagactccatctcaaaaataaataaataagtaaaatttcatcatatagccacactagacacatttcaagtgatcgacagccacacacaactagtggctaccatactggacagtgtagacatagaacactttcatcattataaaaagttctacttgataatgctggtcttataagataatgtaagaaaggtgggacgaggcacagtggttcacgctgcaatcctagcacttcgggaggtcaaggtggtaggatcccttgagcccaggaagtcgagaccagtttggccaacacagtgacatctcatctctacaaaaaataaacaaaattagctggcatggtagcacacaccagtggtcccagttaggaaggaggttgaggtaggaggatcacctgagcccaggaggtcgaggctgcagtgagctgaaatcacgccattgcactccggcctggacgacagagcgagactctgtctcaaaaaaataaaaagaaaagtgaatacaaaaagaaaaatggttaacagatggcactccagcattattttttgaaaccaaaagtagagataacctaaatgtctctcaatatgggcataaataaatcaaggtatagtcataaaactgattatcagtcacttttttaaaaaatgacttatgtatacatacaatcaagaaaagatatctaccatattttacatgaaaaagcaaactgcaaaacaatattatatcattggtacattttaagaaaaagcatatgggaatcacatacgcatatatatatataaatataggcacaatcatactggattagggcattacaaaatacatgcacaaaaaacagtctagaagaatagataccaaacccaacaatttcttctgaaagtattattaagtgagaagacagggtagggagtatcagaatcagaaggctttttattttattttatatttatctctaccatctggatttttttgtttgttttttgtttttatttttttgagacagactctcgctctgtggcccaggctggagtgcagtggcgtgaccttggctcaagtgcagcagtacgatctcagctcactccaacctccacctcctaggttcaagcaattctcatgcttcagcctcctgagtagctgggactatagtcgcgcgccaccacgcccagctaatttttgtatttttagtagagacaaggtttcactatgttggccaggctggtgtcgaattcccgacctcaggtgatccagccgccttggcctcccaaagtgctgggattacaggtatgagcgagccactgtgcccggccaagctatctggatttttttaaccagaaaacattaaaattttttgcttaattataaaagataaattatgccctaactatctggattttttaaccagaaacattaaaaatttgttgcttaattataaaaggtaaattaagaagttatacaatgattttctgagaggcagagtacaacatagtaaacgtaattttaagtcacatgaattagtccctgcatttctttttctagtcaaattgcttatttctactcaactatcttcacgaagaagagaaatgggcagactgcaggaaaactgcctttatgaatcccaaggcacattctgatcttactattttcaaaattcacaaataatcgtaaggaaacacttaccggcaatgaggacattgagctctctgctctgtcagccagcgctaagggggcaaaagattagatatcgattagaaaacatcattactatatatatatattacttaatattttatatttatatataatatatataatattcttaatatatataatatataaatatataatattcataatatataatatataatatattattatataacatatcatatatatataatgtataatgtataataatgtataatatattatacattatatatataatatatataatgtataatatattatacattatatatataatatatatataatgtataataatgtataatatattatataatatttatatatataaaatactacaaatctaatgggtttaactctctagtgcttagttttagagatacttataaaagtaacaggcaatctactgtttccctcactgtcatcctcatagggataaacttttgaagggacaaaactcaacacaaaatagctgttaaaaggctactctcacacaggaaaagcaatgatttcaggactaactgattctcacagggaacgtctatataatcttattattgtcttataataatggcactgtctcctttgcactcaaaaataaaccacagtgatgatttatttctatctagtttactactaaatttattttttgaaaagctaattcgaaaaaaaaaaggaagctaattctgagttacatacattatagacatgtaaattaaaccgaaaaaaatgaataagcaatacagcacaacttttaagtcttaaaacagtttcacagggctgggcacggtgtctcacgcctgtaatcccagcaatttgggaggccaaggtgggcagatcacctgaggtcagctgatggagaccagcctggccaacatggtgaaaccccgtctctactaaaaacacaaaaattagccaggcatggtgatggtggcaggcgcccataatcccagctactcgggaggctgaggcaggagaattgcttgaaccaaggaggcagaggttgcagtgagccgagatcctgccactgcactctagcctgggcaacagagcaagactctgtctcaaaaaaaaaaaaacaaaaaaaacggtttcatagttcagtaattgaagcttctagatatcttggtttcctcataattataaaaaggttttagaagagaataatgtctctactaaaccaaaaattatattaataatttattaggaaactgagagcaatgttataccacatataaaggagatgttaagattttctgactagttttcaggaaacaacaggtttataataaaaaaaactttatgttttaattgtctgataaataggttcagaggtaaataactatgatcctacaccaacgtactcccaatatctgttatttgtttatctctttctactttttaaaatgaacatgagctttaacttgatgccaaataattaacttagaaataaaattcccaaggaaaccttaaaggacagtaaaagacttaaaatgatagcaagcaaggtacttcagagcaaagaaaataaagctgaaagaaaaagcatacagagcacgcctgtaatcccagcactttgggaggctgaggcgggtggatcacgaggtcaggagttcgagaccagcctggccaacatagtgaaatcccgtctctactaaaaatacaaattgagccaggtgtggtggcatgagcatgtagtcccagctactcaggaggctgaggcaggagaactgcttgaacccaggaggtggaggctgcagggagccgagacagcgccacggcactacagcctgtgtaacagagcatgattctgtctcaaaaaataaaataaaaataaaataaaaagcctatggaaaaggattcctccattcttaataaccttgaacattattattaccttggtccctaggcaaaaacgaaatttgggtcaggtttcttcccctgaaacaatggcatgaccatgccactcttctgttaaagtcacagctcctctccagaactggtctcatgaactcctctccagaactggccctataacccagcaaggtaatgtctttacatgtttcctctctctgcctaagatattcttcaaaacctttcttcatctgggttactcctactcattgtttagatttcagttcataagttgctttctccaaaaagctttccctgacactctgcagactattttagatacaattgccatgctctaccacagcaacctacacttcccccttcagatactttattacgacgacttatttgttggtctcctttgccctgctagactataaaacttataaagactgggactatgatagggagtaaccagatcgtattttaaatgtctccctccctcccagtcaaaacaggcttactgctgcactatataacactgtcgatatatctgtgcatttgcttatatggtccctccctccatatcccgtctcatagaaatgtcactggatcatgtccataaaatttcctgcctttcccaggctatagtgagttttacattctctgaatatttgtggtatttttatcttgtattccacaatctaatttatttatttattttttgagacagagtctcactccctcccccaggcttgagtgcagtggtgcaatctcaactcactgcaacctctgcctcccgggttcaagcaattctcatgcctcagcctctcaagcagctgggattacaggcatgcgacgccacacccagatggttttcgtatttttattagagatggggttttcccacgttggccaggctagtctccaactctgacctcaagtgatctgcccgcctgggcctcccaaagtgctgggattacaggcatacaggcatgaaattgctcagcctccacaatctaatttaattacaaaataaatatgacaatgttaatactttgagaaaagcaatgtttctctcaaaccaaaaccaaagaaccagtggtacaatattaatgcataatattaaacaaatatctttagagcattccccctcattcaattaaaaaatcataaacataaaataaaattaaaattaaaaattattttctctccttcattaaactggaagaatcacaagctttagaaagtattcagatctgtgtctgattcccagctctggcacttatggaccacatgacattggacaagtgactgagctcgtctcctcatatgcaacaagagaatcaacatatctaccctgcaaaaccgctctaagggttagagagaagctgttagcaatatatactaagtatctaagataatcttaaaaaaaaaacaaactggctgctaacatttatagttcaattactactggatgtgaggaaaaaggaagattaaggtgagctaggtttttggataaaaaactgggttcctgactggaaaagaaacagatttggcatggtgaaaaacaaaaacaaaaacaaaaacaaaacaaaaaaaaacctcttatagagacgatatatttcagatgcatactacaagtccaaatgaaggtagttagatactggaggcagagaaaaggccaggactgaaaagataaagttgggaagtttaagtctagaggtagtgttaaaagtcatggaccagtcgggtgtggtggctcacacctttaatcccagcactttggcaggccaaggtgggaggattgcttaaggccaggagctccagatcagcctgggtcacatagcaagataccatctctacaataaatttaaaaattagccgggcatggtggacacgcctgtagtcccagctactcctcaggaggctgaggtgggaggatcaactgagtccagaggtcagggctgcagtgagccatgatcatgccactgtctctagcctggatgacagagcaagactcggtctcttaaaaaaaaaaaatccatagaccatgaagttactaggaagatggtatagctattatggcaaagataccaaagataacgtttcaagaaaaacagatgaagtaatttatgctgaatgctccgagaaggaaagataaggccaacaaaaggttcattggatttagcaaaactgagtctacactttgcaggagctatgtgagtagtgtggagggagctaaagccaagttagaatgggttgaagggtaagtagatggcaaggttgcagggaaatggagtggaaaacactcttttaaatacttttagctataagagaaaacaggcagtaaataggaaatttgttaacaaaggacttttattcccaaacagctcaaaattgcaaggaaactaggaggccgatcatagaggatcttagatgccttgtgaaagcatacaaaatttcttacatcccctaatgtactgtttgaggcaaactatatatacatacaaaaacatgctaaatactttctggggttgtttttgtttttgagacaagtctcactctgtcactcaggcaggagagcagtggcccaaacacagctcactgcagcctcaacctcttcatctcaagaggtcctcccacctgaccctctgaagtagctgaggctacaggtgtgcccaccacatccagctaattttttaattttttttgcagagatgggatcttgccatgttgctcagcctggtcttgaagtcctgggctcaaacaatcctccatctttgggcctcccaacgtgctgggactacaggcatgagccactgtgcccagactaaaatagtttcttacaaacatctataccactaaactataacaaaagtccacttcttatggtatgtctaccaaagaaagggaaaagctgtccttttgttcatttccaaaaacctgtaagaatgagtaattatcataaagattgtatatattaattatctagcatacagaactaaataagccagatggtaaataaaattactgttagtgtaacaggagaccaaaaaaagagagtttaaaaaaaaaacatcagccgggcacagtggctcacgcctgtaatctcaatactttgggaggccgaggtgggtggatcacctggagtcaggagttcgagaccagcctggccaacatggtgaaaccccgcctctactaaaaatataaaaattagccaggcatggtggtgggtgcctgtaatcccagctactcgggaggctgaggcaggagaatcgcttgaacccaggaggcagaggttgcaatgagccaagatcgtgccactgcactacagcctgggtgacagtgagactctgtctcaaaaaaaaaaaaacaaaaaaaaaaaaaccaactttgtaacagaaaacaaggtctttaagaaacattaattacagcaattattaaaaatcttagatcatgggctcatgattgattaaatactatttccaatgcctaatcctgcatttgaaattaattaaaaatataagtatttattaaattccttccatttcttcagaatataatcagagatatcattattgacatgtactgtgcatcctcattcataatgctatatgaaaaattttaatgtatatgtggtctgggagtttaagatatttactagagaaattgtgctagggaaatcctcaacaaaatcccaataaaactaattcaacagactatttaaaaaattatacactgtggccaagtgggatttattcctagaatagaaagatgattcaactacaaaaatcattcaatgtaataaaccacttcatggaaaacccccactatgatcatctcaattgatgcagaaaaggcatctgacaaaatccaatatcctttcatgataaaaatactcaatagaaatagaagggaacttcctcaacttgataaaggccatatataaaaactcataaaaatcatagtcaacgattcaaaggctaaaaactcttccccttaagatcaggaaaaattccccttaagatcaggaaaaagacaaggatgccagcttttaccatttctatttgacataatacaggtactaaaaattccagccagagtacttaggcaagataaagaaataaaaagtatcctaattaaaaaagaagtgaaattatctttttgcagatgacatgatcttatctgtaaaaatccctaaagagtccacaaaatattgttaaagctaataaacacaacagttacaggagacaaacacacaaaaaacactggcatttctataagcttgcaatgaacaatctaaaaacgaaattaagaaaataacacatttgcaacagcatcaaaaggaataaaatgcttagggaaaaaaaaaaccaagaatttgaaagacatacatggaggctgagtgtggtggctcacacctataatcccagcattgggaggccaagacaagagggtctcttgaacccaggaagttgaggctgcagtgagccatgatcgcatcactgcactccagcctgggtgacagtgagatcctgtctcaaaacaataaaaaacaaaaagaaagacttatacgtgcaaaactacaatacactgctcaaagaagttaaagaagtcctaaataaatggaaagacacccatgttcaaggatcataagatttaatattgttaagacagcaatattactcaaaggcatgtacagaatcaatgtaatccctactaaaactccaatggtactttttatattggtagaaaactgcatttaaaattcacatggaatctcaagggatgccaaatagtcaaaactatcttgaaaaagttggaggactcacacttcctaatttcaaaacttactacaaagctacagtaatcaaaacagtgtggtactaccataagaacagaaatagagactaatagaacagaatagagaggtagaaataaacaaaaatattttatgattccatttatatgatgtacttagaatagtcaatgtaaagacagaaggtagaatagtggttaacaggggctagggggaagagaataatgagaatttattgtttatcaagtacagagtttcagtttgggatgatgaaaaagtactaaaggtggatagtggtgatggctgcacaacaatgtgaatgtacttaatgccgctaaacagtacacttaaaaatacagttaaaattgcaaatttggctgggccaagtggctcacatctgtaatcctagcacttgggaggccaaggcaggcagatcacttgagcccaggagtttgaggccagctttggcaacaaagtaaaaccctgtctctacaaaaaataaaaaaattagccaggcacgatggcacacacttgtggtcccagctactcaagagtgtgaagcaggccaactgcttgagcccaagagattgaggctgcagtgagccatgattgtgtcactgtgctccaacgtgggcaacagagtgagacactgtctcatttaaaaaaaaaaaaaaaaaaaggctaattttacattaggtatattttaccacaattaaaaaaaaattaacagaataccatatgacccagcaatttcacttctagatatatattcaaaagaaatgaaagcaggaactcaaacagataattgttaacaacagaactattaacaatatccaatatccaaaaagtgaaaacaacccaagtgtccatcaagagataaaaatggataaataggctaggcgcggtggctcaacgcctgtaatcccagcactttgggaggccaaggagggtggatcaccaggtcaagagatcgacaccatcctggccaacatggtgaaaccccatctctactaaaaatacaaaaattagctgggtgtactggcgcacacctgtagtcccagctactcgggaggctgaggcagcagactcgcttgaacctcggaggtggagggtgtggtgagccgagatcgcacgattgcactccagcctggcgacagagcgaggctctgtctcaaaaaacaaaaaaacttaagaaaaaggataaacaaaaagtggtatatagccatgtgtcacataaagatgtttcggtaaatgatggaccgcatatactacagtagtctcgtaagattagaatggagctgccctacataggtgtaccattttttatcttttatactgtatttttaatataccttttctatgtttacttatgtttattttcttttttctttttttttaatgggtacagactttctgtttggggtgatgaaagattttggaaatagtgatatggttgtataacattatgaatgtaattaaagacaccgaactgtacccttaaatatgggtaaaatggaaaattttgttatatttattttagcacaaaatggggggcactgaacagaccccttaccaaagacaacatatggatggtaaataactcctggaaaagatgctaacatcattactcaacagagaaacgctaactaaaacaaggagatacctgtaagaatggctacacgtccctgagaatggctaaaattcacaaaaccgaacacaccaactgttagcattaatgtggaggaattggaactctcataaacttctggtgagaatgtaaaattataaaactatttccttttttttctttttgagacagagtttcgctcttgttgcccaggctggagtgcaatggtgcgatctcggctcaccacaacctccgccttctggattcaagtgattctcctgcctcaggctcccaaggagctgggattacagacatgtaccaccaggcctggctaattttgtatttttggtagagacagagttctccatgttggtcaagctggtctcgaactcccgaattcaggtgatccatccacttcggcctcccaatcctttttttaatttaaccttcatccctctcctcctctccctcttttgaagtccccagtgtctagcatttccccctttatgtccatgtatgctcattgtttagctcccagatacaagtattttagaaaatagcttggcaattttcaaaatgttaaacacacatctaccacataatctctatccgttccattactaattatttacccaagagaaaagaaagcttcttgtacacgaatgttcgcagcaattttcactgtaatagcaaaaactgtaaacatgccatatgcccatcaacaggtgaatgcataaacaaattgtggtaaatacatacaatggaatactacttagcaataaaaaggaatactactgatacatacagtatttttgctgagtgaaaaaaagccagatccgtaagagtacatattgtatgattttcatttgtataaaattccaaaacatgcaaacttatcttttttctctttttccatcccccttatctctgaagcaaacttatctatcgtgacagaaagcagatcaatggttgcctggggacaaggcaagtagagggaggaattaccaagaggcacaaggaaacttttggggatgacagatatattcactaacttggatgcaatgattgtctcacaagaatacatatatgtcaaaactgtacacttggccaggctcagtgactcacatctgtaatcctagcactttgggaggctgaggtgggtggatcacctgaggtcaggagtttgagaccaacctgaccaacatggtgaaaccctgtttctactaaaaatacaaaattagtcaagcgtggtgatgcatgcctataatcccagctacttgggaggctgaggcaggagaatcactcgaacccaggagatggaggttgcagtgagcggagatcgcgtcattgcactccaggctgggcaacaagagcaaaacgccgtctcaaaaaaaaaaaaaaaagaacaaaacaaacaaaaaaactgtacactttaaatatgtgtagtttactgcatgtcagttatacctaataaaaaatgactggcttgaaaacaagtaggcaagcaatcaatatttgctgaatgaatgaacaaatgattagtgcattcaggaaccaagacagccctagaaagaaaggtcagaaccagatctggatggtctgaacacaaggctaacattcttggattttatcctttacataatacatttctgaggcttctggggaaaagaaagagatatcagtgaaaaatattaatatagtagcaggacagaaaatggactggagataagaaaagactcaaacctgagagatcagacagaaagtaattaaataatctaagcatgaggtaaccaatgttcatacttctctattagcatggtaatgataaaaacagaagggtaagacaaatataagtgaaacagggagataataactaattgcacttaaaaatcattggtataggccagaggcagcggctcatgcctgtaatcccagcactttgggaggccaaggagggaagatcagttgaggtcaggagtttgagaccagcctgggcaacatagtgaaaccccatctctacaaaaaaaaaaaaaaaaaaaaaaatatatatatatatatatacacacacacacacacacacacacacatatatacatatacatgtttatatatatacatatatacacacacatatatacatatatatatacaaaaatagccgggcatcatggagtgcgctgatactcccagcaacttgagaggctgaggcaggaaaatctcttgaacctgggaggcggaggtcgtagtgacccaagatcacgccactgcactttagcctgggcaacagagcaagaatccatctcaaaaaaaaataaaaaataaaaaataaaaaaatcattggtataaagtttccagtttgagtgactagcagaacagaacaaaaagaatgaacttagaatcggtttgagaaaataatatgaataatctgatcttgaacaaagttcaagatgctagaaggacaagacatccaaaagtcagtgctgaaggaatagatttaggagccatacacacagagatgatagctaaagctgctaaagtaaatcaaatttcccaaagtaggtgaatactcccagaaacctcagagtagagaacgacagcaacaagatcttaggaaattaagaaataaatgcatttaaggggcaggaggaagaagatgtaacaatgaaaaaatacagaatcaatgaggcataaggtaacaaccagagaccataagcacatgggaccttcatcaaaagaaatgcgcccagatccagattctaccacttaattaactataacggagtccaattacttaatttatctagcatcagtttcactgtttattaaaacatgggtaagtgttaatgtgtattagttgctattttaacaataaaataacaccttacaaacaagtagatatagtatttcataatttctgaattactccttaggttatctcatttaatgccataaccaccctgtaaagcaaatcatatctgaattctacagctgaagaaagaaggccaagaaagagtgaatctcttagttttagaagtatgagaactaacatcatatttaactgggtcttctgcgcatggtacagtgacaaagaacaggctatagagttcttgaacttaatgggttcaaatccagtaccatcaattgtgagctgtgtgactagaaaaaaattatttaagtgttacggataatactcttgacaaaggcatatgtatcttccttaggcaagaataaggaaacagaaattttttttttttttgagacgaagtctcgctctgtcgccaggctggagtgcagtggcaccatctctgctcactgcaacctctgcctcccgggttcaagtgattctcctgtctcagcctctcgagtagctgggactacaggagcatgccaccatgcctggctaatttttgtatttttagcagagacagggtttcaccatattggtcaggatggtctcgaactcctgacctcaggtgatccacacacctcagcctcccaaaatgccgggattacaggcgtgagctacctactgcgcccagctgaaacagaaagtttgaggtgaagagcatgtgacctctttgattttctcaggaaaataaataattgaaggggaatgtgaacaaggcagttttcaactatgctctgtagatgtgctattggactctcgcatacacaaccaaaatcttggacttcctcattttgttattttgttttttgtttttttgagacagagtctcgctctgtcacccaggctggagtgcactggcatgatctcggctcactgcaacctccgcctcctgggttcaagcgattctcctgcctcagcctcctgagtagctgggattacaggcacacaccaccatgcccagctaatttttgtatttttagtagagatggggtttcaccatgttggtcaggctgttctcgaactcctgacaccatgatccacccgcctcggcctcccaaagtgctgggattacaggcctgggccaccgcgctcaggccaacattcaagttttttttttttttttgagacggagtctcactctgtcgccaggctggagtgcagtggcgctctctcggctcactgcaacctctgccctcccgggttcaaacaattctcctgcctcagcctcccaagtagctgggacacaggtacatgccaccatgcccagctaatttttgtatttttagtagagacagggtttcaccatgttggccaggatggtctcgatctcttgacctcgtgatccacctacctcggcctcccaaagcaccaacactcaagttttaaagccactagattatattcatctctagagtgtcttgtaattgtaagattaaacactttttctctatcttactcaccaaacacatataaaccagtcctcttctgagccccacagcacttctttagtgcctccttatgataccgcacattcttcaaaataaacattctaaactccattccctcaattagatttaagctccttcaaggcaggcaatttgtcatttatcttagtattcctcaaccccaagcacagtgcaagcaagcactttagggcaaaaaatgtaaaataaaaagataagggaaatggttaatccttacaatatatactatatatactaactgcatgtaaaaagaaatacaacttaccctaatacagctgaaacaacacagtttggagcaatgaggacacaggcgtgcatcccgcaatttctccatacaaatgaaacatcggaaaacctcagcaatgctctgaaaacagtaaaagatgtaaggtccaccagtttaggctactgaatcaagagtaaaaggcaagctcaaaacaacttgacatttacatttattttgggctgatctctcaattttaactattccatatcaggatttggttggtcaagatacagagtacctcaaaacatctggaaaatgtactcccgtttggtttcacgatgattctattctaaggaaaatataggaaatgcaaacacagacatgcagaaaaattttcactaaagcagtaattataatggcaagaatctaggtacagcccaaatgtccaaatattaagtaaattatggcagttacacatagattacagtcaccagaaatacttcttcatctaatgatatagaaaaatgcatataatgtataacagcagggaaaaaaggcccaggcatgatggttcattactgtaatcctagcactttgggaggctgaggcaggtggatcactcgaggtcgggagttcgagaccaacctggccaaaatggttaaaccccgtctctactaaaaatacaaaaattagccaggcgtggtggtgtgcacctgtaatcccagctacacaggaggctaaggcagaagaattgcttgaacctaggaggcggaggttgcagtgagccaagagggtgccactgcactctagcctgggtgacagagtgagattctgtctcaaaaaaaaaaaaaaagaaaaaaggaaaaaaggcagactatatgactgaatacacaacatgaacttttaactttttctgcaatttccccaaaaggtataaactgattttataatttagaggaaaaagaagtttaaaaagacacatcctaaaggcagtgtactgtgacccagaaaaaaatgaattcagaatccagaaatctgaattgctgccctgatgttacaacagactagctacttatttgaacctgagattctgccccaattgaaaatgttgacggtagttcaaagcgatcttgggaacaacaacaacaaaaatatgtaaatacaagaaacagtattatgtttgcaagcctcaatttaatcaatttaggaagtaactgaaaagtttttaacaagaaaaaggatacaattaaacgtatacttattgtattgtatactttgtttttatattactgacatttactttcactccattctagaattaaaccctcctaaaaccaagataagtttacaaaacctttataatactttatatgaggaattaaggagaaaattactcattaatctcatcatgactctgtagggccagaaagaacctgggagatgcagattctgtaacctgcaacggggtggacaataagactactgaatgactagtttcaagtggaatatccataacatagttaccccttactaaaaagcaaggatttcctatttcagtatctgaaaggttcaaaatttgataatgattacacaattatgttcaataaacaatcttttaaaatatattcctagcctttacttacatatcctacatatcccaactattttaactcacagcttacaagaatcagttataaaaagaagagtttgttctggaaactacacatgagataatatatgctgattcaatctaggaaggaagggcagaagcctgcaaaatgtgccactgagagggaaagatgactatccatttggaaggagatttagatcagctcaagaactcgcaaggactggtggcgagtggcagggtacatccatctgatgaatctgcagttttggtcggaaaaggatgctgtttgcaaacacccggtggggatgagtacctgtgccaagacatccttgaaaactccaggatacgcaccacctgactgcggaacagtgagcacaagacacccggagcgctgcagaatgggcacggcatccttggtaccgggccagccctctccacagcggcaggggcggggtaggggtgccctactggagggaggacatgggcacgactccccgaataaaaaccgctcatcgggtgggggcggggactaaccaccaccctcacaaccccctcacctccacgctctgttcatccattgcctccggctctcggcggggccgctggcgacccgcaggctccgcagtctgacctcttaggcgccggcccgaggtcgccagatcaaatcgccgataaaagcccggcgcccacgtcagggggctctgacaaccgccccacctgcgcgccccatctcttcaggtccagcgccgcctacccccatttcgccatttttaccggcgcgcccgccccgaggcgcagaagtagggcgaacggtggccgcagctcctttctcccggctcagccgccggccagcagccgcgccggaacctcggcccacgtgacgcgggcgcgcgcctatggaactgacggtggagttcagcgaagaaggtgccgcagagaattcgcaaacaccaaccgtaaccagagcagctgggggcgcggcggcgagagaagctgc

SEQ ID NO: 12 - DNA, 108679 bp, Homo sapiens
gcagcttctctcgccgccgcgcccccagctgctctggttacggttggtgtttgcgaattctctgcggcaccttcttcgctgaactccaccgtcagttccataggcgcgcgcccgcgtcacgtgggccgaggttccggcgcggctgctggccggcggctgagccgggagaaaggagctgcggccaccgttcgccctacttctgcgcctcggggcgggcgcgccggtaaaaatggcgaaatgggggtaggcggcgctggacctgaagagatggggcgcgcaggtggggcggttgtcagagccccctgacgtgggcgccgggcttttatcggcgatttgatctggcgacctcgggccggcgcctaagaggtcagactgcggagcctgcgggtcgccagcggccccgccgagagccggaggcaatggatgaacagagcgtggaggtgagggggttgtgagggtggtggttagtccccgcccccacccgatgagcggtttttattcggggagtcgtgcccatgtcctccctccagtagggcacccctaccccgcccctgccgctgtggagagggctggcccggtaccaaggatgccgtgcccattctgcagcgctccgggtgtcttgtgctcactgttccgcagtcaggtggtgcgtatcctggagttttcaaggatgtcttggcacaggtactcatccccaccgggtgtttgcaaacagcatccttttccgaccaaaactgcagattcatcagatggatgtaccctgccactcgccaccagtccttgcgagttcttgagctgatctaaatctccttccaaatggatagtcatctttccctctcagtggcacattttgcaggcttctgcccttccttcctagattgaatcagcatatattatctcatgtgtagtttccagaacaaactcttctttttataactgattcttgtaagctgtgagttaaaatagttgggatatgtaggatatgtaagtaaaggctaggaatatattttaaaagattgtttattgaacataattgtgtaatcattatcaaattttgaacctttcagatactgaaataggaaatccttgctttttagtaaggggtaactatgttatggatattccacttgaaactagtcattcagtagtcttattgtccaccccgttgcaggttacagaatctgcatctcccaggttctttctggccctacagagtcatgatgagattaatgagtaattttctccttaattcctcatataaagtattataaaggttttgtaaacttatcttggttttaggagggtttaattctagaatggagtgaaagtaaatgtcagtaatataaaaacaaagtatacaatacaataagtatacgtttaattgtatcctttttcttgttaaaaacttttcagttacttcctaaattgattaaattgaggcttgcaaacataatactgtttcttgtatttacatatttttgttgttgttgttcccaagatcgctttgaactaccgtcaacattttcaattggggcagaatctcaggttcaaataagtagctagtctgttgtaacatcagggcagcaattcagatttctggattctgaattcatttttttctgggtcacagtacactgcctttaggatgtgtctttttaaacttctttttcctctaaattataaaatcagtttataccttttggggaaattgcagaaaaagttaaaagttcatgttgtgtattcagtcatatagtctgccttttttccttttttctttttttttttttttgagacagaatctcactctgtcacccaggctagagtgcagtggcaccctcttggctcactgcaacctccgcctcctaggttcaagcaattcttctgccttagcctcctgtgtagctgggattacaggtgcacaccaccacgcctggctaatttttgtatttttagtagagacggggtttaaccattttggccaggttggtctcgaactcccgacctcgagtgatccacctgcctcagcctcccaaagtgctaggattacagtaatgaaccatcatgcctgggccttttttccctgctgttatacattatatgcatttttctatatcattagatgaagaagtatttctggtgactgtaatctatgtgtaactgccataatttacttaatatttggacatttgggctgtacctagattcttgccattataattactgctttagtgaaaatttttctgcatgtctgtgtttgcatttcctatattttccttagaatagaatcatcgtgaaaccaaacgggagtacattttccagatgttttgaggtactctgtatcttgaccaaccaaatcctgatatggaatagttaaaattgagagatcagcccaaaataaatgtaaatgtcaagttgttttgagcttgccttttactcttgattcagtagcctaaactggtggaccttacatcttttactgttttcagagcattgctgaggttttccgatgtttcatttgtatggagaaattgcgggatgcacgcctgtgtcctcattgctccaaactgtgttgtttcagctgtattagggtaagttgtatttctttttacatgcagttagtatatatagtatatattgtaaggattaaccatttcccttatctttttattttacattttttgccctaaagtgcttgcttgcactgtgcttggggttgaggaatactaagataaatgacaaattgcctgccttgaaggagcttaaatctaattgagggaatggagtttagaatgtttattttgaagaatgtgcggtatcataaggaggcactaaagaagtgctgtggggctcagaagaggactggtttatatgtgtttggtgagtaagatagagaaaaagtgtttaatcttacaattacaagacactctagagatgaatataatctagtggctttaaaacttgagtgttggtgctttgggaggccgaggtaggtggatcacgaggtcaagagatcgagaccatcctggccaacatggtgaaaccctgtctctactaaaaatacaaaaattagctgggcatggtggcatgtacctgtgtcccagctacttgggaggctgaggcaggagaattgtttgaacccgggagggcagaggttgcagtgagccgagagagcgccactgcactccagcctggcgacagagtgagactccgtctcaaaaaaaaaaaaaaaacttgaatgttggcctgagcgcggtggcccaggcctgtaatcccagcactttgggaggccgaggcgggtggatcatggtgtcaggagttcgagaacagcctgaccaacatggtgaaaccccatctctactaaaaatacaaaaattagctgggcatggtggtgtgtgcctgtaatcccagctactcaggaggctgaggcaggagaatcgcttgaacccaggaggcggaggttgcagtgagccgagatcatgccagtgcactccagcctgggtgacagagcgagactctgtctcaaaaaaacaaaaaacaaaataacaaaatgaggaagtccaagattttggttgtgtatgcgagagtccaatagcacatctacagagcatagttgaaaactgccttgttcacattccccttcaattatttattttcctgagaaaatcaaagaggtcacatgctcttcacctcaaactttctgtttcagctgggcgcagtaggtagctcacgcctgtaatcccggcattttgggaggctgaggtgtgtggatcacctgaggtcaggagttcgagaccatcctgaccaatatggtgaaaccctgtctctgctaaaaatacaaaaattagccaggcatggtggcatgctcctgtagtcccagctactcgagaggctgagacaggagaatcacttgaacccgggaggcagaggttgcagtgagcagagatggtgccactgcactccagcctggcgacagagcgagacttcgtctcaaaaaaaaaaaaaatttctgtttccttattcttgcctaaggaagatacatatgcctttgtcaagagtattatccgtaacacttaaataatttttttctagtcacacagctcacaattgatggtactggatttgaacccattaagttcaagaactctatagcctgttctttgtcactgtaccatgcgcagaagacccagttaaatatgatgttagttctcatacttctaaaactaagagattcactctttcttggccttctttcttcagctgtagaattcagatatgatttgctttacagggtggttatggcattaaatgagataacctaaggagtaattcagaaattatgaaatactatatctacttgtttgtaaggtgttattttattgttaaaatagcaactaatacacattaacacttacccatgttttaataaacagtgaaactgatgctagataaattaagtaattggactccgttatagttaattaagtggtagaatctggatctgggcgcatttcttttgatgaaggtcccatgtgcttatggtctctggttgttaccttatgcctcattgattctgtattttttcattgttacatcttcttcctcctgccccttaaatgcatttatttcttaatttcctaagatcttgttgctgtcgttctctactctgaggtttctgggagtattcacctactttgggaaatttgatttactttagcagctttagctatcatctctgtgtgtatggctcctaaatctattccttcagcactgacttttggatgtcttgtccttctagcatcttgaactttgttcaagatcagattattcatattattttctcaaaccgattctaagttcattctttttgttctgttctgctagtcactcaaactggaaactttataccaatgatttttttattttttattttttatttttttttgagatggattcttgctctgttgcccaggctaaagtgcagtggcgtgatcttgggtcactacgacctccgcctcccaggttcaagagattttcctgcctcagcctctcaagttgctgggagtatcagcgcactccatgatgcccggctatttttgtatatatatatgtatatatgtgtgtgtatatatgtatatatataaacatgtatatgtatatatgtgtgtgtgtgtgtgtgtgtgtgtgtatatatatatatatatatttttttttttttttttttttttgtagagatggggtttcactatgttgcccaggctggtctcaaactcctgacctcaactgatcttccctccttggcctcccaaagtgctgggattacaggcatgagccgctgcctctggcctataccaatgatttttaagtgcaattagttattatctccctgtttcacttatatttgtcttacccttctgtttttatcattaccatgctaatagagaagtatgaacattggttacctcatgcttagattatttaattactttctgtctgatctctcaggtttgagtcttttcttatctccagtccattttctgtcctgctactatattaatatttttcactgatatctctttcttttccccagaagcctcagaaatgtattatgtaaaggataaaatccaagaatgttagccttgtgttcagaccatccagatctggttctgacctttctttctagggctgtcttggttcctgaatgcactaatcatttgttcattcattcagcaaatattgattgcttgcctacttgttttcaagccagtcattttttattaggtataactgacatgcagtaaactacacatatttaaagtgtacagtttttttgtttgttttgttcttttttttttttttttgagacggcgttttgctcttgttgcccagcctggagtgcaatgacgcgatctccgctcactgcaacctccatctcctgggttcgagtgattctcctgcctcagcctcccaagtagctgggattataggcatgcatcaccacgcttgactaattttgtatttttagtagaaacagggtttcaccatgttggtcaggttggtctcaaactcctgacctcaggtgatccacccacctcagcctcccaaagtgctaggattacagatgtgagtcactgagcctggccaagtgtacagttttgacatatatgtattcttgtgagacaatcattgcatccaagttagtgaatatatctgtcatccccaaaagtttccttgtgcctcttggtaattcctccctctacttgccttgtccccaggcaaccattgatctgctttctgtcacgatagataagtttgcttcagagataagggggatggaaaaagagaaaaaagataagtttgcatgttttggaattttatacaaatgaaaatcatacaatatgtactcttacggatctggctttttttcactcagcaaaaatactgtatgtatcagtagtattcctttttattgctaagtagtattccattgtatgtatttaccacaatttgtttatgcattcacctgttgatgggcatatggcatgtttacagtttttgctattacagtgaaaattgctgcgaacattcgtgtacaagaagctttcttttctcttgggtaaataattagtaatggaacggatagagattatgtggtagatgtgtgtttaacattttgaaaattgccaagctattttctaaaatacttgtatctgggagctaaacaatgagcatacatggacataaagggggaaatgctagacactggggacttcaaaagagggagaggaggagagggatgaaggttaaattaaaaaaaggattgggaggccgaagtggatggatcacctgaattcgggagttcgagaccagcttgaccaacatggagaactctgtctctaccaaaaatacaaaattagccaggcctggtggtacatgtctgtaatcccagctccttgggagcctgaggcaggagaatcacttgaatccagaaggcggaggttgtggtgagccgagatcgcaccattgcactccagcctgggcaacaagagcgaaactctgtctcaaaaagaaaaaaaaggaaatagttttataattttacattctcaccagaagtttatgagagttccaattcctccacattaatgctaacagttggtgtgttcggttttgtgaattttagccattctcagggacgtgtagccattcttacaggtatctccttgttttagttagcgtttctctgttgagtaatgatgttagcatcttttccaggagttatttaccatccatatgttgtctttggtaaggggtctgttcagtgccccccattttgtgctaaaataaatataacaaaattttccattttacccatatttaagggtacagttcggtgtctttaattacattcataatgttatacaaccatatcactatttccaaaatctttcatcaccccaaacagaaagtctgtacccattaaaaaaaaagaaaaaagaaaataaacataagtaaacatagaaaaggtatattaaaaatacagtataaaagataaaaaatggtacacctatgtagggcagctccattctaatcttacgagactactgtagtatatgcggtccatcatttaccgaaacatctttatgtgacacatggctatataccactttttgtttatcctttttcttaagtttttttgttttttgagacagagcctcgctctgtcgccaggctggagtgcaatcgtgcgatctcggctcaccacaccctccacctccgaggttcaagcgagtctgctgcctcagcctcccgagtagctgggactacaggtgtgcgccagtacacccagctaatttttgtatttttagtagagatggggtttcaccatgttggccaggatggtgtcgatctcttgacctggtgatccaccctccttggcctcccaaagtgctgggattacaggcgttgagccaccgcgcctagcctatttatccatttttatctcttgatggacacttgggttgttttcactttttggatattggatattgttaatagttctgttgttaacaattatctgtttgagttcctgctttcatttcttttgaatatatatctagaagtgaaattgctgggtcatatggtattctgttaatttttttttaattgtggtaaaatatacctaatgtaaaattagcctttttttttttttttttttaaatgagacagtgtctcactctgttgcccacgttggagcacagtgacacaatcatggctcactgcagcctcaatctcttgggctcaagcagttggcctgcttcacactcttgagtagctgggaccacaagtgtgtgccatcgtgcctggctaatttttttattttttgtagagacagggttttactttgttgccaaagctggcctcaaactcctgggctcaagtgatctgcctgccttggcctcccaagtgctaggattacagatgtgagccacttggcccagccaaatttgcaattttaactgtatttttaagtgtactgtttagcggcattaagtacattcacattgttgtgcagccatcaccactatccacctttagtactttttcatcatcccaaactgaaactctgtacttgataaacaataaattctcattattctcttccccctagcccctgttaaccactattctaccttctgtctttacattgactattctaagtacatcatataaatggaatcataaaatatttttgtttatttctacctctctattctgttctattagtctctatttctgttcttatggtagtaccacactgttttgattactgtagctttgtagtaagttttgaaattaggaagtgtgagtcctccaactttttcaagatagttttgactatttggcatcccttgagattccatgtgaattttaaatgcagttttctaccaatataaaaagtaccattggagttttagtagggattacattgattctgtacatgcctttgagtaatattgctgtcttaacaatattaaatcttatgatccttgaacatgggtgtctttccatttatttaggacttctttaacttctttgagcagtgtattgtagttttgcacgtataagtctttctttttgttttttattgttttgagacaggatctcactgtcacccaggctggagtgcagtgatgcgatcatggctcactgcagcctcaacttcctgggttcaagagaccctcttgtcttggcctcccaatgctgggattataggtgtgagccaccacactcagcctccatgtatgtctttcaaattcttggttttttttttccctaagcattttattccttttgatgctgttgcaaatgtgttattttcttaatttcgtttttagattgttcattgcaagcttatagaaatgccagtgttttttgtgtgtttgtctcctgtaactgttgtgtttattagctttaacaatattttgtggactctttagggatttttacagataagatcatgtcatctgcaaaaagataatttcacttcttttttaattaggatactttttatttctttatcttgcctaagtactctggctggaatttttagtacctgtattatgtcaaatagaaatggtaaaagctggcatccttgtctttttcctgatcttaaggggaatttttcctgatcttaaggggaagagtttttagcctttgaatcgttgactatgatttttatgagtttttatatatggcctttatcaagttgaggaagttcccttctatttctattgagtatttttatcatgaaaggatattggattttgtcagatgccttttctgcatcaattgagatgatcatagtgggggttttccatgaagtggtttattacattgaatgatttttgtagttgaatcatctttctattctaggaataaatcccacttggccacagtgtataattttttaaatagtctgttgaattagttttattgggattttgttgaggatttccctagcacaatttctctagtaaatatcttaaactcccagaccacatatacattaaaatttttcatatagcattatgaatgaggatgcacagtacatgtcaataatgatatctctgattatattctgaagaaatggaaggaatttaataaatacttatatttttaattaatttcaaatgcaggattaggcattggaaatagtatttaatcaatcatgagcccatgatctaagatttttaataattgctgtaattaatgtttcttaaagaccttgttttctgttacaaagttggtttttttttttttgtttttttttttttgagacagagtctcactgtcacccaggctgtagtgcagtggcacgatcttggctcattgcaacctctgcctcctgggttcaagcgattctcctgcctcagcctcccgagtagctgggattacaggcacccaccaccatgcctggctaatttttatatttttagtagaggcggggtttcaccatgttggccaggctggtctcgaactcctgactccaggtgatccacccacctcggcctcccaaagtattgagattacaggcgtgagccactgtgcccggctgatgttttttttttaaactctctttttttggtctcctgttacactaacagtaattttatttaccatctggcttatttagttctgtatgctagataattaatatatacaatctttatgataattactcattcttacaggtttttggaaatgaacaaaaggacagcttttccctttctttggtagacataccataagaagtggacttttgttatagtttagtggtatagatgtttgtaagaaactattttagtctgggcacagtggctcatgcctgtagtcccagcacgttgggaggcccaaagatggaggattgtttgagcccaggacttcaagaccaggctgagcaacatggcaagatcccatctctgcaaaaaaaattaaaaaattagctggatgtggtgggcacacctgtagcctcagctacttcagagggtcaggtgggaggacctcttgagatgaagaggttgaggctgcagtgagctgtgtttgggccactgctctcctgcctgagtgacagagtgagacttgtctcaaaaacaaaaacaaccccagaaagtatttagcatgtttttgtatgtatatatagtttgcctcaaacagtacattaggggatgtaagaaattttgtatgctttcacaaggcatctaagatcctctatgatcggcctcctagtttccttgcaattttgagctgtttgggaataaaagtcctttgttaacaaatttcctatttactgcctgttttctcttatagctaaaagtatttaaaagagtgttttccactccatttccctgcaaccttgccatctacttacccttcaacccattctaacttggctttagctccctccacactactcacatagctcctgcaaagtgtagactcagttttgctaaatccaatgaaccttttgttggccttatctttccttctcggagcattcagcataaattacttcatctgtttttcttgaaacgttatctttggtatctttgccataatagctataccatcttcctagtaacttcatggtctatggattttttttttttaagagaccgagtcttgctctgtcatccaggctagagacagtggcatgatcatggctcactgcagccctgacctctggactcagttgatcctcccacctcagcctcctgaggagtagctgggactacaggcgtgtccaccatgcccggctaatttttaaatttattgtagagatggtatcttgctatgtgacccaggctgatctggagctcctggccttaagcaatcctcccaccttggcctgccaaagtgctgggattaaaggtgtgagccaccacacccgactggtccatgacttttaacactacctctagacttaaacttcccaactttatcttttcagtcctggccttttctctgcctccagtatctaactaccttcatttggacttgtagtatgcatctgaaatatatcgtctctataagaggtttttttttgttttgtttttgtttttgtttttgtttttcaccatgccaaatctgtttcttttccagtcaggaacccagttttttatccaaaaacctagctcaccttaatcttcctttttcctcacatccagtagtaattgaactataaatgttagcagccagtttgttttttttttaagattatcttagatacttagtatatattgctaacagcttctctctaacccttagagcggttttgcagggtagatatgttgattctcttgttgcatatgaggagacgagctcagtcacttgtccaatgtcatgtggtccataagtgccagagctgggaatcagacacagatctgaatactttctaaagcttgtgattcttccagtttaatgaaggagagaaaataatttttaattttaattttattttatgtttatgattttttaattgaatgagggggaatgctctaaagatatttgtttaatattatgcattaatattgtaccactggttctttggttttggtttgagagaaacattgcttttctcaaagtattaacattgtcatatttattttgtaattaaattagattgtggaggctgagcaatttcatgcctgtatgcctgtaatcccagcactttgggaggcccaggcgggcagatcacttgaggtcagagttggagactagcctggccaacgtgggaaaaccccatctctaataaaaatacgaaaaccatctgggtgtggcgtcgcatgcctgtaatcccagctgcttgagaggctgaggcatgagaattgcttgaacccgggaggcagaggttgcagtgagttgagattgcaccactgcactcaagcctgggggagggagtgagactctgtctcaaaaaataaataaataaattagattgtggaatacaagataaaaataccacaaatattcagagaatgtaaaactcactatagcctgggaaaggcaggaaattttatggacatgatccagtgacatttctatgagacgggatatggagggagggaccatataagcaaatgcacagatatatcgacagtgttatatagtgcagcagtaagcctgttttgactgggagggagggagacatttaaaatacgatctggttactccctatcatagtcccagtctttataagttttatagtctagcagggcaaaggagaccaacaaataagtcgtcgtaataaagtatctgaagggggaagtgtaggttgctgtggtagagcatggcaattgtatctaaaatagtctgcagagtgtcagggaaagctttttggagaaagcaacttatgaactgaaatctaaacaatgagtaggagtaacccagatgaagaaaggttttgaagaatatcttaggcagagagaggaaacatgtaaagacattaccttgctgggttatagggccagttctggagaggagttcatgagaccagttctggagaggagctgtgactttaacagaagagtggcatggtcatgccattgtttcaggggaagaaacctgacccaaatttcgtttttgcctagggaccaaggtaataataatgttcaaggttattaagaatggaggaatccttttccataggctttttattttatttttattttattttttgagacagaatcatgctctgttacacaggctgtagtgccgtggcgctgtctcggctccctgcagcctccacctcctgggttcaagcagttctcctgcctcagcctcctgagtagctgggactacatgctcatgccaccacacctggctcaatttgtatttttagtagagacgggatttcactatgttggccaggctggtctcgaactcctgacctcgtgatccacccgcctcagcctcccaaagtgctgggattacaggcgtgctctgtatgctttttctttcagctttattttctttgctctgaagtaccttgcttgctatcattttaagtcttttactgtcctttaaggtttccttgggaattttatttctaagttaattatttggcatcaagttaaagctcatgttcattttaaaaagtagaaagagataaacaaataacagatattgggagtacgttggtgtaggatcatagttatttacctctgaacctatttatcagacaattaaaacataaagttttttttattataaacctgttgtttcctgaaaactagtcagaaaatcttaacatctcctttatatgtggtataacattgctctcagtttcctaataaattattaatataatttttggtttagtagagacattattctcttctaaaacctttttataattatgaggaaaccaagatatctagaagcttcaattactgaactatgaaaccgttttttttgtttttttttttttgagacagagtcttgctctgttgcccaggctagagtgcagtggcaggatctcggctcactgcaacctctgcctccttggttcaagcaattctcctgcctcagcctcccgagtagctgggattatgggcgcctgccaccatcaccatgcctggctaatttttgtgtttttagtagagacggggtttcaccatgttggccaggctggtctccatcagctgacctcaggtgatctgcccaccttggcctcccaaattgctgggattacaggcgtgagacaccgtgcccagccctgtgaaactgttttaagacttaaaagttgtgctgtattgcttattcatttttttcggtttaatttacatgtctataatgtatgtaactcagaattagcttccttttttttttcgaattagcttttcaaaaaataaatttagtagtaaactagatagaaataaatcatcactgtggtttatttttgagtgcaaaggagacagtgccattattataagacaataataagattatatagacgttccctgtgagaatcagttagtcctgaaatcattgcttttcctgtgtgagagtagccttttaacagctattttgtgttgagttttgtcccttcaaaagtttatccctatgaggatgacagtgagggaaacagtagattgcctgttacttttataagtatctctaaaactaagcactagagagttaaacccattagatttgtagtattttatatatataaatattatataatatattatacattattatacattatatatatattatatatataatgtataatatattatacattatatatattatatatataatgtataatatattatacattattatacattatacattatatatatatgatatgttatataataatatattatatattatatattatgaatattatatatttatatattatatatattaagaatattatatatattatatataaatataaaatattaagtaatatatatatatagtaatgatgttttctaatcgatatctaatcttttgcccccttagcgctggctgacagagcagagagctcaatgtcctcattgccggtaagtgtttccttacgattatttgtgaattttgaaaatagtaagatcagaatgtgccttgggattcataaaggcagttttcctgcagtctgcccatttctcttcttcgtgaagatagttgagtagaaataagcaatttgactagaaaaagaaatgcagggactaattcatgtgacttaaaattacgtttactatgttgtactctgcctctcagaaaatcattgtataacttcttaatttaccttttataattaagcaacaaatttttaatgtttctggttaaaaaatccagatagttagggcataatttatcttttataattaagcaaaaaattttaatgttttctggttaaaaaaatccagatagcttggccgggcacagtggctcgctcatacctgtaatcccagcactttgggaggccaaggcggctggatcacctgaggtcgggaattcgacaccagcctggccaacatagtgaaaccttgtctctactaaaaatacaaaaattagctgggcgtggtggcgcgcgactatagtcccagctactcaggaggctgaagcatgagaattgcttgaacctaggaggtggaggttggagtgagctgagatcgtactgctgcacttgagccaaggtcacgccactgcactccagcctgggccacagagcgagagtctgtctcaaaaaaataaaaacaaaaaacaaacaaaaaaatccagatggtagagataaatataaaataaaataaaaagccttctgattctgatactccctaccctgtcttctcacttaataatactttcagaagaaattgttgggtttggtatctattcttctagactgttttttgtgcatgtattttgtaatgccctaatccagtatgattgtgcctatatttatatatatatatgcgtatgtgattcccatatgctttttcttaaaatgtaccaatgatataatattgttttgcagtttgctttttcatgtaaaatatggtagatatcttttcttgattgtatgtatacataagtcattttttaaaaaagtgactgataatcagttttatgactataccttgatttatttatgcccatattgagagacatttaggttatctctacttttggtttcaaaaaataatgctggagtgccatctgttaaccatttttctttttgtattcacttttctttttatttttttgagacagagtctcgctctgtcgtccaggccggagtgcaatggcgtgatttcagctcactgcagcctcgacctcctgggctcaggtgatcctcctacctcaacctccttcctaactgggaccactggtgtgtgctaccatgccagctaattttgtttattttttgtagagatgagatgtcactgtgttggccaaactggtctcgacttcctgggctcaagggatcctaccaccttgacctcccgaagtgctaggattgcagcgtgaaccactgtgcctcgtcccacctttcttacattatcttataagaccagcattatcaagtagaactttttataatgatgaaagtgttctatgtctacactgtccagtatggtagccactagttgtgtgtggctgtcgatcacttgaaatgtgtctagtgtggctatatgatgaaattttacttatttatttatttttgagatggagtctcactctttcgccaggctggggtgcagtggcgcaatcttggctcactgcaacctctgactccctggttcaagtgattctcctgcctcatcctcccaagtagctgggattacaggcacgtgccaccacgcccagctaatttttgtatttttagtagagacagcgtttcaccatgttggccaggatggtcttgatctcctgaccttgtgatccacccgctgtggcctcccaaactgctgggattacaggtgtgagccactgcacctggccaaaattttatttttatttaattttaacttaaaagagtcacatgtggctagtggctatcgttttggacagcacagctgtggatttgtgttttgttttgttttgtttttgagacaggctctcactctgttccccaggctggagtgcagaggcatgatcttggctcactgcagcctcaatctccctggctcatgtggtcctcctacctcagcttcatgggtagctgggactgtaggcacatgccattgcacttggctaattttttatagagacagggtttcatcatgttgcccaggctggtctcgaacacctgggctcaagcaatctacctgcctcagcctcccaaagtgctgggattacaggcgcgagccactacgccaacagtacagctttagactcagaagttgagaattcagtccatattctgaatcatatgtttttgtttagctttttgtttcttttcataaatgacgtgttaatgtggtgaaaataagtcacagataatatttttcattgtctttttttttattttttatttttttagttttaatagtattatggggccaggtagtggctcatacctgtaatcctagtactttgggaggatcactggaggccaggagttcaagaccagcctgggaaatatagcaagattcccgtctctacaaaaaattttaaaattagctgggtttgatggtgtgctgctgtagtcccagccactcaggaggctgaggcaggaggatcgcttgagcctaggaggtcaaggctacagtgaaccatgatcatgcatgccgctgcattccaccctgggtcatagagtgagactcatctcttaaaaaaaaaaaatagtgttatggtatgagtattattgtgctctctattttgagaaacgtatttattttataaaataatctcaaattcctgaattagtgtatgtgtatggattttctccccagtgctccactccagctacgagaactagtaaattgtcgttgggcagaagaagtaacacaacagcttgatactcttcaactctgcagtctcaccaaacatgaagaaaatgaaaaggacaagtatgtcctcaatttctttgttcaatattgaatggattttgcctttgtattgtttaatgacactatattggtagttgccttttagttatttatgaataagtagtgttatatttcttgtttttaattctttcagtcatgcgctgttactgttaagtaacatattcttattgtgtttatttaacaacagttatttcatgccatgtatacacagatacctctcgccctcccctccccccacacagaaagtactttgctagatgataggcatacaaaggtaaatgagacatgatttctgttaactgtttaatggggacacagtgataatacagtgttaagtcatttaagggacaagaatgaataaatggtcttatgttccatgattgaagaattttgaacatatctggagagttgttatggggagcaggtaaagaaaggaaagagtgatgtggcagtgattggtttttatagatgaagaagctacaacatggttaattgtaaggcttcactgtccagcaataataaaataacttgaattaggtgagattccaggacccctgaatgtttagaaataaagccttgtcactttctgtagagtcacaacatggtaaagcaagaggttgaaacctcacaggtcattagaaaaaaatttatctgtttcttcattatactgaggaaatagaaccatagaaataagagaacagggtgtggtggtgtgcatctgtagtcccagatacttgggaggctcaggcaggaggatctcttgagcccagaggatggcttaggtaacatagcaagaacccatctcttaaaaaaagaaaaaaaaaaaaaaaaagaaagaggcttaattatggtagtataacacattaatgcataatcagaattaaaacctactattcgggcgggtgtggtggctcatgcctataatctcagcactttgggaagccgaggtgggtggattacctgaggtcaggagttcaagaccagcctggccaacatggagaaccctgtctctactaaaaatacaaaaattagctgggcgtcgtggtggacgcctataatcccagctacttggggggctgaggcaggagaatcgcttgagcccaggaggcgtaagttgcagtgagccgagatcgtgccattgcattccagcctgggtaacgagcgagactccatctcccaaaaaaacaaaaacaaaaccaaaaacctgctattcaagtttaactggacttttaaggaattctgtcacttgtagcattgtagaggtctcaccaaaaataattatttaatttcttttctatacaatggcacatttctttggtgaacacttaggtgtttttcctctgaagtggtgggtattactgtttggaaagaccttttacctctttttgggcctcttctattagagcctctcaaaaggaatagactgctacaatttgctaacctgctagtctttcttacttgctgtttgatgcctactggcctccccttgctgcaagtgaatacaggctgttaattaagaggagttttggcaaattttctatattgtaaaagctatattataaatttggtcttttggaagtcttgctaaatgagcttccaaacagtacctgatgtattctaactcttattcatttcccattttatattctgattacttatgaattagaaatataaaatataaacttatgtaagtattttctctaagctcagtgtccttcatcaatgaaatgctttttgaaaaatgcaagagtgggccaggcacagtggctcatgcttgtaatcccagcactttgggaggccggggtgggtggatcacctaaggtcaggagttcgagaccagcctggccaacatggtgaaaccctgtctgtattaaaaatacaaaaattagctgggcgtggtggcatgtgcctgtaatcccagctacttgggaggctgaggcagcaagaattgcttgaacctagaaggtggaggttgtagtgggccaagatcgcgccacttcattccagcctggatgacagagctagactccgtctcaaaaaaaaaaaaaaaaattgcaagagtatgtctactttataaacaagagaatacttaaaacattttaattatggtaaaatatacataaaatttaccatcttaatcattttttaatgtacagttcagttatgttaagcatgttcacatgttcagccaatctccagaactttttaaacttgcaaatctgaaactgtacccattaaacaactccccattccttcttcgcaccagcccctggcagccaccattgtactttctgtttctatctgtttgagtactctagatactgcatataagtggaaccatacagtacctctctttttgtaactggtttattttacttagcataatgtcctcaaggttcatccatgtgtcagaatttcccacctttttaagggttaataatattatattgtatgtatatgtcacattttttttgtccattcatctctcagtgaaaattggtgtcgcttccaccttttggctgtagtgaataatgctgctatgactatggatgttcatgtctttgagactctgctttcagttcttttggttatgtaatcagaagtagaattgctagatcatgtggtaattctatttttaatttaagtttttgaggaatctccatactgttttccatagtgactgtaccattttacagtcccaccagtagtgcacaagggttccagtttctccacatcctggccaacacttgctcttttttgtttttttgatagcaaccatcctaatatggtatgaggttaggattcacatttctctagtgattagtagtaatgagcatcttttcatatgcttactggagatcttttgctcatttttaaattagagtttttaaagtttatttgttgagttacaggtgttatttatatattctggatgttaaaccttcattcttttcttttttttttgaggcagagtcttgctctgttgcacaggctggagtgcagtggtgtgattatcagctcactgcaacctctgcctcccgggttcaagcgattctcatgcctcagccttctgagtagctggggttgtaggagtgcaccaccacgcccagctaattttttgtatttctagtagagaaggagtttcactatattggccaggccagtctcaaactcctggcttcaagtgttccacccgccttggcctcctaaagtgctgggattgcgggcatgagccaccgctgcacccggcctgtagtttatttgtgtatatggtataaagtgagggtctgtattagtctgctaggtctttcataacaatactgctaactgagtagcttaaacaatagaaatttattttctcacagttctgataggtgttaagtccaagattaaatgctagcagtgttggtttctagtgaggcctccctttctccttgacttatagatggccaccacctagctctgtcctcacatgatctttgctatgtgcacgtgcatgcctggtgtctcctcctctttatagaggacaccagtcctgttggatttgggtcctgcccttgtgatccttacttaaccttaattacctccttaaaggctctgtctgcaaatacattcacactgggggttaaggctttaacctatgaatttgaggggggacatagttaatccataacagagtctaactttattgtttttcatgtgaatatccagtttaaaagaacatttaaaattgagttgttcattttccatgagggtctagtattaaatcgtgcagttaatcatgtgtcttgattattgttatcgttgaccacatgtaggaaaagtacttgtgcatgctcatgtaggtatagatgcataatcaatatgattattaagcacatgcccaaaatgtagtagtacaatagaatatatcatctgtagtgctgtgtggaaatgatgcaatatgtgttatgtttttaagaagcaaatctgtatttcgcatgtgattataaagtccagatcaatgaaaacagtactttacaaaaaattgtccgcataaacttctttagcttccttttcagaagccatggagtacattttatactgtgtttcttgagatataatcatttaaaactgacctgtttgcccaagcttaagaatttgtggaggtgattatgttccaatttaatttataacttcattttcttttcataatgtatagatgtgaaaatcaccatgaaaaacttagtgtattttgctggacttgtaagaagtgtatctgccatcagtgtgcactttggggaggaatggtgagcagaacaaattcagtattttttttaagttagaggctagaaggcactgaaatacttattcagatagaaaatagtgtcattatgctctcgtagaatgaaagttaaaatgcatctgctttctatacgctttattaaaggtattgtacttttattccagaagaatcttgtttgtgtggtttgatggaccccttatgtaaatattttaatctgtaacacctataaaacatatcctctattcatttgcagatcttttcgttgtaggatctaggtcacttttttcattaaaggtcagttgatcagatgtactggccaacatttccaattttagttcctttaaaatggagcacgaacttagacacttgtgaaacaacatgagtacgcctatctagatcttataattattttttattttttagattattttttccaattgaattcaagagcagtttttaaagacagtctaccattattgagcctgaagcattgaagttagctttcttagaagcagcttttttattttaaagtcgtgttctgttgtttactgaatactttttttttttttttgagaatttcgctcttgttgcccaggctggagtgcaatggtgggatctcggctcactgcaacctccgcctcccgggttcaagtgattctcctgcctcagcctccctagtagctaggattacaggcatgtgccaccaccacgcctggctaattttgtatttttagtagagacggggtttctccatgttggtcaggctggtcttgaactgctgacctcaggtgatccacctgacttggcctcccaaagtgctgggattacaggggtaagccaccgcgcccagcctctgaatacttaattaaatgatctccttacaataataagtacaattggcataaataggttgagaataaacacaatagattcttaggaaaaataagttatgggtgaaatggaagtgggtaggtatctagagttgctataagaattgatcattctgtgggcatcagtcagtatgttttacggagagagtggcaggccagattggttgagtagagctcaattcagagctttctgctagaatttgaatattaatgtgctaactagaaaaaattaagttctcacatttaatactttaaatacataggttttgtaagagacagtagaaatgtttttttctatacttaaagtgaacatgtaaaaatctacatcttttttggagacaataatgggatactttggctaaataagacaattctagtaagaacttataggaggtttcttatattaaaatttaataattctggttacaaatcaaaattatttttaggtagttagctataaaaattgttctttgagctgaatgcaactggatttgtgaggctaacttgtatcagtcttctgtctctgtagtaactctgtgggatggaaaatgggttcattgcttttgttaattttgtatcctgggctttttcttcaagtgtctttctaattcattcagctgtggtaaagactagggatttggtgatgaacatatcaaacaattacatactcaattggcttgataaattataggaataaatacagagagctgatgaggatgcataactatagcagctgatcctgtaaatctgagctaagtgcaaaggatgagtaggattaagacaaaccaagatgggagagggaagatggagaattattattattatttgttttgttttgtttttttgagacagagtctcactctgtcacccaggctggagtgcagtggggtgatctcagctcactccaacctccgcctcccggcttcaagtggttctccagcctcagcctcctgagtagctgggattacaggcgcatgccaccacgcccagctaatttttgtgttttttagtagagatggggtttcaccatgttggccaggctggtctcgaactcctgacctcaggtgatccacccgcctcagcctcccacagtgctaggattacaggcgtgagccaccatgcccagcattattattattattattattatttttttttttttatttttttttttttttggtttttattattaaaaaattattattttttgggggggacagagtctctgtcacccaggctggagtacagtggcccgatcacagctcactgtaagctctgcctcccgggttcaagcaattctcatgtctcagcctcttgagtagctgggattacaggtgtgcgccaacatgcgtggctacattttgtattttagtagagacgtgtttttgccgtgttggccaggccagtcatgaattcctcagttcaagtgatctgccctcctcagcgtcccaaagtactgggattagaggcatgagccaccacccctggctaataattattttttaataagagggagtaatttttgtaaagtcctggtggaaagaagtttgaggacatggaagaagactagtttgccttagagaagagtgactgactgataaggttgtggcagatgatttgggctgtggttaaagatcttggttctttttaataagttaatttctagttaaatgaatattttgctaaagccctttatttataatcttgaaagagttcctacttggaccaagtgattttaagcgggcactggcctgcttcaacaaaattaaattagctaataaggtaaagttcatttgagagatttaccagtttattagtgtccatagagtgttacaaaggaatgtaaatgcagatgagatatgtgaaagtgttctaaagctactctaatttaaatagcatggcggacatacctttaaacctttggcagaaatttatgagcaacacgtcactaaagtgaatgaagaggtagccaaacttcgtcggcgtctcatggaactgatcagcttagttcaagaagtggtaagactactactaaaatataattattttgtttttaatctgtaatatataataatatatttatataatataagttaatcagatgttgaaatcaaggttttcaagatttgcaaatttgataggctgtacctatagtcaaattccataaatttcagagtatctgagaaatttgcatacaaagcccctggcttgtaaggaagcagttggagtcagagaattaccacttatgcagaattagaaaatcagtgaatttaaaaagtctgtttgttacagagtcaataattaacttattttttcttttttttgagatggagtttcactcttgtcgcccaggctggagtgtaatggcgcgatctcagctcactacaacctccacctcccgggctcaagtgattcttttgcctcaccctcccaagtagctgggattacaggtgcccaccaccacgcccagctaatttttgtatttttagtagagacagggtttcaccatattggccaggctggtttcaaactcctgacgtcgtgatccacctgccttggcctcccaaagtgctaagattacaggtgtaagccactgtgcctggccccaataatttacttctaaacagaatcagtattgacttttacagtcattcattaagttatacactctttggatccccagcttttgggtgagtttcttttactaagcaatgaactatttgtgatgtcctgatttagaactccacagacttgaaagtgcaatatttaacattactgtttagatattgtttaggtcaaacagctttagtgagctatttataatgttatttctgtgaaaaaatgtgataagcccccaagaatgaaatactaagcatgtttttagaatacattttgtttcaagttgttagaatgacagtcataaatgaggtttatggaccatttcctaacttaaaatgaagtttgtggaccatatcctaaattaaaagcagatgtcttctttgttttacctctttaggaatcaagaaccaagtgatcatttataacattctttatgcagaaaatttgtgtttttgattgcatgtctgccattttgaaatataagcaagttgggtcatttccatttgaccttcctctaactacctttttcatattatctattcttggcaatattataatgtggtgcttctgtttttaaaatttacccaattaaaattattcttacctctttatcttctaggaaaggaatgtagaagctgtaagaaatgcaaaagatgagcgtgttcgggaaattaggaatgcagtggagatgatgattgcacggttagacacacagctgaagaataagcttataacactgatgggtaagtgtttatgtatgctgctggggtacctggtgctttcagcttctttgagattcagtctttgaagaataagggtgatcatcctaggaaggaatcttgagattttaggcaactccatgttgttacaatttagacagataaatgttagacttgtatttagaaatgataggagagaaaaattttcatagccatttggatgaaaatccaagttttcatagccatttggatgaaaatcctaatggccagtgggcatcattgttaggtagctttttctccaaatctaacagaattccttctactaatgttccatatcctttccatccattttcattggggatgaagaaactgacatttgtaaacatcggcgtcatcccctgccagcattttcttagagatttaaagtctcagcttttgatttctagtagggttcattttacatatgcttaattatatttattgctgacctttttgcttttcgaagattccttcattcttctttgattgtggagcctggtattgaacttaaattttaagtaagaatttaatccaatgctatggtttgttttgtttgatttgttttggtgatcttagtccttccagtgttttgctgactaaaaaccagctttgcattttttatcatagtccaaagtgaaatatatatactttaaataaggtgtgaacaaagaattttttttttttttttggctatcttgattctccaagattttagccatgtctttgtgtcatccaaaataatgattttatccattttatttttaactttttaaaaatgagatgcaaatatctttgagtttgtatatttgtatatcatttcatgaattgcttttctattataatcctatactatttaatatctgatttttagtttttttcccacagaccaagtagcaaatagtcttaaacaatcattcttctagtagttagattggaaagcacagttcatccacccagtggagcaattagacaaaacctgtaggtattgattgtactggtcacaatgccacttatatatttcatgtaaatgaaaatcgtaattgttcttagatcctcagttgcagtcatacatataggtgatcttagtttttcagaactctagataatatttgagatctctttaagttcattaagccatgtagagtactggatgttgatacgtattaggactttaaaattgaggggatcttgactgttgatgtgatcttcagtcgtagccaaataaacatttgcatgtggtcttgtttttcttgagtgagtttgttcattgcttcatttatttaattgaatcaactaaaagtcaaataataccacagtacagatctcttaagtttattaattttactacctgtataactgtgaaataacttccctttctaataactgaacttgccctgcttttgtggctttcaaattatttttcattatgaaaaattataatttgtgatttccaaggtagcttttatgattaaataggattctttctaaggataagaggtggggaaaaggatattttcagggttagtttatctttagaatagtcttataagcctcagatttttaattcataccataatatctgccactaatttattattattattattattatttgagatggagtctcgctctgtcattcaggctggagtgcagtggcactatctcggctcattgcaccctccgcctcctgggttcaagcaattctcctgcctcagcctcctgagtagctgggactacaggcacgtgccaccacccccagctaattttttgtatttttagtagagatggggtttcgccatgttggccaggctagtcttgagctcctgacctcaggtgatccacccacctccgcctcccgaagtgctgggataacaggcgtgagccaccacacctgacctctgccactaatttttttttttttttttttttgagatggagtctcgttctgttgcccaggctggagtgcagtggcgtgatcttggctcactgcaacctctgcttcccaggttcaagagattcttctatctcagcctcccaagtagctgggactacaggcacgtgccaccacgccctgctaatttttgtatttttagtagagacagggttttaccatattggccaggctggtctcaaactcctgaccttgtgatctgtctgccttggcctcccaaagtgctaggattacagacgtgagccaccacccccggcctctgccactcatttttaagtttaattatgtattgtaatgttgtccataggctaatgttttaggattgtgggatgtggttatagtagaaattatgtctgataatattagattctagctaatactttattttaactctttttgcaacctagaaattctgagttttgtttcaaaaataaaagttctaggctgaggcaggtggatctcttaagcccagcctgggcaatgtggtgaaaccccatctctaccaaaaaaaaaaaaaaaagaaaaattagccaggtgtgatagtgtgttcctgtagtcccagctactcaggaggctgaggtgggaggatcgcttgagcctgagaggtggaggttgcagtgagccgagatcacgccactatactccagcctgggcaacagaatgagaccctgtctcaaaaaaataaataaaaataaataaaggccgggcgccttggctcatgcctgtaatcccagcactttgggaggccgaggcgggtggatcatctgaggttgggagtttgagaccagcctgaccaacatggagaaaccccatctctactaaaaatacaaaattaaccgggcatggtggcacatgcctgtaatctcagctactcaggagactgaggcaggagaatcacttgaacccaagaggcaaaggttgcggtgagctgagttcgtgccattgcattctagcctgggcaacaagagtgaaaccccatctcaaaaaataaataaataaaataaataaaagttctacctattaaatgtttttccttatcaaataacactacagattgataaaacttttgctgtcctcatggatgtacttccagtttttcaaattttgggtttacaaagaaaatggcctgatttggtaagcatacctatcttctttttgtttcaaaacatataaagcactttcttccttttctataaggataatttctgtaattataatttttttgtttgtaatttgagacagggtctcactctgttgcccaggaaggactgcagtggtgcaatcaaggctcattgcatgactgcatgcagccttgaccgcttgggctgaagtgatcctcatgcctcagcctcttgagtaaattgggctgcaggcatgcaccaccactcctggctaattttttacttttttgtagagactgggtctcaccatgttgcctaggctggtctcaaatacctaggctcaagttatcctcttgctttggcctcccaaagtgctgggattacaggcatgagccatggccccggccagtaataatttgtttatccttttctgtctccaactttttgagactgttcacctctgtgggcatttttttttttaaacaggtccttgtatattcacaggacagaccttttgctgacatttaacctaaaaatctggcccactttgtattctcttttaagccacagttaaatttggtgaaaaatgatttcagaatagctgattgaatgaaattcatgttattgcttcttcccaagggaatatgaaaatctagaaaagtaaattttttgcttaaagtatattgtcattgcacatttcgtttcttagatgaatttgatctcagtggtatcttgtctaaaactaattagaattggttctgaagttccattttcaaggctactataatattcagcctcttaaagttgaatgagaatttttagggtagctgtaatgaagaatttttgcttacttgaagtgtatttcttgttattaaatatgttcattggaagttaatttcattcttaaaccaataatgaacccaagtgttgatgattcactatttttcatcaggtcagaagacatctctaacccaagaaacagagcttttggaatccttacttcaggaggtggagcaccaggtgatgaaatattaaatgtaatgaaatagtctttatccttttttctctgtatactgtagttgtttttttttttttttttttttgagatggagtctctctctgtcgcccaggctggagtgcagtggcgcgatctcggctcactgtaaactccgcctcctgggttcacaccattctcctgcctcagcctcctgagtagctgggactacaggcgcccaccactacgcccggctaatttttttgtattttcagtagagacggggtttcaccatgttagctgggatggtctcgatctcctgacctggtgatctgcccgcctcagcctcccaagctgctgggattacaggcgtgagccaccgtgcccggccttctctgtatattataaacgttttttttttgacagagattatctgtatgagatcaaaagatctgttgatcttttgtgttataatggagatttttatgaaattaggaatcagctgattcttgccaagttttttcttcatgtttgggaactgcattagaactacattctgctttgtaagaacagaactatttccatagaaatctgaatgtataagaggttaaaatctccccacactgtgacttaagtggtggctttgagatattattgccagttactttacatgtagcgattattactctaaaatttagcatttttatttattgaggaaatattaagtcctaaatagatagcaggcatcattgtagatattggtgatacagcagtgaacaaaatggtaaaattctgtcctcatagagtttatattttagattagggtgggtgaatatgaagcatatagtaaatgagtaaataaaatatgtagtgttgcataaatatctgatacattcacttgtctggaatttgctttttgtaatttctctctatgccttatacacagctgtctcctgagatttttatcttaatcattctgggaattcccttcacatctttcttgtattgaatctcatgtttcctgattcctatggtttccactttcttggtttacttgttttgttttaacatcacacttccctcagtagctttccaggaagggtagtcagaacacatttttcttgactgcattgtctgaaaatatttttattttatctttgtacttgattgacaatttggcttgtaggattcagcacataaatatttactaaatactcttatttttcttcattatccccattcttttttccttttctttttacatacacacatatatatctgtagggcattggccccaggaccttaatggataccaaaatccgcagatactcaagtcccttatataaaatggcatagtattctcatataacctatacacatctcccatatacaaacatgtgtcaaattatgaccttttggtcaatgacagactgcatatgtgacagtggtcccacaagattaaaatggagctgaaatacttctgttgcctagtagtgatgtctaatgacatcatagccattgtaatgtagtagcacaaagcattaatcatgtgtttattgtgatgctagtgtaaacacacatactgcactgctagttgtataaaaatataacacattattatgtatggtatataatacttgataagcataataaacaactgtgttactggtttatgtatttactataccataccattcattattctagaatatattctagagtttactcctactttttaaaaaaacgttgactatattccagaagaaggcattgttagcattgtagatccatatgtgctagtacccgtgaagacctttcaatgggacaagatgtggaggtggaaggtggtgatattgatgatcctgaccatgtgtaggcctaggccattgtcttagtttttaacaaaacagtattgagatggggtgtacacctgtagtcccagctactcaggaagctgaggtggaaggattgcctcagcccaggaattcaaggcttcagtgtgctatgattgtacctgtgaatagccacggcgctcgcctgggaagtatagcaagacccatctgtttaaaaaagtaaagaaataggccgggcgtggtggctcatgcctgtgatctcagtactttcagaggccaaggtgggcagatcacttgagtgagttcaagaccagcctgggcaacatggtgaaaccccgtctctactaaaaatacaaaacttagctgggcgaggtggcacatgcttggagtcccagccactcgggaggctaaggcatgaaaattgcttgaacccgggaggcagaggctgcggtgagccgagatcgtgccactgcactccagcctgggcgacagagcgagaccctgtctcaaaaaaaaaaaaagtaaataatattaactgaaaaatgtaggctacatggtgatatagcctgatagtgtttatgtaaagtttgaaaacattcagaacattgtttatggatgtatgtacacacatagtaattgtggaaacacatgtatagtgataataaacttctgttgaagggagcaagttgcttttggaagaaatggtgtcaaggaaggatatacacagtaagcattcaattaatataagttaaatttttttgtattatgtgtgtatgtatgtacatatacacagtgatttttagcataccttaaaaatcttaagcaaacagttcaaataactttagatcattttatataaattgttcaaaaaaccgtaatgccttgggcaatgcctgacacataataatatgaatgacaaaatatgcagctgtttgttgttttctgctgttaccacttaccattatgtcattggttgatgcagtagctcaaacatgatatcattattcttggtaacccaagactataaactttgatatatatcatatgatatatgatacttttttttttttttgagacagagtcttgccttgtcgcccacgctggagtgcagtggcgtgatcttggctcactgtaacctccgcctcctgggttcaagcaattcttgtgcctcagcctcctgagtagctgggactacaggcacgtgccaccacgcccagctaattgttatatttttagtagagatggggtttcaccatgttggccaggccttgaactagccaggtcatgaggtcttgaactcctcaccctaagtgatccacccgcctcagcctcccaaagtcctgggattacaggtgtgagctattgtgcccagccatatgatatgttatttttggtgagccttaagcaactgttatttaaaagatataaactcacctgtaggtgggtttatgaatatcattcatatggcatttgatagtatattagcttgctaggcctgtcataacaaaataccaatgactgtgagacttaaacaatagaaatttgttttctcagtattctaggggctggaagtctaagattcaggtgttggcaggtttcgtttcttctgaggtctctctccttggcctgtagatgactgccttcttactgtgtcctcacatggacttttctgtgtggttgtgcagccctagagtctcttcctcttaataaggacaccagtcatgttagattaaggccctaccccagtggcctcattttaacataatcacctctttaaagattatctccagatagactgaggtactggaggttgagcctgcagtatataaattatggaggaaaggggcacaaaattcagcacataacagatagtttacaaagctttttcactatagcttcataatccttataacaaacttcacgaagtaagcagtggtggatattactattttagttttgtatgtaaacagactcagagttatgtaattagtcagtggcagaattgggagttacttcaaactggtttttttgaagaaaaaattagtagtcttatcttttatctaccatggttatgttactgaagttagatttgttactttttcccaaaatagtgtttcccaaattagtatcagatagtgcgtgtgtgtgtgtgtacacacgggtgtatgtgtgtacgttttgtattctgtcacctagaatacaatctgagaaattgcttttaaaagctttcaccttggtcgggcacggtggctcacgcatgtaatcccagcacttggggaggccgaggcaggtggatcatgaggtcaggagttcaagaccagtgtgaccaacatggagaaaccctgtctctactaaaaaatacaaaaattacctgggtatggtggcacccacctgtaatcccagctattcaggaggctgaggcaggagaatcgcttgaacccaggaggcggaggttgcagtgagccgagatcgcgccattgcactccagcctgggcaacagagcaagactctgtctcaaaaaaaaaaaaaaaaaaaagctttcaacttaccggtttgttcattaatacattcataagaacttcactgtaccagcctgaaagtatatttgtacttgcttaaaacctcatttatcctaaaatgtaaggagagaaaaatgcattagtattaagtatacttattcattgactttactgttagaatattatagaaataaacctagattctttgtacattcttaattctcaagctcactttgagttatttttatcatttttttcagtttataatcagaataatttgtaatttcttaagaattttgaggtggcagtgaatagtaattttgtttgttcagataatttttttcctcacacagttgcggtcttgtagtaagagtgagttgatatctaagagctcagagatccttatgatgtttcagcaagttcatcggaagcccatggcatcttttgttaccactcctgttccaccagactttaccaggtatgacacagtttttaatttcattttcatttgaaatttatgtggaaatgcatatacttgtttcaaaagaatgtttttttttttaatgccatctctctctctcttttcttttatagatagggtctcacttgttgcccaggctggtctcaaactcctggactcaagtgattttcccaccttgaccccccaaattactagggatcatagacatgagccaccacacttggccactccatagctttttaaggtcttgtttataatggagtggtctggcaagctctgttgtatacatacaaatctagttctcctgaaaagtctcacagactgatacttttgactcttaagagaaaatgacaatgaactagaaatcatttttttttccaagagctccatcttccgtagtatagaaaaaaataattgattacattagcttcatcagtgtaccttttgaattgaactatatctcctaacacaaaactcagaaactgatgaaatgccaaaagaatttgaaggtgacgccatgttatctccggatactaaaagatagagagaggaaataattatggcaaaaagtatcctagttttcatcttgggctaaaccttagaatttttctgaacagcatattccttcaagcatgtttgtaatcattctgtggtgttagactggtggtttctgactagatgttggaatcacttgggatttggctgaaaattaatcagtgctgggtcataccataccccaggccatttgaaagaaaatctttgggtgtgtattagtctacttgggctgctataacaaaataccatatactgggtgcctttaaacaacagaaatgtattttctcacagttcgagaaacaagaattccaagatcagggtgtcagaaaattcacttttttgcctcagcttgcagacagctgccttctttctgtgtcttcacctgaccttttctctgttcttgcttggagagagatcagtctctggtgtctctttctctttttatgaggacacaagccttattgtattagacccaccattatcaactcatttagccttaattatcttcctaaaggccctggctatagatacagtcacattgggggttagggctttaacatgggaatttggggagacacaattcagcctgtgacatagggttcagatggacattttcagtttgttcttttaaaatctccttagcttattctatgatgaggggtgaagccactgcttcagataaacattttggtgtgacagactttatcttagagagatgacttatggaatactatcaagttttcctaccatggtgagggctgtggcacatttagaaaacagtctttgttataaaaatgtttatatgttgagctgccttttctttttcctctaatatgtgatgcctctgatgaccacagagcattcattgtactattttattttttatttttttaatttattattttatttatttattattatactccatctcactctgtcacccaggctggaatgcagtggcgtgatctcggctcactgcaacctccacctcccggattcaagcaattctcacacctcagcctcctgggtagctgggaatataggcatgcaccaccacgcccagctaagttttgtatttttagtagagacggggtttctccatgttggccaggctggtctcaaactcctcacctcaagtgatttgcctgcctcgacctcccaaagtgctgggattataggtgtgagccaccatgcctggccgcattcattgtactagagtatatcatataagccagggcttttcaatcttggcattgttgacattttaagcagaataattatttgtggggtggggtgagctttcctgtgttttataagatgtttagcagtatcccagtagcactccccaccccaccaccttgcagttgtgacaaaaatatttccagacattgccaaatgtccttggggacaaaatcacccctgactgagaatcactgttataagcccttcttactcgttatagatgccacagtcctataaacagaagggcaaagtttttctgacagctagaactccagtggttctcattgaccagtagaagtcttcctttctttttgattaacaacctcataaaatattcatatctttttaaagcactgacttttttgtttaaatctcatttctgaatgacagcactgaatattattaaaaggactatgtatttgctgtcattaagtagatttggttgaagaatgttcaccaaaatccctaggctgcttcattgatactgtggtcagctatttcagttttgaagatttgttttctgaaccatttcatcccctgtggaatgaaacagattttgacctaagggtcatatataaaaattagtatttttcaattcaaagtgtggtgcatggatgacctaagtcagggctatgtgtggcatttgtctgaaatgcaaattcccaggccaatgtccccagaatctttagaggtggtattcagaaatgcgtcttgagcactctaagtaataatactgcactaaagtttgggaaccattgatgtatgtaaattcccattcttataccactttcagcagctcatcaggcaggcaacaatgctcgctagtctgttaattaatcactttcagggccttctctctttctccagtaatgctcactaccttattggaaactttggcttttaatttaattcttattagaaacctgttttattagttgcaacatacaggaaaagagggccaaataagaaagatcagcattcgtgacattaagttacacatgaaactttagatatgcaaagcagtggtgtttctattgcctttagttctatcagttagtaattggtgatggagccacaatggagaagaaaaggtcactaaatcagtgaagtgttatttattaaagactatgtgcccagtattgttgtaggtgctgcatgggagaagaggaactaaatattttactgtgggctgggcgcggtggctcatgcctgtaaccccagcaccttgggaggccgaggcgggcggatcacctgaagtcaggagttccagagcagtctgaccaacatggagaaaccccatctctactaaaaatacaaaattagccgggtgtggtggtgcatgcctataatcccagctactccggaggctgaggcagaaaagaaaaaataaaagatacaatagtatagctgtcagttcatagagagttttatgtttcagggagaaatagtcaagttgataaggagaaccagtttttatatagctcagcagtttatttccttaagaaacaaatggatacttgtggttctcccttttttccctttgttgatgaagttatgatgaggttactggtctgaagatttgcagatcagtgacttccagattcctgggctagagatggtttgagggtagtttcccatattctgtgaagacaaagattcactgacaccatcatgagatgaatccagtcatcataaccagagcagtctacacttctaagagaggaggagaaaattttaaaactatgaaaagataaagcagtgcctcatctgcagacacagaatgataacatcagtatgattgaagataatctctaaatgaatgagcataatatggagatgtggtggtggaattagatggcctagcagtagcaatagctttgtatatacatataaagaatgcaaaatcagtgagccgagatcgcgccactgcactccagcctgtgtgacagagcgagactgcgtctcaaaaaaataaataaataaataaataaataaaatttaaatttaaaaagcaaagtaatttctttcgcaaaagaaatttttgagcaaatatttccaactctacgaatctgccagcaagtatttataaacattcattcagtgaatatatgccagctatattttaggcatgtgttaagtgctagcattaagcataatacagagctctttgggtttataattatgatttagaaaaacaggttattggaacagtaataaaattaatgtggtaactggtgataggggaagtaagtatcctgtttaatgctgccccataccactaatttgtcattatactatttgtgtccaaaaaattttccctaggagaatgtacatatgtaaaagtatatcacagtacctgttttctgtgctctgaatgtggtcattttctcctagcaagtgagactagggtaaactggactgtactctcttggttcatgagtgttagtaacctatgctctttagcctggaagctttctacttcaccattctgagttgaaaatggcatctcttgttgtttaatttgggttctgctgaagtcttttgacccttaatggccaataaaatatgataatgatagtctggacttagagcagaaataacagtaacaacaacacaggaaataaattacactggacatggcagaagaaataatatgcagattagcaggcaactggggattgattaaagtctttgcatgttgtggaggatattctgtacttgatgagagacctgattgaaagttgcacaaaatttcaaaaacatgtcaaatttgaaggctatactgggaagactcccttagagctcttactaggtttatggttccagaaaggcttcctaggcactaattgcattggtcctgctaattggagtttgtgtatgttgagtgctaggagatcatataatgggaagaccttagagcaaacctccaagagtatgtgacttctaatctgttatctgaagaaatagtaagggttaaccaggcaaaaggatatggggatgaggaatgtgagtcttctaagaaggataacaactgactagggatatcataggaattcttgacatagtcaatttggatagtagctatacagttaatcacagggcaaagtacatctgcttactgtgtaaaagacgtgacctatttagctatacgtacttacattgaaataaagaattatttgtctatattgtattgcaagccactaatttctttagacactatcatagtgtggcagtgctatagatgtatattttatcttgttttgataataaccaaaattatgatgaattgaactttagaaaatatatctgaagatgtggtgaaatggtttgcagtttattgttttgggaaatttgtaattttatatatcaaatttataacactattaaaacatcgtatgatagtctttttcatagtatagtgttttaatgagttttgaggtgttctaatgaaagtgtacaaaccatccacatgattaaaggtagttagcagcaaagtgacttttagtccattctgtgattataatacagttctctctgaagaagattggatgggactaaagttgcttgaagtagttttgacttgccaagaaagtgtgagtgtgctagtccatcacagttgccccttaggtgatgaggtattcatcctataaaaatgtgtagatttatttgctagtaaatagtttcaaagtatttgaggtctgttaatatttcccataatccattccagagtaggaagtttggggattaattgtgttatcactgactgttgtttcattttttaaaggatgttttatatctgtttcataattcagattcacaccctaagcgtcagtttctttttcccatttccatgagtactttttaaagtgtatttcttgtacttagtttaataattatataatgtgttgcctttttttcttttaggaaaaaaagcttttataaagtatttatttattgatttattggttgactgagacagagtctggctctgtcgccctcgctggagtgtggtagtgcaatcttgattcaccgcaacctccgcatctggggttcaagcgattctcgtgcctcagcctcccaagtagctgggattgcaggcgcacaccaccacatttgggtaatttttgcgtatttttaatagagatgtgtggtagtgcaatctcgaatcaccgcaacctccacctctagggttcaagtgattctcgtgcctcagcctcccaagtagctgggattgcaggtgcacaccaccacatctgggtaatttttgtgtatttttaacagagatggggttttaccatgttggccatgctggtcttgagctcccgacctcagatgatccacccaccttggcctcccagagtgctgggattataggggtgagccacctcacccagccaaaaaacttttataaagtatttgtatctccaatttaattgcaaacttgatggttttatgaattcatttgatttaatgttatgttcaataaagatttaaaaatatttcttagattttagtttaataaagtttttaagatactagtgagggacttggcttttttaggcaaatggaattttaaacatgtaagctaaataattttttttgcctttttttttttagtgaattagtgccatcttacgattcagctacttttgttttagagaatttcaggtaagagtttttgacaattctggtttttttgtatgtgtatataaggaagagccagaaaggtattctttggtaagacagtaatctagtgaactgtgtcactcactttagaagtcttagaagaataagtttcctattagaaatgtagagataatttgtgctggattatattgtatctttagactgactgcttctctagtctgggaaaaggaaccttttctttttattttgagactgggttttgctctgtcacccaggctggagtgcagtggcgtgatcattgctcactgcagcctcaacctcctgggctgcagcaatcctcccacctcagcctcccaagtaactgggactacaagcgtgtgccaccacacagatggggtctcactgtgtttcctaggctggtctcaaactcctgggctccagtaattctcccacctcagcctcccaaagtgctggaattataggcattagccacggtgccctgcccaaagtgaggaaccattttaaataagagagatcacttagctttgactaaggtggggggctggacagttagaggttgatgagccccctcttacctatcctcagaaataaaagtgctgggaagaaatccattgagatccatttatctcagctcaaagtgaagaattgtggataaggggccattattgtaagaaatgtgtaggctttgtcagttgtatgttaattttgttagcttttactactttttctcttaagatcagaagtgaagatcgtaacatgcaaaggttcttagcaatttgtctttgagggcagcatggaagtggaaacaaggactataaggtcttcaggagagaaactccaaataataggcaaaggccatcaaaataaggccacactgggcagggcatggtggcttaagcttgtaatcccagcactttgggaggccaaggcgggtggatcatcgaggtcaggagttcaagaccagcctggccaacatggtaaaccccgtctctactaaaaatacaaaaattagctgggcgtggtggtgggcgcctgtaatcccagctacttgggaggctgaggcaggagaattgcctgaaaccgggaggtggaggttgcagtgagctgagatcgtgccactgcgctccagcctgggcaacaagagtaaaactctgtctgaaaaaactctatctcaaaaaaaaaaaaagccacacagaatttatccctcttcccatttaatttgtcttttcttagggttacttttgtggctaaaacatagtctctttgaggtttttcccacacgtattaaggtttagtgtactcttgttcttcaatattaaaagatgattatactgcagagaagcgaggaagaaacattctgggttttgttttatttttaatggtgaagactgcttgttgaagcattaattaattttgggagacttggtgatacattccttttcccttaaatgtcacaattttgagggttttttttcccctgaaacttaaaagatattgctgaatatttcagtatctttaaattttctttatttcaggatgtaagcatatctaaacttttacagtggaattttgacctgttcagtcaccttgttatttgtctaggatttcaagtatgtaacaaagaaactggttggtctttctttttgcctgttgccatttcacaacagttttgatcttgggatttggttggttttctttttcgttagcactttgcgtcagagagcagatcctgtttacagtccacctcttcaagtttcaggactttgctggaggttaaaagtttacccagtaagtttttcatatttctaataaattttgaaaccaaggtctttctttcagagttctgtagtatactaatttgattactgagaactgttaactcttcttttttttgttacatatgctgtccgcattccctgttccccttcttcccccatattagacaactgtttgtttgtccacaccttgttctctaatacattctgctggaaggcctattggaaacaatgttccctgagttctcatatgttgaaaacagtttttgccttttatattctaaggctagtattgctggatataaaatccttggctcatattttctttcattgggtatcttcaatatgcaattccattttctttaggaaaagtctgataataaactaattttctttacctttaaagatgtttggtgatattgcctagatgctaaataatctttttgagtccagtaatttactattacacattttagccctcctgggttagtattctcagttacacattgaagtctttcaatatgtagtttcagattatttttttttttttagaaaagttttgtataatggcattacacaataatatatcatcttttatgtctggctttattcacctagctgatgagtcatttattcattgatatctactgagtgattatcatgtcgaacctcaaaagttgcctcaggtcctttcaagcaaagtagactaaaaatgaaggggttaatagtacttgtacctctctgtagtcatatcccagtagacactggagatgtataaggatctctgttgtcagccttcattgtctcgttgggggaacagatgtttaaacaagtagttataataaatgtgattcttttaaaacatagacacctgaagtactagggaaatacataggaagtaattctctttttttaaaagagtcttgccaaattaaacaaaaagggagggagtaattatctttataaaaataaaatctatagtagagatttaaacaacttctcaatctctagacagaatcttatatggaattctaatattaaaatcatatagaaattatatttaattactttcagcatagggtattacctttttgtatgttggtcttaaaaattaatttttggtttaaaatttttttttgcttcactcatttctaatgaaattatttttcccattagggtcttgtttttttattaaggttgatttgtaagagttctatatatgttaaaactcttaacctttgttgtggttgttgatatttcccttcatttttgtgccttaatagtttcactttttttttaatgcacaaaagtttaaagttgttaagaactcaaatctattggtgtttttaattatttcttccattgctttgaggcttagaagtacttctcaatctagatatcagatattatctgcattttgttttattgtggcttaatttttttacactcaaactgattattcttcttttaggatggaaatggagttgtgcgaggttactacttatctgtgtttctggagctctcagctggcttgcctgaaacttctaagtaagaagtgtaattaaataacaaccttaatgtgcatttacataccttccccctagcaatttttgttaatttttatttttgttaattactgtattggtatgtctgttaagatgtagaataatatgttcttggcatcataggatggtatttttctaaaaacaaaatatttttaagaaaacttgtattgctttgcgtttttagaaatctctttactatctggcttagaagaagactactggattctcatgtctgcttttatattcaatttttttaaaatatcattttgattgaaatacatgtatgaaatctgtctgcagagttctacaaccatcaccaacatctaattttataacattctatttactccacaaggaatccctgtactccttagcagtcacttcccattccccgtttccctcagtccctgagaaccacaaatgtactttttgacaaggaggaatattttttaaagtggttttagataattgtggatcttctttgatattacatccaaactgaacaagtggtaggttgttaaatgttatttacaatgtgggatctggaatcatattaatgatttttttttttaactctgttacatgaaatctgttagtttagtatggaatgtctgctttcagctaacatgaagcaagagggatgagatctacccattttcctgcaacaaccaaaaaaaaaaaaagacctcatgaaacaacattttctagacactggacatggtggtaacaaatgaaagtagtccctgagagatgagaaacacaaactaactctacaatttctccagcttacagccttgagagagttttcaggctgcaacacagggaggagctgggactgagggagaatctggcagacttagttgaggggatagtgctgagagtctagaaaggacaatacagctagagcgcattggacaaaataccagagaggagaaagctgcagagactgaagtctagagatccgtaatgggttccccttgagtattcagcagagtacttatcagggcatatgtctgaggaaactacctgagattggggaaagaactccttgacaggattagagggaatagtgagtgactggtgttcacacggggccaggaactgtgtctattgcaacctgccacggtggagaaatttataattcatgggtcactgattgggtataacactaaggaagtttttgtctcgatagcagaaattagccctagactgaacactgctctggacccacctaacaaaagtgagacctgaaaggatcaaattgttttcaagtaacttaactgcatcccagaaggaagctcaagaaaatttataaggttgctgaagtatctaacatccaacagagtaaaatttacagtgtcttgtgttcagtcaaaggttaatatgccagactggctggcccatacctgtaatcccagcactttgggaggccaaggcaggaggattgcttgagaccaggaatttgagaccagcctgggcaatatggtgagaccctgcatacgtctcagctactcaggaggctgaggcaggaggattgcttgagcccaggaagtcgaggctgcaatgagctgtgtccgcaccactgcactccagcctgaatgacagagtgagaccctgtctcaatttatttatttacttaatgtattttgagtctcactttatcacccagcttggagtacagtggcatgatctcagctcactgcaacctctgcctcccaggttcaagcaattctcctgcctcagcctcccaaaatagctgggattacaggcatgcaccaccacgcccagccaatttttgtatttttagtagaaacggggtttcaccatattggccaggctggtcttgaactcgtatcctcaggtgatccgcccacctcagcctcccaaaatgctgggattacaggtgtgtgccactgtgcccggtcaagaattctttatatattttggatactaaacctttatcagacatatgatttgcaaatagtttctcccatttggtaggttttcttttcattttcttgataatgtgcattaatgcacaaaagtttataggctatcttctttgtctttttgtaaacctgaaaattttacaagattggatatataaattagaatatattggctttcccaatgtatacttcttttctttttgagacagggtcttgctgtgtcactgaggctggagcacagtggcacaattatggctcactgcagcctcaacctcctgggcccaggtgatcctcctgcctcagtctcccaagtagctgggactacaggaatgcactaccgtacccagctaatttttttgttttttgtttttatggagacggggtttcgccatgttgcccaggctggtctcagactcctgggctcaggcgatctgcccacctcggtctcttaaagtgctgggattacaggtgttagccactgtgtccagcttacttattttctttttcctttttcttttctttttaacttttattttaggtttggtgttacgtatgaaggtttgttacataggtaaactcatgtcatgggggttcattgtacagattatttcatcacccaggaattaagcccagtacccaatagttatcttttctgctcctctcctttttcccaccctctgccctcaagcataccccagtgtctgttgtttccttctttgtgttcataagttctcatcatttagctcccacaatataagtgagaacacgtggtatttggttttctgttcctgtgttagtttgctgaggataatagcctccagctccatccatccatgttgcacaaaaaacataatctcgttcttttttatggctgcatacttattttctattaggaaattttatagcctttgattttctgaatacacaataatttgctttcttatcacttgtactggcaaaatacagtagagtatttcaaaatttatattttaaaattgcaaggtgataaacctcaaaggataatagaaataacaatatttatgaggaaacatatgtagtgaatatatggaaaaaatattgcattagtaaaacaaaagcaactttgaattacttgtttattcaaaatttatttattttttgaggtgttgttttatacctgatatatcggtaaacatctttacgaggaaaaaaggacctattgccattcatgttttggtggttcgttcaaaatactgctgttttggctgggcgcagtggctcacacccgtaatcccagccctttgggaggtcaaggagggtggatcatctgaggtcaagagtttgatactagcctgtccaacattatgaaaccctgtctctactaaaaatacaaaaaattagctgggcatggtggtgtgcacttgtaatctcagctacttgggaggctgaggcagcagaatcacttgaactggggggcagaggttgcagtgagctgagaccccgccactgccctctagcctgggctacagagtgagactctatctcaaaaaagcaaacaaaaaaatactgctctttttctggccgggtgtggtggctcacgcctgcaatcccagcacactgggaggccaaggtgggtggatcacctgaggtcaggagttcaagaccagcctggccaacatgaacccccgtctccactaaaaatacaaaattagctaagcatggtacacgcctgtaatctcagctactctgtaggccgaggcaggagaatcacttgaacctggagtcagaggatgcagtgagctgaaattgcaccattgcactccagcctgggcaacaagagtgaaactccatcttaaataaataaaaaaaccccaaaatactgctgtttttataaatgataaaagaaatgtaactgcgtatatcaataaggtttcttctcaggtaattacttagtgaccttaatgtttagatgagtttgttttttattcttactttactaactgtatagtaatggtctgtttttcaattttagatatgaatatcgtgtagagatggttcaccagtcctgtaatgatcctacaaaaaatatcattcgagaatttgcatctgactttgaagttggagaatgctggggctataatagatttttccgtttggacttactcgcaaatgaaggatacttgaatccacaaaatgatacagtgattttaaggtaacaggaaaaatttgatgttgtggtttttattgtggggaaataatatcatcagaactatatttcaaagatcatttggttaacaaagatgaatattgaaattcatactaactataaccttatcacctttttttttttttttttttttttttttttttggagacatagtctcgctctgtcgccctggcgcgatctcggctcactacaagctccgcctcccgggttcacgccattctcctgcctcagcctcccgagtagctgggactacaggcgcccgccaccacgcctggctaattttttactatttttagtagagacggggtttcactgttttagccaggatggtctcgatctcctgacctcgtgatccgcccgcctcggcctcccaaagtgctgggattacaggcgtgagccaccgtgcccggccccttatcactttcttaactgttttatttaagtggataataatcaaaaatttgtagtcaaaaagtcaaactttttttgacatctagttagcaattccattaaagataagctgggtgcagtggtgcgccggtgtttccatctactatggaggctaagatgggaggacggcttgggcccaggagtttgagaccagcttaggaaacattatgagaccccccatctttttaaaaagaaaaaaaaaagggggtgggtgggaaactacaacttccagtaagtatagtaatgttccatttgtctgttgaacttctagtgacctcaccatttggaaatctaagaatagtgagggataaagtgctgatttttttcacttcacagtgcatattatatttgagtgactggttttacaacttaattttataaagcaactatcagttctttgtcaataacagcaaattaaaagtagaagagtcaagatgaaagttgacgaaaaccagcaattttctacttttttcttaaacttttattttaggttcaggggtacatgtgtaggtttgttatacaggtaaactcacatcatggtgatttggtgtacggattatttcatcacccagatactaagcctagtacccaatagttattttttcttatcctctccctcctcccaccctccaccctcaagtaggccccagtgtctgttgttgttacccctcgttatatccttgagttctcatcatttagcttttttttttttttttttttttttgagatggagtctccctctgtcgcccagaggcgacatatcggctcactgcaagctctgcctcccgggttcacaccattctcctgcctcagcctcccaagtagctgggactacaggtgcctgccactgcatctggctaattttgtttttgtatttttagtagagatggggttccaccgtgttagccaggatggtctcgatctcctgacctcttgagcctcctgcctcagcctccaaagtgctgggattacaggcctgagccactgcgcccggcccatttagctcttaaataataagtgagaacatgtggtatttgcgaaaaccatcaatttttttttttttttttttttttttttgagacagagtttctagttgcccaggctggagcgcaatggcgcgatcttggctccctgcaacctttgcctcccaggttcaagcgattctcctgcctcagcctcccgagtagatgggattgcaggcacgcgccaccatgcctggctaattttgtacttttagtagagatggggtttctccgtgttgatcaggctggtttctaactcccgacctcaggtgatccacccacttcggcctcccatagtgctgggattataggcatgagccaccacgtctggccaaaaccatcaatttttattgttgattgttggtgtagtatttacaagaaaggatagaacgtaaatactgactcaaacaacagccctatttagccaggtgtggtggctcacgcctgtaatcccagcactttgggaggctgaggcaggcagatcacttgggctcaggagtttgagagcagcctggccaatatggtgaaatcctatctctactaaaaatacaaaaattagccgggctgtggtggcacgcgcctgtaatcccagctactcaggaggccaaggcaggagaatcgcttgaatccggcaggtggaggttgcagtgaaccgagattatgccactgcaccccagcctgggcgacatagcaagaccctatctcaaaaataataataaagtaaataaaaacaacagccctgtttaacgtggggtagtctttacaggtcagaagtttttgggcattggctgggtgccgtggctcacgcctgtaatcccagcactttgggaggctgaggtgggtggatcacctgaggtcaggagttcaagatcagcctgggcaacatggtgaaaccctgtctctactaaaaatacaaaaattagctgggcatggtggcaggcacttgtaatcccagctacttgggaagctgaggcagttgaatcgcttgagtccgggaggcagatgttgcagtgagccgagatttcaccagtgcactccagcctgggcgacaagagtgaaactccatcttaaaaaaacaaaaaacaaacaaaaaaaggtttttgggtatcattacatgatgttataatttgatgatcaaaataagaacttgaatatttaaaaaagattaaaatatgttctgaactgagaagtttcagagtatttaacagtttaaaaagttctcatcacaacaattttaaacatttttttcttgagagtgatttaatgtactcctgaatttcccttattcggagctctttgttcttgagcatactggacagatgagttgctatcatatcagcatttactcttatgtgttttccgtagcttttctggaagtataaagaagtttaacaagggatatgattttcagtgtagggaaaaagatatgtttttgttattgttttgttttgtttagagtcagtgtcttgctatattgcccagtctagtctagaattctcgggctcaagtgatcctcctgtctcaacttcccaagtaggtaggattacaggtgcatgccacggtgcctggaatcatgttttttgaggggagggattattttagttttcttcgtatagcttttcagacttttggggtttaactattcaccaagagaaataatttattgctatttcatgcttcagccatagagagaattacaaaattattcaaacatcaattttaccaacctataaaagacccattttcttatagccttgatttcatccttattagcgttacttattagtacagaaacaaattcctgttatgacatcattttttttaagcaacattgagtctataccatatgcctgacactgagttgctaataagaaaaatatagacatggtttttgtccacagggaactcaagatgaaattggcagataaaaacagaatttactactctcttattataaaataatgctatagtagcaccgtaagtaaattgctatgaagtttgtggatagggggcttcacattatcctattaatgagacattgctcaaactttaaaccttttatttcaagattctggtggaattttgtatgtatttattagaaagttatttaaggcttttctataactatgaatgtatgtttcattctttcattaaaacagaaagttaagaaattttacctgctgcttggttttgttttaatggcaaaatattagaactaatgtgcagagttaaccagtggtttaaatcattcaaagaaaataatagtgtttagacggtcttactttaatgtcttaccagtgggtacttcctagactgtgaatcgtctctgctcagttgagtgaatcatctctactcagttccgttcagaacgtaatttaatctcttgatttgatttgatttgatttgatttgatttgatttgatttgattttttgagacagagtttcgctctgttgcccaggctggagtgcagtggtgcgatctcggctcactgccacctttgcctcccagattcaagcgattctcctgcctcagcctcctgagtagctgggattacaggcgcccgccaccatacccggctaatttttgtacttttagtagagacggggtttcaccatgttggccacgctggtctcgaactcctgaccttaggtgatctgcccacctcggcctcccaaagtgctaagattacaggtgtgagccactgtgcccggccaatctctctctttttttaatactcaagttctcattttgatcatccagttataacatataatgatgcccaaaaacttctgacctataaagactatcccaacattaaataggactgttgttttatagtcagtgtttatgttctgtcctttcttgcattagtattcttcactaaaactagatatctactgccagataactgttaaatggtcagtaggttcttacaataaggtgtaaaatcctagaaaaaaatgaacagctaacattgtgtagccgggtaaaagggctggaaggaatgagagagatgtagatgtccaaattagtcttgccttgtggtttttcctttcttcaaaaacaccttcacacatgcactgcaattttttcaccttaaatatagtcacgttaggctatgtaacaaaggtagaggtttgtttgctttagtaatagttttttttttttttttaattaaaacaactctggatagattaagattctgtcatttatatattaatacgattttatgtaggtgcagtactttttttcctaacctaagctgaaataatgccatattgtaataagggcagttttgattgctatcttgaagggttcaggatatagtttatttagagcatgtaactccttgggggatagtaaagcaatcctgggtggttgagccaggaagtgattttttttctgaggggcaggaaatacgggaacgtgagatggaatagagaaatataaagagcagcacttaaaactttgtattttgctacaaaaaattagccgggcgtagtggcgggcgcctgtagtcccagctacttgggaggctgaggcaggagaatggcgtgaacccgggaggtggagcttgcagtgagccgagatcccgccactgcactccagcctgggcgacagagcgagactccgtctcaaaaaaaaaaaaaactttgtattttgctggggcctcatttagggaagaaagggataagagttatcttcctccttataggaggaaactgcatagtatatggctctcttcttcccaactcatgatcatcattgtattttagttacattattataataaatatgcctgagtaggttagtttgggaagcttaactacaatgtacaatgttatttctttagaaaggtagtttgaggacaactggcttttcagaaccttgtgtcataatctgtggactacatctataaacaacaaatactagcagctctaagtttatgtgaattttggccttataatcaagttaacccaaattgcagaattcagtgctagactgataatgttttactatttgaattttttttttaatgtggcacaattcttttaaggtttcaggtacgttcaccaactttctttcaaaaatcccgggaccagcattggtacattactcagttggaagctgcacagactagttatatccaacaaataaacaaccttaaagaggtaagaaaatatctatttctgttttggtgtttcctattgtccttttggcttgttatatttgtaattagattttatatattttattgctatttgtaattttgtaatttgtaattagaaaaaaaatctagttatcttgttccccatccttaccagttctgcagtgtccagtttagtagacactagccacatacggccatgtaaatttttattttactttatttatttattttttttgagaaggagtctcgctctgttgcccaggctggagtgcagtggtgcaatctcggctcactgcaagctccgcctcctgggttcacgccattctcctgcctcagcctcctgagtagctgggactacaggcgcctgccaccacaccgggctaatttttttgtatttttagtagagacagggtttcacagtgttagccaggatggtctcgatgtcctgacctcgtgatctgcccacctcggcctcccaaagtgctgggattacaggcatgagccaccacgccctgccactttatttttatttttttttattttggagacaggtcggactcactcttgttgccaggcacaatttccactaactgcagcttcaacttcccaagctcgggtgatcttcccaccttagcctcccaggtagctgggactacgagcatgtgtcatggctagtttttgtttttgtggagatgggtttctccatgttgcccaagctggtcttgaactcctgggctcaagcagtctgcctgccttggcctcccaaagtgctgggattacaggtgtgagccactgtgcctggtctgtaaatttagcttaattaaaattaaaaatttagttccttagttgcagtagccacatttcaagtgcttgcattggttactgcaaatctagagcatttccatcattgcataaagttgttttgaacagtgtggctgtagtttgaggtaagctgtttgtgtttctgtcagtgacttagtttctcttccacaagttatcttggatttagaggattctaggctatagcacaattaaaagttgtcccatcttgaactcttactattgttgaatgagcactggggaagattaattgatgaatttttattttacagtggtaaactaaggaatgtttcatttcccttaggaagttagggtgtggcaggaggtttggagtgctgagagtcgttaataggtcagggaggaaaatgtgaataaatacatgctttcagagattttcatgttaaatgtgctatttaaaactgcctgtattcactatactgtttgctaattgtatgtttagcttaggctggatatgaaagaaggaggatgagcataaatggggcagtttatctttgtcatcaatttttttttataaataaaaggcagtagggattcagagaaatggtttaagaaatggtgccaagtgaggtggctcattcctgtaatcccagcatgttgggaggccgaggccagaggaacatttgagcccaggggttcaaaaccagcttgagcaacatagcaagataccgtctctaaaaataaataaaaatgaaattagccaagcatggtggcacacacctccagtcccagccacttgagaggctgaggcagtaggatcgtttgagcccagtagttcaaggcttgcagtgagctatgatggcaccactgcacttcagcctgggcaacagagtgagaccattctcaaaaaataaataaagcaatggttttagaaacatctggatgcatttttttttcacttgagacatcttttactgtgccagtgagctgtggctaatatttgtgcttttcttgttttaaaagagacttactattgagctgtctcgaactcagaagtcaagagatttgtcaccaccagataaccatcttagcccccaaaatgatgatgctctggagacacgagctaagaagtctgcatgctctgacatgcttctcgaaggtggtcctactacagcttctgtaagagaggccaaagaggatgaagaagatgaggagaagattcagaatgaagattatcatgtaataatgaggccagttttagatttttgtgtttgattaaaaaaaaacagtagcatcatggcttttaacatattatgtgctaaaacagtaggctaaaaatattgtccattgatttccattagtaatgtatgtatgcccttatgtaaatgttaataagtcagaagttgttgaatcaaagcatttcaaggttgtgaaaaaaacaatacattgtataaaacaaaccgtgatacacattttaaaacctaaactaccatacattgtggattttaaaaatgtgttctggccgggtgcggtggctcacacctgtaatcccagcactttgggaggccaaggtgggtggatcacatgaggtcaggagttcgagactatcctggccaacatggccaaaccctgtctctacgaaaaatacaaaaattagctgggtgtggtggtgcttgcctgtaatcccagctacttgggaggctgaggtaggagaatcacttgaacctaggaggcggaggttgcagtgagccaagattgtgccactgcactccagcctgggagacagagcaagactctgtctcaaagaaaaagaaaaaaaaaaggtgttctattttcattcactgaaactagtacatgccttatcattacttacgtaggtagactgatttaggattcggtagctagccaggttctatcccagctaaacaacagtcagaatccagaataagagttcaagaagtagttgatatttttacataagaagggatgcatagacagttggtttttttgtagtaatttttaagatgagagacttcagttttaagttaaaatgaaggacccagtgggtcgagagaatgcacagattaagtgctttgataaactagagataatagggtttggaatggagcagtcaggtgaagcagttaacctttgataaactgagttaaaggaagggtggagtttgcaggtaaggcagctgaaaagttggggaagtttttgcttaatagtgggtattttttttctgtgcagataggtgacagtgttaattaatgaccttgataatggtaaaattaggtagaattgaggagtgatggtttgaaacgttactctgtgaggaagctgatgtggaatatgtgaaggattccagtcacactgagggcccagctgaaggcggcacgcataagttagtaaaatgatatcagtcagtgtggttgtgtgatttttctctacagggctcagcagctgaggcacaggagcagaaaatagatttttgaaaactgtattattctttggtgcttggtttatgggctggatggggtgatcaactgagtagaaacatttggaatagacgtcttgttttcttggcattgatctagcacgagctttcagatggagatctggatctggatcttgtttatgaggatgaagtaaatcagctcgatggcagcagttcctctgctagttccacagcaacaagtaatacagaagaaaatgatattgatgaagaaactatgtgagtttcccactttactactgtaaagcatttaagtggtaattattagaagcatattaactaactcacacttaaatgtagggatattcagcacttttttgaaagattgaatcaatagtcagttttctctctctaatctctgggctcttagccaataatttttgtttgtgttttaacttttaaatttgaattttgaaattattttatacttaagaaaagttcagaatttcatgtttccttcacacagattcattccctatgaatattttgctttatatttctctgtatataattttttctgaaacacttgagatcgaatatacttctcattgtgtacttttctaaaaacaaggatattcttacataactatagaatagttatcaaaatctggaaattaacattgatgtaatactgtaaccttctaaaaatttgccagtaatgtcctttgtcaggatcatgttgcatttaattgttaaatcgatttagttatcttcaatctgggttacttttttttttttcctttttttttgagacggagtctcgctctgtcgcccaggctggagtgcagtggcatgatctcagctcactgcaagctccaccttccggattacgccattctcctgcctcagcctcccgagcagctagaactacaggcgcccgccacctcgcccagctaattttttgtatttttagtagagacggggtttcaccgtgttagccaggttggtcttgatctcctgacctcatgatccgtccacctcggcctcccaaagtgttgggattacaggcgtgagccaccgcacctggccaatctgggttacttttttaaaaatttgtttttcggctgggcacggtggctcacgcctgtaatcccagcactctgggaggctgaggcgggtggatcacgaggtcaggagatccagaccatcctggctaacacggtgaaaccccgtctctactaaaaatacaaaaaagaaattagccaggtgtggtggcaggcgcctgtagtcccagctacttggtaggctgaggcaggagaatggggtgaacccgggaggcggagcttgcagtgagctaagatcacgccacggcactccagcctgggcgacagttgagactctgtctcaaaaaaaaaaaaaaactgtttttcatgactaatactttctgagaaatacagaatagtttattttgtagaaagtccctcagtttggattagtctgaagtttcctcatgattagatagaggttacacacttttggcagggatactacggaaatgataatgtatccttttcactgcgtcatacagagtggagcatgatgtcagtatgtcccattacttgtggttaattttgatcacttagttgaggtagcatccgccaggtttcttcactccaaaatttgtactttttcctttataattaataagtatcttatgtatgggactgtaaatatcctatttttcatcctactttttcctatgaattttgacaccctttgatgagtgttgcctgaaaaaggttactgtgttgattgccaagtggtgtttttctaattccattatttcttactcattcattagttgcaatttaaggaagagcttttccttctgccctgttaattgatgaactaatgaatttcattaatgtggataattttttttttttgagatggagtcttgctctgtcacccaggctggcatgcagtgtcacgatcttggctcactgcaacctccatctcctgggttcaagtgatccttccacctcagcctcccgagtagcaaggattacaggcgtgcaccaccacgcctggctatttttttgtatttttagtggagacggggtttcaccatgttggccaggctggtcttgaactcctgacttcaggtgatctgcccacttcagcctcccaaagtgctaggattacaggtgtgagccactgtgcctggcgatttttggatttttaaaccaagcttgcattcctgggacacatcccacttggtcatagtgtgtaattttttttcttttttcttttttttttttttgtgacggagtttcgcactgtcacccgggcttaagtgcagtggcatgatctcggctcactgcaacctccgcctcccgggttcaggcgattctcctgcctcagcctcccaagtagctgggattacaggcacccaccaccacacccagctaattttttgtatttttagtagagacgaggtttcactgcgttggccaggctggtctcaaacacctgacctcgtgatccacccgtcttggcctcccaaagtgctggaattacaggtgtgatccacttcgcccagccgtaattttttttttcttttttttttatttgaaacggagtctcgctctgtcccccaggctggagtgcagtggcgcaatatccgctcactgcaagctccgccttctggattcacaccattctcctgcctcagtctcccaagtagctgggactacaggtgcccaccaccttgcccggctaattttgttttgtatttttaatagagatggggtttcaccgtgttagccaggatgatctcaatctcctgaccttgtgatctgcctgcctcggcctcccaaagtgctgggattacaggcatgagccacggcacccagcaattttttttttttcctaagagaatctggctttgtggccaggctggagtatggtggcatgattatagctccctgtaaccttcaactctggcctcaagtgatccacctgcctcagcctccatagtagctaggactactggtgtgcatcatcacactcggctcatgattctttttatatgttactgcattctgtttgctagtacagttgtccctcagtatccttgcggtattggttctaggaccccgcccttggatactaaaatctgaggatgctcaagtatcttatataaaatggcagagtagttgcatataacctatgcacatcctcccctatacttttaaatcatctctagattacttataatacctaatacaatgcaaatgctatgtaagtagttgttatactctattgtatacagaataatgacaaggggaaaatgtctgcgcatgactttattttgttttgttttatttatttttttttgagacggagtttcgctcttgtcgcccaggctggagtgcagtggcacaatctcagctcactgcagcctccgtctcctgggttcaagcagttcccctgcctcagcctcctgagtagctgggattatacacgcctgccactacgcccgactaattttttgtagttttagtagagacagggttttgcaatgttgaccaggctggttttgaactcctggcctcaggtgagccacagcacccggccggctttattttttgagacggggtcttgccctgtcacccaagctggagtgcagtggtcgcgtgatcacagctcactgcaacctcgacctcctgggctcaagtgatcctcccacctcagttttccaagtagctaggactacaggtgcatgccaacacacctggctaattttttaatattttgtagagacagggtctcactatgttgccttactatgttctaggctggcccggagctcctaggctcaagaagtccctccagcctcggcttcccaaagtgctgggattacaggcatgaaccaccacaccagggcttaaaaaatatttttgatccacagttggttgaatgcacagatgcagagcccacagataaagaaggccaactatatttcgttgatgctcttaatagtgttgtatgtttctctgagtgttgtatgtgtctctgcttctccccataggcaaaatctctgagccaaagctttgaagttggagtaaggataaaggtgtacctctcttactttagaagctgagctaactggatgttggggacagtagccttagttctttccttttcaggcttgggacctccaccttatgaataagctggggcagggatgatctgggccccagtattctcatcatgctgcatccaggtagagcatctagtccacaagtgggtctcagcagaagggagtctccacctcttggccgtgtgtgcctgtactatagcatcagcaataggtagctgggggcagaatgagagatgctgataccttgccactcccaggaatatagccctcctcttggaagttcgggaagtgggaggcctgtgttcttcactgcaccagtctggagtggaatttctgtaacaccaagttaggaagtggagtgatcttggttcaaatactgcagatgcccacttgtcttacccagtttttatagatttttttgagtagatgtttcttcatttgctgtatgcccttgggaccatttccagagtcgttaagtggttgtttttaaaataattgtcaccagtttgactaaagagcagacctacagagctccttacattgtcaagctgggtgcttatcttgagatttccttaaatacccaagactgtcattgactggtactgtttcaagtagtatgttgtttaaaataaagtaatagtgattgttttttaacatgagagactttgtatggccgagtaggaagtaataccctctttgctgaaatattatttttgaaaaatggtatttagtcatatacagtaatttgggctttttttggatgttaaaatgtagtgttttaagcctaacactacatgtagtgttttaagcttattgccattaagctaccagttctctgaagctgtaatttcttctaacagtggttcttgtatttattagtgtttctaacactaattaaataggagagattaatctcttctaatagtggttcttagattttttagtgtttcatttatttctatttttttgcctgacacagttctttttttttaatcttcctttcctttcctttcctgtcctgtcctgtcctccttgagacagagtctcgctctgtcgtccaggctgaagtgcagtagtgcaatcttggctcactacaacctccacctcccgggttcaaccgattctgctgcctcagcctcccgaggagctgggattacaggtccccaccacaacgcccagctaatttttgtatttttaggagagacagggttttgccatgttggccaggctggtctcgaaatcctgacctcaggtgatcctcccaccttagcctcccaaagttctgggatttcaggcgtgagtcactgcgcccggccttgagatatatatatatatatatatatatatttttttttttttttttttaaattcctttttttcttagctttctaagaagaactggctgattttttttttttttttgagatggagtctcgctctgtcgcccaggctggagtgcagttgtgcaatcttggctcactgcaagcttgcctcctgggttcacgccattctcctgattcagcctcctgagtagctgggactacaggtgcctgccaccacacccagctaattttttgtatttttagtagagacagggtttcaccgtgttagccaggatggtctcgatctcctgacctcgtgatccgcctgcctcagcctcccaaagtgctgggattacaggcatgagccaccgcgcccggccgatattttttaaaggttgtttaaacaataacattaacgctacttaaatttggaaaatgacaaaaatctttctcctttttgtatttagaaatatcttatttcacagaaaaatctgcatatcataaaatttattgaggtgtgaaaaacaacgtttattgtatcactctatataaatgatggattttgtatattcttaggtctggagaaaatgatgtggaatataacaacatggaattagaagagggagaactcatggaagatgcagctgctgcaggacccgcaggtaatgaggtcactccactgcctccaagccattctgttttctctctgctcttctctgactgtattatattttaaataggtggtagccactgcttgtacatggttcatggtgttggaagtggtatatcttataatatggattttttagagtttattactaaagatatttaaaacaatagctaacatttgaaaaatgttttatgtaccaggcactgtgagaagcactttaagtaccttatattaataaaattctgtgtgatacattcactgatgcatagagaaattaaagcaacttgccaacttttaacaatttagtaaatgagagtaattgaatccccagatttgtttgtatcccatgctcatattttatactattctttctgatataaagtcttatttgtatatcttgctagactatagaatgatctcagtggaatcattgactcttagacttacgaaggatcttaaagagctcattttatagttttcagttacctggtcatatagctggttgttagtacaaccagactagaaccccagattgtcctgggtttgtggacccagcatttttctcccaaactaaaacactgcttctcctgggaagaactgacttatataatgagtacttttttgtgtgttctcggatctatttaattaagtagtgaatcttggttagagtaattaaaagttagctttctactgtttctgttttacccatgttctcctcagtatgaacactggcatattctttttttttttttgagacggagtctcgctctgtcgcccaggtcggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtcttgatctcctgacctcatgatccacccgcctcggcctcccaaagtgctgggattacaggcgtgagccaccgcgcccggcctggcatattcttaatatgctgggataactaaagatttcttaaatactagtcctcaccatttcaactccaacctaagctctatattctttctactcctgactttaatcctgggtaatcttatccacacctgtatttcagttactgtctatatgctgattacttttcaagttctttctttagcctttgtctttccatagtttccaaacccatgtcttggtgtgtgattcagcacttcccaaacacaaattactgtcttttcatctgccagcctgcctgtcagcagtagccaccgtgactgataatgctatacattctttacttcttcctctccctaaaactttacctttagtgtattaccaagatctgttggttctgtgtcctaaatcgtgcataaattcatttggtactcccactcctttgccatttctagtctaggcctctgtctttcacctgggttccttgtatttattattgttcccctccattgtatttttacagcatggattcagaatgagggtgtttttttctttctgattaatatctaatcatgttacttctctaccagtcttggtgactcacgcctgtaatctcagcactttgggaggctgatgtgggtgcatcgcttgagtccaggagtttgagaccagcctgggcaacgtggtgaaaccttgtctctacaaaaaaatacaaaaaaattagccagatgtggtggctaaattgctgcctgtggtcccagctatttgggaagctgaggtgggaggatcacctgagcccaggaagcggagcctgcagtaagctgagatagcaccactgtactccagcctgggcaacagagtgagaccctgtgtcaaaaatagaaataaaaataatgaaaaataatatctaatcatgttatttctctacttaaaaactctttaaggattatatttattataggataaagtccaaacttgttaacatggcacataaattcctttgtgtctggattcctgtttgtttccttagccttatttctgtcattcctctgtatcattctttagtcaggaggaacttcaaagaatcacaagatctaccaatttggagagaagagctttaattcttataaagactggccatcctgggccgggtgtggtggctcacacctgtaatcccagcacgttgggaggccgaggtgggtggatcacgatgtcagcagattgagaccatcctggctaacatgatggaaccccctctctactaaaaatacaaaaattagccaggtgtgatggcaggcgcctgtagtcccagctactcgggaggctgaggcaggagaatgtcgtgaacctgggaggcggagcttgcagtgagctgagattgcgccaccgcactccaacctgggtgacagagcgagactccatctcaaaaaaaaaaaaaagactggccatcctgacaggctgggaagtgtggcctctggcaaagactaaaagcaggcactttgcaggagggaagggtaacataggaattgatgctgaataagttggccaagtacaactaaatcgattcagcaggctataggaggagctgtgaacatcatgaaaagggaaacacacgcatgcatagtagacaaacatgcatgttacatgcatttcatgtttactttggagtggggactgaacaatttaaatgcattacaattaggccctgttgtcaaaaggtaaagccaaggacacgaaggcactcaatgctcagcctctgtatacgggccagaaccagtccatggttggcgactcctatcaggagaaagttactgaaatcagtctcttgtccagtcaaagctgtagttacggctgtggaacagggagtgagtcagtcagcatctggcagtcagtaagctgcaattgtttcaatattgcttatcttgaggccagtgcttgcttagcttctagaaaaaagaagaaattctgttgcagttagaatgtagtttattctttaagtatagggagcgtgtgacttaatccttgtctggtatggcattaggtcttactgactgactaacttttcagccttgtgtgttttcttttagttaggtccattctttcctctcttaaactgtgtggtagagagattcttgtatataacatttatatacacttagggtaaagaagagtttacatttttgaattatatattcatcaaatatttactgagcacttgagctgtgtacagttttactatctagagatacaatattgaataacaagatatccctgttcttatggagcctatgttataattctgaatattctgtaataatccaggcactgtattctagatgttgaagacataataatgaaccagtttatttttattatatgctttgggaaatttatgtatttggcagaaatttgttaaatatcatgcctacctggaacatacaacactaagtaatttataacctaagggtgagtatatttatgaacaattagctagaatatgacaatgtacatacaaaagaattccaaatcatttaaatactcccttctcaaggagatgaaatataacttctaccccttaaatgtaggctgtgcttagttacttgcttctgaatagtacagtttcaaaagggggaaacaactttgaagtagagaaacttggcaaacattacctacccaggtgattaaatttagtattgtaagtgataggtcatgttgatagcatgttgacagtattgtaagtgataagtcatgttgatatgttgacaatggtactttatatctgtagtatcttccccctaaaaacccacaaacacaatctaatcatgagggaaaaaatcatacccttaattgaggaacattctacaaaatatttgacatactttcagacataaggaaagtctgagagactgtcatagaccagaagagtctaaaaagacaggagcaactaaatgtatttcagtactctaggtgagactctggaacagaaaaagaacattaggggaaaactactaaaatctcaataaagtataggatttagttaataatatatcagtattggttcattagttgtgacaaatataccatagtaatgaaaggtattaacattggggaaattggatctgggttgtacaggaactctctgtatttttacctttcctgtaaacctaaaactatactaaaataaaaaaattaaaaaagccttttttttttttttttttttttttttttttttgagacagagtcttgctctgttgcccgggctggagtgcagtggcatgatcttggctcactgcaagctccgcctcccaggttcaccccattctcctgcttcagcctcccgaatagctgggactacaggcgcccgccaccacgcccggctgattttttgtatttttagtagagatggggtttcaccgtgttagccaggatggtctcgatctcctgatcttgtgatctgccctccttggcctcccaaagtactgggattacaggtgtgatccactgcacctggccaataaaaagctttttaaaccaaggcaatgaaaaagctagatcggggtgacactgggcagagtgtggaaatgcttttttttaatatgcttattatgaaggtccatttcaagcctcgaatggtttatgtgttttttgtttttgttttgttttgttttgttttttgagatggagccttgctgtattgcccaggctggagtgcaatggcacgatctcggctcactgcaacctctgcctcccgggttcaagccattctcttacctcagcctcccaggtagttgggactacaggcatgcaccaccattccaagctaacttttttgtatttttagtagagatggggtttcaccatgctggccaggctgctctcaaactcctgacctcaagtgatccacccgcctcagcctcccaaagtgctgggattacagatgtgagctaccacacctggcctggttaatgttttagaactacacaattgtctaaagagttatgtgatctaaagaattacgatgttaagttatgcgttaaaaaggggatgattgggcttatataaaagctaggtataaagttaaatgtttacttaataatcttattttatagaccatattaaataaggagatagcttgaaacaagaaatatttattgactcaactggctgtagtcctggctacttgggagactgtggccgtaggatcacttgagcctaggagttcgagggtacagtgagccatgattctgccaccgtactccagcctgggtgacagagcaagaccctgtctcttaaaacacacgcgtgcacacacacacacacacacacacacacacacacacacccccagcaaatacaaactagatttttcctactcactggattcaatgagccaataatctggattagaatgatacttaaaacctttttagcacaaattttttttaacataaattgtctttcatttaacttcaaataactgaataaactaaaagcagttctcataagaagacgtctgcagatgctaccacgcccaactaattttttatagagacggagtcttgctctcttgccctggctggtctccaactcctggcctcaattagtcctcctgcctcagcctcccaaagtgttgggattataggcgtgagctattgcacctggaccacagatgttagaagaatgaatgagcataatttttaaatgaaaatccagcactgatcattacaagattttgtgatggtaaatatcggtatgtaatttgtaacaaagtgaaggatgtgaaaatgtagaatattctggaaaggtagtttgagactagactataatagggccttgagtgtcatgataatgcatacaggcagtatgctgtaatgaactcaagtatataagtgtgacattttagcaagatccatatggcttggcacaaatggagaagaacctgtcaccgatccgtctgtctttttatgaggttttttttctatgtgaacttcagaagcatggtttttgttgtttttttgtttttttggttttttgtttttttgtttttttgaatctcgctctgttgcccaagctggaatgcagtggcacgatctcagctcactgcaacctctgcctcctgggttcaagcaattctgcctcagcctcccgagtagctgggattacaggtgccctccaccacggccagctaatttttgtatttttagtagagacggtttcaccattgtggccaggctggtcttgaactcctgacctcgtaatccacctgcctcggcctctgaaagtgctgggattacaggcgtgaaccaccgcgcctggctgtatagctttattcttaatagccaaaagctagaagcaacccaaattgttttccaatagaataaagaaataaattggatatattcataaatggatgctgcacaataataatgaaaatgaggcagtggctcatgtctctaatcccagcactttgggaggctgaggcatgtggatcacttgaggccaggagttcaagaccagcctggccaacatggcgaaacctcatttctactgaaagtagaaaaaattagcttggtgcaatggcgcacgcttgtaaccccagctactcgggaggctgaggcacaagaatcatttgaaactgggaggtggaagttgcggtgagccaagattgtgccactgtactccagcctgggtgacaaagcaagactttgtctcaaagaaaaaaagaaaaatatttaaaaagaaaatgaataagatacttctatatacagccatatagatgaatctcatgaataatgttgaccaaagaagaagagcataagagcatactctgttactacttttatacaaatacaaaacaggcaaaacccatctgtaatttaagttaggatgaaggttatttttgtagagtaggagtagtgagcaattttaggaggcataggagggggttctggggtactgataatatatttcttcatctgggtgtgatcactttgtgagcattcattgggatgtacatttatatatgagacacacagaatagcttggaaagagagaaactgaaatcagtgccaacccaaagtctttgatgcctgtggggaaatgtgatagtgggtagaaaggtgggaacataggccgggcaccatggctcctgcctgtaatcccagcactttcggaggctgaggcgggccaatcacctgaggtcgggagttctaaggccagcctaaccaacatggagaaaccccgtctctactaaaaatacaaaattagccaggcatggtgctgcatgcctgtaatcccagctactagggaggctgaggcaggagaattgcttgaacctgggaggcaagactccgtctcaaaaaaacaaaaaagaaaagaaagctgggaacatttgaaaggggagtagccaaaggtcatacccacattaaaaaaaaaagcagtttttgttcagtagtgagaataatggcctgaaaaagcagtgagaaacaggagacaccgctttctctactgtaagtgcctttaacatggaagaatgagcaacttacatttttaaaggagaatcagatattagggcaatagattccattcactgggctactggacttaactatacataagaacgatctgagtagctttttgggaaaaagtcctggatctcacctccgtatcctttttttttttttgagatggagtctcactctgtcaaccagactagagtgcagtggtgtgatcttggctcactgtaacctctgcctcccaggttcaagcaattctcctgcctcagcctcccgagtagctgggattacaggtgcctgccaccacacctggctattcttttgtattttggtttcaccatgttggcaaggctggtctctaactcttgacctcaggtgatctgcacaccttggcctcccaaagtgctgggataacaggcgtgagccactgcacccagcttctcaccccagtattcttgttcagtgtatctaggatgtaattttaaaaactccttcagctgactgacatacaacccaatttgggaattccttagtaaaagaaaagaggtaaaaacagttttcagctgggtgcggtggctcacacctgtaatcccagcactttgggaggctgaggcgggcggatcactaggttaggagatccagaccatcctaacacggtgaaaccccgtatctactgaaaatacaggtgtggtggtgcatgcctgtagtccccactacgggaggctgaggcaggagaatcgcttgaacccaggaggcggaggttgccgtgagtggagatcgggccgctgcactccagcctgggcaacagtgagactgtgtctcaaaacacaacaaagaacagttttcaatgtgaaggggaaatcgttttctgttatttaataggaattgcagtaatacagaggctgggaagaaaaactgtcagtgagataataatccttagaagaggaggcagaagtgggagacagtgagaacatgaggatgtgaaaagcataaattattaattggcttgaagcttctgaacaatggaactttcctcctaagttatatcctgctggatctctctgaaatactcatgggtagttggaacctgactttttaatattttatattatctactttcctgcgtgtagcattgttttaaaatgttctatctctttaaaatatatttaaacagctttattgaagaaggtcagttttaagcaacatttccatatctggtatttgttatgcattatactaggtgctagagaaaagtcagatgcttttcttggcttaaagagcatagtccaatataggggtataggcataatatctgcctacagatgtctgtaatgtgcatcagtcattaacatgacagtccaaaaacttcaaattgtaccccattatttaatcaaagtagaaaaagtctaccaaactgtcatgacctatacattttactgtccagagacttgggggagatttctattttggtgtctaattctttgtaatctgttatactatgggaatttgtcttcttttatctaaaaccaggtggtcatgggatataatctccttctccttacccttacctctcatccccccaattgtgcacatcgtacacattgtcatctgtttttttctagggctcaggatggggttgaggcttgcttctgaattctaagtactaatgaggccaccactcaatcactgtgctaatgtacaactgtctggttccttctttcttttttttttttttttttttttgagacggagtctcgctctgtcgcccaggccggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccctggttccttctttctgcttcttatcatgaacctgaactcctaagagataggtccatagacaagtattcatataatgttaaccagttcattttttcatttaagtatagctaagtttgtgcatatatatatcattttctacgcctcttttgcaggtagtagccatggttatgtgggttccagtagtagaatatcaagaagaacacatttatgctccgctgctaccagtagtttactagacattgatccattaattttaatacatttgttggaccttaaggaccggagcagtatagaaaatttgtggggcttacagcctcgcccacctgcttcacttctgcagcccacaggtaaaataataacaatgataatttcctttttttctgtggttctctaaaagaactcttggattttcatttaagtatttcagctatgtgtggaacatggtgtatttctgtgtttttcattgtataccataaaaatgacatttatgtcaaccaaattagtttttaaattaccgttcaaaggtgatttgtgcaaaaacaaagtaaaaatgcctgcaagaaggttttttgtgacttgctactcagattttgcctgacagtttgatccatatgaaatacggttatcttctttatgaatggactttttttggtcaggcttcatgaattcccttacttccaagtggaactatatctaaattgaacgtagaaaaaggtgacttactcattctttgatgttcagtagcttaaaaatgtatgccagttggcagcactgttttctgtgcaaaggttatttgttaaatagaaattacatttgtgttactataataaaatggagatttgtatagtcaaatatgggcttttttaaactctcggggatagagtcattttggacaactaagtatttgcctttaaaagctacattttaaaaaccatttttgttggaatgctttctaaagtatagtcagtatattcaaaaatatatgccaaatatttttctaccttcttaaacaatagtattatatacatcaaatcaatgatttagaactctgattttgatgtagttttaaggcaagataactcatttttcaatcaaagtccaaggcagaacctctgtgtaaataacagagaaatagagctcttctactggtgtagggacatggagcccagattatttaggcttcttctttcctaagagccagatatacttcctgaaagtctggatcctcagcttaaaagactatcttcaggttatgaaacagttgccttttatgtgtgcgccaagtatgctttatataaagttttacttaaatgttaagtgttgtttgcaaaggcccagtgaaacagacaggttctgattctgtaggtagatatgtaagttggtttaacctttttgaaggacaattttgtagtagtttaaaaatatctatatcctttaaacttgtaatttcactttgaggagtatattccaaagagaaaacaagcagatgtttaaaaatattcaacaatacagttatagctgaataaattacagtgtaaccatgcaatggtatattttatagacaagtaaaatcgtgtttttgaaaataatcagtatcattgggaaaaatgcaaactaaatagtaagatttctgtgtaaaactagttttctgtgtaaaaacaatacaaatgagatgatctgaatttcaaatttaggtgataaaatgctggcaataatgagggcggggaaaatagtagaaagaaaaagcaagttttggatttaaacatcttgagttgggcataacagtgtgacatcaaattagaaaggaccagtagacagctaaaaattaagaactggggctgggcgcagtggctcacgcctgtaatctcaacacttggggaggctgaggcgggtggatcacctgaggtcaggagttcgagaccagcctagccaacatggtgaaaccctgtctctactaaaaatacaaaaaattagccggccattgtggcaggtgcctgtaatcccagctacctgggaagctgaggcaggagaatggcttaaacctgggagtcggaggttgcagtgagccgagatcgtgccattgcactccagcctgggcaacgagtgaaactccatgaaaaaaaaaaatgaactggaatacagacagttatttttctggaggtgatactggataattaaaaccaagggtgtgagattgcaaaggagttttaagaatattttaaaagaagagagccaggaacataatcctgatgaatatctgtttttactgagtgtcaggaagatgagccagataaagagcttttagaaaggagaaaaaatactcatggaagccaaatgaaaatacctttcagtgaagggatgttaataaaagctagtgtgcagaaatatatgactttctttttattgaaaaggcttgcttgggtcattttttgtttgattgattgtttgtttgtttttgagacagagtctcactctgttgcccaggctggagtgctctacaattttttgtagagacaggtttcaccatgttggccaggtttgtctcaaaactcctgagctcaagtgatccacctgcctcggcctcccaaagtgtgctgggattataggcgtgagccactgcacgtggcctgcttgggctttgatgtttatttttttctgtgtatttatttaacacatatttagtgaacatttgtcagattaacttcatgttcttaaccatgtaaaggagtactgtaaaaggtatcaggtaatctagttgaaccagattttaaggtacatatagtggtaagtagaactagagtcatgttgggtccattctatgaaaggacttgcatgctagccggaggaatttattcatactaggcatggagagctattgttttattttagaagtggttagattcaaaccgtatttcagaaagctgacagttctgagaaaattatcagaaatggaagttctaataaaaaaatttttttttgtacagatagggtgtcactatgctcccctggctgatcttgaacttctggcctcaagcgattctctcacctcagcctcctgaagtgctgggattacaggcataagccactgcacctggccagaaatatgtatttttaatataatgttatatatggcagatttgacaaagtatttggtttctagatttttctttgtcttttttttcaattataaacataataaaagctctgtagaaagtttcagaaaagtggagaagtttaaagaagaaagcacaaggcagtagggcagcttttaaacttttagaattaaatgtgtaattcgaacttgagtatgatgtgactcagattctaactctgaaggtgtgaataaggtaggattctttatctttattttcattaatggaatttagtgcatgttttattgttgataccacgtaaagcacgctagcttattttacacatcaaatttgcatatattcatggtacaaatgatttcattaatattaacgtttatgttaacatgacttgtcaatttctctgaagcatcatattctcgaaaagataaagaccaaaggaagcaacaggcaatgtggcgagtgccctctgatttaaagatgctaaaaagactcaaaactcaaatggccgaagttcgatgtatgaaaactgatgtaaagaatacactttcagaaataaaaagcagcagtgctgcttctggagacatgcagacaagccttttttctgctgaccaggcagctctggctgcatgtggaactgaaaactctggcagattgcaggatttgggaatggaactcctggcaaagtcatcagttgccaattgttacatacgaaactgtaagtaatatgttcccagtgtcatttctccacacgttatttgggaaactagatgactggttttttcaaaagaataatatttaaataccactcttccagtgagttaagactacaaattacatgtgacttttttcttcatccaaaattgattccgtatatttttactatataatagaactaacatttcttgattgcttactctgtatttggcaataaacaagacaaaattcttaacatcatagatcttgtattctagtagtgggaatcagacattaaaaagtgtagacaagtaagtaaagatcatttcagtgggtaaaaggtgctgtgaaggaaacaaccgaggagtgggatgggaggagcagatgagacatgttctattggggtagtcagagaaagtctctgtgaggaggcagcacttgacctgagacctgaagtttaggaaggtggcagctatttatagatctagggcaagcagtgttctgggtaaacagaacagaaactgcaaagtcactgacacaggatttattttgcgtatttgaaggattgaaggaagaccagtatggcaaagaaagcagagagaggagactagcaggagacgaagtcagagagaggtaggcagaatttagatcagggagagtcttgcaaaacctcaaaaaggagttcgagttttattatgatgtaaggaggaagcctctagagaataaaaataaggggtgtttataatctgatttacactttacaaagatgactttgcttgttgtgtggagaagggtctgaaaagggttgaggacaggaattgtgagaccagtaacagcctattacattatcttgtgtgaaatatgatggcattttctggagttacaataatggatagaggggcttgttcacagtctagcagcctttcttgactgaggttcccatctttcttagttctcttaaggatgtgctattctattctagatgcataggagggaagttaatccagtcttagatcagcagggctgagttctttctcagaaccatagttgaaaaagcctaaatagaattttaggaaagttctatttagaaagaaactaagaattatgattaagttttggcctaagcaacttaataggcagtggtatcatttattgagaagcaaatcagataagaagcaggttatggggcttgggaggaggtaagggcagaaagttgggtattcttttttaaacatgtttaatttgagacacctgctagatatcctagtaaaatgtcatagacacgtgaatggtacacaactttgaaactcagagagaggtcaatgctggatataaacagttgggagtcaatagtattttatattatttaaatccagaagactgaatagggtcaagtttggagggagtttcaatggagaagcaaaatttttgactctgtggcacttaaacatttaaagatctgataaataggagaggccagcaaaggaaattgaaagagcaatcattaaggtaggagaaaaactagaagggataatatctcaaaaatcaaaaataatgcatctcaagaagtagagtggttacccctgtgttaagtgtgttaagtatcactgagaagtgtcaaggcaagaggccaagtggcctttttaagaactattcccatggagtagtgtagcagaagagtgaatgaggtcagggaccagagcacaaatgcacaacttctttaaaagaagtttcacaatgaagtgggtagaagctggaggaggtgcggggtcaatagtttcttgttggcaggggtgtccttttttgtgtcctctccaatgtctttcatctgtgaattaaatccattttaaaattgggttgggttgtttatttgtagttgacttttaggagttctgtatatattttaaatatttatcttttatcacatatatgatttgcagatgttttctcccagtgtgtgggttgccttttccctctgtggattgtgtcatttaacatttgaagtttttaatttgaagaagcccattttatgtgtttttccttttgttccctgtgcttttggtgtcatagccaagaaaacattgcctaatccagtgtcatgaagctttctcctgttttcttccaagagttttagttcttgcttttagtcttacttttagtctttcatccattaagaattaaattttgtttatggtgtcagggaagggtctgacttgcttgcccatggatatccagttttcccagcactatttgttgaaaagacttttctttcctcattgaatggttttagcaccattgttgaaatcatttgaccatatagacgagggtttgtttctgggttctctttgtgtgtgttgtttattaaaattggctgtattaatgtttgatagtgatgtgaataatccaataaagatgggttggtgaggagataatagcaaaatatatagaattctcttaatataaggaagacaatttagtagaatgaataaaggttatgaataggccattaacataagaaatacagatgtacaataaaaatatgaaaagatattcggcctcacctgctattcaaggaaatggtatattaaaccaaatggtatattaaaaccaaaagtacagctttttgggtggactttctcaaaaagcctttgttacatattttgcagatacagttgacccttgaacagcacaggcttgttgtactgtacaggtccacttaatattcgaatttttttcaaccagagaaagattgaaaatacagtattcgggtgatgccaaacctgcatataggagggccaactttcgtataggtgggttctgcaggtcttacggtgggacttcagtcgaggattttagtatacgcaggggtcctggaaccaacccccacatataccaagggacaactgtctcttccccattgccacctttttttcctgctaatcacagagttttaaaattttatcttagacaactgtattaataatattcatccatgaatattaataatataattaacattcatgacatgaatattaataatattataattaataataatattcatggcaataatattcatgggtcttatttttatttggaaggctttttaactcccaggttaataaaaataacccttaatttcttctggtactttttgttttctaacttttttacatataggtcattaattcatataaagttaattttatatgatgctgtgtgaggtaaatggaatgatgtttttccccaaatgaatgaaacatgttttttgaaatatgatgcaatatgaaatccatcctttattatagaccagattcccaaacatggttctgtttttgaactcatagaggtcgaaacaactgaaattgccagtactcaagttttggacatacaaaaacgcaatttggtattgtttaacccagtatttgtcatgtcctctaccagactaacactttttttaactactgtagttttatagtttgtaatactatgataggatatgtttattctgttcttggttttcaaaatgttattggtggttcttgcacttttaatcctcctccaaagtattttgactggaattgctttacatttaaagactagtttaggggtgctgggcaccatggctcctgcctgttaatcccagcattttgggatgctgatgagggaggatggcttcttgagcccaggagttcaacatcatatggcaagaccttaatctctacaaaaataaatttttttgataagccagtcatggtggtatgtacctgtaaccccagctacttgggaggctgaggtgggaggattacttgaggtcaggaatttgaggtgcagtgagccgtgatcgtgccactgcactccaatctgggcaacagtctctctctctctcttaaaaaaagagagactaatttgggcaattgagatcttttcagcatcaaacattcatgtctaggaatgtggtacttctctccatttattcaggtcatctgttatattcatatccttttttagaactctcagttttttccatataagttctatatatgtcttgttaaattcatttctcaatgtattatatcttaggtagctgttgtgagtggaatctctttttctctgatagtattttctaattgttactgacataaaagacaaaaatattgatttttatgtttattctgtattcaaccaatgtggcaatttttttttttttttttttttttttgatacggaatttcactcttgttgcccagcctggagtgcgatggggcgatctcggctcactgcaacctccccctcctgggttcaagtgattcttctttgtcagcctcccaagtagctgggattagaggcacatgccaccacgcccggctagtttttgtatttttagtagagacggggtttcatcatattcgtcaggctggtcttgaactcctgacctcaggtgatccacccacctcggcctcccaaagtgctgggattacaagcgtgagccaccacacctggcctggtgaatttgttttattaacagtttttcaattagttattttgattttctaagtatttatatcatcagcagataatcataatttgtttattcattttctgcttactttttgtattgcattgtctaggcaggtacctcaacagtttcttagtaacagtggtaggatttttataatttttcaactttatttaggaattcctttagtttttttctcattaaatttgaggtttgctgtatgtttcaataggtacttttttgtttttgtgtttcttttttttgagacagagtcttgctctgtcacccaggctagggtacagtagctctatctcggcttactgcaagctccgcctcctgggttcatgctattctccagcctcagcctcccgagtagctgggactacaggtgcctgccaccacgcctggctaatttttttgtattttttagtagagacggggtttcaccatgttagccaggatggtcttgatctcctgacctcgtgatctgcctgtcttggcctcccaaagattacaggcctgtttctatcaagttaaaaaagctgtgtgcaatggctcacgcctgtaatcccagcactttgggaggccaagtcgtgcagattacttgaggccaggagttcgagaccagcctggccaacatggagaaaccccatgtctactaaaaatacaaaaaaattacctgggcatgatggcatatgcctgtaatcccagctactcaggaggccaagccactagaatcgcttgaacccaggaggtggaggttgcagtgagccaagattgcgccaccacactccagcctaggtgacaagtgagactgtctcaaaaataaataaataaataaataaaaaagttaaaaggtacatccttctatttatagtaatagattctatagtatagtatagtaatatattcaatctttctctggttgaaaaaaattcgaatattaagtggacctgtacagtacaacaagcctgtgctgttcaagcgtcaactgtatctgcaaaatatgtaacaaaggctttttgagaaagttcacccaaaaagctgtacttttggttttaatataccatttggtttaatataccatttccttgaatagcaggtgaggccgaatatcttttcatatttttattgtacatctgtatttcttactaaagaaatttagtaactaaaggttactaaacctgtttagtaacaacaataccaataatggctaacatttactaagtgttaactgtgtgccagacattattctaactattctaagtcacatgcattgactcacttaatcttaacaagaagtctaagaggtaaggtaagtcaatttcattccagatttacagatgaggaaaccaaggcagcaggcattgaataatgttgctagtttcacattggtaattggcaaagccaaaaacttgaacttcagagcctatgcccttcaccctaataccaaactgcctcttttcttactacgagaatttcttttctttttaaactgaagctgtgtagaattatatcaaatgatgttttgacatttgtcaaaatgatttactattttgttccattactttgttatcataaagtaaattatattatttttctaatgttgaccattctggaaatactggaatgcatcctatttagtttgggcctatactttttttaaaaaaacactgctgtattcagtttgctaatattttacttaggatccttgactacatagtcataagtgaaatcgatctatttcattacactatccttaacccactttagttctaattctgtgcttgttttacagattaaattcagatgtttttgtccttttctatgctataaaattattttaactatatgttatttgctcctatgctgtattaaactttatgtctaataaaaagcattcataaaactagtatagtaactcagagttagaactttaacagcctttttaatttctgttggatttgttggtttcctcatatttttaatttttttaatttttaatttttgtgggtacattgtaggtatatatatttataggatatatgagatattttgatacacgcttacaatgcataataatcacatcagggtaaatggagtatccatcatctcaagcatttatcctttgtgttaaaacaatccaattgtactcttttagttattttcagaagtacaataaattattgtagactgtagtcactgttgtactaacgaatactagatcttattcattctatctaactatatttttgtacccattaaccatccccccagccacccactacactccctagcctctggtaaccattgctgtattatcttgagttcagttgttttaatttttagcccccacaaataagtgagaatgtgtgaagtttgtctttctgtgcctgatgtatttcacttaacataacgatctccagttccacccatgttgttgcaaatgacaggatctcattccttttttatggctgaatagtattccatgtgtatatgtaccacattttttttttgttttttgagatagagtctcactctgttgcccaggctggagtgcagtggtgcaatctcggctcactgcaacctctgcctcctggattcaagtcattctcctgcctcggcctcccagctagctgcgactacaggcatgcgccaccacgcccagctaatttttttgtattttttagtagagatggggtttcaccttgttggtcaggctggtctcaaactcctgacctcaaatgatccacttgagtcgggcttacaaagtgctgagattacgggcgtgagctaccacgcccaacctaacattttctttatccatttgtctgtggatcttaggttgcttccaaaccatggttattgtgaataatgctgtagtaaacatgggaatgcagatacctctttgacatactaatttgctttcttttgggtatataaccagcagtgggattgctggatcatatggtagatctatttttagtttttctgaggaacctccaaactgttttccgtagtggttttactaacttacattcccaccaatggtgtatgagagttgccttttctctacatcttcaccagcatttgttattgcctgtgttttggataaaagccattttaactgggatgagatggtatcttattgtagttttgatttgcatttctctgatgatcagtgatgttgagcactttttcatatgcctgtttgctatttgtgtgtcgtcttttaagaaatatctattcagatttttacccatttttaatcagattattgaattttttcctatagagttgtttgagatcctggtcattaatctcttgtcagatgtagttttctcatacttctgctgctacttaagtcagttactatagttttccaagaaaaactaattatatttgggtctttaaatttattgatatacagtcatttattctattttcttaaatttttaaaacttcttgtgcatatgggttgttaacttctttttcacttattttattgcttacttctgtttttttttctcctttgatttgacctgctcaagggatttgctaattttgttcatctcctcaaagaccaagttcttagatttatttcatccattgtcttttaccatttcattatatttctgcttttgtctttgttaatgcttttttttttttttttttttttttttttttgagacagagtctcgctctgtccaccaggctggagtgcagtggcgtgatcttggctcactgcaacctccgtctcttgggctcaagcacttctcctacctcagcctcctgagtagctgggattacaggtgtgtgccaccacaccgggctaatttttgtatttttagtagagatggggtttcaccatgctggccaggctggtctcgaactcctgacctcaggtaatccacccactttggcctcctgaagtgctgggattactggcatgagccaccgcacccagactgttaatgcattcttttttccttaaccaataattcatttatccttttagtggagaaattggtcatcatttttcgtaagtctagaacatttatctaagtctaagaacattttcattaaccctctccctagaactccaaacccattaaaagtcgttccccatcttccccttctactatcttctggtaaccactaatctactttttccctatggatttttcttcttctggacttttttttttttgagacggagtctctccctgttacccaggctggagcgtagtggcacaatctcggctcactgcaagctccgcttcccgggttcacgccattctcctgcctcagcctcccaagtagctgggactacaggtgtgcgccaccatgcccagctaacttttgtatttttagtagagacgaggtttcaccatgttggccaggttggtctcaatctcttgacctcatgcgccacccacctcagcctcccaaagtgctgggattacagatgtgagccactgcgcccagcctggactttttatatagactcataccatatgaggtcttctatgtctggcttatttcacttaacacagccctttcaaggttcattcatgttgtggcatatattcctttttatgtctgcatgatattttattgtatggctgtactacatactgtttatccactcattagttgatggatatttgggttgtttttactttttggctattatcagtagtgctgctatgaacgttcatgtacaagtttttgtttggacatatggttttgtttgagtatatgcttagaagtggaattaatgggttacatggcaactgtgtttaactttttgaggaactaccagtgccagactattttccaaagtagctgcatcattttatattcccagcagcagtatatgagggctccaggttttccataccgttggcaacacttattgtccctctttttttccttgcccattgcattgtcttggcatccttgtcaaaaatcagtggaccataaatataagggtttgtttctagactctcaattctgttttgatgatatatatgtgtatatggcagtaccacatagtcttgattacttagttatagtaacttttgaaatcagaagtgagtgttccatttaagattgttttgggtattgtgggtctcttacattttcaaatggattttaggattagcttgtcaatttctgcaaaaaaaaaaaaacacagctactattattttagaaattgtgttgaatcggtagatcaattttaagaatattgctttgttaacagtattaagtattctaatcagtgatgaacacaggatgtctttgctgttgtctctattttctttcaagaatgtgtggtagttttcagtggacaagtctcacatttcttttgttaaataaattcctaagtattttaattgtattttatgctattaaaaatgaaattgttttcttaacttcattctcacattgtctgctagtgcagagaaatacaataagttattttgtcttatatcctgcaaccatgctaaacctgtttattacctattaatgaattttttatttttattgttttttacaattttatttttattctttttttaaaatgtatttttaaaagagatttatttaactcacagttctgcaggctgaattcaagggcagggccctgccttctggcaatagcattcttgcttgcttatttttattgtcttcctagcttttgaatctatgcatattaggtcatatagtcccttttttccctaattaattcattttaaggccgtacattttcttttcactactggtttggccacagacattttgatttcaagtaccataattgtaatttgtttctaactcagcattgatttttctttttaatccaataatactttataatttccaaatgtttgacgggttatatatttgttaatagtttaactgcattgaaatttgtatgatttttcttaaggagctcacagataatcttttaagttaagattttacgggcggtgtgcggtggcccatgcctgtaatcccagcattttgggaggccgcggcaggtggatcacctgagttcaggtgttcgagacccagcctggccaacatgacaaaaccccgtctctactaaaaatacaaaaattagctgggcatggtggcgagtgcctgtaatctgagctactcgggaggctgaggcaggagaattgcttgaacccaggaggtggaggttgtagtgagccaagatcgctccattgcactccagcctgggcaacaagagcaagactccatctgggggaaaaaaaaaaaagattctgctctacaagcaacaagatgggaatgttttatgacaaaagtctaaaccaattggggctacagaatgttgtttatttttagacagtagagagaacagcttccatgcaaggtaagattaaaactttttctgacagactaaatagtgagttttgtgctgtagtagcctattcagaggtgttcttaaatgtctttgcagccacaaataagaagagtaattcgcccaagccagctcgatccagtgtagcaggtagtctatcacttcgaagagcagtggaccctggagaaaatagtcgttcaaagggagactgtcagactctgtctgaaggtaaatttgagggattcagaatgtacttttagtggggtaaatatttattacctgaaattgatgttatcgtggagctttagatcttgaagggcaccaaaaatgctaaagttggatttgaaagtttctttttttgaaacagggtctcactttgtcacccaggctggagtgcagtggtgcgatcttggctcaccgtggcctcgaccggtggtttaagagatcctcctgcctcagccccaccaagtagctgggactacaggtgtgcaccactacgcctagctaatttttgtattttttgtagagacaggttgcccaggctggtcttaaactcctgagctcaagcaatccgcctgcctcaacctcccaaagtgctcggattacaggaatgagccccaccacacctggctggaaagtttcttgatctcaacagaacagacccttaacaaaatacagccattagactatgtattctaaacaccttaattttatttattctttatttttgttgttttcctgagacagagtcttgctctgttttccaggctagggatgcagtggtgcaatcatagctcactgtagccttgacctcccagacaaaagtgatccttccacctcagcctcccaagtagctgggaccacagacacagacacatgcagccatacctggctttttttttttttttttttttttttttttttgccgagacagggttttgctacattgtccaggctggtcttgaacttttgggctcaaacaatcttcctgtctcagcctcccaaagtgtaaaattacaggtgtgagccacaacacttggcctggactccttaattttagatgcttggcataaaaaacaagggatatgtttcaaaattatttttgaagtttttttttttttttttttgagatggagttttttcctctagttgcccaggctggagtgcaatggtgcgatctcggctcaccgcaacctccgcccgccgtgttcaagtgattctcctgcctcagtctcctgagtaactgggactacaagtgcacgccaccacgcctggctaatttttgtatttgtagtagagatggggtttcaccacattggccaggctggtcttgaactcctgacctcaggtgatccatccgccttagcctcccagagtgccaggattacaggcatgagccaccacacctggcctatttttgaagtttttatgtagttttaaaatgatatatatagtgagattttctaatgtaactgagagagacctttcatgtttatttgccttttttctgattatccctccttttattttattttattttattttttggagacagagtcttgctctgttgtccaggctggagtgtagcacaatctcagctcactgcaacctccgcctcccaggttcaagtgatccttgtgtttcaacctcccaagtagctgggattacaggcgcacaccaccatgcccagctaatttttgtattccatcttcctttttttgaaggctccccaggaagctctcagtctgggagcaggcacagttctccccgagccttgatacatggcagtatcggtgatattctgccaaaaactgaagaccggcagtgtaaagctttggattcagatgctgttgtggttgcagttttcagtggcttgcctgcggttgagaaaaggaggaaaatggtcaccttggggtaaattaagttgttttgtatatgtaatggtataatagctaatagctttgtttggaactttaagtatgcatcatgatgaacaaattaatttctaccttgttaattttttagtggacagaaaatacatttattgcactaaattttaacttttttaattttttttattttttttaacagagcctcgctctgtcacccaggctggagtgcagtggcgtgatctcagctctctgcaacctctgcctccccggtttaagtgattctcctgcctcagcctcccaagtagctgggattacaggtgcccgccaccacaccctgctaatttttgtatttttagtagagacgaggttttgccatgttggccaggctggtcttgaactcctgagacctcaagtgatacgcccacctcagcctcccagagtgttgggattacaggcgtgagccactgcacctggcctaaattttaacttttttaaagtatcagagattttacttttttttttttaatgggatgagatagtactttttttttttttgagatggagtctcgctctgttgcccaggctggagtgcagtggcatgatctcagctcactgcacgctccgcctcccaggttcatgccattctcctacctcagcctctccaagtagccaggactacaggcgcccaccaccacgcccagctgatttttttttttttttggtatttttagtagagatggggtttcaccatggtctcaagagatagtactttttttaatgggatgaaatagtaccggaagtttagtttaaatatgttagagctcattttcacaatgatacgtaaccttatctgaactctgccttgtaaatttgtatgtgttggcggtaggggggcggtcagccttttttttttttttcagtctatcctacacttgtcaaagatgttagtgttgggctatattatgtgtcacaggcatttatatatattttgtatgaaacaaagcacttacagcaactttaacactgcttttctgcttttatctcatctttcttctctcctgttatcaaagtatcctcaaaacttatagttggaatttcccaaaaagatatatagtagttaacacgtttatctctcccctaggagacataaactcaaatccccggcatggcaattaaacttccatctattcaaaagatagagtgattatggatattggctatggatgtggcagatgattagccattaatatccatctgcttatctattttatggatattaaacctatttctaaatttaaacaatgaaatagttctattttggagaccctgaaaagaattttacctaaattggagaaagattaacaccctttaagtttacttaattttatatagtcaaggtctggctcattaccaaaactacaagatcagctggtggtttagctttctggctcaagtttcagtagtagaaaggtgttaaaatcactggagattatcttgtcttccctacgtactacctaacctgatgttttatacatttcttcaaattaaagtgcaactagctcttatattttatttgctatggagatatttttatctgcctttagtcaccacaattaaatatacatgaacttagtttttttttttctagagtgtaaaatcctgtgcaagtgtgtgcagcttgatcctcattttgcagttgattttatctgccagcctggttcatgtaggtgctgtccagctcatgtctttttttttcctgcctgatttaaagtagtgtaagcaaaaagtttaaatactaatgaaaggctgggcacagtggcacacacctgtaatcccagcacttttggaggctgaggtgggaagatggcctgagcccaggagtttgagaacaccctaggcaacatagcaagaccttgcctctgtttattttttaaataaaaataaatattaaatactaatgataagtgtttcaaaatgaaaggagaaaagaataagtgaaaaatttcccatccttaactcccatgacctagttttccaaccccctgcccacacatattcatgctgttattaatttttcatgtgtgctgtgtcttctcagtttttgtgtatatttaaggaactgtgactatgtagcttttttaatacagaagatgtactttattttctcattcaataatatctctcaggccaggcacggtggctcacacctgtaaccccagcactttgggaagctgaggtgggcagatcacttgagatcaggagttcgagccagcctggccatcatggtgaaacactgtctctactaaaaacaaagtacaaaaattgccgggcgtggtgctggacacctgtaatcccagctacttgggaggctgaggcaggagaatcgcttgagcccaggaggcagcggttgcagtgagctgagatcacatcactgcactctagcctgggtgacagagtgagactctatctcaaataatagcaataataatatatctcaaaagtctttccctattagaacatattatttaaaacttctattggtatttataagttaaaattttattgagctaattataggttcacattcagttgtaagaaataatgcagagagatcctgtgtatttttctcccctaatgataacattttgcaaaactgtagtacaatatcacaaccaggataccaacattgatgtaatccaccatttcataacatttttttgtgaatatttagtgaaaaaaaaattttttttttttttgagacagagtctcactctgttgcccaggctggagtgcaatggtgcaacctcggctcactgcaacctccacgtcccgggttcaagaaattctctgcctcagcctcccgagtagctgggattacaggcgcccaccaccatatctggctaatttttgtatttttagtggagatggggtttcaccatcttggccaggctggtcttgaactcctacctcaggtgatccacccaccttggcctcccaaagtgctgggattacaggcataagccactgtgccaggaccttagtgaaatattttcagataaaatttgaaatttagtattaagcaccagagcacgtaccctacttagtttggtgctcagttcaaaaagaaaatatagattattgtgtcactagttatagagaaatatcaagttgttttcttctttgagggctaatgctaaaggaggtcatctggaaggactgcagatgactgatttggaaaataattctgaaactggagagttacagcctgtactacctgaaggagcttcagctgcccctgaagaaggtaaggaatgataaacttataagtaagcaggaaatgagaattctgtcccttttttggtggtgggtggtgggtggtggtggtggtggtggtggtggtggtgctgctgctgctggtggtagtgatagggatagggagtggttggtttaatggaaatgttactgagatctgctattcgacatgcttgatctgaacacaacattccttgtgccactgaatttgatttgctaagataccaaatacagaggaaggcaaaatgggtaagttactatataaaaggacaggccttactagaacaggcatttgtatggggaaaaaatctgcatatagttcatctgttcggtcaataaatatgttaaggcacctacataccaggcatcgaatttttgctagatttattaatacaaagttgaattagatatgttcacaatctaatggggaagatatgccctagaacaactaattagaatagtaaatgttaagtattttttaaagtcagtcttaattagtgtctgtagattgctttattcttcttaaagaagcattccaagttttgaccattttttaaatcctgtatgatgactgacttcagcttgttctcagctgtactttctagtgtttcttttgcatgttcttccaagttcattattgtttatctaaaagcatagaagacattttgcaatttggggcaacagaggtgcttagaacaaggctatgtgtattgcccttgctgtgtcctttataaattggtagaaaagaatgtcaactctcttaatgatatttggccttgtaattttttcatttcttagtgaaactccaccaccttccacctctgcactcatttgaatttcaatgattgatgaattgtagctcaaagcatacatacaggtgccagcatctactcagctgccctaagaagagggtgtgtaggtggtggcattttcaggtttctcagttaaagaacactaaatattttggttttcttaattggtgcttctgtgatgggcagcattatttcagtagaagagattaaatagaaaagatttttgtaaatgatgtgtttattgttaaaacatcaagaaagaaatattatgattgaagatctttatagaatgggcctgtggggaatactttctttttctgttgttttttttttttccccgagacggagtctagctctgttgcccagtctggagtgcagtggcacagtcttggctcactgcagcctccaactcccgggttcaagcgattctcctgcctcagcctctgcatagctgggattacaggcgcttgccaacacacctggctaatttttgtatttttagtaagagacgggatttcactgtgttggccaggctggtcttgaactcctgacctcgtgaaccactcgcctcagcctcccaaagtgctaggattacaggcgtgagccaccaaggccggccggggaatagttactttctatgtgtctccatacatactggattgtagtagcttaaattactacagaatatgctggaattacaagctcatttttcttccaccaagcgcagtggctcatgcctgtaatcccagtactttgggaggctgaggcaggcggatcacttgaagtcaggagttcgagaccagcctggcaaacatagtgacacctcatctctactaaatatagaaatattagccagatgtggtagcagacacctgtaatcccagctacttgggaggctgaggcaggagaattgcttgaactgggaggcagaggttgcagtgagctgagatcacgccactgcactccagcctggacgacagagtgagactctatctcaaatgacagtaataacaagctcatttttttcatgcaacatttatgaaatgcttctaatttaccggatgttgcatttgatgccagtgataaaattcctgaacttactatctagttcagactttctccacctttgcaccattgccatttaggccacataattctttattttacagggactgttctctgcactgtggaatgtttagcagcatcctcagctgttaaccactagataccagtagtgtttccctccggttacaacaaccaaaaatgtctctagtcattgccacatgtttcctgggaggcaaaatcatccccagtcaagaaccaatagttgaactaaataattctcaaccagaggtgaccactccttgagaatcacttgccagagtgggccacagttattaatagaaataagttgtagctctcaggtatgctaggtatgtgtggtggtattggtaggagggcggcaaggagagcagttgaaaaccaattgttaggtgtcaggggagactaatagccagttgtaatataggacgatgagtgctgtgataaggtttataggcagctcaaaaatgagatacgtaataaaaataatagtaatactaagttcttactgtgttaaagactttaccagtattctgttattctcagaataactctacatggtagttgcttttgttaacctagttttacaggtgagtaagctgaggcctggagatgtaatttaccttacccaaagtcacagggttagaaattggaaggccagggccgggcgcagtggctcatgcctgtagtcccaacactttgggaggccaaggagggtggatcacctgagttcagaagttcaagaccagcctggtcaacatggtgaaaccccatctttactaaatatacaaaaattagctgagtgtggtggcgggcacctgtaatcccatctacttgggaggctgaggcaggagaatcgcttgaacccgggaggcggaggttacagtgagctgagatcgcaccattgcgctccagcctgggcaacaagagtgaaacttcgtctcaaaaaaaaaaaaaaaaaaagaaaagaaattggaaggttgggcatgttggctaatgcctgtaatcccagcactttgggaaggccgaggtgggcagatcacctgaggtcaggagtttgagaccagcctggccaacatggtgaaaccccatctctactaaaaatagaaaaatcagccaggcatggtggcacactcctataatcccagctactcgagaggctgaggcaggagaatcacttgaaccaaggaggcagaggttgcaatgagctgagattgtggccattgcactccagcctgggtgacagagcgagactctgtctcaaaaaaaagaagaaaagaaaaaaaagaacttggaagagctagaatttgaacctagagccagtgctcttaactgtcgcctctctctgctaacctagtccctaatccacccttggaaagatcatcctaagggatgagtaggagttagccagatgaagactggtgaaaggccagaagggagacagaccagaggccagatcatgaagagttacgtggaatgtcccagctagaatttgaactttgtcatttgaataggatctaagttagaagtgacactttaacatttctactctagctgcattctggagaatggatttgggggtgggaatagcatatgtggaactagaggcaggaaaattggtaagaagcatggttaggtgaaatgatttggacttctttatttttaattttttatagagacttggtcttgctgtatgctcagactggtcttaaactcctgggctcaaatgatccttctgcctcagcttcccaaagtgctgggttacagacatgagccaccacacccggccgcccgatttggacttaatttcagcctccaatttatcaatttacattatctcagcactaatttttatatataaaatggctggtcagcacagaaaacactggaactttttgacaccagtttcatttcttatttaaagctaattgacagcttgtcctaaactgaagaaaggcatttgcttggccatagtgaagatcggtacattttccaagtctttgaaagaatggagttattcatgtaggatttagagttaaatggtcagtgttattcagtaacacattttatctttatatgtggggtgttgtgctataaatagcacctagaactcaataagaaaaattgctgtgaatgtgcaaatcaacagggacacagaagtacacccttgtaatttgttggtatctgaacagcttgtttacctccagagtcccaggatagactgtgagaaggagactaactgatgaggctgagagaaggtagtacaggagacaaaggaagccctgtgcctcctgtaatcagctttgtttctcgaggagagtgcggccttgctcactgttgccagtattattctatcatcagcaagagcaagttctcaggaaacccttatatacctataagccttagtagacagttgaatggatttgtcttgtatccatgcttaggaaaaacaagactctttattttagctgaatacaaaaagaagacattcggtgtcatatcactgtggggttctctctctttctcaaagagaaggttgtctggaggactttaaggcgttgtttgtggtactggctgggaatttgggactactaaagtgtctcttaattgctcttaagcatcagtttaaaaataacttttttcttagatatgaacataaataggccttttcctttttactggaaccatatgggacaacctttggttgtattagtcgctggtttaatgatctgatgctgaaaaggttgacttattttttctttcaattgatgacctgcacaactgtaggtaggccagtttatttcttgttctttgtaaaactaaaggctttgctctctgacttcttgctgaagtagcacatacccccttcagttaaatcaggggtactcaaaacaccatctggggaaaagccttggcatgcctgttaaaaaccttgctggtggcctctttaagaggaatacaaagcctgatggcctgccattgctgatcatttagggtaattattaagatgcctttaacaaaattcatcgaaaaattaactgacttgaacaaaaaggaaaattaactcttttacattaacaaatctctcaataagaaggtacacctgtggtagagcagttttcaggattagaaaattcagctgctcagtgtcatcaggaacgtgtgtgtgtgtgtgggtgggtgggggtggggtggttttagtgtgtgtgtgtgtgtgtgtgtgtgtgtgtgtgtgtgtgtgtgtgtgttggagggtgggggtggggttgttttatcttttgccctgctattctgagtgagaggcttagctcttctcagggcggtaagatgtttgctgcagttctagacatcagagtcagaacagcattctgggagtggaagaggattgatttattctatatatctccagcaacaacctttctctaagcacctcaacaacttcctcttctatctcgtcagccactgtcaaggaaagtggccaagtccagttatttatttatttatttatttattttttggagacagagtgttgctctgtcgcccaggctggagtgcagtggtgcaatcttgactcactgttacctccacctcccaggttcaagcgattctcctgcctcagcctcccaagtagctgggattacaggcatctgctgccatgcccggctaatttttgtatttttagtagagacggggttttgccatgttagccaggctggtctcgaactcctgacctcgggcaatctgcctgccttggccacccaaagtgctgggattataggcatgagccaccgcgcacccagccagccaagtccagttagttggtctccctcaccaactcgttccacatggatatgtggtaagggaaaatacctgaacaatatcagggtcctcttcgaaaggagaaaggcagataaattgttggtgactagaatataaagtgtcatctgtaccaggtggttaatgtattttccatagcatctgaaattcgagcaacttgatttaagggagttataactcaagttagctttactgagcacatacatagtaggtgctcaaaaaatatggtagatgattgacagagtaataagatgcagatttcttttgcgttaacaatctctgaagctccttacaattaatagtatcttgtaattgctatcattgacgtccagccatttgtctttttctttacattcacaaaggtaggaaacttccagatcttagtgcttgataaaagaccaggcgttttcatgcaggaatttcttcctagcatttcttgggaaatgtgtgttttttttgaattctgaaaaaaattagtcaagaagtttaataaatggaaaacattattctaattgtcttattagaattaattttaacaacacagaaacattactgatttgaaggaatctacttagggcaagttgaaagtaaaaactggaaatactgatacctggaatcaaaggttaaggtcaacaggagctccttggaaagaagtgattacaaatcagtcctcattataaatatcgccgttttatgtgtgttcatggccctgtctgctgctaagtaaataataatgagtttacacatggtatacttaataaaaattgattggtgttccaagaaagattttgaagcaaaatccttatattggcatttttattaacatgtgattctgcatagtactgtaaaccttgctgagttctaaacgtgacattattcacatactacccagtaatacccacatgcagtctagcatttgaatattcactataggcctcagctcttacaaatactaattaaatgatttgttttaaagatacacatgtaggctggatgcggtggctcacacctataatcccagcactttgggaggccgaagcgggtagatcacaaggtcaggagttcgagaccagcttggccagtatggtgaaacacatctctactaaaaatacaaaaaaattagctgggtgtggtggcacatgcctgtaatcccagctacttgggaggctgagacaggagaattgcttgaacctgggaggtggaggttgcagtaggctgagatcacgcagtggcacgccaacctgggtgacagagtgagacgccgtctcaaaaaaaaaagatacacatgtaatgaatagagtttgaaaaatgtatttaataacttttttaaaagaataatgtagatttggggaattccagagaattccaattcattgtttttgaatcctgaaggttggtatttgtcgggaattcagaaacactactaataagaggtcaaatttgtgccttgaagttcaactcagtgtgttttagtgttttcagcaacagttccatgaagggccacaaggtggaggtggtgcttcacagaactagctagttttggcctctttaaccgtctctgctctttttctttttcccctacttcataaaattgttgtaaactcttcactcttttagaacaggaaagtttgtttttttgtttttttgttttgctgaagatggagacgatttctaaatatttaaggctgaaataatgctaaccaaactcttcttaaaaagagaaaaggaagagtgtaccactgaagccatgtggggccacagttgcctttgttggaagctttttaaactagagcagattttatttctttaagagttaaaatacccatcgatttttccttgagtaacctttggtagtttgtacctaggaaagaatttgctcatttcatctgtttttggtttcatgagcatacagttactgatagcattttattatattttactgtttatagaacctgtagttgatgtcctctttcattcctgatattggaaattttgtttgctctctttttgttagtgaatctacctagaggcttttttaactttatcgatcttttcaaagaaacaccattttgtttcattgtggtttttttgtttttctgctgattttattatgttttttattatttccttccttttacttgctttgagtttaatttcctttttctagtttcttaaagtagtagtttagattattgatttgagatattttctcttctaatataaataatgtcatacctttccttttaagcactgctatagtcacatcccacaaattttgatatgttttatttgtatttaattcagaatgttttttataattctacaaaattttcaacttttttccttttatctattgtctctcctttgttatttttgctatctctatggcattctatttgacacaatttagaccagtccctttatccccttcattcttaattcttccttccttcctctgtccctcagggtcttgtgttctgttacctaggctggagtacagtggtacaatcatagctcactgcaggcttgaactcctgggctcaagtgggcctccctcctcagcctccctagtagctaggacaattacaggtgcacaccaatgtgtgcctggctaatttttaaattttttttttttttttttttttttatagagatggggctttgctaagttgcccaggctagtcccaaactcctgggctcaagcgatcctcctgcctcagcctttcaagtagctgggactacaggcatgtgccaccaggcctggctgatgagcattttatctagctttaaagcccttaaataattccagacatctgtgtcatctcatcattgatgtttgttggttgtctttcctggtttttcatatgctgaataatttggggttgtatcctgaacattttcattaggtttttttttttttttttaataagactctgggtcttgttttaatctgccagttttgctgtcattcaaattcagtcttcatcagtatgcctgtttctatttacttttcaaagtaaataactatgaatatccttaaataactacactacccatctctccaggttttattgctactttcagtggaaaagatagagtggaatgtgcttgctccattgtacctagaaccttccttccagagaggattttacttttgctttattcaaataacactagctcctgaacaatttttatgttaatttcttaaaatttgaacctcaaccttaaatcccaaaccgaaattaggcacataaatcttactttgaattttgaggtaagacttttttccccctcttcagagcccagtagaaacagacaaaattccttgctgttttcctatgccagtaggcagacatttttctagtctgttctttcaataggaattttagcattggccaggcatggtggctcatgcctgtaatgccaccactttaggaggccaaagcgggtgaatcacttcagctcaggagttgaagaccagtctgggcaacatggcgaaatcccatgtctactaacaaattacaaaaattagccaggtgtgatggcacaggcctgttgggaggctgaggtgggaggatcatctgagccctgggaggtcaaggctgtggtaagccatgattgcaccactgtactccagcctgggtaacagagtgagaccctgtctcaagaaaacaaacaaaaattttaacattatgcagggggtggagttttagtctcacttttaattccctttctgtggggggttcatttttctatcccaaggcagtgtttaaaaatcctcacaggaagctctccagggcaactgtggtccctgacccactaatgaaagttccttatctttctttgtttctgacactgtggatttcctttctgccttttaatgtgcttgttcattaaaataatgttttacatttatattttatcttgctatttgtaaggatttgtagcagaaagattttcaggttttcttgattgctatcttgctgaaacaaatactttttatagaattattcttttaaaaatcaactgatgtggccaggcacagtgacctgtaatcccagcactgtaagaggctaaagtgggaggatctcttctcaagcccaggatttcaagaccagcctgggcaacatattatagcaagaccctgtccctccaaaaaatttttaaattagcagggcatggtgtcatgtacctgtggacctatctacttgggaggctaaggcggaagtatcccttgagcttaggagtttgaggctgcagtgagctatgatcaggccactgcagcccagcctagggtgacagagcgagaccctgtctctcaaaaaataaaaataaatacaaatcaaccgacatacacaaaaaaattagatgcccaattagaatacaaactattattagaattgccagaatttctaatattcatacaccatgtggatttgtataacacatttatatttatgttttaggctagggaatataattatgtctataatgaatcaggattaaaagagttcctgttatctcttgtgactgaaaaaaaaacagtgtttgatttggaaaaaagtattatcagtataataaaattaatgctggtttcaaataataactttcaggtactatggaaagggcaattttaaactttgagaacagaaataaaaccactctaaatagctaaagagaatcgaggtttgtgcggttacttttgtctttgttttgttagtgtttagtttgttgaattcttggcattccatgtaaaattcaataggaatctcagtttagcagatacggagatttctgtttccttttacttacagtggtttcttttcagaaacatgttctccttccttcctgccaggaatgagtagcgacagtgacattgaatgtgacactgagaatgaggagcaggaagagcataccagtgtgggcgggtttcacgactccttcatggtcatgacacagcccccggatgaaggtgcagcacatgatgtcattttacacagattactaaaaccaccgctgccgcttctctttcttcttcttctgctgctgctacttctactttttccgctctactgcttttgcttcttctgcttctacttctttttttttttttttaattattgtcatttgtgagacaacattctttggttaactattatggtgagtactaaaaaaacacagccagtaatatttggcttggttgcacctaatgagtgctacttactgggcagagacggggtccacaacctgcttttgtatagtctgtgacctgagaatggtttttactttgtaaagagtctttaaaaaccaagcaaaagaaacaaagaagaattttctttgtgggttttttttttctttttttttttttttgccccgagatggagtcttgttctgtcacccaggatggagtgctgtggcgtgatcttggctcactgcaacctccgcctcctgggttcaagcgattctcctgcctcagcctcctgagtagctgggattacaggtatacaccaccacgcctggctaatgtttttgtatttttagtagagatggagtttcaccatgttggccaggctggtctcgaactcttgacctcgtgatccacctgccttggcctcccaaagtcctggtattacaggcgtgagccaccgtgcccagcccaaagaagaattttcaagagagtccatttgtggcctacaatgcctaaatatttacccttcataggaaatgattgccaatccctggggagatttcagatgtttgggttttttttccttttctagcctactgtaagataactttaaccttttaaaataaaaataacttagaaatacatgcagattatgaaataatttttattccctcctacccccaaaaaagttattaggaaaaaaacaagagaaacctactattaatattttcccttgttttcttctgtcttatttgcattgcatttcttcttcttttttttttagacagagtcttgctctgtctcccaagctgaagtgcagtggcacaatctcagctcactgcaacttctgcctcctgggttcaagcaattctcccacctcagcctcctgaatagctgggactacaggcatgtgccaccacatccagctagtttttgtaatttttagtagagacagggtttcaccatgttggccagcctggtctcgaactcctgacctcaggtgatctgcccaccttggcctcccaaagtactggggttacaggtgtgagctaccatgccagccctacctaaatattttataaatagttttttattctgcttttttcacttaaatatatttgtatgttatttaaaatttcataaacttcattttttatggcaacctaatattctatcatgtggatataccataagtattctgctgtcaagcagttttctggaatacagattaatcagtaaaaggatatactgtcaagttagagaaatacacatttaaagttacccagaggccttaataatcaatctgaacattcctctgcaagatatcctggagtgacaacattgttaagcctatgccattttctgctttatattttgcatattttcctggtgctctggtatctttttcaatacagaaccaagcatttgatgatttgtaagtattaaggcatctttagtaacttcctctgcttctaaaataagcatccaaattttgtgcaagttatttggagtgctggtgggtttttgtagtgatacttgaatgcgtagtttaattggctttgaaatatgttttatgtttataaggtcttgaatcttgagaatatacaccattgcttcttgttccctttttaagctgtccccagattgtccccaggaggacaaaatgagacaattcctgtcatcttttaattgtgggtgtctattacacattttgtttttttctcctttatttttcctttgtgtttgcttatacctgactcagatcacaaattctctgaagacagagatttgttcacaacccagcacatctttatcacttaaataacacatttggtaagattatttaaagacttgggaaggaaaatactgcccttgttatatgcttttaaattttaaattttttatttattttttaatcatagatacacattccagttttcctgatggtgaacaaataggccctgaagatctcagcttcaatacagatgaaaatagtggaaggtaattgccaaatcaagagaactgacttgcaagctaccttgaccctgaattttgctgtagttggtgctcaaatttgtcatcagtcagataatcagatttggtcttatttcttcattatctcgacctgaaatagtaatttggaaactgttggaaggtggcacagtttagtctaagacagcagtagtacatgggaaaaacagtatgggaagagttctttgtaatgtaaggaaataacaatgtagttctctattaatttagcaaatttgtacattcacaaaaggcagtttgtctactacagcagaaggctggttaactgccagaaaatgtacctccaggccctgcatgccgtcagtaacccgcccggcattggtgctctactgtctttggctagagcttagttgtgtttaaataatcatctttatatttggggttttaattacagttccattagtgcctgtagattagtgaacagaaaattgctttggaagagattctgccctgtagacactatgtgaataactgaagtaacactagactgaatctcctttttggagtatgtatcttctctcacttgttcaagtacaggcacactgttcaaccgcatggtatctttctgttgtgtgacttctacaaatgtaattttaaatgaaattaagttaacatggattcattacgttcctggccctgtagacacgtgtaagattatttaaaattctttcatttttttctgcctcttactatacgactgtagtgcaacaaatattttaaagcccccttttcttctttattttcattagttgtacattgatttcagtgtcaacacatttaaagattcattcatgttgcacagtggcttacatgaacgtgaaactgtgatataaggttttctttcatactcataattagcccaaaacagttgccaaactttgccattgtgctcctgcatttgtgtttgagctgctatatatttgtggaaattacactgaaagttgactaagagactattgaaaaagcatgaataattaaatatacatgtgagagacatctcatctgctgtattttacttagtgaatattgttcactcttccgtgtctgatgtcttgctgaatgctgtgactcatagtttacttttgttcaaaatagtttgcactttttgttaataaaatcaacttgagaaaa

SEQ ID NO: 13 - DNA, 20 bp, Artificial Sequence
  Artificially synthesized oligonucleotide
aacagagcgtggagagcatt

SEQ ID NO: 14 - DNA, 20 bp, Artificial Sequence
  Artificially synthesized oligonucleotide
cttctgcccaacgacaattt

SEQ ID NO: 15 - DNA, 20 bp, Artificial Sequence
  Artificially synthesized oligonucleotide
tgcaccaccaactgcttagc

SEQ ID NO: 16 - DNA, 21 bp, Artificial Sequence
  Artificially synthesized oligonucleotide
ggcatggactgtggtcatgag

SEQ ID NO: 17 - DNA, 20 bp, Artificial Sequence
  Artificially synthesized oligonucleotide
atgcactcaccttgtcatgg

SEQ ID NO: 18 - DNA, 20 bp, Artificial Sequence
  Artificially synthesized oligonucleotide
acgtccacaaatgagtgcac

SEQ ID NO: 19 - DNA, 22 bp, Artificial Sequence
  Artificially synthesized oligonucleotide
cgagcttagcaaagaagttggg

SEQ ID NO: 20 - DNA, 20 bp, Artificial Sequence
  Artificially synthesized oligonucleotide
tgctcaagaagttccagtgc

SEQ ID NO: 21 - DNA, 20 bp, Artificial Sequence
  Artificially synthesized oligonucleotide
tttcaagccgttggtactgc

SEQ ID NO: 22 - DNA, 20 bp, Artificial Sequence
  Artificially synthesized oligonucleotide
acaatgctgcaacctccttc

SEQ ID NO: 23 - DNA, 20 bp, Artificial Sequence
  Artificially synthesized oligonucleotide
tgcactaatcgtgagcatgg

SEQ ID NO: 24 - DNA, 20 bp, Artificial Sequence
  Artificially synthesized oligonucleotide
ttttgctgaatcggctgctg

SEQ ID NO: 25 - DNA, 20 bp, Artificial Sequence
  Artificially synthesized oligonucleotide
tggacaaaaggcttcaaggc

SEQ ID NO: 26 - DNA, 20 bp, Artificial Sequence
  Artificially synthesized oligonucleotide
ttcaccacagcaaccacatc

SEQ ID NO: 27 - DNA, 19 bp, Artificial Sequence
  Artificially synthesized oligonucleotide
ttccagaattgcagcaatg

SEQ ID NO: 28 - DNA, 20 bp, Artificial Sequence
  Artificially synthesized oligonucleotide
aagctgaagtggttgaagcg

SEQ ID NO: 29 - DNA, 22 bp, Artificial Sequence
  Artificially synthesized oligonucleotide
agatgtcaaccccaagttcctg

SEQ ID NO: 30 - DNA, 20 bp, Artificial Sequence
  Artificially synthesized oligonucleotide
atcgttgcagttgaccatgc

SEQ ID NO: 31 - DNA, 20 bp, Artificial Sequence
  Artificially synthesized oligonucleotide
ggcaaaaacgaaagcgcaag

SEQ ID NO: 32 - DNA, 21 bp, Artificial Sequence
  Artificially synthesized oligonucleotide
caaatgcttctccgctctgag

SEQ ID NO: 33 - DNA, 20 bp, Artificial Sequence
  Artificially synthesized oligonucleotide
tgaacgtggtcaacctgttg

SEQ ID NO: 34 - DNA, 20 bp, Artificial Sequence
  Artificially synthesized oligonucleotide
aggaaggtgtgtttgttgcg

SEQ ID NO: 35 - DNA, 20 bp, Artificial Sequence
  Artificially synthesized oligonucleotide
aaaaagcagtgtcgcccttc

SEQ ID NO: 36 - DNA, 20 bp, Artificial Sequence
  Artificially synthesized oligonucleotide
acttgctctgcatgctgtag

SEQ ID NO: 37 - DNA, 20 bp, Artificial Sequence
  Artificially synthesized oligonucleotide
tgctgaagaaagggcagaag

SEQ ID NO: 38 - DNA, 20 bp, Artificial Sequence
  Artificially synthesized oligonucleotide
acaaggccagcagatttcag

SEQ ID NO: 39 - DNA, 20 bp, Artificial Sequence
  Artificially synthesized oligonucleotide
attggtggagttgggcaatg

SEQ ID NO: 40 - DNA, 20 bp, Artificial Sequence
  Artificially synthesized oligonucleotide
ttacgacatggccaacagtg

SEQ ID NO: 41 - DNA, 21 bp, Artificial Sequence
  Artificially synthesized oligonucleotide
aagacacggcttacctggatg

SEQ ID NO: 42 - DNA, 22 bp, Artificial Sequence
  Artificially synthesized oligonucleotide
aagtgttcatcctcagggtcac

SEQ ID NO: 43 - DNA, 20 bp, Artificial Sequence
  Artificially synthesized oligonucleotide
acatgatggttctgcgtgtg

SEQ ID NO: 44 - DNA, 20 bp, Artificial Sequence
  Artificially synthesized oligonucleotide
agcgcatcaacaagaactgc

SEQ ID NO: 45 - DNA, 22 bp, Artificial Sequence
  Artificially synthesized oligonucleotide
agaactaaaggagctgctgacc

SEQ ID NO: 46 - DNA, 20 bp, Artificial Sequence
  Artificially synthesized oligonucleotide
tgttgctgtccaagttgctc

SEQ ID NO: 47 - DNA, 20 bp, Artificial Sequence
  Artificially synthesized oligonucleotide
acagcaagctgaatgtgtgc

SEQ ID NO: 48 - DNA, 20 bp, Artificial Sequence
  Artificially synthesized oligonucleotide
atcacattccagcaggcttc

SEQ ID NO: 49 - DNA, 19 bp, Artificial Sequence
  Artificially synthesized oligonucleotide
tgacattgctgcccagaac

SEQ ID NO: 50 - DNA, 20 bp, Artificial Sequence
  Artificially synthesized oligonucleotide
acccaatcaacagcaatggc

SEQ ID NO: 51 - DNA, 21 bp, Artificial Sequence
  Artificially synthesized oligonucleotide
actcacccccaattacaaccc

SEQ ID NO: 52 - DNA, 20 bp, Artificial Sequence
  Artificially synthesized oligonucleotide
tggccaaagcgttcaacttg

SEQ ID NO: 53 - DNA, 20 bp, Artificial Sequence
  Artificially synthesized oligonucleotide
tggctgccaatgaccaaaac

SEQ ID NO: 54 - DNA, 21 bp, Artificial Sequence
  Artificially synthesized oligonucleotide
aaggtcaccctggaacctttc

SEQ ID NO: 55 - DNA, 22 bp, Artificial Sequence
  Artificially synthesized oligonucleotide
cgggagctgcttgggtcaagtg

SEQ ID NO: 56 - DNA, 22 bp, Artificial Sequence
  Artificially synthesized oligonucleotide
cagcctaacacccaccttggcg

SEQ ID NO: 57 - DNA, 22 bp, Artificial Sequence
  Artificially synthesized oligonucleotide
caggcacagtggctcacgtctg

SEQ ID NO: 58 - DNA, 22 bp, Artificial Sequence
  Artificially synthesized oligonucleotide
gttggccaggctggtctcgaac

SEQ ID NO: 59 - DNA, 20 bp, Artificial Sequence
  Artificially synthesized oligonucleotide
tggtttcttggcagctactg

SEQ ID NO: 60 - DNA, 20 bp, Artificial Sequence
  Artificially synthesized oligonucleotide
attctagctgctggcctgtg

SEQ ID NO: 61 - DNA, 20 bp, Artificial Sequence
  Artificially synthesized oligonucleotide
atggtgggatgacaaactgc

SEQ ID NO: 62 - DNA, 22 bp, Artificial Sequence
  Artificially synthesized oligonucleotide
atgtatgggccagaaaccagag

SEQ ID NO: 63 - DNA, 20 bp, Artificial Sequence
  Artificially synthesized oligonucleotide
cccatgttcgtcatgggtgt

SEQ ID NO: 64 - DNA, 19 bp, Artificial Sequence
  Artificially synthesized oligonucleotide
ggagcgtgtccatagggtg

SEQ ID NO: 65 - DNA, 25 bp, Artificial Sequence
  Artificially synthesized oligonucleotide
tgttggaatgttaactctatcccat

SEQ ID NO: 66 - DNA, 22 bp, Artificial Sequence
  Artificially synthesized oligonucleotide
atatctcatcaagaccgctgca

SEQ ID NO: 67 - DNA, 21 bp, Artificial Sequence
  Artificially synthesized oligonucleotide
acttaactacgggtgtggctc

SEQ ID NO: 68 - DNA, 20 bp, Artificial Sequence
  Artificially synthesized oligonucleotide
ctggcgtactggtggaaagc

SEQ ID NO: 69 - DNA, 21 bp, Artificial Sequence
  Artificially synthesized oligonucleotide
agggactctagtgcccgacac

SEQ ID NO: 70 - DNA, 20 bp, Artificial Sequence
  Artificially synthesized oligonucleotide
cccacctccaccctacctgc

SEQ ID NO: 71 - DNA, 22 bp, Artificial Sequence
  Artificially synthesized oligonucleotide
taactagctgtgtggccttgag

SEQ ID NO: 72 - DNA, 21 bp, Artificial Sequence
  Artificially synthesized oligonucleotide
atctgagcctcccaacagttc

SEQ ID NO: 73 - DNA, 20 bp, Artificial Sequence
  Artificially synthesized oligonucleotide
tcccatttcgtcctccttgc

SEQ ID NO: 74 - DNA, 22 bp, Artificial Sequence
  Artificially synthesized oligonucleotide
tccttcctgcttcccttctttc

SEQ ID NO: 75 - DNA, 36 bp, Artificial Sequence
  Artificially synthesized oligonucleotide
taaagcggccgccatggatgaacagagcgtggagag

SEQ ID NO: 76 - DNA, 38 bp, Artificial Sequence
  Artificially synthesized oligonucleotide
ccgcggatccttatcttccactattttcatctgtattg

SEQ ID NO: 77 - DNA, 21 bp, Artificial Sequence
  Artificially synthesized oligonucleotide
ttcactggtaaagtctggtgg

SEQ ID NO: 78 - DNA, 21 bp, Artificial Sequence
  Artificially synthesized oligonucleotide
atgacctcctttagcattagc

SEQ ID NO: 79 - DNA, 21 bp, Artificial Sequence
  Artificially synthesized oligonucleotide
aaggagttccagttgtagttc

SEQ ID NO: 80 - DNA, 21 bp, Artificial Sequence
  Artificially synthesized oligonucleotide
ttccagttgtagttcggcagg

SEQ ID NO: 81 - DNA, 21 bp, Artificial Sequence
  Artificially synthesized oligonucleotide
tttagctccgagaactgcttc

SEQ ID NO: 82 - DNA, 21 bp, Artificial Sequence
  Artificially synthesized oligonucleotide
aagcatgtacacgatgtatgg

SEQ ID NO: 83 - DNA, 21 bp, Artificial Sequence
  Artificially synthesized oligonucleotide
aaagggccgaaagtgcagtta

SEQ ID NO: 84 - DNA, 21 bp, Artificial Sequence
  Artificially synthesized oligonucleotide
caaagtcacagtcgaagaggt

SEQ ID NO: 85 - DNA, 21 bp, Artificial Sequence
  Artificially synthesized oligonucleotide
actgtgttcatcatactgtcg

SEQ ID NO: 86 - DNA, 21 bp, Artificial Sequence
  Artificially synthesized oligonucleotide
ttctcgtcctgatactttggg

SEQ ID NO: 87 - DNA, 21 bp, Artificial Sequence
  Artificially synthesized oligonucleotide
atctcctgaccttatgatccg

SEQ ID NO: 88 - DNA, 21 bp, Artificial Sequence
  Artificially synthesized oligonucleotide
aagtggcatgtgattctttgc

SEQ ID NO: 89 - DNA, 21 bp, Artificial Sequence
  Artificially synthesized oligonucleotide
gcagctggtgagaaggcaata

SEQ ID NO: 90 - DNA, 20 bp, Artificial Sequence
  Artificially synthesized oligonucleotide
tgaagctaaggcagctgttt

SEQ ID NO: 91 - DNA, 21 bp, Artificial Sequence
  Artificially synthesized oligonucleotide
ttctccaggtataaatgtagg

SEQ ID NO: 92 - DNA, 21 bp, Artificial Sequence
  Artificially synthesized oligonucleotide
ttccgatccaatctgttctgg

SEQ ID NO: 93 - protein, 449 aa, Artificial Sequence
  Artificially synthesized LK26 Heavy Chain Amino Acid Sequence
QVQLQESGGDLVKPGGSLKLSCAASGFTFSGYGLSWVRQTPDKRLEWVAMISSGGSYTYYADSVKGRFAISRDNAKNSLFLQMSSLKSDDTAIYICARHGDDPAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGK

SEQ ID NO: 94 - protein, 217 aa, Artificial Sequence
  Artificially synthesized LK26 Light Chain Amino Acid Sequence
DIELTQSPALNAASPGEKVTITCSVSSSISSNNLHWYQQKSETSPKPWIYGTSNLASGVPLRFRGFGSGTSYSLTISSNEAEDAATYYCQQWSSYPYMYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 581 aa, Artificial Sequence
  Artificially synthesized Farletuzumab Heavy Chain Amino Acid Sequence
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSGGSGGSGGSGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 96 - protein, 337 aa, Artificial Sequence
  Artificially synthesized Farletuzumab Light Chain Amino Acid Sequence
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGSGGSGGSGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - DNA, 51 bp, Artificial Sequence
  Artifically synthesized non-cysteine linker coding sequence
  CDS (1)..(51)
gagggcgggggaagccgctctgatgaaggagatggcggaggaagccctgga

SEQ ID NO: 98 - protein, 17 aa, Artificial Sequence
  Synthetic Construct
EGGGSRSDEGDGGGSPG

SEQ ID NO: 99 - DNA, 51 bp, Artificial Sequence
  Artifically synthesized cysteine linker coding sequence
  CDS (1)..(51)
gagggcgggggaagccgctctaggaaacgcaggggcggatgcagccctgga

SEQ ID NO: 100 - protein, 17 aa, Artificial Sequence
  Synthetic Construct
EGGGSRSRKRRGGCSPG